(19) United States
(12) Patent Application Publication
(10) Pub. No.: US 2019/0038756 A1
(43) Pub. Date: Feb. 7, 2019

(54) COMPOSITIONS INCLUDING BENZENESULFONAMIDE-CONTAINING NON-STEROIDAL ANTI-INFLAMMATORY DRUGS, SILK FIBROIN, AND A GELLING AGENT AND USES THEREOF

(71) Applicant: Cocoon Biotech Inc., Cambridge, MA (US)

(72) Inventors: Ailis Tweed-Kent, Cambridge, MA (US); Michael Santos, Cambridge, MA (US); Andrew Bellinger, Wellesley, MA (US)

(21) Appl. No.: 16/077,078

(22) PCT Filed: Feb. 10, 2017
(86) PCT No.: PCT/US17/17535

§ 371 Date: Aug. 10, 2018

Related U.S. Application Data
(60) Provisional application No. 62/293,719, filed on Feb. 10, 2016.
(60) Provisional application No. 62/329,131, filed on Apr. 28, 2016.

(51) Int. Cl. A61K 47/42; A61K 9/06; A61K 47/10; A61K 31/415; A61P 29/00

(52) U.S. Cl. CPC A61K 47/42; A61K 9/06; A61P 29/00; A61K 31/415; A61K 47/10

(57) ABSTRACT

Provided herein are compositions that include a benzenesulfonamide-containing NSAID, silk fibroin, and a gelling agent. Also provided are kits that include any of these compositions, and methods of making any of these compositions. Also provided are methods of treating an inflammatory and/or pain condition in a subject and methods of reducing inflammation, pain, or fever in a subject in need thereof, that include administering a therapeutically effective amount of any of these compositions.

### CROSS-REFERENCE TO RELATED APPLICATIONS

This application claims priority to U.S. Provisional Patent Application Ser. No. 62/293,719, filed Feb. 10, 2016, and U.S. Provisional Patent Application Ser. No. 62/329,131, filed Apr. 28, 2016; the entire contents of which are herein incorporated by reference.

### TECHNICAL FIELD

This invention relates to the field of pharmaceutical formulation and methods of treating inflammatory and/or pain conditions.

### BACKGROUND

Non-steroidal anti-inflammatory drugs (NSAIDs) are a class of small molecule drugs designed to reduce much of the inflammation and pain experienced by subjects having an inflammatory and/or pain condition. Cyclooxygenase-1 and Cyclooxygenase-2 (also called prostaglandinendoperoxidase synthase 1 and 2, respectively) are enzymes that are responsible, in part, for some of the inflammation and pain experienced in inflammatory disorders (eMed Expert website, www.emedexpert.com/compare/nsaids.shtml). Currently, subjects having an inflammatory and/or pain condition are orally administered an NSAID. Some subjects experience side effects from the oral administration of NSAIDs including, for example, gastritis, gastric ulceration, renal insufficiency, indigestion, diarrhea, nausea, headaches, and chest pain (Brune, Curr. Med. Res. Opin. 23(12):2985-2995, 2007). Oral administration of high doses of NSAIDs can also result in increased risk of stroke in the subject and an increased risk of having a negative affect on the cardiovascular system of a subject.

Silk fibroin hydrogels have previously been used as a delivery vehicle for monoclonal antibodies (Guziewicz et al., Biomaterials 32(10):2642-2650, 2011). Silk fibroin hydrogel is also known to be biodegradable and biocompatible in mammals (Guziewicz et al., Biomaterials 32(10):2642-2650, 2011).

### SUMMARY

The present invention is based, at least in part, on the discovery that formulation of a benzenesulfonamide-containing non-steroidal anti-inflammatory drug (NSAID) into a composition (e.g., a gel, e.g., a hydrogel) including silk fibroin results in a surprisingly increased duration of sustained delivery of a therapeutically effective amount of the benzenesulfonamide-containing NSAID in a subject following administration of the composition (e.g., gel) to the subject. In view of this discovery, provided herein are compositions (e.g., gels, e.g., hydrogels) that include a benzenesulfonamide-containing NSAID, silk fibroin, and a gelling agent (e.g., any of the gelling agents described herein or known in the art), where the composition releases a therapeutically effective amount of the benzenesulfonamide-containing NSAID over a period of 10 days to about 200 days when incubated in phosphate buffered saline (PBS), 2% Tween-80 in PBS, or 2% Tween-80 and 0.05% sodium azide in PBS at 37° C. Also provided are compositions that include a gel provided herein, kits that include any of the compositions provided herein, and methods of making any of the compositions (e.g., gels) provided herein. Also provided are methods of treating an inflammatory and/or pain condition in a subject and methods of reducing inflammation, pain, or fever in a subject in need thereof, that include administering a therapeutically effective amount of any of the compositions (e.g., gels) provided herein.

Provided herein are methods of treating pain in a subject that include administering a therapeutically effective amount of a composition (e.g., a gel, e.g., a hydrogel or organogel) including a benzenesulfonamide-containing a non-steroidal anti-inflammatory drug (NSAID), a silk fibroin, and a gelling agent to the subject. In some embodiments of any of the methods described herein, the pain is selected from the group of: nociceptive pain, neuropathic pain, psychogenic pain, breakthrough pain, incident pain, back pain, musculoskeletal pain, post-operative pain, operative pain, visceral pain, joint pain, acute pain, inflammatory pain, or chronic pain.

Also provided herein are methods of treating an inflammatory and/or pain condition in a subject that include administering a therapeutically effective amount of a composition (e.g., a gel, e.g., a hydrogel or organogel) including a benzenesulfonamide-containing non-steroidal anti-inflammatory drug (NSAID), a silk fibroin, and a gelling agent to the subject.

In some embodiments of any of the methods described herein, the inflammatory condition is selected from the group of: a joint disease, an ophthalmic disease, retinal disease, psoriasis, Crohn's disease, irritable bowel syndrome, Sjogren's disease, tissue graft rejection, asthma, systemic lupus erythematosus, glomerulonephritis, dermatomyositis, multiple sclerosis, scleroderma, vasculitis, Goodpasture's syndrome, atherosclerosis, chronic idiopathic thrombocytopenic purpura, Addison's disease, Parkinson's disease, Alzheimer's disease, diabetes, septic shock, myasthenia gravis, inflammatory pelvic disease, inflammatory bowel disease, urethritis, uveitis, sinusitis, pneumonitis, encephalitis, meningitis, myocarditis, nephritis, osteomyelitis, myositis, hepatitis, gastritis, enteritis, dermatitis, appendicitis, pancreatitis, cholocystitis, polycystic kidney disease, and cancer.

In some embodiments of any of the methods described herein, the inflammatory condition is a joint disease. In some embodiments of any of the methods described herein, the joint disease is selected from the group of: osteoarthritis, rheumatoid arthritis, spondyloarthritis, systemic juvenile idiopathic arthritis, psoriatic arthritis, gout, ankylosing spondylitis, and juvenile rheumatoid arthritis. In some embodiments of any of the methods described herein, the subject has previously been diagnosed as having an inflammatory condition.

Also provided herein are methods of reducing inflammation, pain, or fever in a subject in need thereof that include administering a therapeutically effective amount of a composition (e.g., a gel, e.g., a hydrogel or organogel) including a benzenesulfonamide-containing non-steroidal anti-inflammatory drug (NSAID), a silk fibroin, and a gelling agent to the subject.

In some embodiments of any of the methods described herein, the pain is nociceptive pain, neuropathic pain, psychogenic pain, breakthrough pain, incident pain, back pain, musculoskeletal pain, post-operative pain, operative pain, visceral pain, joint pain, acute pain, inflammatory pain, or chronic pain. In some embodiments of any of the methods described herein, the inflammation that is reduced is in a joint in the subject. In some embodiments of any of the methods described herein, the method includes intraarticularly administering the composition (e.g., gel, e.g., hydrogel or organogel) to the joint in the subject. In some embodiments of any of the methods described herein, the subject has a joint disease.

In some embodiments of any of the methods described herein, the composition (e.g., gel, e.g., hydrogel or organogel) includes about 0.01% (w/v) to about 50% (w/v) of the gelling agent, or about 0.01% (v/v) to about 50% (v/v) of the gelling agent. In some embodiments of any of the methods described herein, the gelling agent is glycerol or a polyethylene glycol (PEG). In some embodiments of any of the methods described herein, the PEG is PEG300, PEG400, or PEG600, or a PEG having a molecular weight of about 2 kDa, about 4 kDa, about 6 kDa, about 8 kDa, about 10 kDa, about 12 kDa, about 14 kDa, about 16 kDa, about 18 kDa, or about 20 kDa.

In some embodiments of any of the methods described herein, the gelling agent is selected from the group of: sorbitol, triethylamine, 2-pyrrolidone, alpha-cyclodextrin, benzyl alcohol, beta-cyclodextrin, dimethyl sulfoxide, dimethylacetamide (DMA), dimethylformamide, ethanol, gamma-cyclodextrin, glycerol, glycerol formal, hydroxypropyl beta-cyclodextrin, kolliphor 124, kolliphor 181, kolliphor 188, kolliphor 407, kolliphor EL (cremaphor EL), cremaphor RH 40, cremophor RH 60, d-alpha-tocopherol, PEG 1000 succinate, polysorbate 20, polysorbate 80, solutol HS 15, sorbitan monooleate, poloxamer-407, poloxamer-188, Labrafil M-1944CS, Lrabfil M-2125CS, Labrasol, Gellucire 44/14, Softigen 767, mono- and di-fatty acid esters of PEG 300, PEG 400, or PEG 1750, kolliphor RH60, N-methyl-2-pyrrolidone, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil, beeswax, d-alpha-tocopherol, oleic acid, medium-chain mono- and di-glycerides, alpha-cyclodextrin, beta-cyclodextrin, hydroxypropyl-beta-cyclodextrin, sulfo-butylether-beta-cyclodextrin, hydrogenated soy phosphatidylcholine, distearoylphosphatidylglycerol, L-alpha-dimyristoylphosphatidylcholine, L-alpha-dimyristoylphosphatidylglycerol, PEG 300, PEG 300 caprylic/capric glycerides (Softigen 767), PEG 300 linoleic glycerides (Labrafil M-2125CS), PEG 300 oleic glycerides (Labrafil M-1944CS), PEG 400, PEG 400 caprylic/capric glycerides (Labrasol), polyoxyl 40 stearate (PEG 1750 monosterate), polyoxyl 8 stearate (PEG 400 monosterate), polysorbate 20, polysorbate 80, polyvinyl pyrrolidone, propylene carbonate, propylene glycol, solutol HS15, sorbitan monooleate (Span 20), sulfobutylether-beta-cyclodextrin, transcutol, triacetin, 1-dodecylazacyclo-heptan-2-one, caprolactam, castor oil, cottonseed oil, ethyl acetate, medium chain triglycerides, methyl acetate, oleic acid, safflower oil, sesame oil, soybean oil, tetrahydrofuran, and glycerin.

In some embodiments of any of the methods described herein, the composition (e.g., a gel, e.g., hydrogel or organogel) includes about 20% (w/v) to about 50% (w/v) of the gelling agent, or about 20% (v/v) to about 50% (v/v) of the gelling agent. In some embodiments of any of the methods described herein, the composition (e.g., a gel, e.g., hydrogel or organogel) includes about 30% (w/v) to about 50% (w/v) of the gelling agent, or about 30% (v/v) to about 50% (v/v) of the gelling agent. In some embodiments of any of the methods described herein, the composition (e.g., gel, e.g., hydrogel or organogel) includes about 35% (w/v) to about 50% (w/v) of the gelling agent, or about 35% (v/v) to about 50% (v/v) of the gelling agent. In some embodiments of any of the methods described herein, the composition (e.g., gel, e.g., hydrogel or organogel) includes about 0.5% (w/v) to about 30% (w/v) silk fibroin. In some embodiments of any of the methods described herein, the composition (e.g., gel, e.g., hydrogel or organogel) includes about 2% (w/v) to about 30% (w/v) silk fibroin. In some embodiments of any of the methods described herein, the composition (e.g., gel, e.g., hydrogel or organogel) includes about 5% (w/v) to about 30% (w/v) silk fibroin. In some embodiments of any of the methods described herein, the composition (e.g., a gel, e.g., a hydrogel or organogel) includes about 10% (w/v) to about 30% (w/v) silk fibroin. In some embodiments of any of the methods described herein, the silk fibroin has a molecular weight range of about 10 kDa to about 500 kDa.

In some embodiments of any of the methods described herein, the composition (e.g., a gel, e.g., a hydrogel or organogel) includes about 0.5 mg/mL to about 200 mg/mL of the benzenesulfonamide-containing NSAID. In some embodiments of any of the methods described herein, the composition (e.g., a gel, e.g., a hydrogel or organogel) includes about 1 mg/mL to about 100 mg/mL of the benzenesulfonamide-containing NSAID. In some embodiments of any of the methods described herein, the composition (e.g., a gel, e.g., a hydrogel or organogel) includes about 1 mg/mL to about 50 mg/mL of the benzenesulfonamide-containing NSAID. In some embodiments of any of the methods described herein, the composition (e.g., a gel, e.g., a hydrogel or organogel) includes about 1 mg/mL to about 20 mg/mL of the benzenesulfonamide-containing NSAID. In some embodiments of any of the methods described herein, the composition (e.g., a gel, e.g., a hydrogel or organogel) includes about 1 mg/mL to about 10 mg/mL of the benzenesulfonamide-containing NSAID.

In some embodiments of any of the methods described herein, the composition (e.g., a gel, e.g., a hydrogel or organogel) includes a plurality of particles including the benzenesulfonamide-containing NSAID. In some embodiments of any of the methods described herein, the plurality of particles have a d90 of less than 200 μm. In some embodiments of any of the methods described herein, the plurality of particles have a d50 of less than 25 μm. In some embodiments of any of the methods described herein, the plurality of particles have a d10 of less than 10 μm. In some embodiments of any of the methods described herein, the composition is a gel. In some embodiments of any of the methods described herein, the gel is a hydrogel.

In some embodiments of any of the methods described herein, the composition (e.g., a gel, e.g., a hydrogel or organogel) releases at least 15% of the total amount of the benzenesulfonamide-containing NSAID in the composition over a period of about 10 days to about 200 days when incubated under simulated physiological conditions. In some embodiments of any of the methods described herein, the composition (e.g., a gel, e.g., a hydrogel or organogel) releases at least 15% of the total amount of the benzenesulfonamide-containing NSAID in the composition over a period of about 20 days to about 200 days when incubated under simulated physiological conditions. In some embodiments of any of the methods described herein, the composition (e.g., a gel, e.g., a hydrogel or organogel) releases at least 15% of the total amount of the benzenesulfonamide-containing NSAID in the composition over a period of about 30 days to about 200 days when incubated under simulated physiological conditions. In some embodiments of any of the methods described herein, the composition (e.g., a gel, e.g., a hydrogel or organogel) releases at least 15% of the total amount of the benzenesulfonamide-containing NSAID in the composition over a period of about 50 days to about 300 days when incubated under simulated physiological conditions. In some embodiments of any of the methods described herein, the composition (e.g., a gel, e.g., a hydrogel or organogel) releases at least 15% of the total amount of the benzenesulfonamide-containing NSAID in the composition over a period of about 100 days to about 300 days when incubated under simulated physiological conditions.

In some embodiments of any of the methods described herein, the benzenesulfonamide-containing NSAID includes a pyrazole moiety. In some embodiments of any of the methods described herein, the pyrazole moiety is a haloalkyl-substituted pyrazole moiety. In some embodiments of any of the methods described herein, the haloalkyl-substituted pyrazole moiety is a trifluoromethyl-substituted pyrazole moiety. In some embodiments of any of the methods described herein, the benzenesulfonamide-containing NSAID is celecoxib.

Provided herein are compositions (e.g., gels, e.g., hydrogels or organogels) that include a benzenesulfonamide-containing non-steroidal anti-inflammatory drug (NSAID), a silk fibroin, and a gelling agent, for use in the treatment of an inflammatory and/or pain condition in a subject.

Also provided herein are compositions (e.g., gels, e.g., hydrogels or organogels) that include a benzenesulfonamide-containing non-steroidal anti-inflammatory drug (NSAID), a silk fibroin, and a gelling agent, for use in reducing inflammation, pain, or fever in a subject in need thereof.

Also provided herein are compositions (e.g., gels, e.g., hydrogels or organogels) that include a benzenesulfonamide-containing non-steroidal anti-inflammatory drug (NSAID), silk fibroin, and a gelling agent.

In some embodiments of any of the compositions (e.g., gels, e.g., hydrogels or organogels) described herein, the gelling agent is glycerol or a polyethylene glycol (PEG) or glycerol. In some embodiments of any of the compositions described herein, the PEG is PEG300, PEG400, or PEG600, or a PEG having a molecular weight of about 2 kDa, about 4 kDa, about 6 kDa, about 8 kDa, about 10 kDa, about 12 kDa, about 14 kDa, about 16 kDa, about 18 kDa, or about 20 kDa.

In some embodiments of any of the compositions (e.g., gels, e.g., hydrogels or organogels) described herein, the gelling agent is selected from the group of: sorbitol, triethylamine, 2-pyrrolidone, alpha-cyclodextrin, benzyl alcohol, beta-cyclodextrin, dimethyl sulfoxide, dimethylacetamide (DMA), dimethylformamide, ethanol, gamma-cyclodextrin, glycerol, glycerol formal, hydroxypropyl beta-cyclodextrin, kolliphor 124, kolliphor 181, kolliphor 188, kolliphor 407, kolliphor EL (cremaphor EL), cremaphor RH 40, cremophor RH 60, d-alpha-tocopherol, PEG 1000 succinate, polysorbate 20, polysorbate 80, solutol HS 15, sorbitan monooleate, poloxamer-407, poloxamer-188, Labrafil M-1944CS, Lrabfil M-2125CS, Labrasol, Gellucire 44/14, Softigen 767, mono- and di-fatty acid esters of PEG 300, PEG 400, or PEG 1750, kolliphor RH60, N-methyl-2-pyrrolidone, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil, beeswax, d-alpha-tocopherol, oleic acid, medium-chain mono- and di-glycerides, alpha-cyclodextrin, beta-cyclodextrin, hydroxypropyl-beta-cyclodextrin, sulfo-butylether-beta-cyclodextrin, hydrogenated soy phosphatidylcholine, distearoylphosphatidylglycerol, L-alpha-dimyristoylphosphatidylcholine, L-alpha-dimyristoylphosphatidylglycerol, PEG 300, PEG 300 caprylic/capric glycerides (Softigen 767), PEG 300 linoleic glycerides (Labrafil M-2125CS), PEG 300 oleic glycerides (Labrafil M-1944CS), PEG 400, PEG 400 caprylic/capric glycerides (Labrasol), polyoxyl 40 stearate (PEG 1750 monosterate), polyoxyl 8 stearate (PEG 400 monosterate), polysorbate 20, polysorbate 80, polyvinyl pyrrolidone, propylene carbonate, propylene glycol, solutol HS15, sorbitan monooleate (Span 20), sulfobutylether-beta-cyclodextrin, transcutol, triacetin, 1-dodecylazacyclo-heptan-2-one, caprolactam, castor oil, cottonseed oil, ethyl acetate, medium chain triglycerides, methyl acetate, oleic acid, safflower oil, sesame oil, soybean oil, tetrahydrofuran, and glycerin.

In some embodiments of any of the compositions (e.g., gels, e.g., hydrogels or organogels) described herein, the composition (e.g., gel, e.g., a hydrogel or organogel) includes about 0.01% (w/v) to about 50% (w/v) of the gelling agent, or about 0.01% (v/v) to about 50% (v/v) of the gelling agent. In some embodiments of any of the compositions (e.g., gels, e.g., hydrogels or organogels) described herein, the composition (e.g., gel, e.g., a hydrogel or organogel) includes about 20% (w/v) to about 50% (w/v) of the gelling agent, or about 20% (v/v) to about 50% (v/v) of the gelling agent. In some embodiments of any of the compositions (e.g., gels, e.g., hydrogels or organogels) described herein, the composition (e.g., gel, e.g., a hydrogel or organogel) includes about 25% (w/v) to about 50% (w/v) of the gelling agent, or about 25% (v/v) to about 50% (v/v) of the gelling agent. In some embodiments of any of the compositions (e.g., gels, e.g., hydrogels or organogels) described herein, the composition (e.g., gel, e.g., a hydrogel or organogel) includes about 30% (w/v) to about 50% (w/v) of the gelling agent, or about 30% (v/v) to about 50% (v/v) of the gelling agent. In some embodiments of any of the compositions (e.g., gels, e.g., hydrogels or organogels) described herein, the composition (e.g., gel, e.g., a hydrogel or organogel) includes about 0.5% (w/v) to about 30% (w/v) silk fibroin. In some embodiments of any of the compositions (e.g., gels, e.g., hydrogels or organogels) described herein, the composition (e.g., gel, e.g., a hydrogel or organogel) includes about 2% (w/v) to about 30% (w/v) silk fibroin. In some embodiments of any of the compositions (e.g., gels, e.g., hydrogels or organogels) described herein, the composition (e.g., gel, e.g., a hydrogel or organogel) includes about 5% (w/v) to about 30% (w/v) silk fibroin. In some embodiments of any of the compositions (e.g., gels, e.g., hydrogels or organogels) described herein, the composition (e.g., gel, e.g., hydrogel or organogel) includes about 10% (w/v) to about 30% (w/v) silk fibroin.

In some embodiments of any of the compositions (e.g., gels, e.g., hydrogels or organogels) described herein, the silk fibroin has a molecular weight range of about 10 kDa to about 500 kDa.

In some embodiments of any of the compositions (e.g., gels, e.g., hydrogels or organogels) described herein, the composition (e.g., gel, e.g., a hydrogel or organogel) includes about 0.5 mg/mL to about 200 mg/mL of the benzenesulfonamide-containing NSAID. In some embodiments of any of the compositions (e.g., gels, e.g., hydrogels or organogels) described herein, the composition (e.g., gel, e.g., a hydrogel or organogel) includes about 1 mg/mL to about 100 mg/mL of the benzenesulfonamide-containing NSAID. In some embodiments of any of the compositions (e.g., gels, e.g., hydrogels or organogels) described herein, the composition (e.g., gel, e.g., a hydrogel or organogel) includes about 1 mg/mL to about 50 mg/mL of the benzenesulfonamide-containing NSAID. In some embodiments of any of the compositions (e.g., gels, e.g., hydrogels or organogels) described herein, the composition (e.g., gel, e.g., a hydrogel or organogel) includes about 1 mg/mL to about 20 mg/mL of the benzenesulfonamide-containing NSAID. In some embodiments of any of the compositions (e.g., gels, e.g., hydrogels or organogels) described herein, the composition (e.g., gel, e.g., a hydrogel or organogel) includes about 1 mg/mL to about 10 mg/mL of the benzenesulfonamide-containing NSAID. In some embodiments of any of the compositions (e.g., gels, e.g., hydrogels or organogels) described herein, the composition (e.g., a gel, e.g., a hydrogel or organogel) includes a plurality of particles including the benzenesulfonamide-containing NSAID.

In some embodiments of any of the compositions (e.g., gels, e.g., hydrogels or organogels) described herein, the plurality of particles have a d90 of less than 200 μm. In some embodiments of any of the compositions (e.g., gels, e.g., hydrogels or organogels) described herein, the plurality of particles have a d50 of less than 25 μm. In some embodiments of any of the compositions (e.g., gels, e.g., hydrogels or organogels) described herein, the plurality of particles have a d10 of less than 10 μm.

In some embodiments of any of the compositions described herein, the composition is a gel. In some embodiments of any of the compositions described herein, the gel is a hydrogel.

In some embodiments of any of the compositions (e.g., gels, e.g., hydrogels or organogels) described herein, the composition (e.g., gel, e.g., a hydrogel or organogel) releases at least 15% of the total amount of the benzenesulfonamide-containing NSAID in the composition over a period of about 10 days to about 200 days when incubated under simulated physiological conditions. In some embodiments of any of the compositions (e.g., gels, e.g., hydrogels or organogels) described herein, the composition (e.g., gel, e.g., a hydrogel or organogel) releases at least 15% of the total amount of the benzenesulfonamide-containing NSAID in the composition over a period of about 20 days to about 200 days when incubated under simulated physiological conditions. In some embodiments of any of the compositions (e.g., gels, e.g., hydrogels or organogels) described herein, the composition (e.g., gel, e.g., a hydrogel or organogel) releases at least 15% of the total amount of the benzenesulfonamide-containing NSAID in the composition over a period of about 30 days to about 200 days when incubated under simulated physiological conditions. In some embodiments of any of the compositions (e.g., gels, e.g., hydrogels or organogels) described herein, the composition (e.g., gel, e.g., a hydrogel or organogel) releases at least 15% of the total amount of the benzenesulfonamide-containing NSAID in the composition over a period of about 50 days to about 300 days when incubated under simulated physiological conditions. In some embodiments of any of the compositions (e.g., gels, e.g., hydrogels or organogels) described herein, the composition (e.g., gel, e.g., a hydrogel or organogel) releases at least 15% of the total amount of the benzenesulfonamide-containing NSAID in the composition over a period of about 100 days to about 300 days when incubated under simulated physiological conditions.

In some embodiments of any of the compositions (e.g., gels, e.g., hydrogels or organogels) described herein, the benzenesulfonamide-containing NSAID includes a pyrazole moiety. In some embodiments of any of the compositions (e.g., gels, e.g., hydrogels or organogels) described herein, the pyrazole moiety is a haloalkyl-substituted pyrazole moiety. In some embodiments of any of the compositions (e.g., gels, e.g., hydrogels or organogels) described herein, the haloalkyl-substituted pyrazole moiety is a trifluoromethyl-substituted pyrazole moiety. In some embodiments of any of the compositions (e.g., gels, e.g., hydrogels or organogels) described herein, the benzenesulfonamide-containing NSAID is celecoxib.

Also provided herein are kits that include any of the compositions (e.g., gels, e.g., hydrogels or organogels) described. In some embodiments of any of the kits described herein, the kit further includes a syringe, wherein the syringe is filled with any of the compositions provided herein (e.g., gel, e.g., a hydrogel or organogel). In some embodiments of any of the kits described herein, the kit further includes a sealed vial, wherein the sealed vial is filled with any of the compositions (e.g., gels, e.g., hydrogels or organogels) described herein. In some embodiments of any of the kits described herein, the kit further includes instructions for performing any of the methods described herein.

Also provided herein are methods of making a composition (e.g., a gel, e.g., a hydrogel or organogel) that include: (a) providing a first solution including about 1% (w/v) to about 60% (w/v) silk fibroin; (b) providing a second solution including about 0.02% (w/v) to about 100% (w/v), or about 0.02% (v/v) to about 100% (v/v), of a gelling agent, and about 1 mg/mL to about 400 mg/mL of a benzenesulfonamide-containing NSAID; (c) mixing a volume of the first solution with a volume of the second solution, where the ratio of the volume of the first solution to the volume of the second solution is about 5:1 to about 1:5 to provide a mixture; and (d) incubating the mixture for a time sufficient to form the composition (e.g., gel, e.g., a hydrogel or organogel).

Also provided herein are methods of making a composition (e.g., a gel, e.g., a hydrogel or organogel) that include: (a) providing a solid composition including silk fibroin; (b) providing a solution including about 0.02% (w/v) to about 100% (w/v), or about 0.02% (v/v) to about 100% (v/v), of a gelling agent, and about 1 mg/mL to about 400 mg/mL of a benzenesulfonamide-containing NSAID; (c) mixing a volume of the solution with an amount of the solid composition; and (d) incubating the mixture of a time sufficient to form the composition (e.g., gel, e.g., a hydrogel or organogel).

In some embodiments of any of the methods described herein, the gelling agent is glycerol or a polyethylene glycol (PEG). In some embodiments of any of the methods described herein, the PEG is PEG300, PEG400, or PEG600, or a PEG having a molecular weight of about 2 kDa, about 4 kDa, about 6 kDa, about 8 kDa, about 10 kDa, about 12 kDa, about 14 kDa, about 16 kDa, about 18 kDa, or about 20 kDa.

In some embodiments of any of the methods described herein, the gelling agent is selected from the group of: sorbitol, triethylamine, 2-pyrrolidone, alpha-cyclodextrin, benzyl alcohol, beta-cyclodextrin, dimethyl sulfoxide, dimethylacetamide (DMA), dimethylformamide, ethanol, gamma-cyclodextrin, glycerol, glycerol formal, hydroxypropyl beta-cyclodextrin, kolliphor 124, kolliphor 181, kolliphor 188, kolliphor 407, kolliphor EL (cremaphor EL), cremaphor RH 40, cremophor RH 60, d-alpha-tocopherol, PEG 1000 succinate, polysorbate 20, polysorbate 80, solutol HS 15, sorbitan monooleate, poloxamer-407, poloxamer-188, Labrafil M-1944CS, Lrabfil M-2125CS, Labrasol, Gellucire 44/14, Softigen 767, mono- and di-fatty acid esters of PEG 300, PEG 400, or PEG 1750, kolliphor RH60, N-methyl-2-pyrrolidone, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil, beeswax, d-alpha-tocopherol, oleic acid, medium-chain mono- and di-glycerides, alpha-cyclodextrin, beta-cyclodextrin, hydroxypropyl-beta-cyclodextrin, sulfo-butylether-beta-cyclodextrin, hydrogenated soy phosphatidylcholine, distearoylphosphatidylglycerol, L-alpha-dimyristoylphosphatidylcholine, L-alpha-dimyristoylphosphatidylglycerol, PEG 300, PEG 300 caprylic/capric glycerides (Softigen 767), PEG 300 linoleic glycerides (Labrafil M-2125CS), PEG 300 oleic glycerides (Labrafil M-1944CS), PEG 400, PEG 400 caprylic/capric glycerides (Labrasol), polyoxyl 40 stearate (PEG 1750 monosterate), polyoxyl 8 stearate (PEG 400 monosterate), polysorbate 20, polysorbate 80, polyvinyl pyrrolidone, propylene carbonate, propylene glycol, solutol HS15, sorbitan monooleate (Span 20), sulfobutylether-beta-cyclodextrin, transcutol, triacetin, 1-dodecylazacyclo-heptan-2-one, caprolactam, castor oil, cottonseed oil, ethyl acetate, medium chain triglycerides, methyl acetate, oleic acid, safflower oil, sesame oil, soybean oil, tetrahydrofuran, and glycerin.

In some embodiments of any of the methods described herein, the second solution includes about 20% (w/v) to about 100% (w/v) of the gelling agent or about 20% (v/v) to about 100% (v/v) of the gelling agent. In some embodiments of any of the methods described herein, the second solution includes about 40% (w/v) to about 100% (w/v) or about 40% (v/v) to about 100% (v/v) of the gelling agent. In some embodiments of any of the methods described herein, the second solution includes about 60% (w/v) to about 100% (w/v) or about 60% (v/v) to about 100% (v/v) of the gelling agent. In some embodiments of any of the methods described herein, the second solution includes about 80% (w/v) to about 100% (w/v) of the gelling agent or about 80% (v/v) to about 100% (v/v) of the gelling agent. In some embodiments of any of the methods described herein, the solution includes about 20% (w/v) to about 100% (w/v) or about 20% (v/v) to about 100% (v/v) of the gelling agent. In some embodiments of any of the methods described herein, the solution includes about 40% (w/v) to about 100% (w/v) or about 40% (v/v) to about 100% (v/v) of the gelling agent. In some embodiments of any of the methods described herein, the solution includes about 60% (w/v) to about 100% (w/v) or about 60% (v/v) to about 100% (v/v) of the gelling agent. In some embodiments of any of the methods described herein, the solution includes about 80% (w/v) to about 100% (w/v) or about 80% (v/v) to about 100% (v/v) of the gelling agent.

In some embodiments of any of the methods described herein, the second solution includes about 1 mg/mL to about 200 mg/mL of the benzenesulfonamide-containing NSAID. In some embodiments of any of the methods described herein, the second solution includes about 1 mg/mL to about 100 mg/mL of the benzenesulfonamide-containing NSAID. In some embodiments of any of the methods described herein, the second solution includes about 1 mg/mL to about 50 mg/mL of the benzenesulfonamide-containing NSAID. In some embodiments of any of the methods described herein, the solution includes about 1 mg/mL to about 200 mg/mL of the benzenesulfonamide-containing NSAID. In some embodiments of any of the methods described herein, the solution includes about 1 mg/mL to about 100 mg/mL of the benzenesulfonamide-containing NSAID. In some embodiments of any of the methods described herein, the solution includes about 1 mg/mL to about 50 mg/mL of the benzenesulfonamide-containing NSAID.

In some embodiments of any of the methods described herein, the first solution is an aqueous solution. In some embodiments of any of the methods described herein, the solution is an aqueous solution. In some embodiments of any of the methods described herein, the first solution includes about 2% (w/v) to about 60% (w/v) silk fibroin.

In some embodiments of any of the methods described herein, the first solution includes about 10% (w/v) to about 60% (w/v) silk fibroin. In some embodiments of any of the methods described herein, the first solution includes about 20% (w/v) to about 60% (w/v) silk fibroin.

In some embodiments of any of the methods described herein, the solution includes about 2% (w/v) to about 60% (w/v) silk fibroin. In some embodiments of any of the methods described herein, the solution includes about 10% (w/v) to about 60% (w/v) silk fibroin. In some embodiments of any of the methods described herein, the solution includes about 20% (w/v) to about 60% (w/v) silk fibroin. In some embodiments of any of the methods described herein, the silk fibroin has a molecular weight range of about 10 kDa to about 500 kDa.

In some embodiments of any of the methods described herein, the benzenesulfonamide-containing NSAID includes a pyrazole moiety. In some embodiments of any of the methods described herein, the pyrazole moiety is a haloalkyl-substituted pyrazole moiety. In some embodiments of any of the methods described herein, the haloalkyl-substituted pyrazole moiety is a trifluoromethyl-substituted pyrazole moiety. In some embodiments of any of the methods described herein, the benzenesulfonamide-containing NSAID is celecoxib.

Some embodiments of any of the methods described herein further include generating silk fibroin having a molecular weight range of about 10 kDa to about 500 kDa. In some embodiments of any of the methods described herein, the step of generating a silk fibroin includes incubating a silk raw material in a solution including sodium carbonate (Na₂CO₃) for 5 minutes to 300 minutes at about 70° C. to about 100° C. In some embodiments of any of the methods described herein, the raw material is a silk worm cocoon, a silk fiber, or a recombinantly-produced silk fibroin.

In some embodiments of any of the methods described herein, the composition is a gel. In some embodiments of any of the methods described herein, the gel is a hydrogel.

In some embodiments of any of the methods described herein, the composition (e.g., a gel, e.g., a hydrogel or organogel) includes a plurality of particles including the benzenesulfonamide-containing NSAID. In some embodiments of any of the methods described herein, the plurality of particles have a d90 of less than 200 μm. In some embodiments of any of the methods described herein, the plurality of particles have a d50 of less than 25 μm. In some embodiments of any of the methods described herein, the plurality of particles have a d10 of less than 10 μm.

Provided herein are compositions (e.g., gels, e.g., hydrogels or organogels) produced by any of the methods described herein. In some embodiments of any of the compositions described herein, the composition is a gel. In some embodiments of any of the compositions described herein, the gel is a hydrogel. In some embodiments of any of the compositions (e.g., gels, e.g., hydrogels or organogels) described herein, the composition includes a plurality of particles including the benzenesulfonamide-containing NSAID. In some embodiments of any of the compositions (e.g., gels, e.g., hydrogels or organogels) described herein, the plurality of particles have a d90 of less than 200 μm. In some embodiments of any of the compositions (e.g., gels, e.g., hydrogels or organogels) described herein, the plurality of particles have a d50 of less than 25 μm. In some embodiments of any of the compositions (e.g., gels, e.g., hydrogels or organogels) described herein, the plurality of particles have a d10 of less than 10 μm.

Provided herein are methods of making a composition (e.g., a gel, e.g., a hydrogel or organogel) that include: (a) providing a solution including about 1% (w/v) to about 60% (w/v) silk fibroin; (b) providing a suspension including about 0.02% (w/v) to about 100% (w/v), or about 0.02% (v/v) to about 100% (v/v), of a gelling agent, and about 1 mg/mL to about 400 mg/mL of a benzenesulfonamide-containing NSAID; (c) mixing a volume of the solution with a volume of the suspension, where the ratio of the volume of the solution to the volume of the suspension is about 5:1 to about 1:5 to provide a mixture; and (d) incubating the mixture for a time sufficient to form the composition (e.g., gel, e.g., a hydrogel or organogel).

Provided herein are methods of making a composition (e.g., a gel, e.g., a hydrogel or organogel) that include: (a) providing a solid composition including silk fibroin; (b) providing a suspension including about 0.02% (w/v) to about 100% (w/v), or about 0.02% (v/v) to about 100% (v/v), of a gelling agent, and about 1 mg/mL to about 400 mg/mL of a benzenesulfonamide-containing NSAID; (c) mixing a volume of the solution with an amount of the solid composition; and (d) incubating the mixture of a time sufficient to form the composition (e.g., gel, e.g., a hydrogel or organogel).

Provided herein are methods of making a composition (e.g., a gel, e.g., a hydrogel or organogel) that include: (a) providing a suspension including about 1 mg/mL to about 400 mg/mL of a benzenesulfonamide-containing NSAID and about 1% (w/v) to about 60% (w/v) silk fibroin; (b) a solution including about 0.02% (w/v) to about 100% (w/v), or about 0.02% (v/v) to about 100% (v/v), of a gelling agent; (c) mixing a volume of the solution with a volume of the suspension, where the ratio of the volume of the solution to the volume of the suspension is about 5:1 to about 1:5 to provide a mixture; and (d) incubating the mixture of a time sufficient to form the composition (e.g., gel, e.g., a hydrogel or organogel).

Provided herein are methods of making a composition (e.g., a gel, e.g., a hydrogel or organogel) that include: (a) providing a suspension including about 1 mg/mL to about 400 mg/mL of a benzenesulfonamide-containing NSAID; (b) a solution including about 0.02% (w/v) to about 100% (w/v), or about 0.02% (v/v) to about 100% (v/v), of a gelling agent, and about 1% (w/v) to about 60% (w/v) silk fibroin; (c) mixing a volume of the solution with a volume of the suspension, where the ratio of the volume of the solution to the volume of the suspension is about 5:1 to about 1:5 to provide a mixture; and (d) incubating the mixture of a time sufficient to form the composition (e.g., gel, e.g., a hydrogel or organogel).

Also provided herein are methods of making a composition (e.g., a gel, e.g., a hydrogel or organogel) that include: (a) providing a suspension including about 1 mg/mL to about 400 mg/mL of a benzenesulfonamide-containing NSAID; (b) a first solution including about 0.02% (w/v) to about 100% (w/v), or about 0.02% (v/v) to about 100% (v/v), of a gelling agent; (c) a second solution including about 1% (w/v) to about 60% (w/v) silk fibroin; (d) mixing a volume of the first solution, a volume of the second solution, and a volume of the suspension to provide a mixture; and (e) incubating the mixture of a time sufficient to form the composition (e.g., gel, e.g., a hydrogel or organogel).

In some embodiments of any of the methods described herein, the gelling agent is glycerol or a polyethylene glycol (PEG). In some embodiments of any of the methods described herein, the PEG is PEG300, PEG400, or PEG600, or a PEG having a molecular weight of about 2 kDa, about 4 kDa, about 6 kDa, about 8 kDa, about 10 kDa, about 12 kDa, about 14 kDa, about 16 kDa, about 18 kDa, or about 20 kDa.

In some embodiments of any of the methods described herein, the gelling agent is selected from the group of: sorbitol, triethylamine, 2-pyrrolidone, alpha-cyclodextrin, benzyl alcohol, beta-cyclodextrin, dimethyl sulfoxide, dimethylacetamide (DMA), dimethylformamide, ethanol, gamma-cyclodextrin, glycerol, glycerol formal, hydroxypropyl beta-cyclodextrin, kolliphor 124, kolliphor 181, kolliphor 188, kolliphor 407, kolliphor EL (cremaphor EL), cremaphor RH 40, cremophor RH 60, d-alpha-tocopherol, PEG 1000 succinate, polysorbate 20, polysorbate 80, solutol HS 15, sorbitan monooleate, poloxamer-407, poloxamer-188, Labrafil M-1944CS, Lrabfil M-2125CS, Labrasol, Gellucire 44/14, Softigen 767, mono- and di-fatty acid esters of PEG 300, PEG 400, or PEG 1750, kolliphor RH60, N-methyl-2-pyrrolidone, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil, beeswax, d-alpha-tocopherol, oleic acid, medium-chain mono- and di-glycerides, alpha-cyclodextrin, beta-cyclodextrin, hydroxypropyl-beta-cyclodextrin, sulfo-butylether-beta-cyclodextrin, hydrogenated soy phosphatidylcholine, distearoylphosphatidylglycerol, L-alpha-dimyristoylphosphatidylcholine, L-alpha-dimyristoylphosphatidylglycerol, PEG 300, PEG 300 caprylic/capric glycerides (Softigen 767), PEG 300 linoleic glycerides (Labrafil M-2125CS), PEG 300 oleic glycerides (Labrafil M-1944CS), PEG 400, PEG 400 caprylic/capric glycerides (Labrasol), polyoxyl 40 stearate (PEG 1750 monosterate), polyoxyl 8 stearate (PEG 400 monosterate), polysorbate 20, polysorbate 80, polyvinyl pyrrolidone, propylene carbonate, propylene glycol, solutol HS15, sorbitan monooleate (Span 20), sulfobutylether-beta-cyclodextrin, transcutol, triacetin, 1-dodecylazacyclo-heptan-2-one, caprolactam, castor oil, cottonseed oil, ethyl acetate, medium chain triglycerides, methyl acetate, oleic acid, safflower oil, sesame oil, soybean oil, tetrahydrofuran, and glycerin.

In some embodiments of any of the methods described herein, the silk fibroin has a molecular weight range of about 10 kDa to about 500 kDa. Some embodiments of any of the methods described herein, the method further includes generating silk fibroin having a molecular weight range of about 10 kDa to about 500 kDa. In some embodiments of any of the methods described herein, the step of generating a silk fibroin includes incubating a silk raw material in a solution including sodium carbonate (Na₂CO₃) for 5 minutes to 300 minutes at about 70° C. to about 100° C.

In some embodiments of any of the methods described herein, the benzenesulfonamide-containing NSAID includes a pyrazole moiety. In some embodiments of any of the methods described herein, the pyrazole moiety is a haloalkyl-substituted pyrazole moiety. In some embodiments of any of the methods described herein, the haloalkyl-substituted pyrazole moiety is a trifluoromethyl-substituted pyrazole moiety. In some embodiments of any of the methods described herein, the benzenesulfonamide-containing NSAID is celecoxib.

In some embodiments of any of the methods described herein, the composition is a gel. In some embodiments of any of the methods described herein, the gel is a hydrogel. In some embodiments of any of the methods described herein, the composition (e.g., gel, e.g., a hydrogel or organogel) includes a plurality of particles including the benzenesulfonamide-containing NSAID. In some embodiments of any of the methods described herein, the plurality of particles have a d90 of less than 200 μm. In some embodiments of any of the methods described herein, the plurality of particles have a d50 of less than 25 μm. In some embodiments of any of the methods described herein, the plurality of particles have a d10 of less than 10 μm.

Also provided herein are compositions (e.g., gels, e.g., hydrogels or organogels) produced by any of the methods described herein. In some embodiments of any of the compositions described herein, the composition is a gel. In some embodiments of any of the compositions described herein, the gel is a hydrogel. In some embodiments of any of the compositions (e.g., gels, e.g., hydrogels or organogels) described herein, the composition (e.g., gel, e.g., a hydrogel or organogel) includes a plurality of particles including the benzenesulfonamide-containing NSAID. In some embodiments of any of the compositions (e.g., gels, e.g., hydrogels or organogels) described herein, the plurality of particles have a d90 of less than 200 μm. In some embodiments of any of the compositions (e.g., gels, e.g., hydrogels or organogels) described herein, the plurality of particles have a d50 of less than 25 μm. In some embodiments of any of the compositions (e.g., gels, e.g., hydrogels or organogels) described herein, the plurality of particles have a d10 of less than 10 μm.

Provided herein are kits that include any of the compositions (e.g., gels, e.g., hydrogels or organogels) described herein. In some embodiments of any of the kits described herein, the kit further includes a syringe, wherein the syringe is filled with any of the compositions (e.g., gels, e.g., hydrogels or organogels) described herein. In some embodiments of any of the kits described herein, the kit further includes a sealed vial, wherein the sealed vial is filled with any of the compositions (e.g., gels, e.g., hydrogels or organogels) described herein. In some embodiments of any of the kits described herein, the kit further includes instructions for performing any of the methods described herein.

As used herein, the word “a” before a noun represents one or more of the particular noun. For example, the phrase “a benzenesulfonamide-containing NSAID” represents “one or more benzenesulfonamide-containing NSAID.”

The term “subject” means a mammal (e.g., a human, sheep, dog, cat, horse, cow, pig, rabbit, rat, mouse, or monkey).

The term “a benzenesulfonamide-containing NSAID” as used herein includes any of the benzenesulfonamide-containing NSAIDs described herein or known in the art, or a salt (e.g., a pharmaceutically acceptable salt) or solvate thereof.

The term “treating” means reducing one or more of: reducing the number of symptoms of a condition in a subject, reducing the severity of one or more symptoms of a condition in a subject, reducing the duration of one or more symptoms of a condition in a subject, and reducing the frequency of one or more symptoms of a condition in a subject.

The term “inflammatory condition” means a disease results in or is associated with an increased level of inflammation in a subject (e.g., an increased level of inflammation in one or more tissues in a subject) as compared to a control subject not having an inflammatory condition. Non-limiting examples of inflammatory conditions are described herein. Additional examples of inflammatory conditions are known in the art.

The term “pain condition” means a condition in a subject that is associated with or results in one or more different types of pain (e.g., any of the different types of pain described herein). Non-limiting examples of pain conditions are described herein.

The phrase “reducing inflammation” means a detectable reduction in the level/severity of one or more (e.g., two or more) markers/symptoms of inflammation in a subject following administration of a therapeutic agent (e.g., any of the gels or compositions provided herein) as compared to the level/severity of the one or more (e.g., two or more) markers/symptoms of inflammation in the subject prior to administration of the therapeutic agent. Non-limiting examples of symptoms of inflammation include: pain, heat, redness, swelling, and loss of function. Non-limiting examples of markers of inflammation include prostaglandins (e.g., PGE2), erythrocyte sedimentation rate (ESR), white cell count, interleukin (IL)-1, IL-2, IL-3, IL-4, IL-5, IL-6, IL-7, IL-8, IL-9, IL-10, IL-11, IL-12, IL-13, tumor necrosis factor (TNF)-α, TNF-β, tumor growth factor-β, interferon-γ, GCSF, GM-CSF, C-reactive protein, serum amyloid P component, serum amyloid A, complement protein C3, complement protein C5a, factor XII, membrane attack complex (complement system), plasmin, thrombin, mannan-binding lectin, fibrinogen, prothrombin, factor VIII, von Willebrand factor, plasminogen activator inhibitor PAI-1, alpha 2-macroglobulin, ferritin, hecidin, ceruloplasmin, haptoglobin, orosomucoid, α-1-antitrpysin, α-1-antichymotrypsin, histamine, and leukotriene B4.

The phrase “reducing pain” means a detectable decrease in a subject's pain intensity following administration of a therapeutic agent (e.g., any of the gels or compositions provided herein) as compared to the intensity of the subject's pain prior to administration of the therapeutic agent. In some embodiments, the pain is one or more of nociceptive pain, neuropathic pain, psychogenic pain, breakthrough pain, incident pain, back pain, musculoskeletal pain, post-operative pain, operative pain, visceral pain, joint pain, acute pain, inflammatory pain, and chronic pain. The intensity of pain in a subject can be determined, e.g., using a pain scale. Non-limiting examples of pain scales that can be used to determine the intensity of pain in a subject include: Alder Hey Triage Pain Score, Behavioral Pain Scale (BPS), Brief Pain Inventory (BPI), Checklist of Nonverbal Pain Indicators (CNPI), Clinical Global Impression (CGI), Critical-Care Point Observation Tool (CPOT), COMFORT scale, Dallas Pain Questionnaire, Descriptor Differential Scale (DDS), Dolorimeter Pain Index (DPI), Edmonton Symptom Assessment System, Faces Pain Scale Revised (FPS-R), Face Legs Activity Cry Consolability Scale, Lequesne Algofunctional Index, McGill Pain Questionnare (MPQ), Neck Pain and Disability Scale (NPAD), Numerical 11 Point Box (BS-11), Numeric Rating Scale (NRS-11), OSWESTRY Index, Palliative Care Outcome Scale (PCOS), Roland-Morris Back Pain Questionnare, Support Team Assessment Schedule (STAS), Wong-Baker FACES Pain Rating Scale, Visual Analog Scale (VAS), Australian/Canadian Osteoarthritis Hand Index (AUSCAN), Western Ontario and McMaster Universities Hand Index (WOMAC), and Osteoarthritis Research Society International-Outcome Measures in Rheumatoid Arthritis Clinical Trials (OARSI-OMERACT).

The term “fever” means a body temperature that is elevated as compared to a normal body temperature range for a subject (e.g., a human).

The phrase “reducing fever” means a detectable reduction in the body temperature of a subject having fever. Methods for measuring a subject's body temperature are known in the art, e.g., a thermometer (e.g., an oral thermometer).

The term “non-steroidal anti-inflammatory drug” or “NSAID” means a molecule that inhibits the catalytic activity of one or both of cyclooxygenase-2 (COX-2) and cyclooxygenase-1 (COX-1). For example, a NSAID can selectively inhibit the catalytic activity of COX-2. In other examples, a NSAID can inhibit the catalytic activity of COX-1 and COX-2. Exemplary assays for measuring the catalytic activity of COX-1 and COX-2 are described herein. Additional assays for measuring the catalytic activity of COX-1 and COX-2 are known in the art. Additional exemplary properties of NSAID are described herein.

Substantial research has determined that at sites of inflammation, COX-2 is the primary cyclooxygenase responsible for the generation of the pro-inflammatory prostanoid, PGE2. Since PGE2 levels are major drivers of the cardinal signs of inflammation, then inhibition of COX-2 leads to reductions of these signs at sites of inflammation. For example, in an osteoarthritic knee joint, oral administration of a COX-2 inhibitor is effective at reducing pain, swelling, and loss of function. This oral administration comes with an increased risk of side effects due to the systemic exposures necessary to drive local concentrations that are sufficiently high to be pharmacologically active in the local environment of the knee joint.

The term “pharmaceutically acceptable” indicates that the substance or composition is compatible chemically and/or toxicologically, with the other ingredients comprising a formulation, and/or the subject being treated therewith.

The term “hydrido” denotes a single hydrogen atom (H). A hydrido radical can be attached, e.g., to an oxygen atom to form a hydroxyl radical or two hydrido radicals can be attached to a carbon atom to form a methylene (—CH₂—) radical.

The term “plurality” before a noun represents two or more (e.g., three or more, four or more, five or more, ten or more, fifteen or more, twenty or more, thirty or more, or fifty or more) of the particular noun. For example, the phrase “plurality of particles” means “two or more particles.”

Where the term “alkyl” is used, either alone or within other terms, such as “haloalkyl” and “alkylsulfonyl,” it includes linear or branched radicals having one to about twenty carbon atoms or one to about twelve carbon atoms. Alkyl radicals can be “lower alkyl” radicals having one to about ten carbon atoms, or about one to about six carbon atoms. Examples of alkyl radicals include methyl, ethyl, n-propyl, isopropyl, n-butyl, isobutyl, sec-butyl, tert-butyl, pentyl, iso-amyl, hexyl, and the like.

The term “alkenyl” includes linear or branched radicals having at least one carbon-carbon double bond of two to about twenty carbon atoms, or two to about twelve carbon atoms. The alkyl radicals can be “lower alkenyl” radicals having two to about six carbon atoms. Examples of such radicals include ethenyl, n-propenyl, butenyl, and the like.

The term “halo” means halogens, such as fluorine, chlorine, bromine, or iodine atoms.

The term “haloalkyl” includes radicals, wherein any one or more of the alkyl carbon atoms is substituted with halo as defined above. Examples of haloalkyl radicals include monohaloalkyl, dihaloalkyl, and polyhaloalkyl radicals. A monohaloalkyl radical, e.g., can have either an iodo, bromo, chloro, or fluoro atom within the radical. Dihalo- and polyhalo-alkyl radicals can have two or more of the same halo atoms or a combination of different halo radicals.

Haloalkyl radicals can be “lower haloalkyl” radicals having one to six carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl, and dichloropropyl.

The term “hydroxyalkyl” includes linear or branched alkyl radicals having one to about ten carbon atoms, any one of which may be substituted with one or more hydroxyl radicals. For example, hydroxyalkyl radicals can be “lower hydroxyalkyl” radicals having one to six carbon atoms and one or more hydroxyl radicals. Examples of lower hydroxyalkyl radicals include hydroxymethyl, hydroxyethyl, hydroxypropyl, hydroxybutyl, and hydroxyhexyl.

The terms “alkoxy” and “alkoxyalkyl” include linear or branched oxy-containing radicals, each having alkyl portions of one to about ten carbon atoms, such as a methoxy radical. Alkoxy radicals can be “lower alkoxy” radicals having one to six carbon atoms. Examples of lower alkoxy radicals include methoxy, ethoxy, propoxy, butoxy, and tert-butoxy. The term “alkoxyalkyl” also includes alkyl radicals having two or more alkoxy radicals attached to the alkyl radical, that is, to form monoalkoxyalkyl and dialkoxyalkyl radicals. An alkoxyalkyl radical can be a “lower alkoxyalkyl” radical having one to six carbon atoms and one or two alkoxy radicals. Examples of lower alkoxyalkyl radicals include methoxymethyl, methoxyethyl, ethoxyethyl, methoxybutyl, and methoxypropyl. The “alkoxy” or “alkoxyalkyl” radicals can be further substituted with one or more halo atoms, such as fluoro, chloro, or bromo, to provide “haloalkoxy” or “haloalkoxyalkyl” radicals. Examples of such radicals include fluoromethoxy, chloromethoxy, trifluoromethoxy, trifluoroethoxy, fluoroethoxy, and fluoropropoxy.

The term “aryl,” alone or in combination, means a carbocyclic aromatic system containing one, two, or three rings, where such rings may be attached together in a pendent manner or may be fused. The term “aryl” includes aromatic radicals, such as phenyl, naphthyl, tetrahydronaphthyl, indane, and biphenyl.

The term “heterocyclic” includes saturated, partially saturated, and unsaturated heteroatom-containing ring-shaped radicals, where the heteroatoms may be selected from nitrogen, sulfur, and oxygen. Examples of saturated heterocyclic radicals include saturated 3- to 6-membered heteromonocylic group containing 1 to 4 nitrogen atoms (e.g., pyrrolidinyl, imidazolidinyl, piperidino, piperazinyl, etc.); saturated 3- to 6-membered heteromonocyclic group containing 1 to 2 oxygen atoms and 1 to 3 nitrogen atoms (e.g., morpholinyl, etc.); saturated 3- to 6-membered heteromonocyclic group containing 1 to 2 sulfur atoms and 1 to 3 nitrogen atoms (e.g., thiazolidinyl, etc.). Examples of partially saturated heterocyclic radicals include dihydrothiophene, dihydropyran, dihydrofuran, and dihydrothiazole.

The term “heteroaryl” includes unsaturated heterocyclic radicals. Examples of unsaturated heterocyclic radicals, also termed “heteroaryl” radicals include unsaturated 5- to 6-membered heteromonocyclic group containing 1 to 4 nitrogen atoms, e.g., pyrrolyl, pyrrolinyl, imidazolyl, pyrazolyl, 2-pyridyl, 3-pyridyl, 4-pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.), and tetrazolyl (e.g., 1H-tetrazolyl, 2H-tetrazolyl, etc.); unsaturated condensed heterocyclic group containing 1 to 5 nitrogen atoms, e.g., indolyl, isoindolyl, indolizinyl, benzimidazolyl, quinolyl, isoquinolyl, indazolyl, benzotriazolyl, and tetrazolopyridazinyl (e.g., tetrazolo[1,5-b]pyridazinyl, etc.]; unsaturated 3 to 6-membered heteromonocyclic group containing an oxygen atom, for example, pyranyl, 2-furyl, 3-furyl, etc.; unsaturated 5 to 6-membered heteromonocyclic group containing a sulfur atom, e.g., 2-thienyl, 3-thienyl, etc.; unsaturated 5- to 6-membered heteromonocyclic group containing 1 to 2 oxygen atoms and 1 to 3 nitrogen atoms, e.g., oxazolyl, isoxazolyl, and oxadiazolyl (e.g., 1, 2, 4-oxadiazolyl, 1,3,4-oxadiazolyl, 1, 2, 5-oxadiazolyl, etc.); unsaturated condensed heterocyclic group containing 1 to 2 oxygen atoms and 1 to 3 nitrogen atoms (e.g., benzoxazolyl, benzoxadiazolyl, etc.); unsaturated 5- to 6-membered heteromonocyclic group containing 1 to 2 sulfur atoms and 1 to 3 nitrogen atoms, e.g., thiazolyl and thiadiazolyl (e.g., 1,2,4-thiadiazolyl, 1, 3, 4-thiadiazolyl, 1,2,5-thiadiazolyl, etc.); and unsaturated condensed heterocyclic group containing 1 to 2 sulfur atoms and 1 to 3 nitrogen atoms (e.g., benzothiazolyl, benzothiadiazolyl, etc.) and the like. The term also includes radicals where heterocyclic radicals are fused with aryl radicals. Examples of such fused bicyclic radicals include benzofuran, benzothiophene, and the like. A “heterocyclic group” can have, e.g., 1 to 3 substituents, such as lower alkyl, hydroxy, oxo, amino, and lower alkylamino. Examples of heterocyclic radicals include five- to ten-membered fused or unfused radicals. Some examples of heteroaryl radicals include benzofuryl, 2,3-dihydrobenzofuryl, benzothienyl, indolyl, dihydroindolyl, chromanyl, benzopyran, thiochromanyl, benzothiopyran, benzodioxolyl, benzodioxanyl, pyridyl, thienyl, thiazolyl, oxazolyl, furyl, and pyrazinyl.

The term “sulfonyl,” whether used alone or linked to other terms such as alkylsulfonyl, denotes respectively divalent radicals —SO₂—.

The term “alkylsulfonyl” includes alkyl radicals attached to a sulfonyl radical, where alkyl is defined as above. Examples of alkylsulfonyl radicals are “lower alkylsulfonyl” radicals having one to six carbon atoms. Examples of such lower alkylsulfonyl radicals include methylsulfonyl, ethylsulfonyl, and propylsulfonyl. The term “arylsulfonyl” includes aryl radicals as defined above, attached to a sulfonyl radical. Examples of such radicals include phenylsulfonyl.

The terms “sulfamyl,” “aminosulfonyl,” and “sulfonamidyl,” whether alone or used with terms such as “N-alkylaminosulfonyl,” “N-arylaminosulfonyl,” “N,N-dialkylaminosulfonyl,” and “N-alkyl-N-arylaminosulfonyl,” denotes a sulfonyl radical substituted with an amine radical, forming a sulfonamide (—SO₂NH₂). The terms “N-alkylaminosulfonyl” and “N,N-dialkylaminosulfonyl” denote sulfamyl radicals substituted, respectively, with one alkyl radical, or two alkyl radicals. Examples of alkylaminosulfonyl radicals are “lower alkylaminosulfonyl” radicals having one to six carbon atoms. Examples of such lower alkylaminosulfonyl radicals include N-methylaminosulfonyl, N-ethylaminosulfonyl, and N-methyl-N-ethylaminosulfonyl. The terms “N-arylaminosulfonyl” and “N-alkyl-N-arylaminosulfonyl” denote sulfamyl radicals substituted, respectively, with one aryl radical, or one alkyl and one aryl radical. Examples of N-alkyl-N-arylaminosulfonyl radicals are “lower N-alkyl-N-arylsulfonyl” radicals having alkyl radicals of one to six carbon atoms. Examples of such lower N-alkyl-N-aryl aminosulfonyl radicals include N-methyl-phenylaminosulfonyl and N-ethyl-phenylaminosulfonyl.

The terms “carboxy” or “carboxyl,” whether used alone or with other terms, such as “carboxyalkyl,” denotes —CO₂H. The terms “alkanoyl” or “carboxyalkyl” include radicals having a carboxy radical as defined above, attached to an alkyl radical. The alkanoyl radicals can be substituted or unsubstituted, such as formyl, acetyl, propionyl (propanoyl), butanoyl (butyryl), isobutanoyl (isobutyryl), valeryl (pentanoyl), isovaleryl, pivaloyl, hexanoyl, or the like.

The term “carbonyl,” whether used alone or with other terms, such as “alkylcarbonyl,” denotes —(C═O)—. The term “alkylcarbonyl” includes radicals having a carbonyl radical substituted with an alkyl radical. In some examples, the alkylcarbonyl radicals are “lower alkylcarbonyl” radicals having one to six carbon atoms. Examples of such radicals include methylcarbonyl and ethylcarbonyl. The term “alkylcarbonylalkyl,” denotes an alkyl radical substituted with an “alkylcarbonyl” radical. The term “alkoxycarbonyl” means a radical containing an alkoxy radical, as defined above, attached via an oxygen atom to a carbonyl radical. The term “lower alkoxycarbonyl” includes alkoxy radicals having one to six carbon atoms. Examples of such “lower alkoxycarbonyl” ester radicals include substituted or unsubstituted methoxycarbonyl, ethoxycarbonyl, propoxycarbonyl, butoxycarbonyl, and hexyloxycarbonyl. The term “alkoxycarbonylalkyl” includes radicals having “alkoxycarbonyl,” as defined above substituted to an alkyl radical. In some examples, the alkoxycarbonylalkyl radicals are “lower alkoxycarbonylalkyl” having lower alkoxycarbonyl radicals as defined above attached to one to six carbon atoms. Examples of such lower alkoxycarbonylalkyl radicals include methoxycarbonylmethyl, tert-butoxycarbonylethyl, and methoxycarbonylethyl.

The term “aminocarbonyl” when used by itself or with other terms, such as “aminocarbonylalkyl,” “N-alkylaminocarbonyl,” “N-arylaminocarbonyl,” “N,N-dialkylaminocarbonyl,” “N-alkyl-N-arylaminocarbonyl,” “N-alkyl-N-hydroxyaminocarbonyl,” and “N-alkyl-N-hydroxyaminocarbonylalkyl,” denotes an amide group of the formula —C(═O)NH₂—. The terms “N-alkylaminocarbonyl” and “N,N-dialkylaminocarbonyl” denote aminocarbonyl radicals which have been substituted with one alkyl radical and with two alkyl radicals, respectively. Examples include “lower alkylaminocarbonyl,” which have lower alkyl radicals as described above attached to an aminocarbonyl radical.

The terms “N-arylaminocarbonyl” and “N-alkyl-N-arylaminocarbonyl” denote aminocarbonyl radicals substituted, respectively, with one aryl radical, or one alkyl and one aryl radical. The term “aminocarbonylalkyl” includes alkyl radicals substituted with aminocarbonyl radicals. The term “N-cycloalkylaminocarbonyl” denotes aminocarbonyl radicals which have been substituted with at least one cycloalkyl radical. Examples of N-cycloalkylaminocarbonyl are “lower cycloalkylaminocarbonyl” which have lower cycloalkyl radicals of three to seven carbon atoms, attached to an aminocarbonyl radical. The term “aminoalkyl” includes alkyl radicals substituted with amino radicals. The term “alkylaminoalkyl” includes aminoalkyl radicals having the nitrogen atom substituted with an alkyl radical.

The term “amidino” denotes an —C(═NH)—NH₂ radical. The term “cyanoamidino” denotes an —C(═N—CN)—NH₂ radical. The term “heterocyclicalkyl” includes heterocyclic-substituted alkyl radicals. In some examples, the heterocyclicalkyl radicals are “lower heterocyclicalkyl” radicals having one to six carbon atoms and a heterocyclic radical. Examples include such radicals as pyrrolidinylmethyl, pyridylmethyl, and thienylmethyl.

The term “aralkyl” includes aryl-substituted alkyl radicals. Examples of aralkyl radicals are “lower aralkyl” radicals having aryl radicals attached to alkyl radicals having one to six carbon atoms. Examples of such radicals include benzyl, diphenylmethyl, triphenylmethyl, phenylethyl, and diphenylethyl. The aryl in the aralkyl may be additionally substituted with halo, alkyl, alkoxy, halkoalkyl, and haloalkoxy. The terms benzyl and phenylmethyl are interchangeable.

The term “cycloalkyl” includes radicals having three to ten carbon atoms. In some examples, the cycloalkyl radicals are “lower cycloalkyl” radicals having three to seven carbon atoms. Examples include radicals such as cyclopropyl, cyclobutyl, cyclopentyl, cyclohexyl, and cycloheptyl. The term “cycloalkenyl” includes unsaturated cyclic radicals having three to ten carbon atoms, such as cyclobutenyl, cyclopentenyl, cyclohexenyl, and eyeloheptenyl. The term “alkylthio” includes radicals containing a linear or branched alkyl radical, of one to ten carbon atoms, attached to a divalent sulfur atom. An example of “alkylthio” is methylthio, (CH₃—S—).

The term “alkylsulfinyl” includes radicals containing a linear or branched alkyl radical, of one to ten carbon atoms, attached to a divalent —S(═O)— atom. The term “aminoalkyl” includes alkyl radicals substituted with amino radicals. In some examples, the aminoalkyl radicals are “lower aminoalkyl” having one to six carbon atoms. Examples include aminomethyl, aminoethyl, and aminobutyl. The term “alkylaminoalkyl” includes aminoalkyl radicals having the nitrogen atom substituted with at least one alkyl radical. In some examples, the alkylaminoalkyl radicals are “lower alkylaminoalkyl” having one to six carbon atoms attached to a lower aminoalkyl radical as described above. The terms “N-alkylamino” and “N, N-dialkylamino” denote amino groups which have been substituted with one alkyl radical and with two alkyl radicals, respectively. In some examples, the alkylamino radicals are “lower alkylamino” radicals having one or two alkyl radicals of one to six carbon atoms, attached to a nitrogen atom. The term “alkylamino” can be mono- or di-alkylamino, such as N-methylamino, N-ethylamino, N,N-dimethylamino, N,N-diethylamino, or the like.

The term “arylamino” denotes amino groups which have been substituted with one or two aryl radicals, such as N-phenylamino. The “arylamino” radicals may be further substituted on the aryl ring portion of the radical. The term “aralkylamino” denotes amino groups which have been substituted with one or two aralkyl radicals, such as N-benzylamino. The “aralkylamino” radicals may be further substituted on the aryl ring portion of the radical. The terms “N-alkyl-N-arylamino” and “N-aralkyl-N-alkylamino” denote amino groups which have been substituted with one aralkyl and one alkyl radical, or one aryl and one alkyl radical, respectively, to an amino group. The terms “N-arylaminoalkyl” and “N-aralkylaminoalkyl” denote amino groups which have been substituted with one aryl radical or one aralkyl radical, respectively, and having the amino group attached to an alkyl radical. In some examples, the arylaminoalkyl radicals are “lower arylaminoalkyls” having the arylamino radical attached to one to six carbon atoms. Examples of such radicals include N-phenylaminomethyl and N-phenyl-N-methylaminomethyl. The terms “N-alkyl-N-arylaminoalkyl” and “N-aralkyl-N-alkylaminoalkyl” denote N-alkyl-N-arylamino and N-alkyl-N-aralkylamino groups, respectively, and having the amino group attached to alkyl radicals.

The term “acyl,” whether used alone, or within a term such as “acylamino,” denotes a radical provided by the residue after removal of hydroxyl from an organic acid. The term “acylamino” includes an amino radical substituted with an acyl group. Non-limiting examples of an “acylamino” radical are acetylamino or acetamido (CH₃C(═O)—NH—), where the amine may be further substituted with alkyl, aryl, or aralkyl. The term “arylthio” includes aryl radicals of six to ten carbon atoms, attached to a divalent sulfur atom. An example of “arylthio” is phenylthio. The term “aralkylthio” includes aralkyl radicals as described above, attached to a divalent sulfur atom. An example of “aralkylthio” is benzylthio. The term “aryloxy” includes aryl radicals, as defined above, attached to an oxygen atom. Examples of such radicals include phenoxy. The term “aralkoxy” includes oxy-containing aralkyl radicals attached through an oxygen atom to other radicals. In some examples, the aralkoxy radicals are “lower aralkoxy” radicals having phenyl radicals attached to lower alkoxy radical as described above.

The term “haloaralkyl” includes aryl radicals as defined above attached to haloalkyl radicals. The term “carboxyhaloalkyl” includes carboxyalkyl radicals as defined above having halo radicals attached to the alkyl portion. The term “alkoxycarbonylhaloalkyl” includes alkoxycarbonyl radicals as defined above substituted on a haloalkyl radical. The term “aminocarbonylhaloalkyl” includes aminocarbonyl radicals as defined above substituted on a haloalkyl radical. The term “alkylaminocarbonylhaloalkyl” includes alkylaminocarbonyl radicals as defined above substituted on a haloalkyl radical. The term “alkoxycarbonylcyanoalkenyl” includes alkoxycarbonyl radicals as defined above, and a cyano radical, both substituted on an alkenyl radical. The term “carboxyalkylaminocarbonyl” includes aminocarbonyl radicals substituted with carboxyalkyl radicals, as defined above. The term “aralkoxycarbonylalkylaminocarbonyl” includes aminocarbonyl radicals substituted with aryl-substituted alkoxycarbonyl radicals, as defined above.

The term “cycloalkylalkyl” includes cycloalkyl radicals having three to ten carbon atoms attached to an alkyl radical, as defined above. In some examples, the cycloalkylalkyl radicals are “lower cycloalkylalkyl” radicals having cycloalkyl radicals attached to lower alkyl radicals as defined above. Examples include radicals such as cyclopropylmethyl, cyclobutylmethyl, and cyclohexylethyl. The term “aralkenyl” includes aryl radicals attached to alkenyl radicals having two to ten carbon atoms, such as phenylbutenyl, phenylethenyl, or styryl.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a graph showing the cumulative release of lomoxicam over time from the silk fibroin hydrogels shown in Table 1. The term “silk” in this figure refers to silk fibroin.

FIG. 2 is a graph showing the cumulative release of diclofenac sodium salt over time from the silk fibroin hydrogels shown in Table 2. The term “silk” in this figure refers to silk fibroin.

FIG. 3 is a graph showing the cumulative release of meloxicam over time from the silk fibroin hydrogels shown in Table 3. The term “silk” in this figure refers to silk fibroin.

FIG. 4 is a graph showing the cumulative release of tenoxicam over time from the silk fibroin hydrogels shown in Table 4. The term “silk” in this figures refers to silk fibroin.

FIG. 5 is a graph showing the cumulative release of celecoxib over time from the silk fibroin hydrogels shown in Table 5. The term “silk” in this figure refers to silk fibroin.

FIG. 6 is a graph showing the cumulative release of celecoxib over time from the silk fibroin hydrogels shown in Table 6. The term “silk” in this figure refers to silk fibroin.

FIG. 7 is a graph showing the cumulative release of celecoxib over time from the silk fibroin hydrogels shown in Table 7. The term “silk” in this figure refers to silk fibroin.

FIG. 8 is a schematic diagram of the acute knee inflammation and pain animal model study described in Example 2.

FIG. 9 is a graph showing gait analysis score (0-6) of the acute knee inflammation and pain rat model over time following no treatment or the intraarticular administration of vehicle, 100 mg/mL celecoxib, a 6.5% (w/v) silk fibroin gel, a 6.5% (w/v) silk fibroin gel including 100 mg/mL celecoxib, a 3.5% (w/v) silk fibroin gel, or a 3.5% (w/v) silk fibroin gel including 100 mg/mL celecoxib.

FIG. 10 is a graph of the area under the curve (AUC) of the cumulative gait score of the acute knee inflammation and pain rat model for days 1-4 following no treatment or the intraarticular administration of vehicle, 100 mg/mL celecoxib, a 6.5% (w/v) silk fibroin gel, a 6.5% (w/v) silk fibroin gel including 100 mg/mL celecoxib, a 3.5% (w/v) silk fibroin gel, or a 3.5% (w/v) silk fibroin gel including 100 mg/mL celecoxib.

FIG. 11 is a graph showing the Von Frey Filament measurement (g) of the acute knee inflammation and pain rat model over time following no treatment or the intraarticular administration of vehicle, 100 mg/mL celecoxib, a 6.5% (w/v) silk fibroin gel, a 6.5% (w/v) silk fibroin gel including 100 mg/mL celecoxib, a 3.5% (w/v) silk fibroin gel, or a 3.5% (w/v) silk fibroin gel including 100 mg/mL celecoxib.

FIG. 12 is a graph of the area under the curve (AUC) of the Von Frey Filament measurement (g) of the acute knee inflammation and pain rat model for days 1-9 following no treatment or the intraarticular administration of vehicle, 100 mg/mL celecoxib, a 6.5% (w/v) silk fibroin gel, a 6.5% (w/v) silk fibroin gel including 100 mg/mL celecoxib, a 3.5% (w/v) silk fibroin gel, or a 3.5% (w/v) silk fibroin gel including 100 mg/mL celecoxib.

FIG. 13 is a graph showing the knee caliper measurement of the acute knee inflammation and pain rat model over time following no treatment or the intraarticular administration of vehicle, 100 mg/mL celecoxib, a 6.5% (w/v) silk fibroin gel, a 6.5% (w/v) silk fibroin gel including 100 mg/mL celecoxib, a 3.5% (w/v) silk fibroin gel, or a 3.5% (w/v) silk fibroin gel including 100 mg/mL celecoxib.

FIG. 14 is a graph of the area under the curve (AUC) of the knee caliper knee measurement of the acute knee inflammation and pain rat model for days 3-10 following no treatment or the intraarticular administration of vehicle, 100 mg/mL celecoxib, a 6.5% (w/v) silk fibroin gel, a 6.5% (w/v) silk fibroin gel including 100 mg/mL celecoxib, a 3.5% (w/v) silk fibroin gel, or a 3.5% (w/v) silk fibroin gel including 100 mg/mL celecoxib.

FIG. 15 is a graph showing the molecular weight of fibroin solution after different times of degumming at 100° C. and autoclave conditioning.

FIG. 16 is a graph showing the release kinetics of celecoxib from a suspension control and silk fibroin hydrogels containing 30 mg/mL and 100 mg/mL celecoxib. The term “silk” in this figure refers to silk fibroin.

FIG. 17 is a graph of the molecular weight from Treeway cocoons, Treenway facial grade cocoons, and SOHO silk yarn after degumming or autoclave as determined by size exclusion chromatography.

FIG. 18 is a graph of the cumulative percentage release of celecoxib from silk fibroin hydrogels (100 mg/mL celecoxib and 6.5% (w/v) silk fibroin) prepared using Treenway cocoons, Treenway facial grade cocoons, and SOHO silk yarn over 28 days.

FIG. 19 is a graph of the celecoxib release curves from silk fibroin hydrogels made using SOHO silk yarn at 3.5% (w/v) and 6.5% (w/v) silk fibroin and 100 mg/mL celecoxib over 28 days.

FIG. 20 is a graph of the celecoxib release profile from silk fibroin hydrogels prepared with either a solution or suspension of celecoxib.

FIG. 21 is a graph of the celecoxib release profile from silk fibroin hydrogels with varying silk fibroin concentrations (0.375% (w/v), 0.75% (w/v), 1.5% (w/v), 3% (w/v), or 6% (w/g) silk fibroin).

FIG. 22 is a graph of the cumulative percentage release of celecoxib from 6.5% fibroin hydrogels made with the gelling agents at the concentrations listed (50% (v/v) PEG300, 40% (v/v) PEG300, 40% (w/v) 8 kDa PEG; 30% (w/v) Sorbitol, or 40% (w/v) Sorbitol). All formulations contained 100 mg/mL celecoxib.

FIG. 23 is a graph of the serum concentration of celecoxib (ng/mL) in rats after intraarticular injection of a 6.5% (w/v) silk fibroin hydrogel including 100 mg/mL celecoxib, or a 3.5% (w/v) silk fibroin hydrogel including 100 mg/mL celecoxib, as described in Example 10.

### DETAILED DESCRIPTION

Provided herein are compositions (e.g., gels, e.g., hydrogels) that include a benzenesulfonamide-containing NSAID, silk fibroin, and a gelling agent (e.g., any of the gelling agents described herein or known in the art). In some examples, the composition (e.g., gel, e.g., hydrogel) releases a therapeutically effective amount of the benzenesulfonamide-containing NSAID over a period of 10 days to about 200 days when incubated in phosphate buffered saline (PBS) or 2% Tween-80 in PBS at 37° C. Also provided are compositions that include a gel (e.g., hydrogel) provided herein, kits that include any of the compositions provided herein, and methods of making any of the compositions (e.g., gels, e.g., hydrogels) provided herein. Also provided are methods of treating an inflammatory and/or pain condition in a subject and methods of reducing inflammation, pain, or fever in a subject in need thereof, that include administering a therapeutically effective amount of any of the compositions (e.g., gels, e.g., hydrogels) provided herein.

Administration of compositions (e.g., gels, e.g., hydrogels) provided herein allows for the extended release of a therapeutically effective amount of a benzenesulfonamide-containing NSAID for about 10 days to about 360 days, about 340 days, about 320 days, about 300 days, about 280 days, about 260 days, about 240 days, about 220 days, about 200 days, about 190 days, about 180 days, about 170 days, about 160 days, about 150 days, about 140 days, about 130 days, about 120 days, about 110 days, about 100 days, about 90 days, about 80 days, about 70 days, about 60 days, about 50 days, about 40 days, about 30 days, or about 20 days; about 20 days to about 360 days, about 340 days, about 320 days, about 300 days, about 280 days, about 260 days, about 240 days, about 220 days, about 200 days, about 190 days, about 180 days, about 170 days, about 160 days, about 150 days, about 140 days, about 130 days, about 120 days, about 110 days, about 100 days, about 90 days, about 80 days, about 70 days, about 60 days, about 50 days, about 40 days, or about 30 days; about 30 days to about 360 days, about 340 days, about 320 days, about 300 days, about 280 days, about 260 days, about 240 days, about 220 days, about 200 days, about 190 days, about 180 days, about 170 days, about 160 days, about 150 days, about 140 days, about 130 days, about 120 days, about 110 days, about 100 days, about 90 days, about 80 days, about 70 days, about 60 days, about 50 days, or about 40 days; about 40 days to about 360 days, about 340 days, about 320 days, about 300 days, about 280 days, about 260 days, about 240 days, about 220 days, about 200 days, about 190 days, about 180 days, about 170 days, about 160 days, about 150 days, about 140 days, about 130 days, about 120 days, about 110 days, about 100 days, about 90 days, about 80 days, about 70 days, about 60 days, or about 50 days; about 50 days to about 360 days, about 340 days, about 320 days, about 300 days, about 280 days, about 260 days, about 240 days, about 220 days, about 200 days, about 190 days, about 180 days, about 170 days, about 160 days, about 150 days, about 140 days, about 130 days, about 120 days, about 110 days, about 100 days, about 90 days, about 80 days, about 70 days, or about 60 days; about 60 days to about 360 days, about 340 days, about 320 days, about 300 days, about 280 days, about 260 days, about 240 days, about 220 days, about 200 days, about 190 days, about 180 days, about 170 days, about 160 days, about 150 days, about 140 days, about 130 days, about 120 days, about 110 days, about 100 days, about 90 days, about 80 days, or about 70 days; about 70 days to about 360 days, about 340 days, about 320 days, about 300 days, about 280 days, about 260 days, about 240 days, about 220 days, about 200 days, about 190 days, about 180 days, about 170 days, about 160 days, about 150 days, about 140 days, about 130 days, about 120 days, about 110 days, about 100 days, about 90 days, or about 80 days; about 80 days to about 360 days, about 340 days, about 320 days, about 300 days, about 280 days, about 260 days, about 240 days, about 220 days, about 200 days, about 190 days, about 180 days, about 170 days, about 160 days, about 150 days, about 140 days, about 130 days, about 120 days, about 110 days, about 100 days, or about 90 days; about 90 days to about 360 days, about 340 days, about 320 days, about 300 days, about 280 days, about 260 days, about 240 days, about 220 days, about 200 days, about 190 days, about 180 days, about 170 days, about 160 days, about 150 days, about 140 days, about 130 days, about 120 days, about 110 days, or about 100 days; about 100 days to about 360 days, about 340 days, about 320 days, about 300 days, about 280 days, about 260 days, about 240 days, about 220 days, about 200 days, about 190 days, about 180 days, about 170 days, about 160 days, about 150 days, about 140 days, about 130 days, about 120 days, or about 110 days; about 110 days to about 360 days, about 340 days, about 320 days, about 300 days, about 280 days, about 260 days, about 240 days, about 220 days, about 200 days, about 190 days, about 180 days, about 170 days, about 160 days, about 150 days, about 140 days, about 130 days, or about 120 days; about 120 days to about 360 days, about 340 days, about 320 days, about 300 days, about 280 days, about 260 days, about 240 days, about 220 days, about 200 days, about 190 days, about 180 days, about 170 days, about 160 days, about 150 days, about 140 days, or about 130 days; about 130 days to about 360 days, about 340 days, about 320 days, about 300 days, about 280 days, about 260 days, about 240 days, about 220 days, about 200 days, about 190 days, about 180 days, about 170 days, about 160 days, about 150 days, or about 140 days; about 140 days to about 360 days, about 340 days, about 320 days, about 300 days, about 280 days, about 260 days, about 240 days, about 220 days, about 200 days, about 190 days, about 180 days, about 170 days, about 160 days, or about 150 days; about 150 days to about 360 days, about 340 days, about 320 days, about 300 days, about 280 days, about 260 days, about 240 days, about 220 days, about 200 days, about 190 days, about 180 days, about 170 days, or about 160 days; about 160 days to about 360 days, about 340 days, about 320 days, about 300 days, about 280 days, about 260 days, about 240 days, about 220 days, about 200 days, about 190 days, about 180 days, or about 170 days; about 170 days to about 360 days, about 340 days, about 320 days, about 300 days, about 280 days, about 260 days, about 240 days, about 220 days, about 200 days, about 190 days, or about 180 days; about 180 days to about 360 days, about 340 days, about 320 days, about 300 days, about 280 days, about 260 days, about 240 days, about 220 days, about 200 days, or about 190 days; about 190 days to about 360 days, about 340 days, about 320 days, about 300 days, about 280 days, about 260 days, about 240 days, about 220 days, or about 200 days; about 200 days to about 360 days, about 340 days, about 320 days, about 300 days, about 280 days, about 260 days, about 240 days, or about 220 days; about 220 days to about 360 days, about 340 days, about 320 days, about 300 days, about 280 days, about 260 days, or about 240 days; about 240 days to about 360 days, about 340 days, about 320 days, about 300 days, about 280 days, or about 260 days; about 260 days to about 360 days, about 340 days, about 320 days, about 300 days, or about 280 days; about 280 days to about 360 days, about 340 days, about 320 days, or about 300 days; about 300 days to about 360 days, about 340 days, or about 320 days; about 320 days to about 360 days or about 340 days; or about 340 days to about 360 days; in a subject.

Administration of the compositions (e.g., gels, e.g., hydrogels) provided herein also results in a decrease in the risk of adverse side effects associated with systemic administration of an approved dose of an NSAID.

### Benzenesulfonamide-Containing NSAIDs

The compositions (e.g., gels, e.g., hydrogels) provided herein include a benzenesulfonamide-containing NSAID. In some examples, the benzenesulfonamide-containing NSAID inhibits the catalytic activity of COX-2. In other examples, the benzenesulfonamide-containing NSAID inhibits the catalytic activity of COX-1. In other examples, the benzenesulfonamide-containing NSAID inhibits the catalytic activity of both COX-1 and COX-2. For example, the benzenesulfonamide-containing NSAID can inhibit the catalytic activity of one or both of COX-1 (e.g., human COX-1) and COX-2 (e.g., human COX-2) with an IC50 of about 0.05 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, about 70 nM, about 65 nM, about 60 nM, about 55 nM, about 50 nM, about 45 nM, about 40 nM, about 35 nM, about 30 nM, about 25 nM, about 20 nM, about 15 nM, about 10 nM, about 5 nM, about 1 nM, about 0.5 nM, or about 0.1 nM; about 0.1 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, about 70 nM, about 65 nM, about 60 nM, about 55 nM, about 50 nM, about 45 nM, about 40 nM, about 35 nM, about 30 nM, about 25 nM, about 20 nM, about 15 nM, about 10 nM, about 5 nM, about 1 nM, or about 0.5 nM; about 0.5 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, about 70 nM, about 65 nM, about 60 nM, about 55 nM, about 50 nM, about 45 nM, about 40 nM, about 35 nM, about 30 nM, about 25 nM, about 20 nM, about 15 nM, about 10 nM, about 5 nM, or about 1 nM; about 1 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, about 70 nM, about 65 nM, about 60 nM, about 55 nM, about 50 nM, about 45 nM, about 40 nM, about 35 nM, about 30 nM, about 25 nM, about 20 nM, about 15 nM, about 10 nM, or about 5 nM; about 5 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, about 70 nM, about 65 nM, about 60 nM, about 55 nM, about 50 nM, about 45 nM, about 40 nM, about 35 nM, about 30 nM, about 25 nM, about 20 nM, about 15 nM, or about 10 nM; about 10 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, about 70 nM, about 65 nM, about 60 nM, about 55 nM, about 50 nM, about 45 nM, about 40 nM, about 35 nM, about 30 nM, about 25 nM, about 20 nM, or about 15 nM; about 15 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, about 70 nM, about 65 nM, about 60 nM, about 55 nM, about 50 nM, about 45 nM, about 40 nM, about 35 nM, about 30 nM, about 25 nM, or about 20 nM; about 20 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, about 70 nM, about 65 nM, about 60 nM, about 55 nM, about 50 nM, about 45 nM, about 40 nM, about 35 nM, about 30 nM, or about 25 nM; about 25 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, about 70 nM, about 65 nM, about 60 nM, about 55 nM, about 50 nM, about 45 nM, about 40 nM, about 35 nM, or about 30 nM; about 30 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, about 70 nM, about 65 nM, about 60 nM, about 55 nM, about 50 nM, about 45 nM, about 40 nM, or about 35 nM; about 35 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, about 70 nM, about 65 nM, about 60 nM, about 55 nM, about 50 nM, about 45 nM, or about 40 nM; about 40 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, about 70 nM, about 65 nM, about 60 nM, about 55 nM, about 50 nM, or about 45 nM; about 45 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, about 70 nM, about 65 nM, about 60 nM, about 55 nM, or about 50 nM; about 50 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, about 70 nM, about 65 nM, about 60 nM, or about 55 nM; about 55 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, about 70 nM, about 65 nM, or about 60 nM; about 60 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, about 70 nM, or about 65 nM; about 65 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, about 75 nM, or about 70 nM; about 70 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, about 80 nM, or about 75 nM; about 75 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, about 85 nM, or about 80 nM; about 80 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, about 90 nM, or about 85 nM; about 85 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 95 nM, or about 90 nM; about 90 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, or about 95 nM; about 95 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, or about 100 nM; about 100 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, or about 150 nM; about 150 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, or about 200 nM; about 200 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, or about 250 nM; about 250 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, or about 300 nM; about 300 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, about 400 nM, or about 350 nM; about 350 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, about 450 nM, or about 400 nM; about 400 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, about 500 nM, or about 450 nM; about 450 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, about 550 nM, or about 500 nM; about 500 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, about 600 nM, or about 550 nM; about 550 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, about 650 nM, or about 600 nM; about 600 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, about 700 nM, or about 650 nM; about 650 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, about 750 nM, or about 700 nM; about 700 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, about 800 nM, or about 750 nM; about 750 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, about 850 nM, or about 800 nM; about 800 nM to about 50 μM, about 45 μM, about 40 μM, about 35 μM, about 30 μM, about 25 μM, about 20 μM, about 15 μM, about 10 μM, about 5 μM, about 1 μM, about 950 nM, about 900 nM, or about 850 nM; about 850 nM to about 50 μM, about 45 μM, about 40 μM, about 35 μM, about 30 μM, about 25 μM, about 20 μM, about 15 μM, about 10 μM, about 5 μM, about 1 μM, about 950 nM, or about 900 nM; about 900 nM to about 50 μM, about 45 μM, about 40 μM, about 35 μM, about 30 μM, about 25 μM, about 20 μM, about 15 μM, about 10 μM, about 5 μM, about 1 μM, or about 950 nM; about 950 nM to about 50 μM, about 45 μM, about 40 μM, about 35 μM, about 30 μM, about 25 μM, about 20 μM, about 15 μM, about 10 μM, about 5 μM, or about 1 μM; about 1 μM to about 50 μM, about 45 μM, about 40 μM, about 35 μM, about 30 μM, about 25 μM, about 20 μM, about 15 μM, about 10 μM, or about 5 μM; about 5 μM to about 50 μM, about 45 μM, about 40 μM, about 35 μM, about 30 μM, about 25 μM, about 20 μM, about 15 μM, or about 10 μM; about 10 μM to about 50 μM, about 45 μM, about 40 μM, about 35 μM, about 30 μM, about 25 μM, about 20 μM, or about 15 μM; about 15 μM to about 50 μM, about 45 μM, about 40 μM, about 35 μM, about 30 μM, about 25 μM, or about 20 μM; about 20 μM to about 50 μM, about 45 μM, about 40 μM, about 35 μM, about 30 μM, or about 25 μM; about 25 μM to about 50 μM, about 45 μM, about 40 μM, about 35 μM, or about 30 μM; about 30 μM to about 50 μM, about 45 μM, about 40 μM, or about 35 μM; about 35 μM to about 50 μM, about 45 μM, or about 40 μM; about 40 μM to about 50 μM or about 45 μM; or about 45 μM to about 50 μM.

Exemplary assays for determining the catalytic activity of COX-1 and COX-2 are described in Berg et al., Inflammation Res. 48(7):369-379, 1999; Noreen et al., J. Nat. Prod. 61(1):2-7, 1998; Beretta et al., Pharmacol. Res. 52:302-306, 2005; Beretta et al., Pharmaceutical Res. 52(4):302-306, 2005; and Ben-Chetrit et al., Rheumatol. Int. 25:332-335, 2005.

In some examples, the molecular weight of the benzenesulfonamide-containing NSAID is about 50 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, about 500 g/mole, about 475 g/mole, about 450 g/mole, about 425 g/mole, about 400 g/mole, about 375 g/mole, about 350 g/mole, about 325 g/mole, about 300 g/mole, about 275 g/mole, about 250 g/mole, about 225 g/mole, about 200 g/mole, about 175 g/mole, about 150 g/mole, about 125 g/mole, or about 100 g/mole; about 100 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, about 500 g/mole, about 475 g/mole, about 450 g/mole, about 425 g/mole, about 400 g/mole, about 375 g/mole, about 350 g/mole, about 325 g/mole, about 300 g/mole, about 275 g/mole, about 250 g/mole, about 225 g/mole, about 200 g/mole, about 175 g/mole, about 150 g/mole, or about 125 g/mole; about 125 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, about 500 g/mole, about 475 g/mole, about 450 g/mole, about 425 g/mole, about 400 g/mole, about 375 g/mole, about 350 g/mole, about 325 g/mole, about 300 g/mole, about 275 g/mole, about 250 g/mole, about 225 g/mole, about 200 g/mole, about 175 g/mole, or about 150 g/mole; about 150 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, about 500 g/mole, about 475 g/mole, about 450 g/mole, about 425 g/mole, about 400 g/mole, about 375 g/mole, about 350 g/mole, about 325 g/mole, about 300 g/mole, about 275 g/mole, about 250 g/mole, about 225 g/mole, about 200 g/mole, or about 175 g/mole; about 175 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, about 500 g/mole, about 475 g/mole, about 450 g/mole, about 425 g/mole, about 400 g/mole, about 375 g/mole, about 350 g/mole, about 325 g/mole, about 300 g/mole, about 275 g/mole, about 250 g/mole, about 225 g/mole, or about 200 g/mole; about 200 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, about 500 g/mole, about 475 g/mole, about 450 g/mole, about 425 g/mole, about 400 g/mole, about 375 g/mole, about 350 g/mole, about 325 g/mole, about 300 g/mole, about 275 g/mole, about 250 g/mole, or about 225 g/mole; about 225 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, about 500 g/mole, about 475 g/mole, about 450 g/mole, about 425 g/mole, about 400 g/mole, about 375 g/mole, about 350 g/mole, about 325 g/mole, about 300 g/mole, about 275 g/mole, or about 250 g/mole; about 250 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, about 500 g/mole, about 475 g/mole, about 450 g/mole, about 425 g/mole, about 400 g/mole, about 375 g/mole, about 350 g/mole, about 325 g/mole, about 300 g/mole, or about 275 g/mole; about 275 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, about 500 g/mole, about 475 g/mole, about 450 g/mole, about 425 g/mole, about 400 g/mole, about 375 g/mole, about 350 g/mole, about 325 g/mole, or about 300 g/mole; about 300 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, about 500 g/mole, about 475 g/mole, about 450 g/mole, about 425 g/mole, about 400 g/mole, about 375 g/mole, about 350 g/mole, or about 325 g/mole; about 325 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, about 500 g/mole, about 475 g/mole, about 450 g/mole, about 425 g/mole, about 400 g/mole, about 375 g/mole, or about 350 g/mole; about 350 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, about 500 g/mole, about 475 g/mole, about 450 g/mole, about 425 g/mole, about 400 g/mole, or about 375 g/mole; about 375 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, about 500 g/mole, about 475 g/mole, about 450 g/mole, about 425 g/mole, or about 400 g/mole; about 400 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, about 500 g/mole, about 475 g/mole, about 450 g/mole, or about 425 g/mole; about 425 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, about 500 g/mole, about 475 g/mole, or about 450 g/mole; about 450 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, about 500 g/mole, or about 475 g/mole; about 475 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, about 525 g/mole, or about 500 g/mole; about 500 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, about 550 g/mole, or about 525 g/mole; about 525 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, about 575 g/mole, or about 550 g/mole; about 550 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, about 600 g/mole, or about 575 g/mole; about 575 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, about 625 g/mole, or about 600 g/mole; about 600 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, about 650 g/mole, or about 625 g/mole; about 625 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, about 675 g/mole, or about 650 g/mole; about 650 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, about 700 g/mole, or about 675 g/mole; about 675 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, about 725 g/mole, or about 700 g/mole; about 700 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, about 750 g/mole, or about 725 g/mole; about 725 g/mole to about 1,000 g/mole, about 975 g/mole, about 950 g/mole, about 925 g/mole, about 900 g/mole, about 875 g/mole, about 850 g/mole, about 825 g/mole, about 800 g/mole, about 775 g/mole, or about 750 g/mole; about 750 g/mole to about 1,000 g/mole, about 975 g/mole, about 950 g/mole, about 925 g/mole, about 900 g/mole, about 875 g/mole, about 850 g/mole, about 825 g/mole, about 800 g/mole, or about 775 g/mole; about 775 g/mole to about 1,000 g/mole, about 975 g/mole, about 950 g/mole, about 925 g/mole, about 900 g/mole, about 875 g/mole, about 850 g/mole, about 825 g/mole, or about 800 g/mole; about 800 g/mole to about 1,000 g/mole, about 975 g/mole, about 950 g/mole, about 925 g/mole, about 900 g/mole, about 875 g/mole, about 850 g/mole, or about 825 g/mole; about 825 g/mole to about 1,000 g/mole, about 975 g/mole, about 950 g/mole, about 925 g/mole, about 900 g/mole, about 875 g/mole, or about 850 g/mole; about 850 g/mole to about 1,000 g/mole, about 975 g/mole, about 950 g/mole, about 925 g/mole, about 900 g/mole, or about 875 g/mole; about 875 g/mole to about 1,000 g/mole, about 975 g/mole, about 950 g/mole, about 925 g/mole, or about 900 g/mole; about 900 g/mole to about 1,000 g/mole, about 975 g/mole, about 950 g/mole, or about 925 g/mole; about 925 g/mole to about 1,000 g/mole, about 975 g/mole, or about 950 g/mole; about 950 g/mole to about 1,000 g/mole or about 975 g/mole; or about 975 g/mole to about 1,000 g/mole.

Non-limiting examples of benzenesulfonamide-containing NSAIDs, as well as methods of synthesizing benzenesulfonamide-containing NSAIDs, are described in U.S. Pat. Nos. 5,466,823, 5,504,215, 5,508,426, 5,510,496, 5,516,907, 5,521,207, 5,563,165, 5,753,688, 5,760,068, 6,156,781, 6,413,960, 6,492,411, 6,586,603, 6,716,991, and 6,951,949; U.S. Patent Application Publication No. 2005/0131050; and WO 95/15316 (each which is incorporated by reference in its entirety). Additional examples of benzenesulfonamide-containing NSAIDs, as well as methods of synthesizing benzenesulfonamide-containing NSAIDs, are described in Krause et al., Arthritis Rheumatism 48:1438-1444, 2003.

Non-limiting examples of benzenesulfonamide-containing NSAIDs have the structure of Formula I:

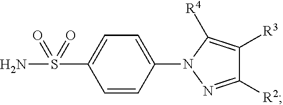

where R² is selected from hydrido, alkyl, haloalkyl, alkoxycarbonyl, cyano, cyanoalkyl, carboxyl, aminocarbonyl, alkylaminocarbonyl, cycloalkylaminocarbonyl, arylaminocarbonyl, carboxyalkylaminocarbonyl, carboxyalkyl, aralkoxycarbonylalkylaminocarbonyl, aminocarbonylalkyl, alkoxycarbonylcyanoalkenyl, and hydroxyalkyl;

where R³ is selected from hydrido, alkyl, cyano, hydroxyalkyl, cycloalkyl, alkylsulfonyl, and halo; and

where R⁴ is selected from aralkenyl, aryl, cycloalkyl, cycloalkenyl, and heterocyclic; where R⁴ is optionally substituted at a substitutable position with one or more radicals selected from halo, alkylthio, alkylsulfonyl, cyano, nitro, haloalkyl, alkyl, hydroxyl, alkenyl, hydroxyalkyl, carboxyl, cycloalkyl, alkylamino, dialkylamino, alkoxycarbonyl, aminocarbonyl, alkoxy, haloalkoxy, sulfamyl, heterocyclic, and amino;

provided R² and R³ are not both hydrido;

further provided that R² is not carboxyl or methyl when R³ is hydrido and when R⁴ is phenyl;

further provided that R⁴ is not triazolyl when R² is methyl;

further provided that R⁴ is not aralkenyl when R² is carboxyl, aminocarbonyl, or ethoxycarbonyl;

further provided that R⁴ is not phenyl when R² is methyl and R³ is carboxyl; and

further provided that R⁴ is not unsubstituted thienyl when R² is trifluoromethyl; or a pharmaceutically-acceptable salt thereof.

Non-limiting examples of benzenesulfonamide-containing NSAIDs include:
4-[5-(4-(N-ethylamino)phenyl)-3-(trifluoromethyl)-1H-pyrazol-1-yl]benzenesulfonamide;
4-[5-(4-(N-ethyl-N-methylamino)phenyl)-3-(trifluoromethyl)-1H-pyrazol-1-yl] benzenesulfonamide;
4-[5-(3-fluoro-4-(N-methylamino)phenyl)-3-(trifluoromethyl)-1H-pyrazol-1-yl] benzenesulfonamide;
4-[5-(3-chloro-4-(N-methyl amino)phenyl)-3-(trifluoromethyl)-1H-pyrazol-1-yl]benzenesulfonamide;
4-[5-(3-methyl-4-(N-methylamino)phenyl)-3-(trifluoromethyl)-1H-pyrazol-1-yl]benzenesulfonamide;
4-[5-(4-(N,N-dimethylamino)-3-fluorophenyl)-3-(trifluoromethyl)-1H-pyrazol-1-yl]benzenesulfonamide;
4-[5-(3-chloro-4-(N,N-dimethylamino)phenyl)-3-(trifluoromethyl)-1H-pyrazol-1-yl]benzenesulfonamide;
4-[5-(4-(N,N-dimethylamino)-3-methylphenyl)-3-(trifluoromethyl)-1H-pyrazol-1-yl]benzenesulfonamide;
4-[5-(4-(N-ethyl-N-methylamino)-3-fluorophenyl)-3-(trifluoromethyl)-1H-pyrazol-1-yl]benzenesulfonamide;
4-[5-(3-chloro-4-(N-ethyl-N-methylamino)phenyl)-3-(trifluoromethyl)-1H-pyrazol-1-yl]benzenesulfonamide;
4-[5-(4-(N-ethyl-N-methylamino)-3-methylphenyl)-3-(trifluoromethyl)-1H-pyrazol-1-yl]benzenesulfonamide;
4-[5-(4-(N,N-diethylamino)-3-fluorophenyl)-3-(trifluoromethyl)-1H-pyrazol-1-yl]benzenesulfonamide;
4-[5-(4-(3-chloro-4-(N,N-diethylamino)phenyl)-3-(trifluoromethyl)-1H-pyrazol-1-yl]benzenesulfonamide;
4-[5-(4-(N,N-diethylamino)-3-methylphenyl)-3-(trifluoromethyl)-1H-pyrazol-1-yl]benzenesulfonamide;
4-[5-(3-(N,N-dimethylamino)phenyl)-3-(trifluoromethyl)-1H-pyrazol-1-yl]benzenesulfonamide;
4-[5-(3-(N-ethyl-N-methylamino)phenyl)-3-(trifluoromethyl)-1H-pyrazol-1-yl]benzenesulfonamide;
4-[5-(4-chloro-3-(N-methyl amino)phenyl)-3-(trifluoromethyl)-1H-pyrazol-1-yl]benzenesulfonamide;
4-[5-(4-methyl-3-(N-methylamino)phenyl)-3-(trifluoromethyl)-1H-pyrazol-1-yl]benzenesulfonamide;
4-[5-(2-(N-ethyl-N-methylamino)-4-methylphenyl)-3-(trifluoromethyl)-1H-pyrazol-1-yl]benzenesulfonamide;
4-[5-(1H-indol-5-yl)-3-(trifluoromethyl)-1H-pyrazol-1-yl]benzenesulfonamide;
4-[5-(7-fluoro-1H-indol-5-yl)-3-(trifluoromethyl)-1H-pyrazol-1-yl]benzenesulfonamide;
4-[5-(1-ethyl-1H-indol-5-yl)-3-(trifluoromethyl)-1H-pyrazol-1-yl]benzenesulfonamide;
4-[5-(7-methyl-1H-indol-5-yl)-3-(trifluoromethyl)-1H-pyrazol-1-yl]benzenesulfonamide;
4-[5-(7-chloro-1-methyl-1H-indol-5-yl)-3-(trifluoromethyl)-1H-pyrazol-1-yl]benzenesulfonamide;
4-[5-(2,3-dihydro-1H-indol-5-yl)-3-(trifluoromethyl)-1H-pyrazol-1-yl]benzenesulfonamide;
4-[5-(7-fluoro-1-methyl-2,3-dihydro-1H-indol-5-yl)-3-(trifluoromethyl)-1H-pyrazol-1-yl]benzenesulfonamide;
4-[3-μminomethyl-5-phenyl-1H-pyrazol-1-yl]benzenesulfonamide;
4-[3-(N-methylamino)methyl-5-phenyl-1H-pyrazol-1-yl]benzenesulfonamide;
4-[3-(N,N-dimethylamino)methyl-5-phenyl-1H-pyrazol-1-yl]benzenesulfonamide;
4-[5-phenyl-3-(N-phenylamino)methyl-1H-pyrazol-1-yl]benzenesulfonamide;
4-[3-(N-benzylamino)methyl-5-phenyl-1H-pyrazol-1-yl]benzenesulfonamide;
4-[3-(N-benzyl-N-methylamino)methyl-5-phenyl-1H-pyrazol-1-yl]benzenesulfonamide;
4-[3-(N-methyl-N-phenylamino)methyl-5-phenyl-1H-pyrazol-1-yl]benzenesulfonamide;
4-[4-methoxy-5-phenyl-3-(trifluoromethyl)-1H-pyrazol-1-yl]benzenesulfonamide;
4-[4-methylthio-5-phenyl-3-(trifluoromethyl)-1H-pyrazol-1-yl]benzenesulfonamide;
4-[4-(N-methylamino)-5-phenyl-3-(trifluoromethyl)-1H-pyrazol-1-yl]benzenesulfonamide;
4-[4-(N,N-dimethylamino)-5-phenyl-3-(trifluoromethyl)-1H-pyrazol-1-yl]benzenesulfonamide;
4-[3-methoxy-5-phenyl-1H-pyrazol-1-yl]benzenesulfonamide;
4-[3-ethoxy-5-phenyl-1H-pyrazol-1-yl]benzenesulfonamide;
4-[3-phenoxy-5-phenyl-1H-pyrazol-1-yl]benzenesulfonamide;
4-[3-benzyloxy-5-phenyl-1H-pyrazol-1-yl]benzenesulfonamide;
4-[3-methylthio-5-phenyl-1H-pyrazol-1-yl]benzenesulfonamide;
4-[3-benzylthio-5-phenyl-1H-pyrazol-1-yl]benzenesulfonamide;
4-[3-(N-methylamino)-5-phenyl-1H-pyrazol-1-yl]benzenesulfonamide;
4-[3-(N,N-dimethylamino)-5-phenyl-1H-pyrazol-1-yl]benzenesulfonamide;
4-[3-(N-benzyl-N-methylamino)-5-phenyl-1H-pyrazol-1-yl]benzenesulfonamide;
4-[5-phenyl-3-(1,1-difluoro-1-phenylmethyl)-1H-pyrazol-1-yl]benzenesulfonamide;
4-[5-phenyl-3-(1,1-difluoro-2-phenylethyl)-1H-pyrazol-1-yl]benzenesulfonamide;
2-fluoro-4-[5-phenyl-3-(trifluoromethyl)-1H-pyrazol-1-yl]benzenesulfonamide;
3-fluoro-4-[5-phenyl-3-(trifluoromethyl)-1H-pyrazol-1-yl]benzenesulfonamide;
2-methyl-4-[5-phenyl-3-(trifluoromethyl)-1H-pyrazol-1-yl]benzenesulfonamide;
3-methyl-4-[5-phenyl-3-(trifluoromethyl)-1H-pyrazol-1-yl]benzenesulfonamide;
4-[5-(4-chlorophenyl)-3-(pyrrolidinocarboxamide)-1H-pyrazol-1-yl]benzenesulfonamide;
4-[5-(4-chlorophenyl)-3-(piperidinocarboxamide)-1H-pyrazol-1-yl]benzenesulfonamide;
4-[5-(4-bromophenyl)-3-cyano-1H-pyrazol-1-yl]benzenesulfonamide;
4-[3-cyano-5-(4-fluorophenyl)-1H-pyrazol-1-yl]benzenesulfonamide;
4-[5-(4-chlorophenyl)-3-cyano-1H-pyrazol-1-yl]benzenesulfonamide;
4-[3-cyano-5-(4-methoxyphenyl)-1H-pyrazol-1-yl]benzenesulfonamide;
4-[3-cyano-5-(4-methylphenyl)-1H-pyrazol-1-yl]benzenesulfonamide;
4-[3-cyano-5-(4-methylthiophenyl)-1H-pyrazol-1-yl]benzenesulfonamide;
4-[5-(3-chloro-4-methoxyphenyl)-3-cyano-1H-pyrazol-1-yl]benzenesulfonamide;
4-[5-(3,5-dichloro-4-methoxyphenyl)-3-cyano-1H-pyrazol-1-yl]benzenesulfonamide;
4-[5-(3-bromo-4-methoxyphenyl)-3-cyano-1H-pyrazol-1-yl]benzenesulfonamide;
4-[3-cyano-5-phenyl-1H-pyrazol-1-yl]benzenesulfonamide;
4-[5-(4-nitrophenyl)-3-(cyano)-1H-pyrazol-1-yl]benzenesulfonamide;
4-[4-chloro-5-(4-fluorophenyl)-1H-pyrazol-1-yl]benzenesulfonamide;
4-[4-chloro-5-(4-chlorophenyl)-1H-pyrazol-1-yl]benzenesulfonamide;
4-[4-bromo-5-(4-chlorophenyl)-1H-pyrazol-1-yl]benzenesulfonamide;
4-[4-chloro-5-phenyl-1H-pyrazol-1-yl] benzenesulfonamide;
4-[4-chloro-5-(3,5-dichloro-4-methoxyphenyl)-1H-pyrazol-1-yl]benzenesulfonamide;
4-[4-bromo-5-(4-methylphenyl)-1H-pyrazol-1-yl]benzenesulfonamide;
4-[4-chloro-5-(4-methylphenyl)-1H-pyrazol-1-yl]benzenesulfonamide;
4-[4-chloro-5-(3-chloro-4-methoxyphenyl)-1H-pyrazol-1-yl]benzenesulfonamide;
4-[4-chloro-5-(4-methoxyphenyl)-1H-pyrazol-1-yl]benzenesulfonamide;
4-[4-bromo-5-(4-methoxyphenyl)-1H-pyrazol-1-yl]benzenesulfonamide;
4-[4-cyano-5-(4-methoxyphenyl)-1H-pyrazol-1-yl]benzenesulfonamide;
4-[4-chloro-5-(3,5-difluoro-4-methoxyphenyl)-1H-pyrazol-1-yl]benzenesulfonamide;
4-[4-methyl-5-phenyl-1H-pyrazol-1-yl]benzenesulfonamide;
4-[4-fluoro-5-phenyl-1H-pyrazol-1-yl]benzenesulfonamide;
4-[5-(4-chlorophenyl)-4-methyl sulfonyl-1H-pyrazol-1-yl]benzenesulfonamide;
4-[4-chloro-5-(4-chlorophenyl)-3-(trifluoromethyl)-1H-pyrazol-1-yl]benzenesulfonamide;
4-[4-ethyl-5-phenyl-3-(trifluoromethyl)-1H-pyrazol-1-yl]benzenesulfonamide;
4-[4-methyl-5-phenyl-3-(trifluoromethyl)-1H-pyrazol-1-yl]benzenesulfonamide;
4-[5-(4-methoxyphenyl)-4-methyl-3-(trifluoromethyl)-1H-pyrazol-1-yl]benzenesulfonamide;
4-[5-(4-chlorophenyl)-4-methyl-3-(trifluoromethyl)-1H-pyrazol-1-yl]benzenesulfonamide;
4-[5-(4-chlorophenyl)-4-ethyl-3-(trifluoromethyl)-1H-pyrazol-1-yl]benzenesulfonamide;
4-[4-ethyl-5-(4-methylphenyl)-3-(trifluoromethyl)-1H-pyrazol-1-yl]benzenesulfonamide;
4-[4-ethyl-5-(4-methoxy-3-methylphenyl)-3-(trifluoromethyl)-1H-pyrazol-1-yl]benzenesulfonamide;
4-[4-ethyl-5-(4-methoxyphenyl)-3-(trifluoromethyl)-1H-pyrazol-1-yl]benzenesulfonamide;
4-[4-cyclopropyl-5-phenyl-3-(trifluoromethyl)-1H-pyrazol-1-yl]benzenesulfonamide;
4-[4-ethyl-5-(3-fluoro-4-chlorophenyl)-3-(trifluoromethyl)-1H-pyrazol-1-yl]benzenesulfonamide;
4-[4-hydroxymethyl-5-phenyl-3-(trifluoromethyl)-1H-pyrazol-1-yl]benzenesulfonamide;
4-[5-(4-fluorophenyl)-4-methyl-3-(trifluoromethyl)-1H-pyrazol-1-yl]benzenesulfonamide;
4-[4-methyl-5-(4-methylphenyl)-3-(trifluoromethyl)-1H-pyrazol-1-yl]benzenesulfonamide;
4-[4-fluoro-5-phenyl-3-(trifluoromethyl)-1H-pyrazol-1-yl]benzenesulfonamide;
4-[4-bromo-5-(4-chlorophenyl)-3-(difluoromethyl)-1H-pyrazol-1-yl]benzenesulfonamide;
4-[4-chloro-5-(3,5-dichloro-4-methoxyphenyl)-3-(difluoromethyl)-1H-pyrazol-1-yl]benzenesulfonamide;
4-[4-chloro-3-(difluoromethyl)-5-phenyl-1H-pyrazol-1-yl]benzenesulfonamide;
4-[4-bromo-3-(difluoromethyl)-5-phenyl-1H-pyrazol-1-yl]benzenesulfonamide;
4-[4-chloro-3-(difluoromethyl)-5-(4-methoxyphenyl)-1H-pyrazol-1-yl]benzenesulfonamide;
4-[4-chloro-3-cyano-5-phenyl-1H-pyrazol-1-yl]benzenesulfonamide;
4-[4-chloro-5-(4-chlorophenyl)-3-cyano-1H-pyrazol-1-yl]benzenesulfonamide;
4-[4-chloro-3-cyano-5-(4-fluorophenyl)-1H-pyrazol-1-yl]benzenesulfonamide;
4-[4-bromo-3-cyano-5-(4-fluorophenyl)-1H-pyrazol-1-yl]benzenesulfonamide;
4-[4-bromo-3-cyano-5-phenyl-1H-pyrazol-1-yl]benzenesulfonamide;
4-[4-chloro-3-isopropyl-5-phenyl-1H-pyrazol-1-yl]benzenesulfonamide;
4-[4-chloro-3-methyl-5-phenyl-1H-pyrazol-1-yl]benzenesulfonamide;
4-[4-chloro-3-hydroxymethyl-5-phenyl-1H-pyrazol-1-yl]benzenesulfonamide;
4-[4-chloro-5-(4-chlorophenyl)-3-hydroxymethyl-1H-pyrazol-1-yl]benzenesulfonamide;
4-[5-(4-chlorophenyl)-3-(trifluoromethyl)-1H-pyrazol-1-yl]benzenesulfonamide;
4-[5-phenyl-3-(trifluoromethyl)-1H-pyrazol-1-yl]benzenesulfonamide;
4-[5-(4-fluorophenyl)-3-(trifluoromethyl)-1H-pyrazol-1-yl]benzenesulfonamide;
4-[5-(4-cyanophenyl)-3-(trifluoromethyl)-1H-pyrazol-1-yl]benzenesulfonamide;
4-[5-(2,4-difluorophenyl)-3-(trifluoromethyl)-1H-pyrazol-1-yl]benzenesulfonamide;
4-[5-(2,6-difluorophenyl)-3-(trifluoromethyl)-1H-pyrazol-1-yl]benzenesulfonamide;
4-[5-(4-methoxyphenyl)-3-(trifluoromethyl)-1H-pyrazol-1-yl]benzenesulfonamide;
4-[5-(3,4-dichlorophenyl)-3-(trifluoromethyl)-1H-pyrazol-1-yl]benzenesulfonamide;
4-[5-(4-bromophenyl)-3-(trifluoromethyl)-1H-pyrazol-1-yl]benzenesulfonamide;
4-[5-(2,4-dichlorophenyl)-3-(trifluoromethyl)-1H-pyrazol-1-yl]benzenesulfonamide;
4-[5-(3-chlorophenyl)-3-(trifluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[5-(4-trifluoromethylphenyl)-3-(trifluoromethyl)-1H-pyrazol-1-yl]benzenesulfonamide;
4-[5-(4-trifluoromethoxyphenyl)-3-(trifluoromethyl)-1H-pyrazol-1-yl]benzenesulfonamide;
4-[5-(4-nitrophenyl)-3-(trifluoromethyl)-1H-pyrazol-1-yl]benzenesulfonamide;
4-[5-(2-chlorophenyl)-3-(trifluoromethyl)-1H-pyrazol-1-yl]benzenesulfonamide;
4-[5-(2-fluorophenyl)-3-(trifluoromethyl)-1H-pyrazol-1-yl]benzenesulfonamide;
4-[5-(4-μminophenyl)-3-(trifluoromethyl)-1H-pyrazol-1-yl]benzenesulfonamide;
4-[5-(2-methylphenyl)-3-(trifluoromethyl)-1H-pyrazol-1-yl]benzenesulfonamide;
4-[5-(4-fluoro-2-methylphenyl)-3-(trifluoromethyl)-1H-pyrazol-1-yl]benzenesulfonamide;
4-[5-(3-methylphenyl)-3-(trifluoromethyl)-1H-pyrazol-1-yl]benzenesulfonamide;
4-[5-(4-ethoxyphenyl)-3-(trifluoromethyl)-1H-pyrazol-1-yl]benzenesulfonamide;
4-[5-(3,5-dimethyl-4-methoxyphenyl)-3-(trifluoromethyl)-1H-pyrazol-1-yl]benzenesulfonamide;
4-[5-(3-fluorophenyl)-3-(trifluoromethyl)-1H-pyrazol-1-yl]benzenesulfonamide;
4-[5-(3-fluoro-4-methoxyphenyl)-3-(trifluoromethyl)-1H-pyrazol-1-yl]benzenesulfonamide;
4-[5-(4-methylthiophenyl)-3-(trifluoromethyl)-1H-pyrazol-1-yl]benzenesulfonamide;
4-[5-(4-chloro-3-methylphenyl)-3-(trifluoromethyl)-1H-pyrazol-1-yl]benzenesulfonamide;
4-[5-(4-ethylphenyl)-3-(trifluoromethyl)-1H-pyrazol-1-yl]benzenesulfonamide;
4-[5-(2,4-dimethylphenyl)-3-(trifluoromethyl)-1H-pyrazol-1-yl]benzenesulfonamide;
4-[5-(2-methoxyphenyl)-3-(trifluoromethyl)-1H-pyrazol-1-yl]benzenesulfonamide;
4-[5-(4-methoxy-3-methylphenyl)-3-(trifluoromethyl)-1H-pyrazol-1-yl]benzenesulfonamide;
4-[5-(3-bromo-4-methylthiophenyl)-3-(trifluoromethyl)-1H-pyrazol-1-yl]benzenesulfonamide;
4-[5-(4-hydroxy-3-methylphenyl)-3-(trifluoromethyl)-1H-pyrazol-1-yl]benzenesulfonamide;
4-[5-(3-chloro-4-methylphenyl)-3-(trifluoromethyl)-1H-pyrazol-1-yl]benzenesulfonamide;
4-[5-(3,4-dimethoxyphenyl)-3-(trifluoromethyl)-1H-pyrazol-1-yl]benzenesulfonamide;
4-[5-(3-chloro-4-methoxyphenyl)-3-(trifluoromethyl)-1H-pyrazol-1-yl]benzenesulfonamide;
4-[5-(3-chloro-4-methoxy-5-methylphenyl)-3-(trifluoromethyl)-1H-pyrazol-1-yl]benzenesulfonamide;
4-[5-(3-ethyl-4-methoxyphenyl)-3-(trifluoromethyl)-1H-pyrazol-1-yl]benzenesulfonamide;
4-[5-(4-fluoro-2-methoxyphenyl)-3-(trifluoromethyl)-1H-pyrazol-1-yl]benzenesulfonamide;
4-[5-(4-hydroxymethylphenyl)-3-(trifluoromethyl)-1H-pyrazol-1-yl]benzenesulfonamide;
4-[5-(4-methoxy-3-(1-propenyl)phenyl)-3-(trifluoromethyl)-1H-pyrazol-1-yl]benzenesulfonamide;
4-[5-(3,5-dichloro-4-methoxyphenyl)-3-(trifluoromethyl)-1H-pyrazol-1-yl]benzenesulfonamide;
4-[5-(2,4-dimethoxyphenyl)-3-(trifluoromethyl)-1H-pyrazol-1-yl]benzenesulfonamide;
4-[5-(3-chloro-4-fluorophenyl)-3-(trifluoromethyl)-1H-pyrazol-1-yl]benzenesulfonamide;
4-[5-(4-methoxy-3-propylphenyl)-3-(trifluoromethyl)-1H-pyrazol-1-yl]benzenesulfonamide;
4-[5-(3,5-difluoro-4-methoxyphenyl)-3-(trifluoromethyl)-1H-pyrazol-1-yl]benzenesulfonamide;
4-[5-(3-fluoro-4-methylthiophenyl)-3-(trifluoromethyl)-1H-pyrazol-1-yl]benzenesulfonamide;
4-[5-(3-cyclopropylmethyl-4-methoxyphenyl)-3-(trifluoromethyl)-1H-pyrazol-1-yl]benzenesulfonamide;
4-[1-[4-(aminosulfonyl)phenyl]-3-(trifluoromethyl)-1H-pyrazol-5-yl]benzoic acid;
4-[5-(3-methyl-4-methylthiophenyl)-3-(trifluoromethyl)-1H-pyrazol-1-yl]benzenesulfonamide;
4-[5-(3-chloro-4-methylthiophenyl)-3-(trifluoromethyl)-1H-pyrazol-1-yl]benzenesulfonamide;
4-[5-(4-(N,N-dimethylamino)phenyl)-3-(trifluoromethyl)-1H-pyrazol-1-yl]benzenesulfonamide;
4-[5-(4-methyl-3-nitrophenyl)-3-(trifluoromethyl)-1H-pyrazol-1-yl]benzenesulfonamide;
4-[5-(4-(N-methyl amino)phenyl)-3-(trifluoromethyl)-1H-pyrazol-1-yl]benzenesulfonamide;
4-[5-(3-μmino-4-methylphenyl)-3-(trifluoromethyl)-1H-pyrazol-1-yl]benzenesulfonamide;
4-[5-(3,5-difluorophenyl)-3-(trifluoromethyl)-1H-pyrazol-1-yl]benzenesulfonamide;
4-[5-(2,4,6-trifluorophenyl)-3-(trifluoromethyl)-1H-pyrazol-1-yl]benzenesulfonamide;
4-[5-(2,6-dichlorophenyl)-3-(trifluoromethyl)-1H-pyrazol-1-yl]benzenesulfonamide;
4-[5-(2,4,6-trichlorophenyl)-3-(trifluoromethyl)-1H-pyrazol-1-yl]benzenesulfonamide;
4-[5-(3-methoxyphenyl)-3-(trifluoromethyl)-1H-pyrazol-1-yl]benzenesulfonamide;
4-[5-(3,4-dimethylphenyl)-3-(trifluoromethyl)-1H-pyrazol-1-yl]benzenesulfonamide;
4-[5-(1,3-benzodioxol-5-yl)-3-(trifluoromethyl)-1H-pyrazol-1-yl]benzenesulfonamide;
4-[5-(2-fluoro-4-methoxyphenyl)-3-(trifluoromethyl)-1H-pyrazol-1-yl]benzenesulfonamide;
4-[5-(2-chloro-4-methoxyphenyl)-3-(trifluoromethyl)-1H-pyrazol-1-yl]benzenesulfonamide;
4-[5-(4-chloro-2-methoxyphenyl)-3-(trifluoromethyl)-1-H-pyrazol-1-yl]benzenesulfonamide;
4-[5-(2-methylthiophenyl)-3-(trifluoromethyl)-1H-pyrazol-1-yl]benzenesulfonamide;
4-[5-(3-methylthiophenyl)-3-(trifluoromethyl)-1H-pyrazol-1-yl]benzenesulfonamide;
4-[5-(2-methylsulfinylphenyl)-3-(trifluoromethyl)-1H-pyrazol-1-yl]benzenesulfonamide;
4-[5-(3-methylsulfinylphenyl)-3-(trifluoromethyl)-1H-pyrazol-1-yl]benzenesulfonamide;
4-[5-(4-methylsulfinylphenyl)-3-(trifluoromethyl)-1H-pyrazol-1-yl]benzenesulfonamide;
4-[5-(2-fluoro-4-methylphenyl)-3-(trifluoromethyl)-1H-pyrazol-1-yl]benzenesulfonamide;
4-[5-(4-fluoro-3-methylphenyl)-3-(trifluoromethyl)-1H-pyrazol-1-yl]benzenesulfonamide;
4-[5-(2-chloro-4-methylphenyl)-3-(trifluoromethyl)-1H-pyrazol-1-yl]benzenesulfonamide;
4-[5-(4-chloro-2-methylphenyl)-3-(trifluoromethyl)-1H-pyrazol-1-yl]benzenesulfonamide;
4-[5-(4-hydroxyphenyl)-3-(trifluoromethyl)-1H-pyrazol-1-yl]benzenesulfonamide;
4-[5-(3,4-dihydroxyphenyl)-3-(trifluoromethyl)-1H-pyrazol-1-yl]benzenesulfonamide;
4-[5-(4-isopropylphenyl)-3-(trifluoromethyl)-1H-pyrazol-1-yl]benzenesulfonamide;
4-[5-(4-[N-methyl aminosulfonyl]phenyl)-3-(trifluoromethyl)-1H-pyrazol-1-yl]benzenesulfonamide;
4-[5-(2,5-dichlorophenyl)-3-(trifluoromethyl)-1H-pyrazol-1-yl]benzenesulfonamide;
4-[5-(4-n-butoxyphenyl)-3-(trifluoromethyl)-1H-pyrazol-1-yl]benzenesulfonamide;
4-[5-(4-[aminosulfonyl]phenyl)-3-(trifluoromethyl)-1H-pyrazol-1-yl]benzenesulfonamide;
4-[5-(2,3-difluorophenyl)-3-(trifluoromethyl)-1H-pyrazol-1-yl]benzenesulfonamide;
4-[5-(2,5-difluorophenyl)-3-(trifluoromethyl)-1H-pyrazol-1-yl]benzenesulfonamide;
4-[5-(2,3,4-trifluorophenyl)-3-(trifluoromethyl)-1H-pyrazol-1-yl]benzenesulfonamide;
4-[5-(3,4,5-trifluorophenyl)-3-(trifluoromethyl)-1H-pyrazol-1-yl]benzenesulfonamide;
4-[5-(2,4,5-trifluorophenyl)-3-(trifluoromethyl)-1H-pyrazol-1-yl]benzenesulfonamide;
4-[5-(2,5,6-trifluorophenyl)-3-(trifluoromethyl)-1H-pyrazol-1-yl]benzenesulfonamide;
4-[5-(2,3,4,5-tetrafluorophenyl)-3-(trifluoromethyl)-1H-pyrazol-1-yl]benzenesulfonamide;
4-[5-(2,3,4,6-tetrafluorophenyl)-3-(trifluoromethyl)-1H-pyrazol-1-yl]benzenesulfonamide;
4-[5-(2,3,5,6-tetrafluorophenyl)-3-(trifluoromethyl)-1H-pyrazol-1-yl]benzenesulfonamide;
4-[5-(pentafluorophenyl)-3-(trifluoromethyl)-1H-pyrazol-1-yl]benzenesulfonamide;
4-[5-(2,3,4-trichlorophenyl)-3-(trifluoromethyl)-1H-pyrazol-1-yl]benzenesulfonamide;
4-[5-(3,4,5-trichlorophenyl)-3-(trifluoromethyl)-1H-pyrazol-1-yl]benzenesulfonamide;
4-[5-(2,4,5-trichlorophenyl)-3-(trifluoromethyl)-1H-pyrazol-1-yl]benzenesulfonamide;
4-[5-(2,5,6-trichlorophenyl)-3-(trifluoromethyl)-1H-pyrazol-1-yl]benzenesulfonamide;
4-[5-(2,3,4,5-tetrachlorophenyl)-3-(trifluoromethyl)-1H-pyrazol-1-yl]benzenesulfonamide;
4-[5-(2,3,4,6-tetrachlorophenyl)-3-(trifluoromethyl)-1H-pyrazol-1-yl]benzenesulfonamide;
4-[5-(2,3,5,6-tetrachlorophenyl)-3-(trifluoromethyl)-1H-pyrazol-1-yl]benzenesulfonamide;
4-[5-(2,3,4,5,6-pentachlorophenyl)-3-(trifluoromethyl)-1H-pyrazol-1-yl]benzenesulfonamide;
4-[5-(4-tert-butylphenyl)-3-(trifluoromethyl)-1H-pyrazol-1-yl]benzenesulfonamide;
4-[5-(4-isobutylphenyl)-3-(trifluoromethyl)-1H-pyrazol-1-yl]benzenesulfonamide;
4-[5-(4-chlorophenyl)-3-(difluoromethyl)-1H-pyrazol-1-yl]benzenesulfonamide;
4-[5-(4-trifluoromethylphenyl)-3-(difluoromethyl)-1H-pyrazol-1-yl]benzenesulfonamide;
4-[5-(4-methylthiophenyl)-3-(difluoromethyl)-1H-pyrazol-1-yl]benzenesulfonamide;
4-[5-(4-(1-morpholino)phenyl)-3-(difluoromethyl)-1H-pyrazol-1-yl]benzenesulfonamide;
4-[5-(4-methylphenyl)-3-(difluoromethyl)-1H-pyrazol-1-yl]benzenesulfonamide;
4-[5-phenyl-3-(difluoromethyl)-1H-pyrazol-1-yl]benzenesulfonamide;
4-[5-(4-methoxyphenyl)-3-(difluoromethyl)-1H-pyrazol-1-yl]benzenesulfonamide;
4-[5-(3,4-dimethylphenyl)-3-(difluoromethyl)-1H-pyrazol-1-yl]benzenesulfonamide;
4-[5-(3-fluoro-4-methoxyphenyl)-3-(difluoromethyl)-1H-pyrazol-1-yl]benzenesulfonamide;
4-[5-(2-fluoro-4-methoxyphenyl)-3-(difluoromethyl)-1H-pyrazol-1-yl]benzenesulfonamide;
4-[5-(4-cyanophenyl)-3-(difluoromethyl)-1H-pyrazol-1-yl]benzenesulfonamide;
4-[5-(3-chloro-4-methylphenyl)-3-(difluoromethyl)-1H-pyrazol-1-yl]benzenesulfonamide;
4-[5-(3-chloro-4-methoxyphenyl)-3-(difluoromethyl)-1H-pyrazol-1-yl]benzenesulfonamide;
4-[5-(4-chloro-3-methylphenyl)-3-(difluoromethyl)-1H-pyrazol-1-yl]benzenesulfonamide;
4-[5-(3,4-dimethoxyphenyl)-3-(difluoromethyl)-1H-pyrazol-1-yl]benzenesulfonamide;
4-[5-(3,5-dichloro-4-methoxyphenyl)-3-(difluoromethyl)-1H-pyrazol-1-yl]benzenesulfonamide;
4-[5-(3,5-difluoro-4-methoxyphenyl)-3-(difluoromethyl)-1H-pyrazol-1-yl]benzenesulfonamide;
4-[5-(2-methoxyphenyl)-3-(difluoromethyl)-1H-pyrazol-1-yl]benzenesulfonamide;
4-[5-(3-bromo-4-methoxyphenyl)-3-(difluoromethyl)-1H-pyrazol-1-yl]benzenesulfonamide;
4-[5-(4-methyl sulfonylphenyl)-3-(difluoromethyl)-1H-pyrazol-1-yl]benzenesulfonamide;
4-[5-(5-bromo-2-thienyl)-3-(difluoromethyl)-1H-pyrazol-1-yl]benzenesulfonamide;
4-[5-(5-chloro-2-thienyl)-3-(difluoromethyl)-1H-pyrazol-1-yl]benzenesulfonamide;
4-[5-(1-cyclohexenyl)-3-(difluoromethyl)-1H-pyrazol-1-yl]benzenesulfonamide;
4-[5-(cyclohexyl)-3-(difluoromethyl)-1H-pyrazol-1-yl]benzenesulfonamide;
4-[5-(biphenyl)-3-(difluoromethyl)-1H-pyrazol-1-yl]benzenesulfonamide;
4-[5-(1,4-benzodioxan-6-yl)-3-(difluoromethyl)-1H-pyrazol-1-yl]benzenesulfonamide;
4-[3-(difluoromethyl)-5-(4-methylcyclohexyl)-1H-pyrazol-1-yl]benzenesulfonamide;
4-[5-(methyl-1-cyclohexenyl)-3-(difluoromethyl)-1H-pyrazol-1-yl]benzenesulfonamide;
4-[5-(2-methyl-1-cyclopentenyl)-3-(difluoromethyl)-1H-pyrazol-1-yl]benzenesulfonamide;
4-[5-(benzofuran-2-yl)-3-(difluoromethyl)-1H-pyrazol-1-yl]benzenesulfonamide;
4-[5-(1,3-benzodioxol-5-yl)-3-(difluoromethyl)-1H-pyrazol-1-yl]benzenesulfonamide;
4-[5-(2-pyrazinyl)-3-(difluoromethyl)-1H-pyrazol-1-yl]benzenesulfonamide;
4-[5-(4-(morpholino)phenyl)-3-(difluoromethyl)-1H-pyrazol-1-yl]benzenesulfonamide;
4-[5-(2,5-dimethyl-3-furyl)-3-(difluoromethyl)-1H-pyrazol-1-yl]benzenesulfonamide;
4-[5-(5-methyl-2-furyl)-3-(difluoromethyl)-1H-pyrazol-1-yl]benzenesulfonamide;
4-[5-(1-chloro-1-methyl-4-cyclohexyl)-3-(difluoromethyl)-1H-pyrazol-1-yl]benzenesulfonamide;
4-[5-(3,4-dibromo-4-methylcyclohexyl)-3-(difluoromethyl)-1H-pyrazol-1-yl]benzenesulfonamide;
4-[5-(2-methoxycyclohexyl)-3-(difluoromethyl)-1H-pyrazol-1-yl]benzenesulfonamide;
4-[5-(2-thienyl)-3-(difluoromethyl)-1H-pyrazol-1-yl]benzenesulfonamide;
4-[5-(2,4-dimethyl-3-thienyl)-3-(difluoromethyl)-1H-pyrazol-1-yl]benzenesulfonamide;
4-[5-(2,5-dichloro-3-thienyl)-3-(difluoromethyl)-1H-pyrazol-1-yl]benzenesulfonamide;
4-[5-(benzofuran-5-yl)-3-(trifluoromethyl)-1H-pyrazol-1-yl]benzenesulfonamide;
4-[5-(5-bromo-2-thienyl)-3-(trifluoromethyl)-1H-pyrazol-1-yl]benzenesulfonamide;
4-[5-(5-chloro-2-thienyl)-3-(trifluoromethyl)-1H-pyrazol-1-yl]benzenesulfonamide;
4-[5-(5-indanyl)-3-(trifluoromethyl)-1H-pyrazol-1-yl]benzenesulfonamide;
4-[5-(5-methyl-2-thienyl)-3-(trifluoromethyl)-1H-pyrazol-1-yl]benzenesulfonamide;
4-[5-(2,3-dihydrobenzofuran-5-yl)-3-(trifluoromethyl)-1H-pyrazol-1-yl]benzenesulfonamide;
4-[5-(1-cyclohexenyl)-3-(trifluoromethyl)-1H-pyrazol-1-yl]benzenesulfonamide;
4-[5-(5-benzothienyl)-3-(trifluoromethyl)-1H-pyrazol-1-yl]benzenesulfonamide;
4-[5-(3,4-dihydro-2H-1-benzopyran-6-yl)-3-(trifluoromethyl)-1H-pyrazol-1-yl]benzenesulfonamide;
4-[5-(3,4-dihydro-2H-1-benzothiopyran-6-yl)-3-(trifluoromethyl)-1H-pyrazol-1-yl]benzenesulfonamide;
4-[5-(2-phenylethenyl)-3-(trifluoromethyl)-1H-pyrazol-1-yl]benzenesulfonamide;
4-[5-(4-methyl-1,3-benzodioxol-6-yl)-3-(trifluoromethyl)-1H-pyrazol-1-yl]benzenesulfonamide;
4-[5-(4-methyl-1,3-benzodioxol-5-yl)-3-(trifluoromethyl)-1H-pyrazol-1-yl]benzenesulfonamide;
4-[5-(2-pyrazinyl)-3-(trifluoromethyl)-1H-pyrazol-1-yl]benzenesulfonamide;
4-[5-(biphenyl)-3-(trifluoromethyl)-1H-pyrazol-1-yl]benzenesulfonamide;
4-[5-(1,2,3,4-tetrahydronaphth-6-yl])-3-(trifluoromethyl)-1H-pyrazol-1-yl]benzenesulfonamide;
4-[5-(2-naphthyl)-3-(trifluoromethyl)-1H-pyrazol-1-yl]benzenesulfonamide;
4-[5-(2-thiazolyl)-3-(trifluoromethyl)-1H-pyrazol-1-yl]benzenesulfonamide;
4-[5-(2-oxazolyl)-3-(trifluoromethyl)-1H-pyrazol-1-yl]benzenesulfonamide;
4-[5-(cyclohexyl)-3-(trifluoromethyl)-1H-pyrazol-1-yl]benzenesulfonamide;
4-[5-(cyclopentyl)-3-(trifluoromethyl)-1H-pyrazol-1-yl]benzenesulfonamide;
4-[5-(cycloheptyl)-3-(trifluoromethyl)-1H-pyrazol-1-yl]benzenesulfonamide;
4-[5-(1-cyclopentenyl)-3-(trifluoromethyl)-1H-pyrazol-1-yl]benzenesulfonamide;
4-[5-(2-furyl)-3-(trifluoromethyl)-1H-pyrazol-1-yl]benzenesulfonamide;
4-[5-(2-pyridyl)-3-(trifluoromethyl)-1H-pyrazol-1-yl]benzenesulfonamide;
4-[5-(3-pyridyl)-3-(trifluoromethyl)-1H-pyrazol-1-yl]benzenesulfonamide;
4-[5-(6-methyl-3-pyridyl)-3-(trifluoromethyl)-1H-pyrazol-1-yl]benzenesulfonamide;
4-[5-(4-pyridyl)-3-(trifluoromethyl)-1H-pyrazol-1-yl]benzenesulfonamide;
4-[5-(3-cyclohexenyl)-3-(trifluoromethyl)-1H-pyrazol-1-yl]benzenesulfonamide;
4-[5-(4-cyclohexenyl)-3-(trifluoromethyl)-1H-pyrazol-1-yl]benzenesulfonamide;
4-[5-(4-methylcyclohex-4-ene-1-yl)-3-(trifluoromethyl)-1H-pyrazol-1-yl]benzenesulfonamide;
4-[5-(5-chloro-2-furyl)-3-(trifluoromethyl)-1H-pyrazol-1-yl]benzenesulfonamide;
4-[5-(5-brorno-2-furyl)-3-(trifluoromethyl)-1H-pyrazol-1-yl]benzenesulfonamide;
4-[5-(6-methoxy-2-naphthyl)-3-(trifluoromethyl)-1H-pyrazol-1-yl]benzenesulfonamide;
4-[5-(4-chlorophenyl)-3-(heptafluoropropyl)-1H-pyrazol-1-yl]benzenesulfonamide;
4-[5-(4-chlorophenyl)-3-(chlorodifluoromethyl)-1H-pyrazol-1-yl]benzenesulfonamide;
4-[5-(4-chlorophenyl)-3-(pentafluoroethyl)-1H-pyrazol-1-yl]benzenesulfonamide;
4-[5-(3-chloro-4-methoxyphenyl)-3-(chloromethyl)-1H-pyrazol-1-yl]benzenesulfonamide;
4-[3-(chlorodifluoromethyl)-5-(3-fluoro-4-methoxyphenyl)-1H-pyrazol-1-yl]benzenesulfonamide;
4-[5-(phenyl)-3-(fluoromethyl)-1H-pyrazol-1-yl]benzenesulfonamide;
4-[3-(dichloromethyl)-5-(3-fluoro-4-methoxyphenyl)-1H-pyrazol-1-yl]benzenesulfonamide;
4-[3-(bromodifluoromethyl)-5-(3-fluoro-4-methoxyphenyl)-1H-pyrazol-1-yl]benzenesulfonamide;
4-[5-(4-chlorophenyl)-3-(fluoromethyl)-1H-pyrazol-1-yl]benzenesulfonamide;
4-[5-(4-chlorophenyl)-3-(chloromethyl)-1H-pyrazol-1-yl]benzenesulfonamide;
4-[5-(4-chlorophenyl)-3-(dichloromethyl)-1H-pyrazol-1-yl]benzenesulfonamide;
4-[5-(4-fluorophenyl)-3-(trichloromethyl)-1H-pyrazol-1-yl]benzenesulfonamide;
4-[5-(4-chlorophenyl)-3-(1,1-difluoroethyl)-1H-pyrazol-1-yl]benzenesulfonamide;
4-[5-(4-chlorophenyl)-3-(1,1-difluoropropyl)-1H-pyrazol-1-yl]benzenesulfonamide;
4-[5-(4-chlorophenyl)-3-(1,1-dichloroethyl)-1H-pyrazol-1-yl]benzenesulfonamide;
4-[5-(4-chlorophenyl)-3-(1,1-dichloropropyl)-1H-pyrazol-1-yl]benzenesulfonamide;
4-[5-(4-chlorophenyl)-3-nitro-1H-pyrazol-1-yl]benzenesulfonamide;
4-[5-(4-chlorophenyl)-3-(amidino)-1H-pyrazol-1-yl]benzenesulfonamide;
4-[5-(4-chlorophenyl)-3-(methyl sulfonyl)-1H-pyrazol-1-yl]benzenesulfonamide;
4-[5-(4-chlorophenyl)-3-(N-methyl-aminosulfonyl)-1H-pyrazol-1-yl]benzenesulfonamide;
4-[5-(4-fluorophenyl)-3-(imidazolyl)-1H-pyrazol-1-yl]benzenesulfonamide;
4-[5-(4-fluorophenyl)-3-(2-pyridyl)-1H-pyrazol-1-yl]benzenesulfonamide;
4-[5-(4-chlorophenyl)-3-(N-cyanoamidino)-1H-pyrazol-1-yl]benzenesulfonamide;
4-[5-(4-chlorophenyl)-3-(tetrazolyl)-1H-pyrazol-1-yl]benzenesulfonamide;
4-[5-(4-chlorophenyl)-3-(phenylsulfonyl)-1H-pyrazol-1-yl]benzenesulfonamide;
4-[5-(4-chlorophenyl)-3-(N-phenylaminosulfonyl)-1H-pyrazol-1-yl]benzenesulfonamide;
4-[5-(4-chlorophenyl)-3-(N,N-dimethylaminosulfonyl)-1H-pyrazol-1-yl]benzenesulfonamide;
4-[5-(4-chlorophenyl)-3-(N-methyl-N-phenylaminosulfonyl)-1H-pyrazol-1-yl]benzenesulfonamide;
4-[5-(4-chlorophenyl)-3-(N-ethylaminosulfonyl)-1H-pyrazol-1-yl]benzenesulfonamide;
4-[5-(4-chlorophenyl)-3-(N-isopropylaminosulfonyl)-1H-pyrazol-1-yl]benzenesulfonamide;
4-[5-(4-chlorophenyl)-3-(N-methyl-N-ethylaminosulfonyl)-1H-pyrazol-1-yl]benzenesulfonamide;
4-[5-(4-chlorophenyl)-3-(N-methyl-N-(3-chlorophenyl) aminosulfonyl)-1H-pyrazol-1-yl]benzenesulfonamide;
4-[5-(4-chlorophenyl)-3-(N-methyl-N-(2-pyridyl)aminosulfonyl)-1H-pyrazol-1-yl]benzenesulfonamide;
4-[3-methyl-5-phenyl-1H-pyrazol-1-yl]benzenesulfonamide;
4-[3-isobutyl-5-phenyl-1H-pyrazol-1-yl]benzenesulfonamide;
4-[3-(3-hydroxypropyl)-5-phenyl-1H-pyrazol-1-yl]benzenesulfonamide;
4-[5-(4-fluorophenyl)-3-(3-hydroxypropyl)-1H-pyrazol-1-yl]benzenesulfonamide;
4-[5-(3,5-dichloro-4-methoxyphenyl)-3-(3-hydroxypropyl)-1H-pyrazol-1-yl]benzenesulfonamide;
4-[5-(4-methylphenyl)-3-(2-hydroxyisopropyl)-1H-pyrazol-1-yl]benzenesulfonamide;
4-[3-(3-hydroxymethyl)-5-phenyl-1H-pyrazol-1-yl]benzenesulfonamide;
4-[5-(4-chlorophenyl)-3-(3-hydroxymethyl)-1H-pyrazol-1-yl]benzenesulfonamide;
4-[3-(3-hydroxymethyl)-5-(4-methoxyphenyl)-1H-pyrazol-1-yl]benzenesulfonamide;
4-[5-(3,5-dichloro-4-methoxyphenyl)-3-(3-hydroxymethyl)-1H-pyrazol-1-yl]benzenesulfonamide;
4-[5-(3-chloro-4-methoxyphenyl)-3-(3-hydroxymethyl)-1H-pyrazol-1-yl]benzenesulfonamide;
4-[5-(4-chlorophenyl)-3-(chloro)-1H-pyrazol-1-yl]benzenesulfonamide;
4-[5-(4-chlorophenyl)-3-(bromo)-1H-pyrazol-1-yl]benzenesulfonamide;
4-[5-(4-chlorophenyl)-3-(fluoro)-1H-pyrazol-1-yl]benzenesulfonamide;
4-[3-(difluoromethyl)-4,5-dihydro-7-methoxy-1H-benz[g]indazol-1-yl]benzenesulfonamide;
4-[3-(difluoromethyl)-4,5-dihydro-7-methyl-1H-benz[g]indazol-1-yl]benzenesulfonamide;
4-[4,5-dihydro-7-methoxy-3-(trifluoromethyl)-1H-benz[g]indazol-1-yl]benzenesulfonamide;
4-[4,5-dihydro-3-(trifluoromethyl)-1H-benz[g]indazol-1-yl]benzenesulfonamide;
4-[4,5-dihydro-7-methyl-3-(trifluoromethyl)-1H-benz[g]indazol-1-yl]benzenesulfonamide;
4-[4,5-dihydro-6,8-dimethyl-3-(trifluoromethyl)-1H-benz[g]indazol-1-yl]benzenesulfonamide;
4-[4,5-dihydro-6,8-dimethoxy-3-(trifluoromethyl)-1H-benz[g]indazol-1-yl]benzenesulfonamide;
4-[4,5-dihydro-3-trifluoromethyl-1H-thieno[3,2,g]indazol-1-yl]benzenesulfonamide;
4-[1-phenyl-3-(difluoromethyl)-1H-pyrazol-5-yl]benzenesulfonamide;
4-[1-(4-chlorophenyl)-3-(difluoromethyl)-1H-pyrazol-5-yl]benzenesulfonamide;
4-[1-(4-fluorophenyl)-3-(difluoromethyl)-1H-pyrazol-5-yl]benzenesulfonamide;
4-[1-(4-methoxyphenyl)-3-(difluoromethyl)-1H-pyrazol-5-yl]benzenesulfonamide;
4-[1-phenyl-3-(trifluoromethyl)-1H-pyrazol-5-yl]benzenesulfonamide;
4-[1-(4-chlorophenyl)-3-(trifluoromethyl)-1H-pyrazol-5-yl]benzenesulfonamide;
4-[1-(4-fluorophenyl)-3-(trifluoromethyl)-1H-pyrazol-5-yl]benzenesulfonamide; and
4-[1-(4-methoxyphenyl)-3-(trifluoromethyl)-1H-pyrazol-5-yl]benzenesulfonamide.

In some examples, the benzenesulfonamide-containing NSAID is selected from the group of:
4-[5-(4-chlorophenyl)-3-(trifluoromethyl)-1H-pyrazol-1-yl]benzenesulfonamide;
4-[5-phenyl-3-(trifluoromethyl)-1H-pyrazoh-1-yl]benzenesulfonamide;
4-[5-(4-fluorophenyl)-3-(trifluoromethyl)-1H-pyrazol-1-yl]benzenesulfonamide;
4-[5-(4-methoxyphenyl)-3-(trifluoromethyl)-1H-pyrazol-1-yl]benzenesulfonamide;
4-[5-(4-chlorophenyl)-3-(difluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[4-chloro-5-(4-chlorophenyl)-3-(trifluoromethyl)-1H-pyrazol-1-yl]benzenesulfonamide;
4-[3-(difluoromethyl)-5-(4-methylphenyl)-1H-pyrazol-1-yl]benzenesulfonamide;
4-[3-(difluoromethyl)-5-phenyl-1H-pyrazol-1-yl]benzenesulfonamide;
4-[3-(difluoromethyl)-5-(4-methoxyphenyl)-1H-pyrazol-1-yl]benzenesulfonamide;
4-[3-cyano-5-(4-fluorophenyl)-1H-pyrazol-1-yl]benzenesulfonamide;
4-[3-(difluoromethyl)-5-(3-fluoro-4-methoxyphenyl)-1H-pyrazol-1-yl]benzenesulfonamide;
4-[5-(3-fluoro-4-methoxyphenyl)-3-(trifluoromethyl)-1H-pyrazol-1-yl]benzenesulfonamide;
4-[4-chloro-5-phenyl-1H-pyrazol-1-yl]benzenesulfonamide; 4-[5-(4-chlorophenyl)-3-(hydroxymethyl)-1H-pyrazol-1-yl]benzenesulfonamide; and
4-[5-(4-(N,N-dimethylamino)phenyl)-3-(trifluoromethyl)-1H-pyrazol-1-yl]benzenesulfonamide, or a salt (e.g., a pharmaceutically acceptable salt) or solvate of any of the foregoing.

Additional examples of benzenesulfonamide-containing NSAIDs are:

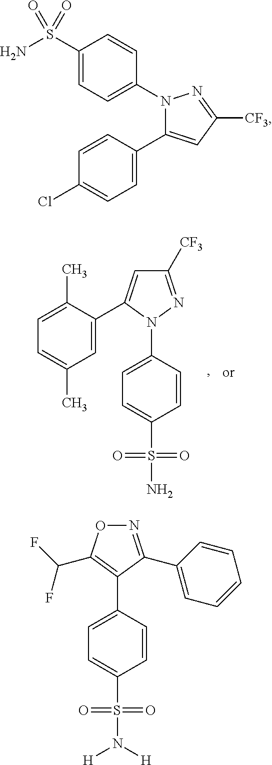

or a salt (e.g., a pharmaceutically acceptable salt) or solvate of any of the above.

An additional example of a benzenesulfonamide-containing NSAID is 4-[5-(4-methylphenyl)-3-(trifluoromethyl)pyrazol-1-yl]benzenesulfonamide (celecoxib), or a salt (e.g., a pharmaceutically acceptable salt) or solvate thereof. The molecular weight of celecoxib is 381.37 g/mole. The structure of 4-[5-(4-methylphenyl)-3-(trifluoromethyl)pyrazol-1-yl]benzenesulfonamide (celecoxib) is shown below:

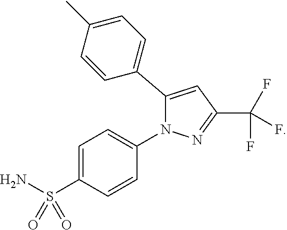 [SMILES: Cc1ccc(-c2cc(C(F)(F)F)nn2-c2ccc(S(N)(=O)=O)cc2)cc1]

Additional examples of benzenesulfonamide-containing NSAIDs and methods of synthesizing benzenesulfonamide-containing NSAIDs are known in the art. For example, the benzenesulfonamide-containing NSAID can be sodium salt (e.g., a hydrated sodium salt) of celecoxib.

### Molecular Weight Ranges of Silk Fibroin

Silk fibroin present in any of the compositions (e.g., gels, e.g., hydrogels) described herein, or used in any of the methods described herein can have, e.g., a molecular weight range or average molecular weight of about 10 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, about 180 kDa, about 170 kDa, about 160 kDa, about 150 kDa, about 140 kDa, about 130 kDa, about 120 kDa, about 110 kDa, about 100 kDa, about 90 kDa, about 80 kDa, about 70 kDa, about 60 kDa, about 50 kDa, about 40 kDa, about 30 kDa, or about 20 kDa (inclusive); about 20 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, about 180 kDa, about 170 kDa, about 160 kDa, about 150 kDa, about 140 kDa, about 130 kDa, about 120 kDa, about 110 kDa, about 100 kDa, about 90 kDa, about 80 kDa, about 70 kDa, about 60 kDa, about 50 kDa, about 40 kDa, or about 30 kDa (inclusive); about 30 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, about 180 kDa, about 170 kDa, about 160 kDa, about 150 kDa, about 140 kDa, about 130 kDa, about 120 kDa, about 110 kDa, about 100 kDa, about 90 kDa, about 80 kDa, about 70 kDa, about 60 kDa, about 50 kDa, or about 40 kDa (inclusive); about 40 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, about 180 kDa, about 170 kDa, about 160 kDa, about 150 kDa, about 140 kDa, about 130 kDa, about 120 kDa, about 110 kDa, about 100 kDa, about 90 kDa, about 80 kDa, about 70 kDa, about 60 kDa, or about 50 kDa (inclusive); about 50 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, about 180 kDa, about 170 kDa, about 160 kDa, about 150 kDa, about 140 kDa, about 130 kDa, about 120 kDa, about 110 kDa, about 100 kDa, about 90 kDa, about 80 kDa, about 70 kDa, or about 60 kDa (inclusive); about 60 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, about 180 kDa, about 170 kDa, about 160 kDa, about 150 kDa, about 140 kDa, about 130 kDa, about 120 kDa, about 110 kDa, about 100 kDa, about 90 kDa, about 80 kDa, or about 70 kDa (inclusive); about 70 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, about 180 kDa, about 170 kDa, about 160 kDa, about 150 kDa, about 140 kDa, about 130 kDa, about 120 kDa, about 110 kDa, about 100 kDa, about 90 kDa, or about 80 kDa (inclusive); about 80 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, about 180 kDa, about 170 kDa, about 160 kDa, about 150 kDa, about 140 kDa, about 130 kDa, about 120 kDa, about 110 kDa, about 100 kDa, or about 90 kDa (inclusive); about 90 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, about 180 kDa, about 170 kDa, about 160 kDa, about 150 kDa, about 140 kDa, about 130 kDa, about 120 kDa, about 110 kDa, or about 100 kDa (inclusive); about 100 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, about 180 kDa, about 170 kDa, about 160 kDa, about 150 kDa, about 140 kDa, about 130 kDa, about 120 kDa, or about 110 kDa (inclusive); about 110 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, about 180 kDa, about 170 kDa, about 160 kDa, about 150 kDa, about 140 kDa, about 130 kDa, or about 120 kDa (inclusive); about 120 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, about 180 kDa, about 170 kDa, about 160 kDa, about 150 kDa, about 140 kDa, or about 130 kDa (inclusive); about 130 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, about 180 kDa, about 170 kDa, about 160 kDa, about 150 kDa, or about 140 kDa (inclusive); about 140 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, about 180 kDa, about 170 kDa, about 160 kDa, or about 150 kDa (inclusive); about 150 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, about 180 kDa, about 170 kDa, or about 160 kDa (inclusive); about 160 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, about 180 kDa, or about 170 kDa (inclusive); about 170 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, about 190 kDa, or about 180 kDa (inclusive); about 180 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, about 200 kDa, or about 190 kDa (inclusive); about 190 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, about 210 kDa, or about 200 kDa (inclusive); about 200 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, about 220 kDa, or about 210 kDa (inclusive); about 210 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, about 230 kDa, or about 220 kDa (inclusive); about 220 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, about 240 kDa, or about 230 kDa (inclusive); about 230 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, about 250 kDa, or about 240 kDa (inclusive); about 240 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, about 260 kDa, or about 250 kDa (inclusive); about 250 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, about 270 kDa, or about 260 kDa (inclusive); about 260 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, about 280 kDa, or about 270 kDa (inclusive); about 270 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, about 290 kDa, or about 280 kDa (inclusive); about 280 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, about 300 kDa, or about 290 kDa (inclusive); about 290 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, about 310 kDa, or about 300 kDa (inclusive); about 300 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, about 320 kDa, or about 310 kDa (inclusive); about 310 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, about 330 kDa, or about 320 kDa (inclusive); about 320 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, about 340 kDa, or about 330 kDa (inclusive); about 330 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, about 350 kDa, or about 340 kDa (inclusive); about 340 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, about 360 kDa, or about 350 kDa (inclusive); about 350 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, about 370 kDa, or about 360 kDa (inclusive); about 360 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, about 380 kDa, or about 370 kDa (inclusive); about 370 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, about 390 kDa, or about 380 kDa (inclusive); about 380 kDa to about 500 kDa, about 490 kDa, about 480 kDa, about 470 kDa, about 460 kDa, about 450 kDa, about 440 kDa, about 430 kDa, about 420 kDa, about 410 kDa, about 400 kDa, or about 390 kDa (inclusive); about 390 kDa to about 500 kDa, about 490 kDa, about 480 kDa, about 470 kDa, about 460 kDa, about 450 kDa, about 440 kDa, about 430 kDa, about 420 kDa, about 410 kDa, or about 400 kDa (inclusive); about 400 kDa to about 500 kDa, about 490 kDa, about 480 kDa, about 470 kDa, about 460 kDa, about 450 kDa, about 440 kDa, about 430 kDa, about 420 kDa, or about 410 kDa (inclusive); about 410 kDa to about 500 kDa, about 490 kDa, about 480 kDa, about 470 kDa, about 460 kDa, about 450 kDa, about 440 kDa, about 430 kDa, or about 420 kDa (inclusive); about 420 kDa to about 500 kDa, about 490 kDa, about 480 kDa, about 470 kDa, about 460 kDa, about 450 kDa, about 440 kDa, or about 430 kDa (inclusive); about 430 kDa to about 500 kDa, about 490 kDa, about 480 kDa, about 470 kDa, about 460 kDa, about 450 kDa or about 440 kDa (inclusive); about 440 kDa to about 500 kDa, about 490 kDa, about 480 kDa, about 470 kDa, about 460 kDa, or about 450 kDa (inclusive); about 450 kDa to about 500 kDa, about 490 kDa, about 480 kDa, about 470 kDa, or about 460 kDa (inclusive); about 460 kDa to about 500 kDa, about 490 kDa, about 480 kDa, or about 470 kDa (inclusive); about 470 kDa to about 500 kDa, about 490 kDa, or about 480 kDa (inclusive); about 480 kDa to about 500 kDa, or about 490 kDa (inclusive); or about 490 kDa to about 500 kDa (inclusive). Methods for determining the molecular weight range or average molecular weight of a protein can include sodium dodecyl sulfate-polyacrylamide electrophoresis (SDS-PAGE), size-exclusion chromatography (SEC)-UV (HPLC), or non-denaturing polyacrylamide gel electrophoresis. Additional methods for determining the molecular weight range or average molecular weight of a protein are known in the art.

### Gelling Agents

Non-limiting examples of gelling agents include a polyethylene glycol (PEG). Non-limiting examples of PEGs include, e.g., PEG300, PEG400, or PEG600, or a PEG having a molecular weight of about 2 kDa, about 4 kDa, about 6 kDa, about 8 kDa, about 10 kDa, about 12 kDa, about 14 kDa, about 16 kDa, about 18 kDa, or about 20 kDa. In some examples, the PEG can have a molecular weight of about 62 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, about 2,400 Da, about 2,200 Da, about 2,000 Da, about 1,800 Da, about 1,600 Da, about 1,400 Da, about 1,200 Da, about 1,000 Da, about 900 Da, about 800 Da, about 700 Da, about 600 Da, about 500 Da, about 400 Da, about 300 Da, about 200 Da, or about 100 Da (inclusive); about 100 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, about 2,400 Da, about 2,200 Da, about 2,000 Da, about 1,800 Da, about 1,600 Da, about 1,400 Da, about 1,200 Da, about 1,000 Da, about 900 Da, about 800 Da, about 700 Da, about 600 Da, about 500 Da, about 400 Da, about 300 Da, or about 200 Da (inclusive); about 200 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, about 2,400 Da, about 2,200 Da, about 2,000 Da, about 1,800 Da, about 1,600 Da, about 1,400 Da, about 1,200 Da, about 1,000 Da, about 900 Da, about 800 Da, about 700 Da, about 600 Da, about 500 Da, about 400 Da, or about 300 Da (inclusive); about 300 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, about 2,400 Da, about 2,200 Da, about 2,000 Da, about 1,800 Da, about 1,600 Da, about 1,400 Da, about 1,200 Da, about 1,000 Da, about 900 Da, about 800 Da, about 700 Da, about 600 Da, about 500 Da, or about 400 Da (inclusive); about 400 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, about 2,400 Da, about 2,200 Da, about 2,000 Da, about 1,800 Da, about 1,600 Da, about 1,400 Da, about 1,200 Da, about 1,000 Da, about 900 Da, about 800 Da, about 700 Da, about 600 Da, or about 500 Da (inclusive); about 500 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, about 2,400 Da, about 2,200 Da, about 2,000 Da, about 1,800 Da, about 1,600 Da, about 1,400 Da, about 1,200 Da, about 1,000 Da, about 900 Da, about 800 Da, about 700 Da, or about 600 Da (inclusive); about 600 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, about 2,400 Da, about 2,200 Da, about 2,000 Da, about 1,800 Da, about 1,600 Da, about 1,400 Da, about 1,200 Da, about 1,000 Da, about 900 Da, about 800 Da, or about 700 Da (inclusive); about 700 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, about 2,400 Da, about 2,200 Da, about 2,000 Da, about 1,800 Da, about 1,600 Da, about 1,400 Da, about 1,200 Da, about 1,000 Da, about 900 Da, or about 800 Da (inclusive); about 800 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, about 2,400 Da, about 2,200 Da, about 2,000 Da, about 1,800 Da, about 1,600 Da, about 1,400 Da, about 1,200 Da, about 1,000 Da, or about 900 Da (inclusive); about 900 kDa to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, about 2,400 Da, about 2,200 Da, about 2,000 Da, about 1,800 Da, about 1,600 Da, about 1,400 Da, about 1,200 Da, or about 1,000 Da (inclusive); about 1,000 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, about 2,400 Da, about 2,200 Da, about 2,000 Da, about 1,800 Da, about 1,600 Da, about 1,400 Da, or about 1,200 Da (inclusive); about 1,200 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, about 2,400 Da, about 2,200 Da, about 2,000 Da, about 1,800 Da, about 1,600 Da, or about 1,400 Da (inclusive); about 1,400 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, about 2,400 Da, about 2,200 Da, about 2,000 Da, about 1,800 Da, or about 1,600 Da (inclusive); about 1,600 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, about 2,400 Da, about 2,200 Da, about 2,000 Da, or about 1,800 Da (inclusive); about 1,800 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, about 2,400 Da, about 2,200 Da, or about 2,000 Da (inclusive); about 2,000 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, about 2,400 Da, or about 2,200 Da (inclusive); about 2,200 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, about 2,600 Da, or about 2,400 Da (inclusive); about 2,400 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, about 2,800 Da, or about 2,600 Da (inclusive); about 2,600 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, about 3,000 Da, or about 2,800 Da (inclusive); about 2,800 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, about 3,200 Da, or about 3,000 Da (inclusive); about 3,000 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, about 3,400 Da, or about 3,200 Da (inclusive); about 3,200 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, about 3,600 Da, or about 3,400 Da (inclusive); about 3,400 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, about 3,800 Da, or about 3,600 Da (inclusive); about 3,600 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, about 4,000 Da, or about 3,800 Da (inclusive); about 3,800 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, about 4,200 Da, or about 4,000 Da (inclusive); about 4,000 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, about 4,400 Da, or about 4,200 Da (inclusive); about 4,200 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, about 4,600 Da, or about 4,400 Da (inclusive); about 4,400 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, about 4,800 Da, or about 4,600 Da (inclusive); about 4,600 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, about 5,000 Da, or about 4,800 Da (inclusive); about 4,800 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, about 5,200 Da, or about 5,000 Da (inclusive); about 5,000 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, about 5,400 Da, or about 5,200 Da (inclusive); about 5,200 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, about 5,600 Da, or about 5,400 Da (inclusive); about 5,400 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, about 5,800 Da, or about 5,600 Da (inclusive); about 5,600 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, about 6,000 Da, or about 5,800 Da (inclusive); about 5,800 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, about 6,200 Da, or about 6,000 Da (inclusive); about 6,000 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, about 6,400 Da, or about 6,200 Da (inclusive); about 6,200 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, about 6,600 Da, or about 6,400 Da (inclusive); about 6,400 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, about 6,800 Da, or about 6,600 Da (inclusive); about 6,600 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, about 7,000 Da, or about 6,800 Da (inclusive); about 6,800 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, about 7,200 Da, or about 7,000 Da (inclusive); about 7,000 Da to about 20,000 Da, about 18,000 Da, about 16,000 Da, about 14,000 Da, about 12,000 Da, about 10,000 Da, about 8,000 Da, about 7,800 Da, about 7,600 Da, about 7,400 Da, or about 7,200 Da (inclusive); about 7,200 Da to about 20,000 Da, about 18,000 Da, about 16,000 Da, about 14,000 Da, about 12,000 Da, about 10,000 Da, about 8,000 Da, about 7,800 Da, about 7,600 Da, or about 7,400 Da (inclusive); about 7,400 Da to about 20,000 Da, about 18,000 Da, about 16,000 Da, about 14,000 Da, about 12,000 Da, about 10,000 Da, about 8,000 Da, about 7,800 Da, or about 7,600 Da (inclusive); about 7,600 Da to about 20,000 Da, about 18,000 Da, about 16,000 Da, about 14,000 Da, about 12,000 Da, about 10,000 Da, about 8,000 Da, or about 7,800 Da (inclusive); about 8,000 Da to about 20,000 Da, about 18,000 Da, about 16,000 Da, about 14,000 Da, about 12,000 Da, or about 10,000 Da (inclusive); about 10,000 Da to about 20,000 Da, about 18,000 Da, about 16,000 Da, about 14,000 Da, or about 12,000 Da (inclusive); about 12,000 Da to about 20,000 Da, about 18,000 Da, about 16,000 Da, or about 14,000 Da (inclusive); about 14,000 Da to about 20,000 Da, about 18,000 Da, or about 16,000 Da (inclusive); about 16,000 Da to about 20,000 Da, or about 18,000 Da (inclusive); or about 18,000 Da to about 20,000 Da (inclusive).

Additional examples of gelling agents include glycerol, sorbitol, hydroxyethyl cellulose, carboxymethyl cellulose, triethylamine, triethanolamine, 2-pyrrolidone, alpha-cyclodextrin, benzyl alcohol, beta-cyclodextrin, dimethyl sulfoxide, dimethylacetamide (DMA), dimethylformamide, ethanol, gamma-cyclodextrin, glycerol formal, hydroxypropyl beta-cyclodextrin, kolliphor 124, kolliphor 181, kolliphor 188, kolliphor 407, kolliphor EL (cremaphor EL), cremaphor RH 40, cremophor RH 60, d-alpha-tocopherol, PEG 1000 succinate, polysorbate 20, polysorbate 80, solutol HS 15, sorbitan monooleate, poloxamer-407, poloxamer-188, Labrafil M-1944CS, Lrabfil M-2125CS, Labrasol, Gellucire 44/14, Softigen 767, mono- and di-fatty acid esters of PEG 300, PEG 400, or PEG 1750, kolliphor RH60, N-methyl-2-pyrrolidone, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil, beeswax, d-alpha-tocopherol, oleic acid, medium-chain mono- and di-glycerides, alpha-cyclodextrin, beta-cyclodextrin, hydroxypropyl-beta-cyclodextrin, sulfo-butylether-beta-cyclodextrin, hydrogenated soy phosphatidylcholine, distearoylphosphatidylglycerol, L-alpha-dimyristoylphosphatidylcholine, L-alpha-dimyristoylphosphatidylglycerol, PEG 300, PEG 300 caprylic/capric glycerides (Softigen 767), PEG 300 linoleic glycerides (Labrafil M-2125CS), PEG 300 oleic glycerides (Labrafil M-1944CS), PEG 400, PEG 400 caprylic/capric glycerides (Labrasol), polyoxyl 40 stearate (PEG 1750 monosterate), polyoxyl 8 stearate (PEG 400 monosterate), polysorbate 20, polysorbate-80, polyvinyl pyrrolidone, polyvinyl pyrrolidone-12, polyvinyl pyrrolidone-17, propylene carbonate, propylene glycol, solutol HS 15, sorbitan monooleate (Span 20), sulfobutylether-beta-cyclodextrin, transcutol, triacetin, 1-dodecylazacyclo-heptan-2-one, caprolactam, castor oil, cottonseed oil, ethyl acetate, medium chain triglycerides, methyl acetate, oleic acid, safflower oil, sesame oil, soybean oil, tetrahydrofuran, and glycerin. Additional examples of gelling agents include acacia, alginic acid, bentonite, Carbopols® (also known as carbomers), carboxymethyl cellulose, ethylcellulose, gelatin, hydroxyethyl cellulose, hydroxypropyl cellulose, magnesium aluminum silicate, methylcellulose, polaxamers, polyvinyl alcohol, sodium alginate, tragacanth, and xanthan gum. Additional examples of gelling agents are known in the art. Some embodiments of any of the compositions described herein can include two or more gelling agents (e.g., Tween-80 in combination with one or both of a PEG (e.g., PEG300) and glycerol).

In some embodiments, a gelling agent can be, e.g., a water-soluble, waxy solid. In some embodiments, a gelling agent can be water-soluble and hygroscopic in nature. In some embodiments, a gelling agent can be a polar molecule. In some embodiments, a gelling agent can have a net positive charge at a physiological pH (e.g., pH of about 7.4). In some embodiments, a gelling agent can have a net negative charge at a physiological pH (e.g., a pH of about 7.4). In some embodiments, a gelling agent can have a net neutral charge at a physiological pH (e.g., a pH of about 7.4). In some embodiments, a gelling agent can be amphipathic. In some embodiments, a gelling agent can be an oil (e.g., castor, corn oil, cottonseed oil, olive oil, peanut oil, peppermint oil, safflower oil, sesame oil, soybean oil, a hydrogenated vegetable oil, hydrogenated soybean oil, and medium-chain triglycerides of coconut oil and/or palm seed oil), an emulsifier (e.g., polyoxyl 40 stearate (PEG 1750 monosterate), polyoxyl 8 stearate (PEG 400 monosterate), polysorbate 20, polysorbate-80, a poloxamer), a surfactant (e.g., a polysorbate, a poloxamer, sodium dodecyl sulfate, Triton X100, and tyloxapol), a suspending agent (e.g., polyvinyl pyrrolidone, polyvinyl pyrrolidone-12, polyvinyl pyrrolidone-17, hydroxyethyl cellulose, and carboxymethyl cellulose), or other inert excipient.

In some examples, a gelling agent can be used to induce gelation of a composition. In some examples, a higher concentration of a gelling agent can dissolve a benzenesulfonamide-containing NSAID in a physiologically acceptable buffer (e.g., phosphate buffered saline). In some embodiments, e.g., a gelling agent can dissolve a benzenesulfonamide-containing NSAID and can be used to result in a solution (e.g., an aqueous solution) including the benzenesulfonamide-containing NSAID. In some embodiments, a gelling agent can result in the formation of a gel (e.g., a silk fibroin hydrogel) including a benzenesulfonamide-containing NSAID. In some embodiments, a gelling agent can act as a surfactant to aid in the stabilization of a particle or a plurality of particles that include one or more benzenesulfonamide-containing NSAID (e.g., any of the benzenesulfonamide-containing NSAIDs described herein) in a fluid (e.g., a pharmaceutically acceptable solvent or buffer, e.g., phosphate buffered saline). For example, Tween-80 is a gelling agent that can be used as a surfactant to stabilize a particle or a plurality of particles that include one or more benzenesulfonamide-containing NSAIDs.

### Compositions

Provided herein are compositions (e.g., gels, e.g., hydrogels) that include a benzenesulfonamide-containing NSAID, silk fibroin, and a gelling agent (e.g., any of the exemplary gelling agents described herein or known in the art). For example, the compositions can be a gel (e.g., a hydrogel or an organogel), such as a gel (e.g., a hydrogel or an organogel) that includes a solution of a benzenesulfonamide-containing NSAID or a gel (e.g., a hydrogel or an organogel) that includes a suspension of a plurality of particles of a benzenesulfonamide-containing NSAID (e.g., any of the exemplary particles described herein), a suspension, a cream, a paste, a powder, a solution, or an emulsion.

The compositions (e.g., gels, e.g., hydrogels) provided herein can, e.g., release at least 15% to about 100%, about 95%, about 90%, about 85%, about 80%, about 75%, about 70%, about 65%, about 60%, about 55%, about 50%, about 45%, about 40%, about 35%, about 30%, about 25%, or about 20%; about 20% to about 100%, about 95%, about 90%, about 85%, about 80%, about 75%, about 70%, about 65%, about 60%, about 55%, about 50%, about 45%, about 40%, about 35%, about 30%, or about 25%; about 25% to about 100%, about 95%, about 90%, about 85%, about 80%, about 75%, about 70%, about 65%, about 60%, about 55%, about 50%, about 45%, about 40%, about 35%, or about 30%; about 30% to about 100%, about 95%, about 90%, about 85%, about 80%, about 75%, about 70%, about 65%, about 60%, about 55%, about 50%, about 45%, about 40%, or about 35%; about 35% to about 100%, about 95%, about 90%, about 85%, about 80%, about 75%, about 70%, about 65%, about 60%, about 55%, about 50%, about 45%, or about 40%; about 40% to about 100%, about 95%, about 90%, about 85%, about 80%, about 75%, about 70%, about 65%, about 60%, about 55%, about 50%, or about 45%; about 45% to about 50% to about 100%, about 95%, about 90%, about 85%, about 80%, about 75%, about 70%, about 65%, about 60%, about 55%, or about 50%; about 50% to about 100%, about 95%, about 90%, about 85%, about 80%, about 75%, about 70%, about 65%, about 60%, or about 55%; about 55% to about 100%, about 95%, about 90%, about 85%, about 80%, about 75%, about 70%, about 65%, or about 60%; about 60% to about 100%, about 95%, about 90%, about 85%, about 80%, about 75%, about 70%, or about 65%; about 65% to about 100%, about 95%, about 90%, about 85%, about 80%, about 75%, or about 70%; about 70% to about 100%, about 95%, about 90%, about 85%, about 80%, or about 75%; about 75% to about 100%, about 95%, about 90%, about 85%, or about 80%; about 80% to about 100%, about 95%, about 90%, or about 85%; about 85% to about 100%, about 95%, or about 90%; about 90% to about 100%, or about 95%; or about 95% to about 100%, of the total amount of the benzenesulfonamide-containing NSAID in the composition (e.g., gel, e.g., hydrogel) over a period of about 10 day to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, about 30 days, about 25 days, about 20 days, or about 15 days; about 15 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, about 30 days, about 25 days, or about 20 days; about 20 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, about 30 days, or about 25 days; about 25 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, or about 30 days; about 30 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, or about 35 days; about 35 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, or about 40 days; about 40 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, or about 45 days; about 45 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, or about 50 days; about 50 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, or about 55 days; about 55 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, or about 60 days; about 60 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, or about 65 days; about 65 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, or about 70 days; about 70 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, or about 75 days; about 75 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, or about 80 days; about 80 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, or about 85 days; about 85 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, or about 90 days; about 90 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, or about 95 days; about 95 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, or about 100 days; about 100 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, or about 105 days; about 105 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, or about 110 days; about 110 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, or about 115 days; about 115 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, or about 120 days; about 120 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, or about 125 days; about 125 days to about 200 days, about 195 days, about 190 days, about 185 days, about 180 days, about 175 days, about 170 days, about 165 days, about 160 days, about 155 days, about 150 days, about 145 days, about 140 days, about 135 days, or about 130 days; about 130 days to about 200 days, about 195 days, about 190 days, about 185 days, about 180 days, about 175 days, about 170 days, about 165 days, about 160 days, about 155 days, about 150 days, about 145 days, about 140 days, or about 135 days; about 135 days to about 200 days, about 195 days, about 190 days, about 185 days, about 180 days, about 175 days, about 170 days, about 165 days, about 160 days, about 155 days, about 150 days, about 145 days, or about 140 days; about 140 days to about 200 days, about 195 days, about 190 days, about 185 days, about 180 days, about 175 days, about 170 days, about 165 days, about 160 days, about 155 days, about 150 days, or about 145 days; about 145 days to about 200 days, about 195 days, about 190 days, about 185 days, about 180 days, about 175 days, about 170 days, about 165 days, about 160 days, about 155 days, or about 150 days; about 150 days to about 200 days, about 195 days, about 190 days, about 185 days, about 180 days, about 175 days, about 170 days, about 165 days, about 160 days, or about 155 days; about 155 days to about 200 days, about 195 days, about 190 days, about 185 days, about 180 days, about 175 days, about 170 days, about 165 days, or about 160 days; about 160 days to about 200 days, about 195 days, about 190 days, about 185 days, about 180 days, about 175 days, about 170 days, or about 165 days; about 165 days to about 200 days, about 195 days, about 190 days, about 185 days, about 180 days, about 175 days, or about 170 days; about 170 days to about 200 days, about 195 days, about 190 days, about 185 days, about 180 days, or about 175 days; about 175 days to about 200 days, about 195 days, about 190 days, about 185 days, or about 180 days; about 180 days to about 200 days, about 195 days, about 190 days, or about 185 days; about 185 days to about 200 days, about 195 days, or about 190 days; about 190 days to about 200 days or about 195 days; or about 195 days to about 200 days; when incubated under simulated physiological conditions, e.g., buffered to a physiological pH, salt, and temperature range, e.g., incubated in a physiologically acceptable buffer (e.g., phosphate buffered saline (PBS), 2% Tween-80 in PBS, saline, a mammalian culture medium, a mammalian biological fluid (e.g., mammalian serum, plasma, cervical fluid, or synovial fluid) at a temperature of about 30° C. to about 40° C., about 39° C., about 38° C., about 37° C., about 36° C., about 35° C., about 34° C., about 33° C., about 32° C., or about 31° C.; about 31° C. to about 40° C., about 39° C., about 38° C., about 37° C., about 36° C., about 35° C., about 34° C., about 33° C., or about 32° C.; about 32° C. to about 40° C., about 39° C., about 38° C., about 37° C., about 36° C., about 35° C., about 34° C., or about 33° C.; about 33° C. to about 40° C., about 39° C., about 38° C., about 37° C., about 36° C., about 35° C., or about 34° C.; about 34° C. to about 40° C., about 39° C., about 38° C., about 37° C., about 36° C., or about 35° C.; about 35° C. to about 40° C., about 39° C., about 38° C., about 37° C., or about 36° C.; about 36° C. to about 40° C., about 39° C., about 38° C., or about 37° C.; about 37° C. to about 40° C., about 39° C., or about 38° C.; about 38° C. to about 40° C. or about 39° C.; or about 39° C. to about 40° C. (e.g., using the assay described in Example 1).

The compositions (e.g., gels, e.g., hydrogels) provided herein can, e.g., release a therapeutically effective amount (e.g., to treat any of the inflammatory and/or pain conditions described herein or to reduce inflammation, pain, or fever in a subject) of the benzenesulfonamide-containing NSAID over a period of about 10 day to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, about 30 days, about 25 days, about 20 days, or about 15 days; about 15 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, about 30 days, about 25 days, or about 20 days; about 20 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, about 30 days, or about 25 days; about 25 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, or about 30 days; about 30 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, or about 35 days; about 35 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, or about 40 days; about 40 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, or about 45 days; about 45 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, or about 50 days; about 50 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, or about 55 days; about 55 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, or about 60 days; about 60 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, or about 65 days; about 65 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, or about 70 days; about 70 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, or about 75 days; about 75 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, or about 80 days; about 80 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, or about 85 days; about 85 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, or about 90 days; about 90 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, or about 95 days; about 95 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, or about 100 days; about 100 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, or about 105 days; about 105 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, or about 110 days; about 110 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, or about 115 days; about 115 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, or about 120 days; about 120 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, or about 125 days; about 125 days to about 200 days, about 195 days, about 190 days, about 185 days, about 180 days, about 175 days, about 170 days, about 165 days, about 160 days, about 155 days, about 150 days, about 145 days, about 140 days, about 135 days, or about 130 days; about 130 days to about 200 days, about 195 days, about 190 days, about 185 days, about 180 days, about 175 days, about 170 days, about 165 days, about 160 days, about 155 days, about 150 days, about 145 days, about 140 days, or about 135 days; about 135 days to about 200 days, about 195 days, about 190 days, about 185 days, about 180 days, about 175 days, about 170 days, about 165 days, about 160 days, about 155 days, about 150 days, about 145 days, or about 140 days; about 140 days to about 200 days, about 195 days, about 190 days, about 185 days, about 180 days, about 175 days, about 170 days, about 165 days, about 160 days, about 155 days, about 150 days, or about 145 days; about 145 days to about 200 days, about 195 days, about 190 days, about 185 days, about 180 days, about 175 days, about 170 days, about 165 days, about 160 days, about 155 days, or about 150 days; about 150 days to about 200 days, about 195 days, about 190 days, about 185 days, about 180 days, about 175 days, about 170 days, about 165 days, about 160 days, or about 155 days; about 155 days to about 200 days, about 195 days, about 190 days, about 185 days, about 180 days, about 175 days, about 170 days, about 165 days, or about 160 days; about 160 days to about 200 days, about 195 days, about 190 days, about 185 days, about 180 days, about 175 days, about 170 days, or about 165 days; about 165 days to about 200 days, about 195 days, about 190 days, about 185 days, about 180 days, about 175 days, or about 170 days; about 170 days to about 200 days, about 195 days, about 190 days, about 185 days, about 180 days, or about 175 days; about 175 days to about 200 days, about 195 days, about 190 days, about 185 days, or about 180 days; about 180 days to about 200 days, about 195 days, about 190 days, or about 185 days; about 185 days to about 200 days, about 195 days, or about 190 days; about 190 days to about 200 days or about 195 days; or about 195 days to about 200 days; when incubated under simulated physiological conditions, e.g., buffered to a physiological pH, salt, and temperature range, e.g., incubated in a physiologically acceptable buffer (e.g., phosphate buffered saline (PBS), 2% Tween-80 in PBS, 2% Tween-80 and 0.05% sodium azide in PBS, saline, a mammalian culture medium, a mammalian biological fluid (e.g., mammalian serum, plasma, cervical fluid, or synovial fluid) at a temperature of about 30° C. to about 40° C., about 39° C., about 38° C., about 37° C., about 36° C., about 35° C., about 34° C., about 33° C., about 32° C., or about 31° C.; about 31° C. to about 40° C., about 39° C., about 38° C., about 37° C., about 36° C., about 35° C., about 34° C., about 33° C., or about 32° C.; about 32° C. to about 40° C., about 39° C., about 38° C., about 37° C., about 36° C., about 35° C., about 34° C., or about 33° C.; about 33° C. to about 40° C., about 39° C., about 38° C., about 37° C., about 36° C., about 35° C., or about 34° C.; about 34° C. to about 40° C., about 39° C., about 38° C., about 37° C., about 36° C., or about 35° C.; about 35° C. to about 40° C., about 39° C., about 38° C., about 37° C., or about 36° C.; about 36° C. to about 40° C., about 39° C., about 38° C., or about 37° C.; about 37° C. to about 40° C., about 39° C., or about 38° C.; about 38° C. to about 40° C. or about 39° C.; or about 39° C. to about 40° C. (e.g., using the assay described in Example 1).

A physiological pH is, e.g., about 6.8 to about 7.6, about 7.5, about 7.4, about 7.3, about 7.2, about 7.1, about 7.0, or about 6.7; about 6.7 to about 7.6, about 7.5, about 7.4, about 7.3, about 7.2, about 7.1, about 7.0, about 6.9, or about 6.8; about 6.8 to about 7.6, about 7.5, about 7.4, about 7.3, about 7.2, about 7.1, about 7.0, or about 6.9; about 6.9 to about 7.6, about 7.5, about 7.4, about 7.3, about 7.2, about 7.1, or about 7.0; about 7.0 to about 7.6, about 7.5, about 7.4, about 7.3, about 7.2, or about 7.1; about 7.1 to about 7.6, about 7.5, about 7.4, about 7.3, or about 7.2; about 7.2 to about 7.6, about 7.5, about 7.4, or about 7.3; about 7.3 to about 7.6, about 7.5, or about 7.4; about 7.4 to about 7.6, or about 7.5; or about 7.5 to about 7.6. A physiological temperature is, e.g., about 30° C. to about 40° C., about 39° C., about 38° C., about 37° C., about 36° C., about 35° C., about 34° C., about 33° C., about 32° C., or about 31° C.; about 31° C. to about 40° C., about 39° C., about 38° C., about 37° C., about 36° C., about 35° C., about 34° C., about 33° C., or about 32° C.; about 32° C. to about 40° C., about 39° C., about 38° C., about 37° C., about 36° C., about 35° C., about 34° C., or about 33° C.; about 33° C. to about 40° C., about 39° C., about 38° C., about 37° C., about 36° C., about 35° C., or about 34° C.; about 34° C. to about 40° C., about 39° C., about 38° C., about 37° C., about 36° C., or about 35° C.; about 35° C. to about 40° C., about 39° C., about 38° C., about 37° C., or about 36° C.; about 36° C. to about 40° C., about 39° C., about 38° C., or about 37° C.; about 37° C. to about 40° C., about 39° C., or about 38° C.; about 38° C. to about 40° C. or about 39° C.; or about 39° C. to about 40° C.

The compositions (e.g., gels, e.g., hydrogels) provided herein can, e.g., release at least 15% to about 100%, about 95%, about 90%, about 85%, about 80%, about 75%, about 70%, about 65%, about 60%, about 55%, about 50%, about 45%, about 40%, about 35%, about 30%, about 25%, or about 20%; about 20% to about 100%, about 95%, about 90%, about 85%, about 80%, about 75%, about 70%, about 65%, about 60%, about 55%, about 50%, about 45%, about 40%, about 35%, about 30%, or about 25%; about 25% to about 100%, about 95%, about 90%, about 85%, about 80%, about 75%, about 70%, about 65%, about 60%, about 55%, about 50%, about 45%, about 40%, about 35%, or about 30%; about 30% to about 100%, about 95%, about 90%, about 85%, about 80%, about 75%, about 70%, about 65%, about 60%, about 55%, about 50%, about 45%, about 40%, or about 35%; about 35% to about 100%, about 95%, about 90%, about 85%, about 80%, about 75%, about 70%, about 65%, about 60%, about 55%, about 50%, about 45%, or about 40%; about 40% to about 100%, about 95%, about 90%, about 85%, about 80%, about 75%, about 70%, about 65%, about 60%, about 55%, about 50%, or about 45%; about 45% to about 50% to about 100%, about 95%, about 90%, about 85%, about 80%, about 75%, about 70%, about 65%, about 60%, about 55%, or about 50%; about 50% to about 100%, about 95%, about 90%, about 85%, about 80%, about 75%, about 70%, about 65%, about 60%, or about 55%; about 55% to about 100%, about 95%, about 90%, about 85%, about 80%, about 75%, about 70%, about 65%, or about 60%; about 60% to about 100%, about 95%, about 90%, about 85%, about 80%, about 75%, about 70%, or about 65%; about 65% to about 100%, about 95%, about 90%, about 85%, about 80%, about 75%, or about 70%; about 70% to about 100%, about 95%, about 90%, about 85%, about 80%, or about 75%; about 75% to about 100%, about 95%, about 90%, about 85%, or about 80%; about 80% to about 100%, about 95%, about 90%, or about 85%; about 85% to about 100%, about 95%, or about 90%; about 90% to about 100%, or about 95%; or about 95% to about 100%, of the total amount of the benzenesulfonamide-containing NSAID in the composition (e.g., gel, e.g., hydrogel) over a period of about 10 day to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, about 30 days, about 25 days, about 20 days, or about 15 days; about 15 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, about 30 days, about 25 days, or about 20 days; about 20 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, about 30 days, or about 25 days; about 25 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, or about 30 days; about 30 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, or about 35 days; about 35 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, or about 40 days; about 40 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, or about 45 days; about 45 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, or about 50 days; about 50 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, or about 55 days; about 55 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, or about 60 days; about 60 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, or about 65 days; about 65 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, or about 70 days; about 70 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, or about 75 days; about 75 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, or about 80 days; about 80 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, or about 85 days; about 85 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, or about 90 days; about 90 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, or about 95 days; about 95 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, or about 100 days; about 100 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, or about 105 days; about 105 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, or about 110 days; about 110 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, or about 115 days; about 115 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, or about 120 days; about 120 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, or about 125 days; about 125 days to about 200 days, about 195 days, about 190 days, about 185 days, about 180 days, about 175 days, about 170 days, about 165 days, about 160 days, about 155 days, about 150 days, about 145 days, about 140 days, about 135 days, or about 130 days; about 130 days to about 200 days, about 195 days, about 190 days, about 185 days, about 180 days, about 175 days, about 170 days, about 165 days, about 160 days, about 155 days, about 150 days, about 145 days, about 140 days, or about 135 days; about 135 days to about 200 days, about 195 days, about 190 days, about 185 days, about 180 days, about 175 days, about 170 days, about 165 days, about 160 days, about 155 days, about 150 days, about 145 days, or about 140 days; about 140 days to about 200 days, about 195 days, about 190 days, about 185 days, about 180 days, about 175 days, about 170 days, about 165 days, about 160 days, about 155 days, about 150 days, or about 145 days; about 145 days to about 200 days, about 195 days, about 190 days, about 185 days, about 180 days, about 175 days, about 170 days, about 165 days, about 160 days, about 155 days, or about 150 days; about 150 days to about 200 days, about 195 days, about 190 days, about 185 days, about 180 days, about 175 days, about 170 days, about 165 days, about 160 days, or about 155 days; about 155 days to about 200 days, about 195 days, about 190 days, about 185 days, about 180 days, about 175 days, about 170 days, about 165 days, or about 160 days; about 160 days to about 200 days, about 195 days, about 190 days, about 185 days, about 180 days, about 175 days, about 170 days, or about 165 days; about 165 days to about 200 days, about 195 days, about 190 days, about 185 days, about 180 days, about 175 days, or about 170 days; about 170 days to about 200 days, about 195 days, about 190 days, about 185 days, about 180 days, or about 175 days; about 175 days to about 200 days, about 195 days, about 190 days, about 185 days, or about 180 days; about 180 days to about 200 days, about 195 days, about 190 days, or about 185 days; about 185 days to about 200 days, about 195 days, or about 190 days; about 190 days to about 200 days or about 195 days; or about 195 days to about 200 days; in a subject (e.g., a human or a non-human mammal, e.g., an animal model of an inflammatory and/or pain condition, or an animal model of pain, inflammation, or fever).

The compositions (e.g., gels, e.g., hydrogels) provided herein can, e.g., release a therapeutically effective amount (e.g., to treat any of the inflammatory and/or pain conditions described herein or to reduce inflammation, pain, or fever in a subject) of the benzenesulfonamide-containing NSAID over a period of about 10 day to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, about 30 days, about 25 days, about 20 days, or about 15 days; about 15 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, about 30 days, about 25 days, or about 20 days; about 20 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, about 30 days, or about 25 days; about 25 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, or about 30 days; about 30 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, or about 35 days; about 35 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, or about 40 days; about 40 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, or about 45 days; about 45 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, or about 50 days; about 50 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, or about 55 days; about 55 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, or about 60 days; about 60 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, or about 65 days; about 65 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, or about 70 days; about 70 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, or about 75 days; about 75 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, or about 80 days; about 80 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, or about 85 days; about 85 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, or about 90 days; about 90 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, or about 95 days; about 95 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, or about 100 days; about 100 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, or about 105 days; about 105 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, or about 110 days; about 110 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, or about 115 days; about 115 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, or about 120 days; about 120 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, or about 125 days; about 125 days to about 200 days, about 195 days, about 190 days, about 185 days, about 180 days, about 175 days, about 170 days, about 165 days, about 160 days, about 155 days, about 150 days, about 145 days, about 140 days, about 135 days, or about 130 days; about 130 days to about 200 days, about 195 days, about 190 days, about 185 days, about 180 days, about 175 days, about 170 days, about 165 days, about 160 days, about 155 days, about 150 days, about 145 days, about 140 days, or about 135 days; about 135 days to about 200 days, about 195 days, about 190 days, about 185 days, about 180 days, about 175 days, about 170 days, about 165 days, about 160 days, about 155 days, about 150 days, about 145 days, or about 140 days; about 140 days to about 200 days, about 195 days, about 190 days, about 185 days, about 180 days, about 175 days, about 170 days, about 165 days, about 160 days, about 155 days, about 150 days, or about 145 days; about 145 days to about 200 days, about 195 days, about 190 days, about 185 days, about 180 days, about 175 days, about 170 days, about 165 days, about 160 days, about 155 days, or about 150 days; about 150 days to about 200 days, about 195 days, about 190 days, about 185 days, about 180 days, about 175 days, about 170 days, about 165 days, about 160 days, or about 155 days; about 155 days to about 200 days, about 195 days, about 190 days, about 185 days, about 180 days, about 175 days, about 170 days, about 165 days, or about 160 days; about 160 days to about 200 days, about 195 days, about 190 days, about 185 days, about 180 days, about 175 days, about 170 days, or about 165 days; about 165 days to about 200 days, about 195 days, about 190 days, about 185 days, about 180 days, about 175 days, or about 170 days; about 170 days to about 200 days, about 195 days, about 190 days, about 185 days, about 180 days, or about 175 days; about 175 days to about 200 days, about 195 days, about 190 days, about 185 days, or about 180 days; about 180 days to about 200 days, about 195 days, about 190 days, or about 185 days; about 185 days to about 200 days, about 195 days, or about 190 days; about 190 days to about 200 days or about 195 days; or about 195 days to about 200 days; in a subject (e.g., a human or a non-human mammal, e.g., an animal model of an inflammatory and/or pain condition, or an animal model of pain, inflammation, or fever).

In some examples, the compositions (e.g., gels, e.g., hydrogels) provided herein can include two, three, four, or five different benzenesulfonamide-containing NSAIDs (e.g., any of the benzenesulfonamide-containing NSAIDs described herein or known in the art). The concentration of the benzenesulfonamide-containing NSAID (or each of two, three, four, or five different benzenesulfonamide-containing NSAIDs) in any of the compositions (e.g., gels, e.g., hydrogels) provided herein can be about 0.5 mg/mL to about 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, about 32 mg/mL, about 30 mg/mL, about 28 mg/mL, about 26 mg/mL, about 24 mg/mL, about 22 mg/mL, about 20 mg/mL, about 18 mg/mL, about 16 mg/mL, about 14 mg/mL, about 12 mg/mL, about 10 mg/mL, about 8 mg/mL, about 6 mg/mL, about 4 mg/mL, about 2 mg/mL, or about 1 mg/mL (inclusive); about 1 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, about 32 mg/mL, about 30 mg/mL, about 28 mg/mL, about 26 mg/mL, about 24 mg/mL, about 22 mg/mL, about 20 mg/mL, about 18 mg/mL, about 16 mg/mL, about 14 mg/mL, about 12 mg/mL, about 10 mg/mL, about 8 mg/mL, about 6 mg/mL, about 4 mg/mL, or about 2 mg/mL (inclusive); about 2 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, about 32 mg/mL, about 30 mg/mL, about 28 mg/mL, about 26 mg/mL, about 24 mg/mL, about 22 mg/mL, about 20 mg/mL, about 18 mg/mL, about 16 mg/mL, about 14 mg/mL, about 12 mg/mL, about 10 mg/mL, about 8 mg/mL, about 6 mg/mL, or about 4 mg/mL (inclusive); about 4 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, about 32 mg/mL, about 30 mg/mL, about 28 mg/mL, about 26 mg/mL, about 24 mg/mL, about 22 mg/mL, about 20 mg/mL, about 18 mg/mL, about 16 mg/mL, about 14 mg/mL, about 12 mg/mL, about 10 mg/mL, about 8 mg/mL, or about 6 mg/mL (inclusive); about 6 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, about 32 mg/mL, about 30 mg/mL, about 28 mg/mL, about 26 mg/mL, about 24 mg/mL, about 22 mg/mL, about 20 mg/mL, about 18 mg/mL, about 16 mg/mL, about 14 mg/mL, about 12 mg/mL, about 10 mg/mL, or about 8 mg/mL (inclusive); about 8 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, about 32 mg/mL, about 30 mg/mL, about 28 mg/mL, about 26 mg/mL, about 24 mg/mL, about 22 mg/mL, about 20 mg/mL, about 18 mg/mL, about 16 mg/mL, about 14 mg/mL, about 12 mg/mL, or about 10 mg/mL (inclusive); about 10 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, about 32 mg/mL, about 30 mg/mL, about 28 mg/mL, about 26 mg/mL, about 24 mg/mL, about 22 mg/mL, about 20 mg/mL, about 18 mg/mL, about 16 mg/mL, about 14 mg/mL, or about 12 mg/mL (inclusive); about 12 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, about 32 mg/mL, about 30 mg/mL, about 28 mg/mL, about 26 mg/mL, about 24 mg/mL, about 22 mg/mL, about 20 mg/mL, about 18 mg/mL, about 16 mg/mL, or about 14 mg/mL (inclusive); about 14 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, about 32 mg/mL, about 30 mg/mL, about 28 mg/mL, about 26 mg/mL, about 24 mg/mL, about 22 mg/mL, about 20 mg/mL, about 18 mg/mL, or about 16 mg/mL (inclusive); about 16 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, about 32 mg/mL, about 30 mg/mL, about 28 mg/mL, about 26 mg/mL, about 24 mg/mL, about 22 mg/mL, about 20 mg/mL, or about 18 mg/mL (inclusive); about 18 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, about 32 mg/mL, about 30 mg/mL, about 28 mg/mL, about 26 mg/mL, about 24 mg/mL, about 22 mg/mL, or about 20 mg/mL (inclusive); about 20 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, about 32 mg/mL, about 30 mg/mL, about 28 mg/mL, about 26 mg/mL, about 24 mg/mL, or about 22 mg/mL (inclusive); about 22 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, about 32 mg/mL, about 30 mg/mL, about 28 mg/mL, about 26 mg/mL, or about 24 mg/mL (inclusive); about 24 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, about 32 mg/mL, about 30 mg/mL, about 28 mg/mL, or about 26 mg/mL (inclusive); about 26 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, about 32 mg/mL, about 30 mg/mL, or about 28 mg/mL (inclusive); about 28 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, about 32 mg/mL, or about 30 mg/mL (inclusive); about 30 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, about 34 mg/mL, or about 32 mg/mL (inclusive); about 32 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, about 36 mg/mL, or about 34 mg/mL (inclusive); about 34 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, about 38 mg/mL, or about 36 mg/mL (inclusive); about 36 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, about 40 mg/mL, or about 38 mg/mL (inclusive); about 38 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, about 42 mg/mL, or about 40 mg/mL (inclusive); about 40 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, about 44 mg/mL, or about 42 mg/mL (inclusive); about 42 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, about 46 mg/mL, or about 44 mg/mL (inclusive); about 44 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, about 50 mg/mL, about 48 mg/mL, or about 46 mg/mL; about 46 mg/mL to about 50 mg/mL or about 48 mg/mL (inclusive); about 48 mg/mL to 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, about 60 mg/mL, or about 50 mg/mL (inclusive); about 50 mg/mL to about 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, about 70 mg/mL, or about 60 mg/mL (inclusive); about 60 mg/mL to about 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, about 80 mg/mL, or about 70 mg/mL (inclusive); about 70 mg/mL to about 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, about 90 mg/mL, or about 80 mg/mL (inclusive); about 80 mg/mL to about 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, about 100 mg/mL, or about 90 mg/mL (inclusive); about 90 mg/mL to about 200 mg/mL, about 190 mg/mL, about 180 mg/mL, about 170 mg/mL, about 160 mg/mL, about 150 mg/mL, about 140 mg/mL, about 130 mg/mL, about 120 mg/mL, about 110 mg/mL, or about 100 mg/mL (inclusive); about 100 mg/mL to about 200 mg/mL, about 190 mg/mL, about 180 mg/mL, about 170 mg/mL, about 160 mg/mL, about 150 mg/mL, about 140 mg/mL, about 130 mg/mL, about 120 mg/mL, or about 110 mg/mL (inclusive); about 110 mg/mL to about 200 mg/mL, about 190 mg/mL, about 180 mg/mL, about 170 mg/mL, about 160 mg/mL, about 150 mg/mL, about 140 mg/mL, about 130 mg/mL, or about 120 mg/mL (inclusive); about 120 mg/mL to about 200 mg/mL, about 190 mg/mL, about 180 mg/mL, about 170 mg/mL, about 160 mg/mL, about 150 mg/mL, about 140 mg/mL, or about 130 mg/mL (inclusive); about 130 mg/mL to about 200 mg/mL, about 190 mg/mL, about 180 mg/mL, about 170 mg/mL, about 160 mg/mL, about 150 mg/mL, or about 140 mg/mL (inclusive); about 140 mg/mL to about 200 mg/mL, about 190 mg/mL, about 180 mg/mL, about 170 mg/mL, about 160 mg/mL, or about 150 mg/mL (inclusive); about 150 mg/mL to about 200 mg/mL, about 190 mg/mL, about 180 mg/mL, about 170 mg/mL, or about 160 mg/mL (inclusive); about 160 mg/mL to about 200 mg/mL, about 190 mg/mL, about 180 mg/mL, or about 170 mg/mL (inclusive); about 170 mg/mL to about 200 mg/mL, about 190 mg/mL, or about 180 mg/mL (inclusive); about 180 mg/mL to about 200 mg/mL or about 190 mg/mL (inclusive); or about 190 mg/mL to about 200 mg/mL (inclusive) (units are in mg of the benzenesulfonamide-containing NSAID per volume of the composition (e.g., gel, e.g., hydrogel)).

The gelling agent included in any of the compositions (e.g., gels, e.g., hydrogels) provided herein can be, e.g., any of the exemplary gelling agents described herein. For example, the gelling agent included in any of the compositions (e.g., gels, e.g., hydrogels) provided herein can be a PEG (e.g., any of the exemplary PEGs described herein). In some examples, the PEG can be PEG200, PEG300, PEG400, PEG500, or PE600, or a PEG that is about 2 kDa, about 3 kDa, about 4 kDa, about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, or about 20 kDa. For example, the PEG included in any of the compositions (e.g., gels, e.g., hydrogels) provided herein can have an average molecular weight of about 100 g/mole to about 900 g/mole, about 800 g/mole, about 700 g/mole, about 600 g/mole, about 500 g/mole, about 400 g/mole, about 300 g/mole, or about 200 g/mole; about 200 g/mole to about 900 g/mole, about 800 g/mole, about 700 g/mole, about 600 g/mole, about 500 g/mole, about 400 g/mole, or about 300 g/mole; about 300 g/mole to about 900 g/mole, about 800 g/mole, about 700 g/mole, about 600 g/mole, about 500 g/mole, or about 400 g/mole; about 400 g/mole to about 900 g/mole, about 800 g/mole, about 700 g/mole, about 600 g/mole, or about 500 g/mole; about 500 g/mole to about 900 g/mole, about 800 g/mole, about 700 g/mole, or about 600 g/mole; about 600 g/mole to about 900 g/mole, about 800 g/mole, or about 700 g/mole; about 700 g/mole to about 900 g/mole, or about 800 g/mole; or about 800 g/mole to about 900 g/mole.

In some examples, the gelling agent in any of the compositions (e.g., gels, e.g., hydrogels) described herein can be, e.g., sorbitol, triethylamine, 2-pyrrolidone, alpha-cyclodextrin, benzyl alcohol, beta-cyclodextrin, dimethyl sulfoxide, dimethylacetamide (DMA), dimethylformamide, ethanol, gamma-cyclodextrin, glycerol, glycerol formal, hydroxypropyl beta-cyclodextrin, kolliphor 124, kolliphor 181, kolliphor 188, kolliphor 407, kolliphor EL (cremaphor EL), cremaphor RH 40, cremophor RH 60, d-alpha-tocopherol, PEG 1000 succinate, polysorbate 20, polysorbate 80, solutol HS 15, sorbitan monooleate, poloxamer-407, poloxamer-188, Labrafil M-1944CS, Lrabfil M-2125CS, Labrasol, Gellucire 44/14, Softigen 767, mono- and di-fatty acid esters of PEG 300, PEG 400, or PEG 1750, kolliphor RH60, N-methyl-2-pyrrolidone, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil, beeswax, d-alpha-tocopherol, oleic acid, medium-chain mono- and di-glycerides, alpha-cyclodextrin, beta-cyclodextrin, hydroxypropyl-beta-cyclodextrin, sulfo-butylether-beta-cyclodextrin, hydrogenated soy phosphatidylcholine, distearoylphosphatidylglycerol, L-alpha-dimyristoylphosphatidylcholine, L-alpha-dimyristoylphosphatidylglycerol, PEG 300, PEG 300 caprylic/capric glycerides (Softigen 767), PEG 300 linoleic glycerides (Labrafil M-2125CS), PEG 300 oleic glycerides (Labrafil M-1944CS), PEG 400, PEG 400 caprylic/capric glycerides (Labrasol), polyoxyl 40 stearate (PEG 1750 monosterate), polyoxyl 8 stearate (PEG 400 monosterate), polysorbate 20, polysorbate 80, polyvinyl pyrrolidone, propylene carbonate, propylene glycol, solutol HS 15, sorbitan monooleate (Span 20), sulfobutylether-beta-cyclodextrin, transcutol, triacetin, 1-dodecylazacyclo-heptan-2-one, caprolactam, castor oil, cottonseed oil, ethyl acetate, medium chain triglycerides, methyl acetate, oleic acid, safflower oil, sesame oil, soybean oil, tetrahydrofuran, or glycerin.

A composition (e.g., gel, e.g., hydrogel) provided herein can include a single gelling agent or can include two or more (e.g., two, three, four, or five) different gelling agents. The concentration of the gelling agent (or each of two, three, four, or five different gelling agents) in any of the compositions (e.g., gels, e.g., hydrogels) provided herein can be about 0.01 (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), about 2% (w/v), about 1.5% (w/v), about 1.0% (w/v), about 0.5% (w/v), about 0.25% (w/v), or about 0.1% (w/v) (inclusive); about 0.1% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), about 2% (w/v), about 1.5% (w/v), about 1.0% (w/v), about 0.5% (w/v), or about 0.25% (w/v) (inclusive); about 0.25% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), about 2% (w/v), about 1.5% (w/v), about 1.0% (w/v), or about 0.5% (w/v) (inclusive); about 0.5% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), about 2% (w/v), about 1.5% (w/v), or about 1.0% (w/v) (inclusive); about 1.0% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), or about 2% (w/v) (inclusive); about 2.0% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), or about 4% (w/v) (inclusive); about 4% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), or about 6% (w/v) (inclusive); about 6% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), or about 8% (w/v) (inclusive); about 8% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), or about 10% (w/v) (inclusive); about 10% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), or about 12% (w/v) (inclusive); about 12% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), or about 14% (w/v) (inclusive); about 14% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), or about 16% (w/v) (inclusive); about 16% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), or about 18% (w/v) (inclusive); about 18% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), or about 20% (w/v) (inclusive); about 20% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), or about 22% (w/v) (inclusive); about 22% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), or about 24% (w/v) (inclusive); about 24% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), or about 26% (w/v) (inclusive); about 26% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), or about 28% (w/v) (inclusive); about 28% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), or about 30% (w/v) (inclusive); about 30% (w/v) to about 50% (w/v), about 48% (w/v), about 46% (w/v), about 44% (w/v), about 42% (w/v), about 40% (w/v), about 38% (w/v), about 36% (w/v), about 34% (w/v), or about 32% (w/v) (inclusive); about 32% (w/v) to about 50% (w/v), about 48% (w/v), about 46% (w/v), about 44% (w/v), about 42% (w/v), about 40% (w/v), about 38% (w/v), about 36% (w/v), or about 34% (w/v) (inclusive); about 34% (w/v) to about 50% (w/v), about 48% (w/v), about 46% (w/v), about 44% (w/v), about 42% (w/v), about 40% (w/v), about 38% (w/v), or about 36% (w/v) (inclusive); about 36% (w/v) to about 50% (w/v), about 48% (w/v), about 46% (w/v), about 44% (w/v), about 42% (w/v), about 40% (w/v), or about 38% (w/v) (inclusive); about 38% (w/v) to about 50% (w/v), about 48% (w/v), about 46% (w/v), about 44% (w/v), about 42% (w/v), or about 40% (w/v) (inclusive); about 40% (w/v) to about 50% (w/v), about 48% (w/v), about 46% (w/v), about 44% (w/v), or about 42% (w/v) (inclusive); about 42% (w/v) to about 50% (w/v), about 48% (w/v), about 46% (w/v), or about 44% (w/v) (inclusive); about 44% (w/v) to about 50% (w/v), about 48% (w/v), or about 46% (w/v) (inclusive); about 46% (w/v) to about 50% (w/v) or about 48% (w/v) (inclusive); or about 48% (w/v) to about 50% (w/v) (inclusive) (units are in % weight of the gelling agent per volume of the composition (e.g., gel, e.g., hydrogel)).

The concentration of the gelling agent (or each of two, three, four, or five different gelling agents) in any of the compositions (e.g., gels, e.g., hydrogels) provided herein can be about 0.01 (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), about 2% (v/v), about 1.5% (v/v), about 1.0% (v/v), about 0.5% (v/v), about 0.25% (v/v), or about 0.1% (v/v) (inclusive); about 0.1% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), about 2% (v/v), about 1.5% (v/v), about 1.0% (v/v), about 0.5% (v/v), or about 0.25% (v/v) (inclusive); about 0.25% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), about 2% (v/v), about 1.5% (v/v), about 1.0% (v/v), or about 0.5% (v/v) (inclusive); about 0.5% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), about 2% (v/v), about 1.5% (v/v), or about 1.0% (v/v) (inclusive); about 1.0% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), or about 2% (v/v) (inclusive); about 2.0% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), or about 4% (v/v) (inclusive); about 4% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), or about 6% (v/v) (inclusive); about 6% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), or about 8% (v/v) (inclusive); about 8% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), or about 10% (v/v) (inclusive); about 10% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), or about 12% (v/v) (inclusive); about 12% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), or about 14% (v/v) (inclusive); about 14% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), or about 16% (v/v) (inclusive); about 16% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), or about 18% (v/v) (inclusive); about 18% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), or about 20% (v/v) (inclusive); about 20% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), or about 22% (v/v) (inclusive); about 22% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), or about 24% (v/v) (inclusive); about 24% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), or about 26% (v/v) (inclusive); about 26% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), or about 28% (v/v) (inclusive); about 28% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), or about 30% (v/v) (inclusive); about 30% (v/v) to about 50% (v/v), about 48% (v/v), about 46% (v/v), about 44% (v/v), about 42% (v/v), about 40% (v/v), about 38% (v/v), about 36% (v/v), about 34% (v/v), or about 32% (v/v) (inclusive); about 32% (v/v) to about 50% (v/v), about 48% (v/v), about 46% (v/v), about 44% (v/v), about 42% (v/v), about 40% (v/v), about 38% (v/v), about 36% (v/v), or about 34% (v/v) (inclusive); about 34% (v/v) to about 50% (v/v), about 48% (v/v), about 46% (v/v), about 44% (v/v), about 42% (v/v), about 40% (v/v), about 38% (v/v), or about 36% (v/v) (inclusive); about 36% (v/v) to about 50% (v/v), about 48% (v/v), about 46% (v/v), about 44% (v/v), about 42% (v/v), about 40% (v/v), or about 38% (v/v) (inclusive); about 38% (v/v) to about 50% (v/v), about 48% (v/v), about 46% (v/v), about 44% (v/v), about 42% (v/v), or about 40% (v/v) (inclusive); about 40% (v/v) to about 50% (v/v), about 48% (v/v), about 46% (v/v), about 44% (v/v), or about 42% (v/v) (inclusive); about 42% (v/v) to about 50% (v/v), about 48% (v/v), about 46% (v/v), or about 44% (v/v) (inclusive); about 44% (v/v) to about 50% (v/v), about 48% (v/v), or about 46% (v/v) (inclusive); about 46% (v/v) to about 50% (v/v) or about 48% (v/v) (inclusive); or about 48% (v/v) to about 50% (v/v) (inclusive) (units are in % volume of the gelling agent per volume of the composition (e.g., gel, e.g., hydrogel)).

In some embodiments of any of the compositions described herein, the final concentration of a first gelling agent of polysorbate-80 in the composition is about 0.1% (v/v) to about 1.0% (v/v) (inclusive) (e.g., about 0.1% (v/v) to about 0.8% (v/v) (inclusive), about 0.1% (v/v) to about 0.6% (v/v) (inclusive), about 0.1% (v/v) to about 0.5% (v/v) (inclusive), about 0.1% (v/v) to about 0.4% (v/v) (inclusive), about 0.1% (v/v) to about 0.3% (v/v) (inclusive), or about 0.1% (v/v) to about 0.2% (v/v) (inclusive)) and the final concentration of a second gelling agent (e.g., a PEG (e.g., PEG300, PEG400, PEG 2 kDa, PEG 4 kDa, PEG 6 kDa, PEG 8 kDa, or PEG 10 kDa), sorbitol, propylene glycol, glycerol formal, or glycerol, or any of the other gelling agents tested in Example 8) in the composition is about 1% (v/v) to about 50% (v/v) (inclusive) (e.g., about 1% (v/v) to about 48% (v/v), about 46% (v/v), about 44% (v/v), about 42% (v/v), about 40% (v/v), about 38% (v/v), about 36% (v/v), about 34% (v/v), about 32% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), or about 2% (v/v) (inclusive); about 2% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), or about 4% (v/v) (inclusive); about 4% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), or about 6% (v/v) (inclusive); about 6% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), or about 8% (v/v) (inclusive); about 8% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), or about 10% (v/v) (inclusive); about 10% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), or about 12% (v/v) (inclusive); about 12% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v) (inclusive); about 14% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 32% (v/v), about 30% (v/v), about 28% (v/v), about 26% (v/v), about 24% (v/v), about 22% (v/v), about 20% (v/v), about 18% (v/v), or about 16% (v/v) (inclusive); about 16% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 32% (v/v), about 30% (v/v), about 28% (v/v), about 26% (v/v), about 24% (v/v), about 22% (v/v), about 20% (v/v), or about 18% (v/v) (inclusive); about 18% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 32% (v/v), about 30% (v/v), about 28% (v/v), about 26% (v/v), about 24% (v/v), about 22% (v/v), or about 20% (v/v) (inclusive); about 20% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 32% (v/v), about 30% (v/v), about 28% (v/v), about 26% (v/v), about 24% (v/v), or about 22% (v/v) (inclusive); about 22% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 32% (v/v), about 30% (v/v), about 28% (v/v), about 26% (v/v), or about 24% (v/v) (inclusive); about 24% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 32% (v/v), about 30% (v/v), about 28% (v/v), or about 26% (v/v) (inclusive); about 26% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 32% (v/v), about 30% (v/v), or about 28% (v/v) (inclusive); about 28% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 32% (v/v), or about 30% (v/v) (inclusive); about 30% (v/v) to about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), or about 32% (v/v) (inclusive); about 32% (v/v) to about 50% (v/v), about 48% (v/v), about 46% (v/v), about 44% (v/v), about 42% (v/v), about 40% (v/v), about 38% (v/v), about 36% (v/v), about 34% (v/v) (inclusive); about 34% (v/v) to about 50% (v/v), about 48% (v/v), about 46% (v/v), about 44% (v/v), about 42% (v/v), about 40% (v/v), about 38% (v/v), or about 36% (v/v) (inclusive); about 36% (v/v) to about 50% (v/v), about 48% (v/v), about 46% (v/v), about 44% (v/v), about 42% (v/v), about 40% (v/v), or about 38% (v/v) (inclusive); about 38% (v/v) to about 50% (v/v), about 48% (v/v), about 46% (v/v), about 44% (v/v), about 42% (v/v), about 40% (v/v) (inclusive); about 40% (v/v) to about 50% (v/v), about 48% (v/v), about 46% (v/v), about 44% (v/v), or about 42% (v/v) (inclusive); about 42% (v/v) to about 50% (v/v), about 48% (v/v), about 46% (v/v), about 44% (v/v) (inclusive); about 44% (v/v) to about 50% (v/v), about 48% (v/v), or about 46% (v/v) (inclusive); about 46% (v/v) to about 50% (v/v), or about 48% (v/v) (inclusive); about 48% (v/v) to about 50% (v/v) (inclusive)), or about 1% (w/v) to about 50% (w/v) (inclusive) (e.g., about 1% (w/v) to about 48% (w/v), about 46% (w/v), about 44% (w/v), about 42% (w/v), about 40% (w/v), about 38% (w/v), about 36% (w/v), about 34% (w/v), about 32% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), or about 2% (w/v) (inclusive); about 2% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), or about 4% (w/v) (inclusive); about 4% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), or about 6% (w/v) (inclusive); about 6% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), or about 8% (w/v) (inclusive); about 8% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), or about 10% (w/v) (inclusive); about 10% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), or about 12% (w/v) (inclusive); about 12% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v) (inclusive); about 14% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 32% (w/v), about 30% (w/v), about 28% (w/v), about 26% (w/v), about 24% (w/v), about 22% (w/v), about 20% (w/v), about 18% (w/v), or about 16% (w/v) (inclusive); about 16% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 32% (w/v), about 30% (w/v), about 28% (w/v), about 26% (w/v), about 24% (w/v), about 22% (w/v), about 20% (w/v), or about 18% (w/v) (inclusive); about 18% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 32% (w/v), about 30% (w/v), about 28% (w/v), about 26% (w/v), about 24% (w/v), about 22% (w/v), or about 20% (w/v) (inclusive); about 20% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 32% (w/v), about 30% (w/v), about 28% (w/v), about 26% (w/v), about 24% (w/v), or about 22% (w/v) (inclusive); about 22% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 32% (w/v), about 30% (w/v), about 28% (w/v), about 26% (w/v), or about 24% (w/v) (inclusive); about 24% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 32% (w/v), about 30% (w/v), about 28% (w/v), or about 26% (w/v) (inclusive); about 26% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 32% (w/v), about 30% (w/v), or about 28% (w/v) (inclusive); about 28% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 32% (w/v), or about 30% (w/v) (inclusive); about 30% (w/v) to about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), or about 32% (w/v) (inclusive); about 32% (w/v) to about 50% (w/v), about 48% (w/v), about 46% (w/v), about 44% (w/v), about 42% (w/v), about 40% (w/v), about 38% (w/v), about 36% (w/v), about 34% (w/v) (inclusive); about 34% (w/v) to about 50% (w/v), about 48% (w/v), about 46% (w/v), about 44% (w/v), about 42% (w/v), about 40% (w/v), about 38% (w/v), or about 36% (w/v) (inclusive); about 36% (w/v) to about 50% (w/v), about 48% (w/v), about 46% (w/v), about 44% (w/v), about 42% (w/v), about 40% (w/v), or about 38% (w/v) (inclusive); about 38% (w/v) to about 50% (w/v), about 48% (w/v), about 46% (w/v), about 44% (w/v), about 42% (w/v), about 40% (w/v) (inclusive); about 40% (w/v) to about 50% (w/v), about 48% (w/v), about 46% (w/v), about 44% (w/v), or about 42% (w/v) (inclusive); about 42% (w/v) to about 50% (w/v), about 48% (w/v), about 46% (w/v), about 44% (w/v) (inclusive); about 44% (w/v) to about 50% (w/v), about 48% (w/v), or about 46% (w/v) (inclusive); about 46% (w/v) to about 50% (w/v), or about 48% (w/v) (inclusive); about 48% (w/v) to about 50% (w/v) (inclusive)).

Methods of purifying silk fibroin are known in the art. See, e.g., the methods described in U.S. Pat. No. 5,252,285 (incorporated herein by reference) and Rockwood et al., Nature Protocols 6(10): 1612-1631, 2011, and Wray et al., J. Biomed. Mater. Res. B Appl. Biomater. 99(1):89-101, 2011. Silk fibroin can also be purchased from a number of different commercial vendors (e.g., Jiangsu SOHO International Group; Simatech, Suzhou, China; Xi'an Lyphar Biotech, Ltd.; Xi'an Rongsheng Biotechnology; Mulberry Farms, Treenway Silks, Sharda Group, Maniar Enterprises, and Wild Fibres). The concentration of silk fibroin in any of the compositions (e.g., gels, e.g., hydrogels) provided herein can be about 0.5% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), about 6% (w/v), about 5% (w/v), about 4% (w/v), about 3% (w/v), about 2% (w/v), or about 1% (w/v); about 1% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), about 6% (w/v), about 5% (w/v), about 4% (w/v), about 3% (w/v), or about 2% (w/v); about 2% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), about 6% (w/v), about 5% (w/v), about 4% (w/v), or about 3% (w/v); about 3% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), about 6% (w/v), about 5% (w/v), or about 4% (w/v); about 4% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), about 6% (w/v), or about 5% (w/v); about 5% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), or about 6% (w/v); about 6% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), or about 7% (w/v); about 7% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), or about 8% (w/v); about 8% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), or about 9% (w/v); about 9% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), or about 10% (w/v); about 10% (w/v) to about 30% (w/v), about 28% (w/v), about 26% (w/v), about 24% (w/v), about 22% (w/v), about 20% (w/v), about 18% (w/v), about 16% (w/v), about 14% (w/v), or about 12% (w/v); about 12% (w/v) to about 30% (w/v), about 28% (w/v), about 26% (w/v), about 24% (w/v), about 22% (w/v), about 20% (w/v), about 18% (w/v), about 16% (w/v), or about 14% (w/v); about 14% (w/v) to about 30% (w/v), about 28% (w/v), about 26% (w/v), about 24% (w/v), about 22% (w/v), about 20% (w/v), about 18% (w/v), or about 16% (w/v); about 16% (w/v) to about 30% (w/v), about 28% (w/v), about 26% (w/v), about 24% (w/v), about 22% (w/v), about 20% (w/v), or about 18% (w/v); about 18% (w/v) to about 30% (w/v), about 28% (w/v), about 26% (w/v), about 24% (w/v), about 22% (w/v), or about 20% (w/v); about 20% (w/v) to about 30% (w/v), about 28% (w/v), about 26% (w/v), about 24% (w/v), or about 22% (w/v); about 22% (w/v) to about 30% (w/v), about 28% (w/v), about 26% (w/v), or about 24% (w/v); about 24% (w/v) to about 30% (w/v), about 28% (w/v), or about 26% (w/v); about 26% (w/v) to about 30% (w/v) or about 28% (w/v); or about 28% (w/v) to about 30% (w/v) (units are in % weight of the silk fibroin per volume of the composition (e.g., gel, e.g., hydrogel)).

In some examples, the composition is a gel (e.g., a hydrogel or an organogel). In some examples, the composition (e.g., gel, e.g., hydrogel) includes a pharmaceutically acceptable solvent (e.g., saline or phosphate buffered saline). In some examples, the compositions (e.g., gels, e.g., hydrogels) include one or more (e.g., two, three, or four) of an antibacterial agent, an antifungal agent, and an analgesic agent. Non-limiting examples of antibacterial agents include amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipeneum, cilastatin, meropenem, cefadroxil, cefazolin, cefalotin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, linomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin, ciprofolaxin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, sulfadimethoxine, sulfamethizole, sulfamethoxazole, sulfanilimide, sulfasalazine, sulfisoxazole, demeclocycline, doxycycline, minocycline, oxytetracycline, tetracycline, clofazimine, dapsone, capreomycin, cycloserine, ethambutol, ethionamide, isoniazid, pyrazinamide, rifampicin, and streptomycin. Non-limiting examples of antifungal agents include: amphotericin B, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, abafungin, amorolfin, butenafine, naftifine, terbinafine, anidulafungin, caspofungin, micafungin, benzoic acid, ciclopirox, flucytosine, 5-fluorocytosine, griseofulvin, haloprogin, tolnaftate, undecylenic acid, and crystal violet. Non-limiting examples of analgesic agents include procaine, amethocaine, cocaine, lidocaine, prilocaine, bupivacaine, levobupivacaine, ropivacaine, mepivacaine, dibucaine, amobarbital, methohexital, thiamylal, thiopental, diazepam, lorazepam, midazolam, etomidate, ketamine, propofol, alfentanil, fentanyl, remifentanil, sufentanil, buprenorphine, butorphanol, diacetyl morphine, hydromorphone, levorphanol, meperidine, methadone, morphine, nalbuphine, oxycodone, oxymorphone, pentazocine, succinylcholine, decamethonium, rapacuronium, atracurium, cisatracurium, rocuronium, vecuronium, alcuronium, doxacurium, gallamine, metocurine, pancuronium, pipecuronium, and tubocurarine.

Any of the compositions (e.g., gels, e.g., hydrogels) described herein (or produced by any of the methods described herein) can include a plurality of particles including one or more (e.g., two or three) of a benzenesulfonamide-containing NSAID (e.g., any of the benzenesulfonamide-containing NSAIDs described herein), silk fibroin (e.g., any of the examples of silk fibroin described herein), and a gelling agent (e.g., any of the gelling agents described herein). In some examples of any of the compositions (e.g., gels, e.g., hydrogels) described herein, the plurality of particles have: a d90 of less than about 200 μm, less than about 195 μm, less than about 190 μm, less than about 185 μm, less than about 180 μm, less than about 175 μm, less than about 170 μm, less than about 165 μm, less than about 160 μm, less than about 155 μm, less than about 150 μm, less than about 145 μm, less than about 140 μm, less than about 135 μm, less than about 130 μm, less than about 125 μm, less than about 120 μm, less than about 115 μm, less than about 110 μm, less than about 105 μm, less than about 100 μm, less than about 98 μm, less than about 96 μm, less than about 94 μm, less than about 92 μm, less than about 90 μm, less than about 88 μm, less than about 86 μm, less than about 84 μm, less than about 82 μm, less than about 80 μm, less than about 78 μm, less than about 76 μm, less than about 74 μm, less than about 72 μm, less than about 70 μm, less than about 68 μm, less than about 66 μm, less than about 64 μm, less than about 62 μm, less than about 60 μm, less than about 58 μm, less than about 56 μm, less than about 54 μm, less than about 52 μm, less than about 50 μm, less than about 48 μm, less than about 46 μm, less than about 44 μm, less than about 42 μm, less than about 40 μm, less than about 38 μm, less than about 36 μm, less than about 34 μm, less than about 32 μm, less than about 30 μm, less than about 28 μm, less than about 26 μm, less than about 24 μm, less than about 22 μm, less than about 20 μm, less than about 18 μm, less than about 16 μm, less than about 14 μm, less than about 12 μm, or less than about 10 μm. Any of the compositions (e.g., gels, e.g., hydrogels) described herein (or produced by any of the methods described herein) can include a plurality of particles having a d50 of less than about less than about 80 μm, less than about 78 μm, less than about 76 μm, less than about 74 μm, less than about 72 μm, less than about 70 μm, less than about 68 μm, less than about 66 μm, less than about 64 μm, less than about 62 μm, less than about 60 μm, less than about 58 μm, less than about 56 μm, less than about 54 μm, less than about 52 μm, less than about 50 μm, less than about 48 μm, less than about 46 μm, less than about 44 μm, less than about 42 μm, less than about 40 μm, less than about 38 μm, less than about 36 μm, less than about 34 μm, less than about 32 μm, less than about 30 μm, less than about 29 μm, less than about 28 μm, less than about 27 μm, less than about 26 μm, less than about 25 μm, less than about 24 μm, less than about 23 μm, less than about 22 μm, less than about 21 μm, less than about 20 μm, less than about 19 μm, less than about 18 μm, less than about 17 μm, less than about 16 μm, less than about 15 μm, less than about 14 μm, less than about 13 μm, less than about 12 μm, less than about 11 μm, or less than about 10 μm. Any of the compositions (e.g., gels, e.g., hydrogels) described herein (or produced by any of the methods described herein) can include a plurality of particles having a d10 of less than about 40 μm, less than about 39 μm, less than about 38 μm, less than about 37 μm, less than about 36 μm, less than about 35 μm, less than about 34 μm, less than about 33 μm, less than about 32 μm, less than about 31 μm, less than about 30 μm, less than about 29 μm, less than about 28 μm, less than about 27 μm, less than about 26 μm, less than about 25 μm, less than about 24 μm, less than about 23 μm, less than about 22 μm, less than about 21 μm, less than about 20 μm, less than about 19 μm, less than about 18 μm, less than about 17 μm, less than about 16 μm, less than about 15 μm, less than about 14 μm, less than about 13 μm, less than about 12 μm, less than about 11 μm, less than about 10 μm, less than about 9 μm, less than about 8 μm, less than about 7 μm, less than about 6 μm, less than about 5 μm, less than about 4 μm, less than about 3 μm, less than about 2 μm, or less than about 1 μm. In some examples, the plurality of particles can include one or more benzenesulfonamide-containing NSAIDS. In some examples, the plurality of particles can include silk fibroin (e.g., any of the silk fibroin proteins described herein). In some examples, the plurality of particles can include at least one gelling agent (e.g., any of the gelling agents described herein). In some examples, the plurality of particles can include a benzenesulfonamide-containing NSAID and silk fibroin (e.g., any of the silk fibroin proteins described herein), e.g., where the benzenesulfonamide-containing NSAID and the silk fibroin are homogenously distributed within the particle (e.g., a co-crystal), or the benzenesulfonamide-containing NSAID and the silk fibroin are not evenly distributed within the particle (e.g., the exterior surface of the particles is coated with silk fibroin and the interior of the particles includes the benzenesulfonamide-containing NSAID).

In some examples, the plurality of particles can include a benzenesulfonamide-containing NSAID and the gelling agent (e.g., any of the gelling agents described herein), e.g., where the benzenesulfonamide-containing NSAID and the gelling agent are homogenously distributed within the particle (e.g., a co-crystal), or the benzenesulfonamide-containing NSAID and the gelling agent are not evenly distributed within the particle (e.g., the exterior surface of the particles is coated with the gelling agent and the interior of the particles includes the benzenesulfonamide-containing NSAID).

In some examples, the plurality of particles can include the benzenesulfonamide-containing NSAID, the gelling agent, and silk fibroin (e.g., any of the silk fibroin proteins described herein), e.g., where the benzenesulfonamide-containing NSAID, the gelling agent, and the silk fibroin are homogenously distributed within the particle (e.g., a co-crystal), or the benzenesulfonamide-containing NSAID, the gelling agent, and the silk fibroin are not evenly distributed within the particle (e.g., the exterior surface of the particles is coated with one or both of silk fibroin and the gelling agent, and the interior of the particles includes the benzenesulfonamide-containing NSAID).

In some examples, the plurality of particles can have an ellipsoidal or oblong shape. In some examples, the plurality of particles can have a spherical or an approximately spherical shape.

Any of the compositions (e.g., gels, e.g., hydrogels or organogels) described herein (or produced by any of the methods described herein) can have a viscosity of about 1 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, about 400 Pa-s, about 350 Pa-s, about 300 Pa-s, about 250 Pa-s, about 200 Pa-s, about 150 Pa-s, about 100 Pa-s, about 90 Pa-s, about 80 Pa-s, about 70 Pa-s, about 60 Pa-s, about 50 Pa-s, about 40 Pa-s, about 30 Pa-s, about 20 Pa-s, about 10 Pa-s, or about 5 Pa-s (inclusive); about 5 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, about 400 Pa-s, about 350 Pa-s, about 300 Pa-s, about 250 Pa-s, about 200 Pa-s, about 150 Pa-s, about 100 Pa-s, about 90 Pa-s, about 80 Pa-s, about 70 Pa-s, about 60 Pa-s, about 50 Pa-s, about 40 Pa-s, about 30 Pa-s, about 20 Pa-s, or about 10 Pa-s (inclusive); about 10 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, about 400 Pa-s, about 350 Pa-s, about 300 Pa-s, about 250 Pa-s, about 200 Pa-s, about 150 Pa-s, about 100 Pa-s, about 90 Pa-s, about 80 Pa-s, about 70 Pa-s, about 60 Pa-s, about 50 Pa-s, about 40 Pa-s, about 30 Pa-s, or about 20 Pa-s (inclusive); about 20 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, about 400 Pa-s, about 350 Pa-s, about 300 Pa-s, about 250 Pa-s, about 200 Pa-s, about 150 Pa-s, about 100 Pa-s, about 90 Pa-s, about 80 Pa-s, about 70 Pa-s, about 60 Pa-s, about 50 Pa-s, about 40 Pa-s, or about 30 Pa-s (inclusive); about 30 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, about 400 Pa-s, about 350 Pa-s, about 300 Pa-s, about 250 Pa-s, about 200 Pa-s, about 150 Pa-s, about 100 Pa-s, about 90 Pa-s, about 80 Pa-s, about 70 Pa-s, about 60 Pa-s, about 50 Pa-s, or about 40 Pa-s (inclusive); about 40 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, about 400 Pa-s, about 350 Pa-s, about 300 Pa-s, about 250 Pa-s, about 200 Pa-s, about 150 Pa-s, about 100 Pa-s, about 90 Pa-s, about 80 Pa-s, about 70 Pa-s, about 60 Pa-s, or about 50 Pa-s (inclusive); about 50 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, about 400 Pa-s, about 350 Pa-s, about 300 Pa-s, about 250 Pa-s, about 200 Pa-s, about 150 Pa-s, about 100 Pa-s, about 90 Pa-s, about 80 Pa-s, about 70 Pa-s, or about 60 Pa-s (inclusive); about 60 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, about 400 Pa-s, about 350 Pa-s, about 300 Pa-s, about 250 Pa-s, about 200 Pa-s, about 150 Pa-s, about 100 Pa-s, about 90 Pa-s, about 80 Pa-s, or about 70 Pa-s (inclusive); about 70 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, about 400 Pa-s, about 350 Pa-s, about 300 Pa-s, about 250 Pa-s, about 200 Pa-s, about 150 Pa-s, about 100 Pa-s, about 90 Pa-s, or about 80 Pa-s (inclusive); about 80 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, about 400 Pa-s, about 350 Pa-s, about 300 Pa-s, about 250 Pa-s, about 200 Pa-s, about 150 Pa-s, about 100 Pa-s, or about 90 Pa-s (inclusive); about 90 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, about 400 Pa-s, about 350 Pa-s, about 300 Pa-s, about 250 Pa-s, about 200 Pa-s, about 150 Pa-s, or about 100 Pa-s (inclusive); about 100 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, about 400 Pa-s, about 350 Pa-s, about 300 Pa-s, about 250 Pa-s, about 200 Pa-s, or about 150 Pa-s (inclusive); about 150 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, about 400 Pa-s, about 350 Pa-s, about 300 Pa-s, about 250 Pa-s, or about 200 Pa-s (inclusive); about 200 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, about 400 Pa-s, about 350 Pa-s, about 300 Pa-s, or about 250 Pa-s (inclusive); about 250 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, about 400 Pa-s, about 350 Pa-s, or about 300 Pa-s (inclusive); about 300 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, about 400 Pa-s, or about 350 Pa-s (inclusive); about 350 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, about 450 Pa-s, or about 400 Pa-s (inclusive); about 400 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, about 500 Pa-s, or about 450 Pa-s (inclusive); about 450 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, about 550 Pa-s, or about 500 Pa-s (inclusive); about 500 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, about 600 Pa-s, or about 550 Pa-s (inclusive); about 550 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, about 650 Pa-s, or about 600 Pa-s (inclusive); about 600 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, about 700 Pa-s, or about 650 Pa-s (inclusive); about 650 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, about 750 Pa-s, or about 700 Pa-s (inclusive); about 700 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, about 800 Pa-s, or about 750 Pa-s (inclusive); about 750 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, about 850 Pa-s, or about 800 Pa-s (inclusive); about 800 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, about 900 Pa-s, or about 850 Pa-s (inclusive); about 850 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, about 950 Pa-s, or about 900 Pa-s (inclusive); about 900 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, about 1,000 Pa-s, or about 950 Pa-s (inclusive); about 950 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, about 1,050 Pa-s, or about 1,000 Pa-s (inclusive); about 1,000 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, about 1,100 Pa-s, or about 1,050 Pa-s (inclusive); about 1,050 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, about 1,150 Pa-s, or about 1,100 Pa-s (inclusive); about 1,100 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, about 1,200 Pa-s, or about 1,150 Pa-s (inclusive); about 1,150 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, about 1,250 Pa-s, or about 1,200 Pa-s (inclusive); about 1,200 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, about 1,300 Pa-s, or about 1,250 Pa-s (inclusive); about 1,250 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, about 1,350 Pa-s, or about 1,300 Pa-s (inclusive); about 1,300 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, about 1,400 Pa-s, or about 1,350 Pa-s (inclusive); about 1,350 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, about 1,450 Pa-s, or about 1,400 Pa-s (inclusive); about 1,400 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, about 1,500 Pa-s, or about 1,450 Pa-s (inclusive); about 1,450 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, about 1,550 Pa-s, or about 1,500 Pa-s (inclusive); about 1,500 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, about 1,600 Pa-s, or about 1,550 Pa-s (inclusive); about 1,550 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, about 1,650 Pa-s, or about 1,600 Pa-s (inclusive); about 1,600 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, about 1,700 Pa-s, or about 1,650 Pa-s (inclusive); about 1,650 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, about 1,750 Pa-s, or about 1,700 Pa-s (inclusive); about 1,700 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, about 1,800 Pa-s, or about 1,750 Pa-s (inclusive); about 1,750 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, about 1,850 Pa-s, or about 1,800 Pa-s (inclusive); about 1,800 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s, or about 1,850 Pa-s (inclusive); about 1,850 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, about 1,950 Pa-s, about 1,900 Pa-s (inclusive); about 1,900 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, about 2,000 Pa-s, or about 1,950 Pa-s (inclusive); about 1,950 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, about 2,200 Pa-s, or about 2,000 Pa-s (inclusive); about 2,000 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, about 2,400 Pa-s, or about 2,200 Pa-s (inclusive); about 2,200 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, about 2,600 Pa-s, or about 2,400 Pa-s (inclusive); about 2,400 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, about 2,800 Pa-s, or about 2,600 Pa-s (inclusive); about 2,600 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, about 3,000 Pa-s, or about 2,800 Pa-s (inclusive); about 2,800 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, about 3,200 Pa-s, or about 3,000 Pa-s (inclusive); about 3,000 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, about 3,400 Pa-s, or about 3,200 Pa-s (inclusive); about 3,200 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, about 3,600 Pa-s, or about 3,400 Pa-s (inclusive); about 3,400 Pa-s to about 5,000 Pa-s, about 4,800 Pa-s, about 4,600 Pa-s, about 4,400 Pa-s, about 4,200 Pa-s, about 4,000 Pa-s, about 3,800 Pa-s, or about 3,600 Pa-s (inclusive); about 3,600 Pa-s to about 5,000 Pa-s, about 4,800 Pa-s, about 4,600 Pa-s, about 4,400 Pa-s, about 4,200 Pa-s, about 4,000 Pa-s, or about 3,800 Pa-s (inclusive); about 3,800 Pa-s to about 5,000 Pa-s, about 4,800 Pa-s, about 4,600 Pa-s, about 4,400 Pa-s, about 4,200 Pa-s, or about 4,000 Pa-s (inclusive); about 4,000 Pa-s to about 5,000 Pa-s, about 4,800 Pa-s, about 4,600 Pa-s, about 4,400 Pa-s, or about 4,200 Pa-s (inclusive); about 4,200 Pa-s to about 5,000 Pa-s, about 4,800 Pa-s, about 4,600 Pa-s, or about 4,400 Pa-s (inclusive); about 4,400 Pa-s to about 5,000 Pa-s, about 4,800 Pa-s, or about 4,600 Pa-s (inclusive); about 4,600 Pa-s to about 5,000 Pa-s, or about 4,800 Pa-s (inclusive); about 4,800 Pa-s to about 5,000 Pa-s (inclusive). The viscosity of a composition (e.g., a gel, e.g., hydrogel or organogel) provided herein can be determined using a rotational viscometer or rheometer. Additional methods for determining the viscosity of a composition (e.g., gel, e.g., hydrogel or organogel) and other properties of the gel are known in the art. See, e.g., the methods described in Wang et al., Acta Biomaterialia 12:51-61 (2015); Fang et al., Chem. Pharm. Bull. 54(2):156-162 (2006); and Pritchard et al., Macromol. Biosci. 13(3):311-320, 2013.

In any of the methods described herein, any of the compositions (e.g., gels, e.g., hydrogels or organogels) described herein can be administered to a subject (e.g., any of the subjects described herein) in a single dose having a volume of about 0.1 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, about 65 mL, about 60 mL, about 55 mL, about 50 mL, about 45 mL, about 40 mL, about 35 mL, about 30 mL, about 25 mL, about 20 mL, about 15 mL, about 10 mL, about 5 mL, about 2.5 mL, about 1 mL, about 0.5 mL, or about 0.2 mL; about 0.2 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, about 65 mL, about 60 mL, about 55 mL, about 50 mL, about 45 mL, about 40 mL, about 35 mL, about 30 mL, about 25 mL, about 20 mL, about 15 mL, about 10 mL, about 5 mL, about 2.5 mL, about 1 mL, or about 0.5 mL; about 0.5 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, about 65 mL, about 60 mL, about 55 mL, about 50 mL, about 45 mL, about 40 mL, about 35 mL, about 30 mL, about 25 mL, about 20 mL, about 15 mL, about 10 mL, about 5 mL, about 2.5 mL, or about 1 mL; about 1 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, about 65 mL, about 60 mL, about 55 mL, about 50 mL, about 45 mL, about 40 mL, about 35 mL, about 30 mL, about 25 mL, about 20 mL, about 15 mL, about 10 mL, about 5 mL, or about 2.5 mL; about 2.5 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, about 65 mL, about 60 mL, about 55 mL, about 50 mL, about 45 mL, about 40 mL, about 35 mL, about 30 mL, about 25 mL, about 20 mL, about 15 mL, about 10 mL, or about 5 mL; about 5 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, about 65 mL, about 60 mL, about 55 mL, about 50 mL, about 45 mL, about 40 mL, about 35 mL, about 30 mL, about 25 mL, about 20 mL, about 15 mL, or about 10 mL; about 10 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, about 65 mL, about 60 mL, about 55 mL, about 50 mL, about 45 mL, about 40 mL, about 35 mL, about 30 mL, about 25 mL, about 20 mL, or about 15 mL; about 15 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, about 65 mL, about 60 mL, about 55 mL, about 50 mL, about 45 mL, about 40 mL, about 35 mL, about 30 mL, about 25 mL, or about 20 mL; about 20 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, about 65 mL, about 60 mL, about 55 mL, about 50 mL, about 45 mL, about 40 mL, about 35 mL, about 30 mL, or about 25 mL; about 25 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, about 65 mL, about 60 mL, about 55 mL, about 50 mL, about 45 mL, about 40 mL, about 35 mL, or about 30 mL; about 30 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, about 65 mL, about 60 mL, about 55 mL, about 50 mL, about 45 mL, about 40 mL, or about 35 mL; about 35 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, about 65 mL, about 60 mL, about 55 mL, about 50 mL, about 45 mL, or about 40 mL; about 40 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, about 65 mL, about 60 mL, about 55 mL, about 50 mL, or about 45 mL; about 45 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, about 65 mL, about 60 mL, about 55 mL, or about 50 mL; about 50 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, about 65 mL, about 60 mL, or about 55 mL; about 55 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, about 65 mL, or about 60 mL; about 60 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, about 70 mL, or about 65 mL; about 65 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, about 75 mL, or about 70 mL; about 70 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, about 80 mL, or about 75 mL; about 75 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, about 85 mL, or about 80 mL; about 80 mL to about 150 mL, about 145 mL, about 140 mL, about 135 mL, about 130 mL, about 125 mL, about 120 mL, about 115 mL, about 110 mL, about 105 mL, about 100 mL, about 95 mL, about 90 mL, or about 85 mL; about 85 mL to about 150 mL, about 145 mL, about 140 mL, about 135 mL, about 130 mL, about 125 mL, about 120 mL, about 115 mL, about 110 mL, about 105 mL, about 100 mL, about 95 mL, or about 90 mL; about 90 mL to about 150 mL, about 145 mL, about 140 mL, about 135 mL, about 130 mL, about 125 mL, about 120 mL, about 115 mL, about 110 mL, about 105 mL, about 100 mL, or about 95 mL; about 95 mL to about 150 mL, about 145 mL, about 140 mL, about 135 mL, about 130 mL, about 125 mL, about 120 mL, about 115 mL, about 110 mL, about 105 mL, or about 100 mL; about 100 mL to about 150 mL, about 145 mL, about 140 mL, about 135 mL, about 130 mL, about 125 mL, about 120 mL, about 115 mL, about 110 mL, or about 105 mL; about 105 mL to about 150 mL, about 145 mL, about 140 mL, about 135 mL, about 130 mL, about 125 mL, about 120 mL, about 115 mL, or about 110 mL; about 110 mL to about 150 mL, about 145 mL, about 140 mL, about 135 mL, about 130 mL, about 125 mL, about 120 mL, or about 115 mL; about 115 mL to about 150 mL, about 145 mL, about 140 mL, about 135 mL, about 130 mL, about 125 mL, or about 120 mL; about 120 mL to about 150 mL, about 145 mL, about 140 mL, about 135 mL, about 130 mL, or about 125 mL; about 125 mL to about 150 mL, about 145 mL, about 140 mL, about 135 mL, or about 130 mL; about 130 mL to about 150 mL, about 145 mL, about 140 mL, or about 135 mL; about 135 mL to about 150 mL, about 145 mL, or about 140 mL; about 140 mL to about 150 mL or about 145 mL; or about 145 mL to about 150 mL.

In some embodiments of any of the methods described herein, the total amount of the benzenesulfonamide-containing NSAID or each of the benzenesulfonamide-containing NSAIDs (for compositions (e.g., gels, e.g., hydrogels or organogels) including two or more benzenesulfonamide-containing NSAIDs) administered to the subject in a single dose of the composition (e.g., gel, e.g., hydrogel or organogel) can be about 5 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, about 100 mg, about 80 mg, about 60 mg, about 40 mg, about 20 mg, or about 10 mg; about 10 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, about 100 mg, about 80 mg, about 60 mg, about 40 mg, or about 20 mg; about 20 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, about 100 mg, about 80 mg, about 60 mg, or about 40 mg; about 40 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, about 100 mg, about 80 mg, or about 60 mg; about 60 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, about 100 mg, or about 80 mg; about 80 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, or about 100 mg; about 100 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, or about 120 mg; about 120 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, or about 140 mg; about 140 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, or about 160 mg; about 160 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, or about 180 mg; about 180 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, or about 200 mg; about 200 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, or about 250 mg; about 250 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, about 500 mg, about 450 mg, about 400 mg, about 350 mg, or about 300 mg; about 300 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, about 500 mg, about 450 mg, about 400 mg, or about 350 mg; about 350 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, about 500 mg, about 450 mg, or about 400 mg; about 400 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, about 500 mg, or about 450 mg; about 450 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, about 550 mg, or about 500 mg; about 500 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, about 600 mg, or about 550 mg; about 550 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, about 650 mg, or about 600 mg; about 600 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, about 700 mg, or about 650 mg; about 650 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, about 750 mg, or about 700 mg; about 700 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, about 800 mg, or about 750 mg; about 750 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, about 850 mg, or about 800 mg; about 800 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, about 900 mg, or about 850 mg; about 850 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, about 1 g, or about 900 mg; about 900 mg to about 7.5 g, about 7 g, about 6.5 g, about 6 g, about 5.5 g, about 5 g, about 4.5 g, about 4 g, about 3.5 g, about 3 g, about 2.5 g, about 2 g, about 1.5 g, or about 1 g; about 1 g to about 7.5 g, about 7 g, about 6.5 g, about 6 g, about 5.5 g, about 5 g, about 4.5 g, about 4 g, about 3.5 g, about 3 g, about 2.5 g, about 2 g, or about 1.5 g; about 1.5 g to about 7.5 g, about 7 g, about 6.5 g, about 6 g, about 5.5 g, about 5 g, about 4.5 g, about 4 g, about 3.5 g, about 3 g, about 2.5 g, or about 2 g; about 2 g to about 7.5 g, about 7 g, about 6.5 g, about 6 g, about 5.5 g, about 5 g, about 4.5 g, about 4 g, about 3.5 g, about 3 g, or about 2.5 g; about 2.5 g to about 7.5 g, about 7 g, about 6.5 g, about 6 g, about 5.5 g, about 5 g, about 4.5 g, about 4 g, about 3.5 g, or about 3 g; about 3 g to about 7.5 g, about 7 g, about 6.5 g, about 6 g, about 5.5 g, about 5 g, about 4.5 g, about 4 g, or about 3.5 g; about 3.5 g to about 7.5 g, about 7 g, about 6.5 g, about 6 g, about 5.5 g, about 5 g, about 4.5 g, or about 4 g; about 4 g to about 7.5 g, about 7 g, about 6.5 g, about 6 g, about 5.5 g, about 5 g, or about 4.5 g; about 4.5 g to about 7.5 g, about 7 g, about 6.5 g, about 6 g, about 5.5 g, or about 5 g; about 5 g to about 7.5 g, about 7 g, about 6.5 g, about 6 g, or about 5.5 g; about 5.5 g to about 7.5 g, about 7 g, about 6.5 g, or about 6 g; about 6 g to about 7.5 g, about 7 g, or about 6.5 g; about 6.5 g to about 7.5 g or about 7 g; or about 7 g to about 7.5 g.

### Methods of Making Compositions—Part A

Also provided herein are methods of making a composition (e.g., a gel, e.g., a hydrogel) including: (a) providing a first solution including about 1% (w/v) to about 60% (w/v) silk fibroin (e.g., silk fibroin having a molecular weight of about 10 kDa to about 500 kDa (or any of the subranges described herein)); (b) providing a second solution including about 0.02% (w/v) to about 100% (w/v), or about 0.02% (v/v) to about 100% (v/v), of a gelling agent (e.g., any of exemplary gelling agents described herein) and about 1 mg/mL to about 100 mg/mL of a benzenesulfonamide-containing NSAID (e.g., any of the exemplary benzenesulfonamide-containing NSAIDs described herein or known in the art); (c) mixing a volume of the first solution with a volume of the second solution, where the ratio of the volume of the first solution to the volume of the second solution is about 5:1 to about 1:5 to provide a mixture; and (d) incubating the mixture for a time sufficient to form the composition (e.g., gel, e.g., hydrogel).

The first solution can be an aqueous solution (e.g., a pharmaceutically acceptable aqueous solution, such as saline or phosphate buffered saline). The first solution can include, e.g., a concentration of silk fibroin (e.g., a concentration of silk fibroin having a molecular weight of about 10 kDa to about 500 kDa (or any of the subranges described herein)) of about 1% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), or about 2% (w/v); about 2% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), to about 4% (w/v); about 4% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), or about 6% (w/v); about 6% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), or about 8% (w/v); about 8% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), or about 10% (w/v); about 10% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), or about 12% (w/v); about 12% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), or about 14% (w/v); about 14% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), or about 16% (w/v); about 16% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), or about 18% (w/v); about 18% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), or about 20% (w/v); about 20% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), or about 22% (w/v); about 22% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), or about 24% (w/v); about 24% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), or about 26% (w/v); about 26% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), or about 28% (w/v); about 28% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), or about 30% (w/v); about 30% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), or about 32% (w/v); about 32% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), or about 34% (w/v); about 34% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), or about 36% (w/v); about 36% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), or about 38% (w/v); about 38% (w/v) to about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), or about 40% (w/v); about 40% (w/v) to about 60% (w/v), about 58% (w/v), about 56% (w/v), about 54% (w/v), about 52% (w/v), about 50% (w/v), about 48% (w/v), about 46% (w/v), about 44% (w/v), or about 42% (w/v); about 42% (w/v) to about 60% (w/v), about 58% (w/v), about 56% (w/v), about 54% (w/v), about 52% (w/v), about 50% (w/v), about 48% (w/v), about 46% (w/v), or about 44% (w/v); about 44% (w/v) to about 60% (w/v), about 58% (w/v), about 56% (w/v), about 54% (w/v), about 52% (w/v), about 50% (w/v), about 48% (w/v), or about 46% (w/v); about 46% (w/v) to about 60% (w/v), about 58% (w/v), about 56% (w/v), about 54% (w/v), about 52% (w/v), about 50% (w/v), or about 48% (w/v); about 48% (w/v) to about 60% (w/v), about 58% (w/v), about 56% (w/v), about 54% (w/v), about 52% (w/v), or about 50% (w/v); about 50% (w/v) to about 60% (w/v), about 58% (w/v), about 56% (w/v), about 54% (w/v), or about 52% (w/v); about 52% (w/v) to about 60% (w/v), about 58% (w/v), about 56% (w/v), or about 54% (w/v); about 54% (w/v) to about 60% (w/v), about 58% (w/v), or about 56% (w/v); about 56% (w/v) to about 60% (w/v) or about 58% (w/v); about 58% (w/v) to about 60% (w/v). Any of the concentrations of silk fibroin described herein may be determined using the methods described in Example 3. The silk concentrations (% w/v) described in the Examples were all determined using the method described in Example 3.

Silk fibroin concentrations in a composition can also be determined using a gravimetric method. For example, in an exemplary gravimetric method, a 1 mL aliquot of silk fibroin solution (after dialysis) can be weighed into a tared container, the container incubated at 60° C. for 16-24 hours to evaporate any water, and the mass of silk fibroin remaining is used to determine the concentration of silk fibroin in a solution (mass silk fibroin/aliquot volume).

The concentration of a benzenesulfonamide-containing NSAID in any of the compositions (e.g., hydrogels or organogels) provided herein can be determined by placing 50 mg of the composition in 5 mL of methanol, and extracting the benzenesulfonamide-containing NSAID by shaking at room temperature for at least 24 hours. Following extraction, the supernatant is separated by either filtration or centrifugation and analyzed by high performance liquid chromatography to determine the concentration of the benzenesulfonamide-containing NSAID present in the composition (e.g., hydrogel or organogel).

The second solution can be an aqueous solution (e.g., a pharmaceutically acceptable aqueous solution, such as saline or phosphate buffered saline). The second solution can include a single gelling agent (e.g., any of the exemplary gelling agents described herein or known in the art). In other examples, the second solution can include two or more different gelling agents (e.g., any combination of the exemplary gelling agents described herein or known in the art). The second solution can have a concentration of the gelling agent (or a final total concentration of two or more gelling agents) of about 0.02% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), about 2.5% (w/v), about 2% (w/v), about 1.5% (w/v), about 1.0% (w/v), about 0.5% (w/v), about 0.1% (w/v), about 0.5% (w/v), or about 0.1% (w/v) (inclusive); about 0.1% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), about 2.5% (w/v), about 2% (w/v), about 1.5% (w/v), about 1.0% (w/v), about 0.5% (w/v), about 0.1% (w/v), or about 0.5% (w/v) (inclusive); about 0.5% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), about 2.5% (w/v), about 2% (w/v), about 1.5% (w/v), or about 1.0% (w/v) (inclusive); about 1.0% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), about 2.5% (w/v), about 2% (w/v), or about 1.5% (w/v) (inclusive); about 1.5% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), about 2.5% (w/v), or about 2% (w/v) (inclusive); about 2% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), or about 2.5% (w/v) (inclusive); about 2.5% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), or about 4% (w/v) (inclusive); about 4% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), or about 6% (w/v) (inclusive); about 6% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), or about 8% (w/v) (inclusive); about 8% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), or about 10% (w/v) (inclusive); about 10% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), or about 12% (w/v) (inclusive); about 12% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), about 16% (w/v), or about 14% (w/v) (inclusive); about 14% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), about 18% (w/v), or about 16% (w/v) (inclusive); about 16% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), about 20% (w/v), or about 18% (w/v) (inclusive); about 18% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), about 22% (w/v), or about 20% (w/v) (inclusive); about 20% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), about 24% (w/v), or about 22% (w/v) (inclusive); about 22% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), about 26% (w/v), or about 24% (w/v) (inclusive); about 24% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 28% (w/v), or about 26% (w/v) (inclusive); about 26% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), or about 28% (w/v) (inclusive); about 28% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v) (inclusive); about 30% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), or about 32% (w/v) (inclusive); about 32% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), or about 34% (w/v) (inclusive); about 34% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), or about 36% (w/v) (inclusive); about 36% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v) (inclusive); about 38% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), or about 40% (w/v) (inclusive); about 40% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), or about 42% (w/v) (inclusive); about 42% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), or about 44% (w/v) (inclusive); about 44% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), or about 46% (w/v) (inclusive); about 46% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), or about 48% (w/v) (inclusive); about 48% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), or about 50% (w/v) (inclusive); about 50% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), or about 52% (w/v) (inclusive); about 52% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), or about 54% (w/v) (inclusive); about 54% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), or about 56% (w/v) (inclusive); about 56% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), or about 58% (w/v) (inclusive); about 58% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), or about 60% (w/v) (inclusive); about 60% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), or about 62% (w/v) (inclusive); about 62% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), or about 64% (w/v) (inclusive); about 64% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), or about 66% (w/v) (inclusive); about 66% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), or about 68% (w/v) (inclusive); about 68% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), or about 70% (w/v) (inclusive); about 70% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), or about 72% (w/v) (inclusive); about 72% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), or about 74% (w/v) (inclusive); about 74% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), or about 76% (w/v) (inclusive); about 76% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), or about 78% (w/v) (inclusive); about 78% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), or about 80% (w/v) (inclusive); about 80% (w/v) to about 100% (w/v), about 98% (w/v), about 96% (w/v), about 94% (w/v), about 92% (w/v), about 90% (w/v), about 88% (w/v), about 86% (w/v), about 84% (w/v), or about 82% (w/v) (inclusive); about 82% (w/v) to about 100% (w/v), about 98% (w/v), about 96% (w/v), about 94% (w/v), about 92% (w/v), about 90% (w/v), about 88% (w/v), about 86% (w/v), or about 84% (w/v) (inclusive); about 84% (w/v) to about 100% (w/v), about 98% (w/v), about 96% (w/v), about 94% (w/v), about 92% (w/v), about 90% (w/v), about 88% (w/v), or about 86% (w/v) (inclusive); about 86% (w/v) to about 100% (w/v), about 98% (w/v), about 96% (w/v), about 94% (w/v), about 92% (w/v), about 90% (w/v), or about 88% (w/v) (inclusive); about 88% (w/v) to about 100% (w/v), about 98% (w/v), about 96% (w/v), about 94% (w/v), about 92% (w/v), or about 90% (w/v) (inclusive); about 90% (w/v) to about 100% (w/v), about 98% (w/v), about 96% (w/v), about 94% (w/v), or about 92% (w/v) (inclusive); about 92% (w/v) to about 100% (w/v), about 98% (w/v), about 96% (w/v), or about 94% (w/v) (inclusive); about 94% (w/v) to about 100% (w/v), about 98% (w/v), or about 96% (w/v) (inclusive); about 96% (w/v) to about 100% (w/v) or about 98% (w/v) (inclusive); or about 98% (w/v) to about 100% (w/v) (inclusive).

The second solution can have a concentration of the gelling agent (or a final total concentration of two or more gelling agents) of about 0.02% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), about 2.5% (v/v), about 2% (v/v), about 1.5% (v/v), about 1.0% (v/v), about 0.5% (v/v), about 0.1% (v/v), about 0.5% (v/v), or about 0.1% (v/v) (inclusive); about 0.1% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), about 2.5% (v/v), about 2% (v/v), about 1.5% (v/v), about 1.0% (v/v), about 0.5% (v/v), about 0.1% (v/v), or about 0.5% (v/v) (inclusive); about 0.5% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), about 2.5% (v/v), about 2% (v/v), about 1.5% (v/v), or about 1.0% (v/v) (inclusive); about 1.0% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), about 2.5% (v/v), about 2% (v/v), or about 1.5% (v/v) (inclusive); about 1.5% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), about 2.5% (v/v), or about 2% (v/v) (inclusive); about 2% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), or about 2.5% (v/v) (inclusive); about 2.5% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), or about 4% (v/v) (inclusive); about 4% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), or about 6% (v/v) (inclusive); about 6% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), or about 8% (v/v) (inclusive); about 8% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), or about 10% (v/v) (inclusive); about 10% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), or about 12% (v/v) (inclusive); about 12% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), about 16% (v/v), or about 14% (v/v) (inclusive); about 14% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), about 18% (v/v), or about 16% (v/v) (inclusive); about 16% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), about 20% (v/v), or about 18% (v/v) (inclusive); about 18% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), about 22% (v/v), or about 20% (v/v) (inclusive); about 20% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), about 24% (v/v), or about 22% (v/v) (inclusive); about 22% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), about 26% (v/v), or about 24% (v/v) (inclusive); about 24% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 28% (v/v), or about 26% (v/v) (inclusive); about 26% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), or about 28% (v/v) (inclusive); about 28% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v) (inclusive); about 30% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), or about 32% (v/v) (inclusive); about 32% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), or about 34% (v/v) (inclusive); about 34% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), or about 36% (v/v) (inclusive); about 36% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v) (inclusive); about 38% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), or about 40% (v/v) (inclusive); about 40% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), or about 42% (v/v) (inclusive); about 42% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), or about 44% (v/v) (inclusive); about 44% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), or about 46% (v/v) (inclusive); about 46% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), or about 48% (v/v) (inclusive); about 48% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), or about 50% (v/v) (inclusive); about 50% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), or about 52% (v/v) (inclusive); about 52% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), or about 54% (v/v) (inclusive); about 54% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), or about 56% (v/v) (inclusive); about 56% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), or about 58% (v/v) (inclusive); about 58% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), or about 60% (v/v) (inclusive); about 60% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), or about 62% (v/v) (inclusive); about 62% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), or about 64% (v/v) (inclusive); about 64% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), or about 66% (v/v) (inclusive); about 66% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), or about 68% (v/v) (inclusive); about 68% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), or about 70% (v/v) (inclusive); about 70% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), or about 72% (v/v) (inclusive); about 72% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), or about 74% (v/v) (inclusive); about 74% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), or about 76% (v/v) (inclusive); about 76% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), or about 78% (v/v) (inclusive); about 78% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), or about 80% (v/v) (inclusive); about 80% (v/v) to about 100% (v/v), about 98% (v/v), about 96% (v/v), about 94% (v/v), about 92% (v/v), about 90% (v/v), about 88% (v/v), about 86% (v/v), about 84% (v/v), or about 82% (v/v) (inclusive); about 82% (v/v) to about 100% (v/v), about 98% (v/v), about 96% (v/v), about 94% (v/v), about 92% (v/v), about 90% (v/v), about 88% (v/v), about 86% (v/v), or about 84% (v/v) (inclusive); about 84% (v/v) to about 100% (v/v), about 98% (v/v), about 96% (v/v), about 94% (v/v), about 92% (v/v), about 90% (v/v), about 88% (v/v), or about 86% (v/v) (inclusive); about 86% (v/v) to about 100% (v/v), about 98% (v/v), about 96% (v/v), about 94% (v/v), about 92% (v/v), about 90% (v/v), or about 88% (v/v) (inclusive); about 88% (v/v) to about 100% (v/v), about 98% (v/v), about 96% (v/v), about 94% (v/v), about 92% (v/v), or about 90% (v/v) (inclusive); about 90% (v/v) to about 100% (v/v), about 98% (v/v), about 96% (v/v), about 94% (v/v), or about 92% (v/v) (inclusive); about 92% (v/v) to about 100% (v/v), about 98% (v/v), about 96% (v/v), or about 94% (v/v) (inclusive); about 94% (v/v) to about 100% (v/v), about 98% (v/v), or about 96% (v/v) (inclusive); about 96% (v/v) to about 100% (v/v) or about 98% (v/v) (inclusive); or about 98% (v/v) to about 100% (v/v) (inclusive).

In some examples, the gelling agent is a PEG (e.g., any of the PEGs described herein) or any of the other exemplary gelling agents described herein. In some examples, the gelling agent is glycerol.

The second solution can include a single benzenesulfonamide-containing NSAID (e.g., any of the exemplary benzenesulfonamide-containing NSAIDs described herein or known in the art). In other examples, the second solution can include two or more different benzenesulfonamide-containing NSAIDs (e.g., any combination of the exemplary benzenesulfonamide-containing NSAIDs described herein or known in the art). The second solution can have a concentration of the benzenesulfonamide-containing NSAID (or a final sum total concentration of two or more benzenesulfonamide-containing NSAIDs) of about 1 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, about 55 mg/mL, about 50 mg/mL, about 45 mg/mL, about 40 mg/mL, about 35 mg/mL, about 30 mg/mL, about 25 mg/mL, about 20 mg/mL, about 18 mg/mL, about 16 mg/mL, about 14 mg/mL, about 12 mg/mL, about 10 mg/mL, about 8 mg/mL, about 6 mg/mL, about 4 mg/mL, or about 2 mg/mL (inclusive); about 2 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, about 55 mg/mL, about 50 mg/mL, about 45 mg/mL, about 40 mg/mL, about 35 mg/mL, about 30 mg/mL, about 25 mg/mL, about 20 mg/mL, about 18 mg/mL, about 16 mg/mL, about 14 mg/mL, about 12 mg/mL, about 10 mg/mL, about 8 mg/mL, about 6 mg/mL, or about 4 mg/mL (inclusive); about 4 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, about 55 mg/mL, about 50 mg/mL, about 45 mg/mL, about 40 mg/mL, about 35 mg/mL, about 30 mg/mL, about 25 mg/mL, about 20 mg/mL, about 18 mg/mL, about 16 mg/mL, about 14 mg/mL, about 12 mg/mL, about 10 mg/mL, about 8 mg/mL, or about 6 mg/mL (inclusive); about 6 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, about 55 mg/mL, about 50 mg/mL, about 45 mg/mL, about 40 mg/mL, about 35 mg/mL, about 30 mg/mL, about 25 mg/mL, about 20 mg/mL, about 18 mg/mL, about 16 mg/mL, about 14 mg/mL, about 12 mg/mL, about 10 mg/mL, or about 8 mg/mL (inclusive); about 8 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, about 55 mg/mL, about 50 mg/mL, about 45 mg/mL, about 40 mg/mL, about 35 mg/mL, about 30 mg/mL, about 25 mg/mL, about 20 mg/mL, about 18 mg/mL, about 16 mg/mL, about 14 mg/mL, about 12 mg/mL, or about 10 mg/mL (inclusive); about 10 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, about 55 mg/mL, about 50 mg/mL, about 45 mg/mL, about 40 mg/mL, about 35 mg/mL, about 30 mg/mL, about 25 mg/mL, about 20 mg/mL, about 18 mg/mL, about 16 mg/mL, about 14 mg/mL, or about 12 mg/mL (inclusive); about 12 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, about 55 mg/mL, about 50 mg/mL, about 45 mg/mL, about 40 mg/mL, about 35 mg/mL, about 30 mg/mL, about 25 mg/mL, about 20 mg/mL, about 18 mg/mL, about 16 mg/mL, or about 14 mg/mL (inclusive); about 14 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, about 55 mg/mL, about 50 mg/mL, about 45 mg/mL, about 40 mg/mL, about 35 mg/mL, about 30 mg/mL, about 25 mg/mL, about 20 mg/mL, about 18 mg/mL, or about 16 mg/mL (inclusive); about 16 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, about 55 mg/mL, about 50 mg/mL, about 45 mg/mL, about 40 mg/mL, about 35 mg/mL, about 30 mg/mL, about 25 mg/mL, about 20 mg/mL, or about 18 mg/mL (inclusive); about 18 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, about 55 mg/mL, about 50 mg/mL, about 45 mg/mL, about 40 mg/mL, about 35 mg/mL, about 30 mg/mL, about 25 mg/mL, or about 20 mg/mL (inclusive); about 20 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, about 55 mg/mL, about 50 mg/mL, about 45 mg/mL, about 40 mg/mL, about 35 mg/mL, about 30 mg/mL, or about 25 mg/mL (inclusive); about 25 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, about 55 mg/mL, about 50 mg/mL, about 45 mg/mL, about 40 mg/mL, about 35 mg/mL, or about 30 mg/mL (inclusive); about 30 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, about 55 mg/mL, about 50 mg/mL, about 45 mg/mL, about 40 mg/mL, or about 35 mg/mL (inclusive); about 35 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, about 55 mg/mL, about 50 mg/mL, about 45 mg/mL, or about 40 mg/mL (inclusive); about 40 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, about 55 mg/mL, about 50 mg/mL, or about 45 mg/mL (inclusive); about 45 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, about 55 mg/mL, or about 50 mg/mL (inclusive); about 50 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, about 60 mg/mL, or about 55 mg/mL (inclusive); about 55 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, about 65 mg/mL, or about 60 mg/mL (inclusive); about 60 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, about 70 mg/mL, or about 65 mg/mL (inclusive); about 65 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, about 75 mg/mL, or about 70 mg/mL (inclusive); about 70 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, about 80 mg/mL, or about 75 mg/mL (inclusive); about 75 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, about 85 mg/mL, or about 80 mg/mL (inclusive); about 80 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, 95 mg/mL, about 90 mg/mL, or about 85 mg/mL; about 85 mg/mL to about 100 mg/mL, 95 mg/mL, or about 90 mg/mL (inclusive); about 90 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, about 100 mg/mL, or 95 mg/mL (inclusive); about 95 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, about 105 mg/mL, or about 100 mg/mL (inclusive); about 100 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, about 110 mg/mL, or about 105 mg/mL (inclusive); about 105 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 115 mg/mL, or about 110 mg/mL (inclusive); about 110 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, or about 115 mg/mL (inclusive); about 115 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, or about 120 mg/mL (inclusive); about 120 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, or about 125 mg/mL (inclusive); about 125 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, or about 130 mg/mL (inclusive); about 130 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, about 140 mg/mL, or about 135 mg/mL (inclusive); about 135 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, about 145 mg/mL, or about 140 mg/mL (inclusive); about 140 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, about 150 mg/mL, or about 145 mg/mL (inclusive); about 145 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, about 155 mg/mL, or about 150 mg/mL (inclusive); about 150 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, about 160 mg/mL, or about 155 mg/mL (inclusive); about 155 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, about 165 mg/mL, or about 160 mg/mL (inclusive); about 160 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, about 170 mg/mL, or about 165 mg/mL (inclusive); about 165 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, about 175 mg/mL, or about 170 mg/mL (inclusive); about 170 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, about 180 mg/mL, or about 175 mg/mL (inclusive); about 175 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, about 185 mg/mL, or about 180 mg/mL (inclusive); about 180 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, about 190 mg/mL, or about 185 mg/mL (inclusive); about 185 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL, about 195 mg/mL, or about 190 mg/mL (inclusive); about 190 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, about 200 mg/mL or about 195 mg/mL (inclusive); about 195 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, about 220 mg/mL, or about 200 mg/mL (inclusive); about 200 mg/mL to about 400 mg/mL, about 380 mg/mL, about 360 mg/mL, about 340 mg/mL, about 320 mg/mL, about 300 mg/mL, about 280 mg/mL, about 260 mg/mL, about 240 mg/mL, or about 220 mg/mL (inclusive); about 220 mg/mL to about 400 mg/mL, about 380 mg/mL, about 360 mg/mL, about 340 mg/mL, about 320 mg/mL, about 300 mg/mL, about 280 mg/mL, about 260 mg/mL, or about 240 mg/mL (inclusive); about 240 mg/mL to about 400 mg/mL, about 380 mg/mL, about 360 mg/mL, about 340 mg/mL, about 320 mg/mL, about 300 mg/mL, about 280 mg/mL, or about 260 mg/mL (inclusive); about 260 mg/mL to about 400 mg/mL, about 380 mg/mL, about 360 mg/mL, about 340 mg/mL, about 320 mg/mL, about 300 mg/mL, or about 280 mg/mL (inclusive); about 280 mg/mL to about 400 mg/mL, about 380 mg/mL, about 360 mg/mL, about 340 mg/mL, about 320 mg/mL, or about 300 mg/mL (inclusive); about 300 mg/mL to about 400 mg/mL, about 380 mg/mL, about 360 mg/mL, about 340 mg/mL, or about 320 mg/mL (inclusive); about 320 mg/mL to about 400 mg/mL, about 380 mg/mL, about 360 mg/mL, or about 340 mg/mL (inclusive); about 340 mg/mL to about 400 mg/mL, about 380 mg/mL, or about 360 mg/mL (inclusive); about 360 mg/mL to about 400 mg/mL or about 380 mg/mL (inclusive); or about 380 mg/mL to about 400 mg/mL (inclusive).

The mixing of the volume of the first solution and the volume of the second solution can be performed by inverting a closed container containing the first solution and the second solution, or vortexing, shaking, or sonicating. The ratio of the volume of the first solution to the volume of the second solution can be, e.g., about 5:1 to about 1:5, about 1:4.5, about 1:4, about 1:3.5, about 1:3, about 1:2.5, about 1:2, about 1:1.5, about 1:1, about 1.5:1, about 2:1, about 2.5:1, about 3:1, about 3.5:1, about 4:1, or about 4.5:1 (inclusive); about 4.5:1 to about 1:5, about 1:4.5, about 1:4, about 1:3.5, about 1:3, about 1:2.5, about 1:2, about 1:1.5, about 1:1, about 1.5:1, about 2:1, about 2.5:1, about 3:1, about 3.5:1, or about 4:1 (inclusive); about 4:1 to about 1:5, about 1:4.5, about 1:4, about 1:3.5, about 1:3, about 1:2.5, about 1:2, about 1:1.5, about 1:1, about 1.5:1, about 2:1, about 2.5:1, about 3:1, or about 3.5:1 (inclusive); about 3.5:1 to about 1:5, about 1:4.5, about 1:4, about 1:3.5, about 1:3, about 1:2.5, about 1:2, about 1:1.5, about 1:1, about 1.5:1, about 2:1, about 2.5:1, or about 3:1 (inclusive); about 3:1 to about 1:5, about 1:4.5, about 1:4, about 1:3.5, about 1:3, about 1:2.5, about 1:2, about 1:1.5, about 1:1, about 1.5:1, about 2:1, or about 2.5:1 (inclusive); about 2.5:1 to about 1:5, about 1:4.5, about 1:4, about 1:3.5, about 1:3, about 1:2.5, about 1:2, about 1:1.5, about 1:1, about 1.5:1, or about 2:1 (inclusive); about 2:1 to about 1:5, about 1:4.5, about 1:4, about 1:3.5, about 1:3, about 1:2.5, about 1:2, about 1:1.5, about 1:1, or about 1.5:1 (inclusive); about 1.5:1 to about 1:5, about 1:4.5, about 1:4, about 1:3.5, about 1:3, about 1:2.5, about 1:2, about 1:1.5, or about 1:1 (inclusive); about 1:1 to about 1:5, about 1:4.5, about 1:4, about 1:3.5, about 1:3, about 1:2.5, about 1:2, or about 1:1.5 (inclusive); about 1:1.5 to about 1:5, about 1:4.5, about 1:4, about 1:3.5, about 1:3, about 1:2.5, or about 1:2 (inclusive); about 1:2 to about 1:5, about 1:4.5, about 1:4, about 1:3.5, about 1:3, or about 1:2.5 (inclusive); about 1:2.5 to about 1:5, about 1:4.5, about 1:4, about 1:3.5, or about 1:3 (inclusive); about 1:3 to about 1:5, about 1:4.5, about 1:4, or about 1:3.5 (inclusive); about 1:3.5 to about 1:5, about 1:4.5, about 1:4 (inclusive); about 1:4 to about 1:5 or about 1:4.5 (inclusive); or about 1:4.5 to about 1:5 (inclusive).

The step of incubating the mixture for a time sufficient to form the composition (e.g., gel, e.g., hydrogel or organogel) can be performed at a temperature of about 10° C. to about 37° C., about 35° C., about 30° C., about 27° C., about 25° C., about 23° C., about 20° C., or about 15° C.; about 15° C. to about 37° C., about 35° C., about 30° C., about 27° C., about 25° C., about 23° C., or about 20° C.; about 20° C. to about 37° C., about 35° C., about 30° C., about 27° C., about 25° C., or about 23° C.; about 23° C. to about 37° C., about 35° C., about 30° C., about 27° C., or about 25° C.; about 25° C. to about 37° C., about 35° C., about 30° C., or about 27° C.; about 27° C. to about 37° C., about 35° C., or about 30° C.; about 30° C. to about 37° C. or about 35° C.; or about 35° C. to about 37° C. The time sufficient to form the composition (e.g., gel, e.g., hydrogel or organogel) can be, e.g., about 10 minutes to about 24 hours, about 22 hours, about 20 hours, about 18 hours, about 16 hours, about 14 hours, about 12 hours, about 10 hours, about 8 hours, about 6 hours, about 4 hours, about 2 hours, about 1 hour, or about 30 minutes; about 30 minutes to about 24 hours, about 22 hours, about 20 hours, about 18 hours, about 16 hours, about 14 hours, about 12 hours, about 10 hours, about 8 hours, about 6 hours, about 4 hours, about 2 hours, or about 1 hour; about 1 hour to about 24 hours, about 22 hours, about 20 hours, about 18 hours, about 16 hours, about 14 hours, about 12 hours, about 10 hours, about 8 hours, about 6 hours, about 4 hours, or about 2 hours; about 2 hours to about 24 hours, about 22 hours, about 20 hours, about 18 hours, about 16 hours, about 14 hours, about 12 hours, about 10 hours, about 8 hours, about 6 hours, or about 4 hours; about 4 hours to about 24 hours, about 22 hours, about 20 hours, about 18 hours, about 16 hours, about 14 hours, about 12 hours, about 10 hours, about 8 hours, or about 6 hours; about 6 hours to about 24 hours, about 22 hours, about 20 hours, about 18 hours, about 16 hours, about 14 hours, about 12 hours, about 10 hours, or about 8 hours; about 8 hours to about 24 hours, about 22 hours, about 20 hours, about 18 hours, about 16 hours, about 14 hours, about 12 hours, or about 10 hours; about 10 hours to about 24 hours, about 22 hours, about 20 hours, about 18 hours, about 16 hours, about 14 hours, or about 12 hours; about 12 hours to about 24 hours, about 22 hours, about 20 hours, about 18 hours, about 16 hours, or about 14 hours; about 14 hours to about 24 hours, about 22 hours, about 20 hours, about 18 hours, or about 16 hours; about 16 hours to about 24 hours, about 22 hours, about 20 hours, or about 18 hours; about 18 hours to about 24 hours, about 22 hours, or about 20 hours; about 20 hours to about 24 hours or about 22 hours; or about 22 hours to about 24 hours.

Some examples of these methods further include, between the mixing and incubating step, a step of sterilizing the mixture. Some examples of these methods further include after the incubating step, a step of sterilizing the mixture. In some examples, the sterilizing step is performed by autoclaving the mixture (e.g., autoclaving the mixture at 121° C. for at least 30 minutes, at least 35 minutes, at least 40 minutes, at least 45 minutes, at least 50 minutes, at least 55 minutes, or at least 60 minutes). Some embodiments, wherein the final concentration of the gelling agent in the composition is about 0.01% (w/v) to about 2% (w/v), 0.01% (v/v) to about 2% (v/v), about 1.8% (w/v), about 1.6% (w/v), about 1.8% (v/v), about 1.6% (v/v), about 1.4% (w/v), about 1.2% (w/v), about 1.4% (v/v), about 1.2% (v/v), about 1.0% (w/v), about 0.8% (w/v), about 1.0% (v/v), about 0.8% (v/v), about 0.6% (w/v), about 0.6% (v/v), about 0.4% (w/v), about 0.4% (v/v), about 0.2% (w/v), about 0.2% (v/v), about 0.1% (w/v), about 0.1% (v/v), about 0.05% (w/v), or about 0.05% (v/v), can include a step of autoclaving the mixture (e.g., using any of the conditions for autoclaving described herein or known in the art), between the mixing and incubating steps, and/or after the incubating step.

Also provided are methods of making a composition (e.g., gel, e.g., hydrogel or organogel) that include: (a) providing a solid composition (e.g., a lyophilized, powder composition) including silk fibroin (e.g., any of the silk fibroins described herein); (b) providing a solution including about 0.02% (w/v) to about 100% (w/v), or about 0.02% (v/v) to about 100% (v/v), of a gelling agent (e.g., any of the gelling agents described herein), and about 1 mg/mL to about 400 mg/mL of a benzenesulfonamide-containing NSAID (e.g., any of the benzenesulfonamide-containing NSAIDs described herein); (c) mixing a volume of the solution with an amount of the solid composition; and (d) incubating the mixture of a time sufficient to form the composition (e.g., gel, e.g., hydrogel or organogel).

In some examples, the solution can include about 0.02% (w/v) to about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), about 2.5% (w/v), about 2% (w/v), about 1.5% (w/v), about 1% (w/v), about 0.5% (w/v), or about 0.1% (w/v) (inclusive); about 0.1% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), about 2.5% (w/v), about 2% (w/v), about 1.5% (w/v), about 1% (w/v), or about 0.5% (w/v) (inclusive); about 0.5% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), about 2.5% (w/v), about 2% (w/v), about 1.5% (w/v), or about 1% (w/v) (inclusive); about 1% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), about 2.5% (w/v), about 2% (w/v), or about 1.5% (w/v) (inclusive); about 1.5% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), about 2.5% (w/v), or about 2% (w/v) (inclusive); about 2% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), about 4% (w/v), or about 2.5% (w/v) (inclusive); about 2.5% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), about 6% (w/v), or about 4% (w/v) (inclusive); about 4% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 8% (w/v), or about 6% (w/v) (inclusive); about 6% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), or about 8% (w/v) (inclusive); about 8% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), or about 10% (w/v) (inclusive); about 10% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), about 24% (w/v), about 22% (w/v), about 20% (w/v), about 18% (w/v), about 16% (w/v), about 14% (w/v), or about 12% (w/v) (inclusive); about 12% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), about 24% (w/v), about 22% (w/v), about 20% (w/v), about 18% (w/v), about 16% (w/v), or about 14% (w/v) (inclusive); about 14% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), about 24% (w/v), about 22% (w/v), about 20% (w/v), about 18% (w/v), or about 16% (w/v) (inclusive); about 16% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), about 24% (w/v), about 22% (w/v), about 20% (w/v), or about 18% (w/v) (inclusive); about 18% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), about 24% (w/v), about 22% (w/v), or about 20% (w/v) (inclusive); about 20% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), about 24% (w/v), or about 22% (w/v) (inclusive); about 22% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), about 26% (w/v), or about 24% (w/v) (inclusive); about 24% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), about 28% (w/v), or about 26% (w/v) (inclusive); about 26% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), about 30% (w/v), or about 28% (w/v) (inclusive); about 28% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), about 32% (w/v), or about 30% (w/v) (inclusive); about 30% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), about 34% (w/v), about 32% (w/v), about 30% (w/v), or about 32% (w/v) (inclusive); about 32% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), about 36% (w/v), or about 34% (w/v) (inclusive); about 34% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), about 38% (w/v), or about 36% (w/v) (inclusive); about 36% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), about 40% (w/v), or about 38% (w/v) (inclusive); about 38% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), about 42% (w/v), or about 40% (w/v) (inclusive); about 40% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), about 44% (w/v), or about 42% (w/v) (inclusive); about 42% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), about 46% (w/v), or about 44% (w/v) (inclusive); about 44% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), about 48% (w/v), or about 46% (w/v) (inclusive); about 46% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), about 50% (w/v), or about 48% (w/v) (inclusive); about 48% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), about 52% (w/v), or about 50% (w/v) (inclusive); about 50% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), about 54% (w/v), or about 52% (w/v) (inclusive); about 52% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), about 56% (w/v), or about 54% (w/v) (inclusive); about 54% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), about 58% (w/v), or about 56% (w/v) (inclusive); about 56% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), about 60% (w/v), or about 58% (w/v) (inclusive); about 58% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), about 62% (w/v), or about 60% (w/v) (inclusive); about 60% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), about 64% (w/v), or about 62% (w/v) (inclusive); about 62% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), about 66% (w/v), or about 64% (w/v) (inclusive); about 64% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), about 68% (w/v), or about 66% (w/v) (inclusive); about 66% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), about 70% (w/v), or about 68% (w/v) (inclusive); about 68% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), about 72% (w/v), or about 70% (w/v) (inclusive); about 70% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), about 74% (w/v), or about 72% (w/v) (inclusive); about 72% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), about 76% (w/v), or about 74% (w/v) (inclusive); about 74% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), about 78% (w/v), or about 76% (w/v) (inclusive); about 76% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), about 80% (w/v), or about 78% (w/v) (inclusive); about 78% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v), or about 80% (w/v) (inclusive); about 80% (w/v) to about 100% (w/v), about 98% (w/v), about 96% (w/v), about 94% (w/v), about 92% (w/v), about 90% (w/v), about 88% (w/v), about 86% (w/v), about 84% (w/v), about 82% (w/v) (inclusive); about 82% (w/v) to about 100% (w/v), about 98% (w/v), about 96% (w/v), about 94% (w/v), about 92% (w/v), about 90% (w/v), about 88% (w/v), about 86% (w/v), or about 84% (w/v) (inclusive); about 84% (w/v) to about 100% (w/v), about 98% (w/v), about 96% (w/v), about 94% (w/v), about 92% (w/v), about 90% (w/v), about 88% (w/v), or about 86% (w/v) (inclusive); about 86% (w/v) to about 100% (w/v), about 98% (w/v), about 96% (w/v), about 94% (w/v), about 92% (w/v), about 90% (w/v), or about 88% (w/v) (inclusive); about 88% (w/v) to about 100% (w/v), about 98% (w/v), about 96% (w/v), about 94% (w/v), about 92% (w/v), or about 90% (w/v) (inclusive); about 90% (w/v) to about 100% (w/v), about 98% (w/v), about 96% (w/v), about 94% (w/v), or about 92% (w/v) (inclusive); about 92% (w/v) to about 100% (w/v), about 98% (w/v), about 96% (w/v), or about 94% (w/v) (inclusive); about 94% (w/v) to about 100% (w/v), about 98% (w/v), or about 96% (w/v) (inclusive); about 96% (w/v) to about 100% (w/v), or about 98% (w/v) (inclusive); or about 98% (w/v) to about 100% (w/v) (inclusive), of the gelling agent.

In some examples, the solution can include about 0.02% (v/v) to about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), about 2.5% (v/v), about 2% (v/v), about 1.5% (v/v), about 1% (v/v), about 0.5% (v/v), or about 0.1% (v/v) (inclusive); about 0.1% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), about 2.5% (v/v), about 2% (v/v), about 1.5% (v/v), about 1% (v/v), or about 0.5% (v/v) (inclusive); about 0.5% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), about 2.5% (v/v), about 2% (v/v), about 1.5% (v/v), or about 1% (v/v) (inclusive); about 1% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), about 2.5% (v/v), about 2% (v/v), or about 1.5% (v/v) (inclusive); about 1.5% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), about 2.5% (v/v), or about 2% (v/v) (inclusive); about 2% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), about 4% (v/v), or about 2.5% (v/v) (inclusive); about 2.5% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), about 6% (v/v), or about 4% (v/v) (inclusive); about 4% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), about 8% (v/v), or about 6% (v/v) (inclusive); about 6% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), about 10% (v/v), or about 8% (v/v) (inclusive); about 8% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), about 12% (v/v), or about 10% (v/v) (inclusive); about 10% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), about 24% (v/v), about 22% (v/v), about 20% (v/v), about 18% (v/v), about 16% (v/v), about 14% (v/v), or about 12% (v/v) (inclusive); about 12% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), about 24% (v/v), about 22% (v/v), about 20% (v/v), about 18% (v/v), about 16% (v/v), or about 14% (v/v) (inclusive); about 14% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), about 24% (v/v), about 22% (v/v), about 20% (v/v), about 18% (v/v), or about 16% (v/v) (inclusive); about 16% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), about 24% (v/v), about 22% (v/v), about 20% (v/v), or about 18% (v/v) (inclusive); about 18% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), about 24% (v/v), about 22% (v/v), or about 20% (v/v) (inclusive); about 20% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), about 24% (v/v), or about 22% (v/v) (inclusive); about 22% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), about 26% (v/v), or about 24% (v/v) (inclusive); about 24% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), about 28% (v/v), or about 26% (v/v) (inclusive); about 26% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), about 30% (v/v), or about 28% (v/v) (inclusive); about 28% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), about 32% (v/v), or about 30% (v/v) (inclusive); about 30% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), about 34% (v/v), about 32% (v/v), about 30% (v/v), or about 32% (v/v) (inclusive); about 32% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), about 36% (v/v), or about 34% (v/v) (inclusive); about 34% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), about 38% (v/v), or about 36% (v/v) (inclusive); about 36% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), about 40% (v/v), or about 38% (v/v) (inclusive); about 38% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), about 42% (v/v), or about 40% (v/v) (inclusive); about 40% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), about 44% (v/v), or about 42% (v/v) (inclusive); about 42% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), about 46% (v/v), or about 44% (v/v) (inclusive); about 44% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), about 48% (v/v), or about 46% (v/v) (inclusive); about 46% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), about 50% (v/v), or about 48% (v/v) (inclusive); about 48% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), about 52% (v/v), or about 50% (v/v) (inclusive); about 50% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), about 54% (v/v), or about 52% (v/v) (inclusive); about 52% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), about 56% (v/v), or about 54% (v/v) (inclusive); about 54% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), about 58% (v/v), or about 56% (v/v) (inclusive); about 56% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), about 60% (v/v), or about 58% (v/v) (inclusive); about 58% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), about 62% (v/v), or about 60% (v/v) (inclusive); about 60% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), about 64% (v/v), or about 62% (v/v) (inclusive); about 62% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), about 66% (v/v), or about 64% (v/v) (inclusive); about 64% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), about 68% (v/v), or about 66% (v/v) (inclusive); about 66% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), about 70% (v/v), or about 68% (v/v) (inclusive); about 68% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), about 72% (v/v), or about 70% (v/v) (inclusive); about 70% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), about 74% (v/v), or about 72% (v/v) (inclusive); about 72% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), about 76% (v/v), or about 74% (v/v) (inclusive); about 74% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), about 78% (v/v), or about 76% (v/v) (inclusive); about 76% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), about 80% (v/v), or about 78% (v/v) (inclusive); about 78% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v), or about 80% (v/v) (inclusive); about 80% (v/v) to about 100% (v/v), about 98% (v/v), about 96% (v/v), about 94% (v/v), about 92% (v/v), about 90% (v/v), about 88% (v/v), about 86% (v/v), about 84% (v/v), about 82% (v/v) (inclusive); about 82% (v/v) to about 100% (v/v), about 98% (v/v), about 96% (v/v), about 94% (v/v), about 92% (v/v), about 90% (v/v), about 88% (v/v), about 86% (v/v), or about 84% (v/v) (inclusive); about 84% (v/v) to about 100% (v/v), about 98% (v/v), about 96% (v/v), about 94% (v/v), about 92% (v/v), about 90% (v/v), about 88% (v/v), or about 86% (v/v) (inclusive); about 86% (v/v) to about 100% (v/v), about 98% (v/v), about 96% (v/v), about 94% (v/v), about 92% (v/v), about 90% (v/v), or about 88% (v/v) (inclusive); about 88% (v/v) to about 100% (v/v), about 98% (v/v), about 96% (v/v), about 94% (v/v), about 92% (v/v), or about 90% (v/v) (inclusive); about 90% (v/v) to about 100% (v/v), about 98% (v/v), about 96% (v/v), about 94% (v/v), or about 92% (v/v) (inclusive); about 92% (v/v) to about 100% (v/v), about 98% (v/v), about 96% (v/v), or about 94% (v/v) (inclusive); about 94% (v/v) to about 100% (v/v), about 98% (v/v), or about 96% (v/v) (inclusive); about 96% (v/v) to about 100% (v/v), or about 98% (v/v) (inclusive); or about 98% (v/v) to about 100% (v/v) (inclusive), of the gelling agent.

In some examples, the solution can include any of the subranges of about 1 mg/mL to about 400 mg/mL of a benzenesulfonamide-containing NSAID described herein.

In some embodiments of these methods, the amount of the solid silk fibroin mixed with the solution can be an amount sufficient to result in, e.g., a final concentration of about 0.5% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), about 6% (w/v), about 5% (w/v), about 4% (w/v), about 3% (w/v), about 2% (w/v), or about 1% (w/v); about 1% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), about 6% (w/v), about 5% (w/v), about 4% (w/v), about 3% (w/v), or about 2% (w/v); about 2% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), about 6% (w/v), about 5% (w/v), about 4% (w/v), or about 3% (w/v); about 3% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), about 6% (w/v), about 5% (w/v), or about 4% (w/v); about 4% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), about 6% (w/v), or about 5% (w/v); about 5% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), or about 6% (w/v); about 6% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), or about 7% (w/v); about 7% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), or about 8% (w/v); about 8% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), or about 9% (w/v); about 9% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), or about 10% (w/v); about 10% (w/v) to about 30% (w/v), about 28% (w/v), about 26% (w/v), about 24% (w/v), about 22% (w/v), about 20% (w/v), about 18% (w/v), about 16% (w/v), about 14% (w/v), or about 12% (w/v); about 12% (w/v) to about 30% (w/v), about 28% (w/v), about 26% (w/v), about 24% (w/v), about 22% (w/v), about 20% (w/v), about 18% (w/v), about 16% (w/v), or about 14% (w/v); about 14% (w/v) to about 30% (w/v), about 28% (w/v), about 26% (w/v), about 24% (w/v), about 22% (w/v), about 20% (w/v), about 18% (w/v), or about 16% (w/v); about 16% (w/v) to about 30% (w/v), about 28% (w/v), about 26% (w/v), about 24% (w/v), about 22% (w/v), about 20% (w/v), or about 18% (w/v); about 18% (w/v) to about 30% (w/v), about 28% (w/v), about 26% (w/v), about 24% (w/v), about 22% (w/v), or about 20% (w/v); about 20% (w/v) to about 30% (w/v), about 28% (w/v), about 26% (w/v), about 24% (w/v), or about 22% (w/v); about 22% (w/v) to about 30% (w/v), about 28% (w/v), about 26% (w/v), or about 24% (w/v); about 24% (w/v) to about 30% (w/v), about 28% (w/v), or about 26% (w/v); about 26% (w/v) to about 30% (w/v) or about 28% (w/v); or about 28% (w/v) to about 30% (w/v) (units are in % weight of the silk fibroin per volume of the composition) of silk fibroin in the resulting composition (e.g., gel, e.g., hydrogel).

Some examples of any of these methods further include generating silk fibroin having a molecular weight range of about 10 kDa to about 500 kDa (or any of the subranges described herein). For example, the step of generating a silk fibroin having a molecular weight range of about 10 kDa to about 500 kDa (or any of the subranges described herein) can include incubating a silk raw material in a solution comprising sodium carbonate (Na₂CO₃) (e.g., 5 mM to about 100 mM sodium carbonate) for 5 minutes to 300 minutes at about 70° C. to about 100° C. (inclusive) (e.g., about 75° C. to about 100° C. (inclusive), about 80° C. to about 100° C. (inclusive), about 85° C. to about 100° C. (inclusive), about 90° C. to about 100° C. (inclusive), or about 95° C. to about 100° C. (inclusive)). In some examples, the silk raw material can be, e.g., a silk worm cocoon, a silk fiber, or a recombinantly-produced silk fibroin.

In some embodiments, the solution comprising sodium carbonate (Na₂CO₃) comprises about 5 mM to about 100 mM, about 95 mM, about 90 mM, about 85 mM, about 80 mM, about 75 mM, about 70 mM, about 65 mM, about 60 mM, about 55 mM, about 50 mM, about 45 mM, about 40 mM, about 35 mM, about 30 mM, about 25 mM, about 20 mM, about 15 mM, or about 10 mM; 10 mM to about 100 mM, about 95 mM, about 90 mM, about 85 mM, about 80 mM, about 75 mM, about 70 mM, about 65 mM, about 60 mM, about 55 mM, about 50 mM, about 45 mM, about 40 mM, about 35 mM, about 30 mM, about 25 mM, about 20 mM, about 15 mM, or about 10 mM; 15 mM to about 100 mM, about 95 mM, about 90 mM, about 85 mM, about 80 mM, about 75 mM, about 70 mM, about 65 mM, about 60 mM, about 55 mM, about 50 mM, about 45 mM, about 40 mM, about 35 mM, about 30 mM, about 25 mM, or about 20 mM; 20 mM to about 100 mM, about 95 mM, about 90 mM, about 85 mM, about 80 mM, about 75 mM, about 70 mM, about 65 mM, about 60 mM, about 55 mM, about 50 mM, about 45 mM, about 40 mM, about 35 mM, about 30 mM, or about 25 mM; 30 mM to about 100 mM, about 95 mM, about 90 mM, about 85 mM, about 80 mM, about 75 mM, about 70 mM, about 65 mM, about 60 mM, about 55 mM, about 50 mM, about 45 mM, about 40 mM, or about 35 mM; 35 mM to about 100 mM, about 95 mM, about 90 mM, about 85 mM, about 80 mM, about 75 mM, about 70 mM, about 65 mM, about 60 mM, about 55 mM, about 50 mM, about 45 mM, or about 40 mM; 40 mM to about 100 mM, about 95 mM, about 90 mM, about 85 mM, about 80 mM, about 75 mM, about 70 mM, about 65 mM, about 60 mM, about 55 mM, about 50 mM, or about 45 mM; 45 mM to about 100 mM, about 95 mM, about 90 mM, about 85 mM, about 80 mM, about 75 mM, about 70 mM, about 65 mM, about 60 mM, about 55 mM, or about 50 mM; 50 mM to about 100 mM, about 95 mM, about 90 mM, about 85 mM, about 80 mM, about 75 mM, about 70 mM, about 65 mM, about 60 mM, or about 55 mM; 55 mM to about 100 mM, about 95 mM, about 90 mM, about 85 mM, about 80 mM, about 75 mM, about 70 mM, about 65 mM, or about 60 mM; 60 mM to about 100 mM, about 95 mM, about 90 mM, about 85 mM, about 80 mM, about 75 mM, about 70 mM, or about 65 mM; 65 mM to about 100 mM, about 95 mM, about 90 mM, about 85 mM, about 80 mM, about 75 mM, or about 70 mM; 70 mM to about 100 mM, about 95 mM, about 90 mM, about 85 mM, about 80 mM, or about 75 mM; 75 mM to about 100 mM, about 95 mM, about 90 mM, about 85 mM, or about 80 mM; 80 mM to about 100 mM, about 95 mM, about 90 mM, or about 85 mM; 85 mM to about 100 mM, about 95 mM, or about 90 mM; 90 mM to about 100 mM, or about 95 mM; 95 mM to about 100 mM; about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM sodium carbonate.

In some embodiments, the incubating can be, e.g., 5 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, about 180 minutes, about 170 minutes, about 160 minutes, about 150 minutes, about 140 minutes, about 130 minutes, about 120 minutes, about 110 minutes, about 100 minutes, about 90 minutes, about 80 minutes, about 70 minutes, about 60 minutes, about 50 minutes, about 40 minutes, about 30 minutes, about 20 minutes, or about 10 minutes (inclusive); about 10 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, about 180 minutes, about 170 minutes, about 160 minutes, about 150 minutes, about 140 minutes, about 130 minutes, about 120 minutes, about 100 minutes, about 90 minutes, about 80 minutes, about 70 minutes, about 60 minutes, about 50 minutes, about 40 minutes, about 30 minutes, or about 20 minutes (inclusive); about 20 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, about 180 minutes, about 170 minutes, about 160 minutes, about 150 minutes, about 140 minutes, about 130 minutes, about 120 minutes, about 100 minutes, about 90 minutes, about 80 minutes, about 70 minutes, about 60 minutes, about 50 minutes, about 40 minutes, or about 30 minutes (inclusive); about 30 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, about 180 minutes, about 170 minutes, about 160 minutes, about 150 minutes, about 140 minutes, about 130 minutes, about 120 minutes, about 100 minutes, about 90 minutes, about 80 minutes, about 70 minutes, about 60 minutes, about 50 minutes, or about 40 minutes (inclusive); about 40 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, about 180 minutes, about 170 minutes, about 160 minutes, about 150 minutes, about 140 minutes, about 130 minutes, about 120 minutes, about 100 minutes, about 90 minutes, about 80 minutes, about 70 minutes, about 60 minutes, or about 50 minutes (inclusive); about 50 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, about 180 minutes, about 170 minutes, about 160 minutes, about 150 minutes, about 140 minutes, about 130 minutes, about 120 minutes, about 100 minutes, about 90 minutes, about 80 minutes, about 70 minutes, or about 60 minutes; about 60 minutes to about 120 minutes, about 100 minutes, about 90 minutes, about 80 minutes, or about 70 minutes (inclusive); about 70 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, about 180 minutes, about 170 minutes, about 160 minutes, about 150 minutes, about 140 minutes, about 130 minutes, about 120 minutes, about 100 minutes, about 90 minutes, or about 80 minutes (inclusive); about 80 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, about 180 minutes, about 170 minutes, about 160 minutes, about 150 minutes, about 140 minutes, about 130 minutes, about 120 minutes, about 100 minutes, or about 90 minutes (inclusive); about 90 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, about 180 minutes, about 170 minutes, about 160 minutes, about 150 minutes, about 140 minutes, about 130 minutes, about 120 minutes or about 100 minutes (inclusive); about 100 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, about 180 minutes, about 170 minutes, about 160 minutes, about 150 minutes, about 140 minutes, about 130 minutes, about 120 minutes, or about 110 minutes (inclusive); about 110 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, about 180 minutes, about 170 minutes, about 160 minutes, about 150 minutes, about 140 minutes, about 130 minutes, or about 120 minutes (inclusive); about 120 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, about 180 minutes, about 170 minutes, about 160 minutes, about 150 minutes, about 140 minutes, or about 130 minutes (inclusive); about 130 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, about 180 minutes, about 170 minutes, about 160 minutes, about 150 minutes, or about 140 minutes (inclusive); about 140 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, about 180 minutes, about 170 minutes, about 160 minutes, or about 150 minutes (inclusive); about 150 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, about 180 minutes, about 170 minutes, or about 160 minutes (inclusive); about 160 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, about 180 minutes, or about 170 minutes (inclusive); about 170 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, about 190 minutes, or about 180 minutes (inclusive); about 180 minutes to about 300 minutes, about 290 minutes, about 280 minutes, about 270 minutes, about 260 minutes, about 250 minutes, about 240 minutes, about 230 minutes, about 220 minutes, about 210 minutes, about 200 minutes, or about 190 minutes (inclusive); about 190 minutes to about 300 minutes, about 290 minutes, about 280 minutes, about 270 minutes, about 260 minutes, about 250 minutes, about 240 minutes, about 230 minutes, about 220 minutes, about 210 minutes, or about 200 minutes (inclusive); about 200 minutes to about 300 minutes, about 290 minutes, about 280 minutes, about 270 minutes, about 260 minutes, about 250 minutes, about 240 minutes, about 230 minutes, about 220 minutes, or about 210 minutes (inclusive); about 210 minutes to about 300 minutes, about 290 minutes, about 280 minutes, about 270 minutes, about 260 minutes, about 250 minutes, about 240 minutes, about 230 minutes, or about 220 minutes (inclusive); about 220 minutes to about 300 minutes, about 290 minutes, about 280 minutes, about 270 minutes, about 260 minutes, about 250 minutes, about 240 minutes, or about 230 minutes (inclusive); about 230 minutes to about 300 minutes, about 290 minutes, about 280 minutes, about 270 minutes, about 260 minutes, about 250 minutes, or about 240 minutes (inclusive); about 240 minutes to about 300 minutes, about 290 minutes, about 280 minutes, about 270 minutes, about 260 minutes, or about 250 minutes (inclusive); about 250 minutes to about 300 minutes, about 290 minutes, about 280 minutes, about 270 minutes, or about 260 minutes (inclusive); about 260 minutes to about 300 minutes, about 290 minutes, about 280 minutes, or about 270 minutes (inclusive); about 270 minutes to about 300 minutes, about 290 minutes, or about 280 minutes (inclusive); about 280 minutes to about 300 minutes or about 290 minutes (inclusive); or about 290 minutes to about 300 minutes (inclusive).

In some embodiments, the incubating can be, e.g., at about 70° C. to about 100° C. (inclusive); at about 96° C. to about 100° C. (inclusive); at about 70° C. (inclusive), at about 75° C.; at about 80° C.; at about 85° C.; at about 90° C.; at about 91° C.; at about 92° C.; at about 93° C.; at about 94° C.; at about 95° C.; at about 96° C.; at about 97° C.; at about 98° C.; at about 99° C.; or at about 100° C.

Also provided herein is a composition (e.g., a gel, e.g., a hydrogel) produced by any of the methods described herein.

### Methods of Making Compositions—Part B

Also provided herein are methods of making a composition (e.g., a gel, e.g., a hydrogel) that include: (a) providing a solution including about 1% (w/v) to about 60% (w/v) (or any of the subranges of this range described herein) silk fibroin (e.g., a silk fibroin having a molecular weight of about 10 kDa to about 500 kDa (or any of the subranges of this range described herein)); (b) providing a suspension including about 0.02% (w/v) to about 100% (w/v) (or any of the subranges of this range described herein), or about 0.02% (v/v) to about 100% (v/v) (or any of the subranges of this range described herein) of a gelling agent (e.g., any of the gelling agents described herein or known in the art), and about 1 mg/mL to about 400 mg/mL (or any of the subranges of this range described herein) of a benzenesulfonamide-containing NSAID (e.g., any of the benzenesulfonamide-containing NSAIDs described herein or known in the art); (c) mixing a volume of the solution with a volume of the suspension, where the ratio of the volume of the solution to the volume of the suspension is about 5:1 to about 1:5 (or any of the subranges of this range described herein) to provide a mixture; and (d) incubating the mixture for a time sufficient to form the composition (e.g., a gel, e.g., a hydrogel).

In some examples of these methods, the solution can include any of the subranges of about 1% (w/v) to about 60% (w/v) silk fibroin (e.g., a silk fibroin having a molecular weight of about 10 kDa to about 500 kDa (or any of the subranges of this range described herein)). In some examples of these methods, the suspension includes any of the subranges of 0.02% (w/v) to about 100% (w/v) of a gelling agent described herein. In some examples of these methods, the suspension includes any of the subranges of 0.02% (v/v) to about 100% (v/v) of a gelling agent described herein. In some examples of these methods, the suspension includes any of the subranges of about 1 mg/mL to about 400 mg/mL of a benzenesulfonamide-containing NSAID described herein. In some examples, the suspension includes a plurality of particles (e.g., any of the sizes and types of particles described herein). For example, the plurality of particles can include one or both of the benzenesulfonamide-containing NSAID and the gelling agent. In some embodiments where the plurality of the particles include both the benzenesulfonamide-containing NSAID and the gelling agent, the benzenesulfonamide-containing NSAID can be present in the interior or core of the particle, while the the gelling agent coats or coats in part the exterior of the particle. In some embodiments, where the plurality of the particles include both the benzenesulfonamide-containing NSAID and the gelling agent, the particles includes a disordered mixture (e.g., a co-crystal) of the benzenesulfonamide-containing NSAID and the gelling agent. In some embodiments, where the plurality of the particles include both the benzenesulfonamide-containing NSAID and the gelling agent, the particles include an ordered mixture (e.g., a co-crystal) of the benzenesulfonamide-containing NSAID and the gelling agent.

Also provided herein are methods of making a composition (e.g., a gel, e.g., a hydrogel) that include: (a) providing a solid composition including a silk fibroin (e.g., a silk fibroin having a molecular weight of about 10 kDa to about 500 kDa (or any of the subranges of this range described herein)); (b) providing a suspension including about 0.02% (w/v) to about 100% (w/v) (or any of the subranges of this range described herein), or about 0.02% (v/v) to about 100% (v/v) (or any of the subranges of this range described herein) of a gelling agent (e.g., any of the exemplary gelling agents described herein or known in the art), and about 1 mg/mL to about 400 mg/mL (or any of the subranges of this range described herein) of a benzenesulfonamide-containing NSAID (e.g., any of the benzenesulfonamide-containing NSAIDs described herein or known in the art); (c) mixing a volume of the solution with an amount of the solid composition; and (d) incubating the mixture of a time sufficient to form the composition (e.g., a gel, e.g., a hydrogel).

In some embodiments of these methods, the amount of the solid silk fibroin mixed with the solution can be an amount sufficient to result in, e.g., a final concentration of about 0.5% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), about 6% (w/v), about 5% (w/v), about 4% (w/v), about 3% (w/v), about 2% (w/v), or about 1% (w/v); about 1% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), about 6% (w/v), about 5% (w/v), about 4% (w/v), about 3% (w/v), or about 2% (w/v); about 2% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), about 6% (w/v), about 5% (w/v), about 4% (w/v), or about 3% (w/v); about 3% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), about 6% (w/v), about 5% (w/v), or about 4% (w/v); about 4% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), about 6% (w/v), or about 5% (w/v); about 5% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), about 7% (w/v), or about 6% (w/v); about 6% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), about 8% (w/v), or about 7% (w/v); about 7% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), about 9% (w/v), or about 8% (w/v); about 8% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), about 10% (w/v), or about 9% (w/v); about 9% (w/v) to about 30% (w/v), about 28% (w/v), about 26% (w/v), about 24% (w/v), about 22% (w/v), about 20% (w/v), about 18% (w/v), about 16% (w/v), about 14% (w/v), about 12% (w/v), or about 10% (w/v); about 10% (w/v) to about 30% (w/v), about 28% (w/v), about 26% (w/v), about 24% (w/v), about 22% (w/v), about 20% (w/v), about 18% (w/v), about 16% (w/v), about 14% (w/v), or about 12% (w/v); about 12% (w/v) to about 30% (w/v), about 28% (w/v), about 26% (w/v), about 24% (w/v), about 22% (w/v), about 20% (w/v), about 18% (w/v), about 16% (w/v), or about 14% (w/v); about 14% (w/v) to about 30% (w/v), about 28% (w/v), about 26% (w/v), about 24% (w/v), about 22% (w/v), about 20% (w/v), about 18% (w/v), or about 16% (w/v); about 16% (w/v) to about 30% (w/v), about 28% (w/v), about 26% (w/v), about 24% (w/v), about 22% (w/v), about 20% (w/v), or about 18% (w/v); about 18% (w/v) to about 30% (w/v), about 28% (w/v), about 26% (w/v), about 24% (w/v), about 22% (w/v), or about 20% (w/v); about 20% (w/v) to about 30% (w/v), about 28% (w/v), about 26% (w/v), about 24% (w/v), or about 22% (w/v); about 22% (w/v) to about 30% (w/v), about 28% (w/v), about 26% (w/v), or about 24% (w/v); about 24% (w/v) to about 30% (w/v), about 28% (w/v), or about 26% (w/v); about 26% (w/v) to about 30% (w/v) or about 28% (w/v); or about 28% (w/v) to about 30% (w/v) (units are in % weight of the silk fibroin per volume of the composition) of silk fibroin in the resulting composition (e.g., gel, e.g., hydrogel).

In some examples of these methods, the suspension includes any of the subranges of 0.02% (w/v) to about 100% (w/v) of a gelling agent described herein. In some examples of these methods, the suspension includes any of the subranges of 0.02% (v/v) to about 100% (v/v) of a gelling agent described herein. In some examples of these methods, the suspension includes any of the subranges of about 1 mg/mL to about 400 mg/mL of a benzenesulfonamide-containing NSAID described herein. In some examples, the suspension includes a plurality of particles (e.g., any of the sizes and types of particles described herein). For example, the plurality of particles can include one or both of the benzenesulfonamide-containing NSAID and the gelling agent. In some embodiments where the plurality of the particles include both the benzenesulfonamide-containing NSAID and the gelling agent, the benzenesulfonamide-containing NSAID can be present in the interior or core of the particle, while the the gelling agent coats or coats in part the exterior of the particle. In some embodiments, where the plurality of the particles include both the benzenesulfonamide-containing NSAID and the gelling agent, the particles includes a disordered mixture (e.g., a co-crystal) of the benzenesulfonamide-containing NSAID and the gelling agent. In some embodiments, where the plurality of the particles include both the benzenesulfonamide-containing NSAID and the gelling agent, the particles include an ordered mixture (e.g., a co-crystal) of the benzenesulfonamide-containing NSAID and the gelling agent.

Also provided herein are methods of making a composition (e.g., a gel, e.g., a hydrogel) that include: (a) providing a suspension including about 1 mg/mL to about 400 mg/mL (or any of the subranges of this range described herein) of a benzenesulfonamide-containing NSAID (e.g., any of the benzenesulfonamide-containing NSAIDs described herein or known in the art) and about 1% (w/v) to about 60% (w/v) (or any of the subranges of this range described herein) silk fibroin (e.g., a silk fibroin having a molecular weight of about 10 kDa to about 500 kDa (or any of the subranges of this range described herein)); (b) a solution comprising about 0.02% (w/v) to about 100% (w/v) (or any of the subranges of this range described herein), or about 0.02% (v/v) to about 100% (v/v) (or any of the subranges of this range described herein), of a gelling agent (e.g., any of the exemplary gelling agents described herein or known in the art); (c) mixing a volume of the solution with a volume of the suspension, where the ratio of the volume of the solution to the volume of the suspension is about 5:1 to about 1:5 (or any of the subranges of this range described herein) to provide a mixture; and (d) incubating the mixture of a time sufficient to form the composition (e.g., a gel, e.g., a hydrogel).

In some embodiments of these methods, the suspension can include any of the subranges of 1 mg/mL to about 400 mg/mL of a benzenesulfonamide-containing NSAID described herein. In some embodiments of these methods, the suspension can include any of the subranges of about 1% (w/v) to about 60% (w/v) silk fibroin. In some examples, the suspension includes a plurality of particles (e.g., any of the sizes and types of particles described herein). For example, the plurality of particles can include one or both of the benzenesulfonamide-containing NSAID and the silk fibroin. In some embodiments where the plurality of the particles include both the benzenesulfonamide-containing NSAID and the silk fibroin, the benzenesulfonamide-containing NSAID can be present in the interior or core of the particle, while the silk fibroin coats, or coats in part, the exterior of the particle. In some embodiments, where the plurality of the particles include both the benzenesulfonamide-containing NSAID and the silk fibroin, the particles includes a disordered mixture (e.g., a co-crystal) of the benzenesulfonamide-containing NSAID and the silk fibroin. In some embodiments, where the plurality of the particles include both the benzenesulfonamide-containing NSAID and the silk fibroin, the particles include an ordered mixture (e.g., a co-crystal) of the benzenesulfonamide-containing NSAID and the silk fibroin. In some embodiments of these methods, the solution can include any of the subranges of 0.02% (w/v) to 100% (w/v) of the gelling agent described herein. In some embodiments of these methods, the solution can include any of the subranges of 0.02% (v/v) to 100% (v/v) of the gelling agent described herein.

Also provided herein are methods of making a composition (e.g., a gel, e.g., a hydrogel) that include: (a) providing a suspension including about 1 mg/mL to about 400 mg/mL (or any of the subranges of this range described herein) of a benzenesulfonamide-containing NSAID (e.g., any of the benzenesulfonamide-containing NSAIDs described herein or known in the art); (b) a solution including about 0.02% (w/v) to about 100% (w/v) (or any of the subranges of this range described herein), or about 0.02% (v/v) to about 100% (v/v) (or any of the subranges of this range described herein) of a gelling agent (e.g., any of the gelling agents described herein or known in the art), and about 1% (w/v) to about 60% (w/v) (or any of the subranges of this range described herein) silk fibroin (e.g., a silk fibroin having a molecular weight of about 10 kDa to about 500 kDa); (c) mixing a volume of the solution with a volume of the suspension, where the ratio of the volume of the solution to the volume of the suspension is about 5:1 to about 1:5 (or any of the subranges of this range described herein) to provide a mixture; and (d) incubating the mixture of a time sufficient to form the composition (e.g., a gel, e.g., a hydrogel). In some embodiments of these methods, the suspension includes any of the subranges of about 1 mg/mL to about 400 mg/mL of the benzenesulfonamide-containing NSAID described herein. In some examples, the suspension includes a plurality of particles of the benzenesulfonamide-containing NSAID (e.g., any of the sizes and types of particles described herein). In some embodiments of these methods, the solution includes any of the subranges of about 0.02% (w/v) to about 100% (w/v) of the gelling agent described herein. In some embodiments of these methods, the solution includes any of the subranges of about 0.02% (v/v) to about 100% (v/v) of the gelling agent described herein. In some embodiments of these methods, the solution includes any of the subranges of about 1% (w/v) to about 60% (w/v) of the silk fibroin described herein.

Also provided herein are methods of making a composition (e.g., a gel, e.g., a hydrogel) that include: (a) providing a suspension including about 1 mg/mL to about 400 mg/mL (or any of the subranges of this range described herein) of a benzenesulfonamide-containing NSAID (e.g., any of the benzenesulfonamide-containing NSAID described herein or known in the art); (b) a first solution comprising about 0.02% (w/v) to about 100% (w/v) (or any of the subranges of this range described herein), or about 0.02% (v/v) to about 100% (v/v) (or any of the subranges of this range described herein) of a gelling agent (e.g., any of the gelling agents described herein or known in the art); (c) a second solution comprising about 1% (w/v) to about 60% (w/v) (or any of the subranges of this range described herein) silk fibroin (e.g., a silk fibroin having a molecular weight of about 10 kDa to about 500 kDa); (d) mixing a volume of the first solution, a volume of the second solution, and a volume of the suspension to provide a mixture; and (e) incubating the mixture of a time sufficient to form the composition (e.g., gel, e.g., hydrogel or organogel). In some embodiments of these methods, the suspension includes any of the subranges of 1 mg/mL to about 400 mg/mL of the benzenesulfonamide-containing NSAIDs described herein. In some examples, the suspension includes a plurality of particles of the benzenesulfonamide-containing NSAID (e.g., any of the sizes and types of particles described herein). In some embodiments of these methods, the first solution includes any of the subranges of about 0.02% (w/v) to about 100% (w/v) of the gelling agent described herein. In some embodiments of these methods, the first solution includes any of the subranges of about 0.02% (v/v) to about 100% (v/v) of the gelling agent described herein. In some embodiments of these methods, the second solution includes any of the subranges of about 1% (w/v) to about 60% (w/v) of the silk fibroin described herein.

In any of the methods described herein, the gelling agent is glycerol or a polyethylene glycol (PEG) (e.g., any of the PEGs described herein). In some embodiments of any of the methods described herein, the PEG is PEG300, PEG400, or PEG600, or a PEG having a molecular weight of about 2 kDa, about 4 kDa, about 6 kDa, about 8 kDa, about 10 kDa, about 12 kDa, about 14 kDa, about 16 kDa, about 18 kDa, or about 20 kDa.

In some embodiments of any of these methods, the gelling agent is selected from the group of: sorbitol, triethylamine, 2-pyrrolidone, alpha-cyclodextrin, benzyl alcohol, beta-cyclodextrin, dimethyl sulfoxide, dimethylacetamide (DMA), dimethylformamide, ethanol, gamma-cyclodextrin, glycerol, glycerol formal, hydroxypropyl beta-cyclodextrin, kolliphor 124, kolliphor 181, kolliphor 188, kolliphor 407, kolliphor EL (cremaphor EL), cremaphor RH 40, cremophor RH 60, d-alpha-tocopherol, PEG 1000 succinate, polysorbate 20, polysorbate 80, solutol HS 15, sorbitan monooleate, poloxamer-407, poloxamer-188, Labrafil M-1944CS, Lrabfil M-2125CS, Labrasol, Gellucire 44/14, Softigen 767, mono- and di-fatty acid esters of PEG 300, PEG 400, or PEG 1750, kolliphor RH60, N-methyl-2-pyrrolidone, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil, beeswax, d-alpha-tocopherol, oleic acid, medium-chain mono- and di-glycerides, alpha-cyclodextrin, beta-cyclodextrin, hydroxypropyl-beta-cyclodextrin, sulfo-butylether-beta-cyclodextrin, hydrogenated soy phosphatidylcholine, distearoylphosphatidylglycerol, L-alpha-dimyristoylphosphatidylcholine, L-alpha-dimyristoylphosphatidylglycerol, PEG 300, PEG 300 caprylic/capric glycerides (Softigen 767), PEG 300 linoleic glycerides (Labrafil M-2125CS), PEG 300 oleic glycerides (Labrafil M-1944CS), PEG 400, PEG 400 caprylic/capric glycerides (Labrasol), polyoxyl 40 stearate (PEG 1750 monosterate), polyoxyl 8 stearate (PEG 400 monosterate), polysorbate 20, polysorbate 80, polyvinyl pyrrolidone, propylene carbonate, propylene glycol, solutol HS 15, sorbitan monooleate (Span 20), sulfobutylether-beta-cyclodextrin, transcutol, triacetin, 1-dodecylazacyclo-heptan-2-one, caprolactam, castor oil, cottonseed oil, ethyl acetate, medium chain triglycerides, methyl acetate, oleic acid, safflower oil, sesame oil, soybean oil, tetrahydrofuran, and glycerin.

In some embodiments of any of the methods described herein, the silk fibroin has a molecular weight range of about 10 kDa to about 500 kDa (or any of the subranges of this range described herein). Some embodiments of these methods further include generating silk fibroin having a molecular weight range of about 10 kDa to about 500 kDa. In some embodiments of these methods, the step of generating a silk fibroin includes incubating a silk raw material in a solution including sodium carbonate (Na₂CO₃) for 5 minutes to 300 minutes (or any of the subranges of this range described herein) at about 70° C. to about 100° C. (or any of the subranges of this range described herein).

In some embodiments of any of the methods described herein, the benzenesulfonamide-containing NSAID includes a pyrazole moiety. In some embodiments of any of the methods described herein, the pyrazine moiety is a haloalkyl-substituted pyrazole moiety (e.g., a trifluoromethyl-substituted pyrazole moiety). In some embodiments of any of the methods described herein, the benzenesulfonamide-containing NSAID is celecoxib.

Some examples of these methods further include, between the mixing and incubating step, a step of sterilizing the mixture. Some examples of these methods further include after the incubating step, a step of sterilizing the mixture. In some examples, the sterilizing step is performed by autoclaving the mixture (e.g., autoclaving the mixture at 121° C. for at least 30 minutes, at least 35 minutes, at least 40 minutes, at least 45 minutes, at least 50 minutes, at least 55 minutes, or at least 60 minutes). Some embodiments, wherein the final concentration of the gelling agent in the composition is about 0.01% (w/v) to about 2% (w/v), about 1.8% (w/v), about 1.6% (w/v), about 1.4% (w/v), about 1.2% (w/v), about 1.0% (w/v), about 0.8% (w/v), about 0.6% (w/v), about 0.4% (w/v), about 0.2% (w/v), about 0.1% (w/v), or about 0.05% (w/v) (inclusive), can include a step of autoclaving the mixture (e.g., using any of the conditions for autoclaving described herein or known in the art), between the mixing and incubating steps, and/or after the incubating step. Some embodiments, wherein the final concentration of the gelling agent in the composition is about 0.01% (v/v) to about 2% (v/v), about 1.8% (v/v), about 1.6% (v/v), about 1.4% (v/v), about 1.2% (v/v), about 1.0% (v/v), about 0.8% (v/v), about 0.6% (v/v), about 0.4% (v/v), about 0.2% (v/v), about 0.1% (v/v), or about 0.05% (v/v) (inclusive), can include a step of autoclaving the mixture (e.g., using any of the conditions for autoclaving described herein or known in the art), between the mixing and incubating steps, and/or after the incubating step.

Also provided herein are compositions (e.g., gels, e.g., hydrogels) produced by any of the methods described herein. In some examples of these compositions (e.g., gels, e.g., hydrogels), the composition (e.g., gel, e.g., hydrogel) includes a plurality of particles of the benzenesulfonamide-containing NSAID. In some embodiments of these compositions (e.g., gels, e.g., hydrogels), the plurality of particles have any of the size distributions or sizes described herein (e.g., one or more of: d90 less than about 200 μm (e.g., less than 60 μm), a d50 of less than about 25 μm, and a d10 of less than about 10 μm).

### Methods of Delivering a Composition to a Subject

In some embodiments, a medical professional (e.g., a nurse, a physician's assistant, or a physician) is provided any of the compositions (e.g., gels, e.g., hydrogels or organogels) provided herein, and the medical professional administers a therapeutically effective amount of the composition to a subject (e.g., any of the subjects described herein).

In other examples, a medical professional is provided with a first solution including about 1% (w/v) to about 60% (w/v) silk fibroin (e.g., silk fibroin having a molecular weight range of about 10 kDa to about 500 kDa (or any of the subranges described herein)) (e.g., any of the first solutions described herein) and a second solution including about 0.02% (w/v) to about 100% (w/v), or about 0.02% (v/v) to about 100% (v/v), of a gelling agent, and about 1 mg/mL to about 400 mg/mL of a benzenesulfonamide-containing NSAID (e.g., any of the second solutions described herein); and the medical professional mixes a volume of the first solution with a volume of the second solution, where the ratio of the volume of the first solution to the volume of the second solution is about 5:1 to about 1:5 to provide a mixture; incubates the mixture for a time sufficient to form the composition (e.g., gel, e.g., hydrogel or organogel); and administers a therapeutically effective amount of the composition (e.g., gel, e.g., hydrogel or organogel) to a subject (e.g., any of the subjects described herein).

In other examples, a medical professional is provided with a double-barreled syringe, where a first barrel in the double-barreled syringe contains a first solution including about 1% (w/v) to about 60% (w/v) silk fibroin (e.g., a silk fibroin having a molecular weight range of about 10 kDa to about 500 kDa (or any of the subranges described herein)) (e.g., any of the first solutions described herein) and a second barrel in the double-barreled syringe contains a second solution including about 0.02% (w/v) to about 100% (w/v), or about 0.02% (v/v) to about 100% (v/v), of a gelling agent, and about 1 mg/mL to about 400 mg/mL of a benzenesulfonamide-containing NSAID (e.g., any of the second solutions described herein); and the medical professional administers a therapeutically effective volume of the first solution and the second solution using the double-barreled syringe to a subject (e.g., any of the subjects described herein), where the ratio of the volume of the first solution administered to the volume of the second solution administered is about 5:1 to about 1:5, and following administration of the first and second solutions to the subject, a composition (e.g., gel, e.g., hydrogel or organogel) forms in a tissue in the subject (e.g., in a joint or proximal to a cartilage or bone).

In some examples, a medical professional is provided with a double-barreled syringe, which includes in one barrel a solution of about 1% (w/v) to about 60% (w/v) silk fibroin (e.g., a silk fibroin having a molecular weight range of about 10 kDa to about 500 kDa (or any of the subranges described herein), and one barrel that is empty, and a separate vial or container including a suspension of about 1 mg/mL to about 400 mg/mL of benzenesulfonamide-containing particles (e.g., any of the sized particles described herein) and about 0.02% (w/v) to about 100% (w/v), or about 0.02% (v/v) to about 100% (v/v), of a gelling agent (e.g., any of the gelling agents described herein). The medical professional is provided instructions to draw up the suspension in the vial or container into the empty barrel of the double-barreled syringe. In other examples, a medical professional is provided with a dried silk fibroin powder (e.g., a powder comprising a silk fibroin having a molecular weight range of about 10 kDa to about 500 kDa, or any of the subranges described herein), and the medical professional solubilizes or reconstitutes the powder with a solution or suspension comprising a gelling agent (e.g., any of the gelling agents described herein) and a benzenesulfonamide-containing NSAID (e.g., any of the second solutions or suspensions described herein); incubates the solubilized or reconstituted powder to form a composition (e.g., gel (e.g., a hydrogel or organogel), a slurry, or a solubilized silk fibroin solution), and administers a therapeutically effective amount of the composition to a subject (e.g., any of the subjects described herein).

### Compositions and Kits

Also provided are compositions (e.g., pharmaceutical compositions) that include any of the gels (e.g., hydrogels or organogels) described herein. Also provided are kits that include any of the compositions (e.g., gels, e.g., hydrogels or organogels) provided herein. In some examples, the kit further includes a syringe (e.g., a syringe that is pre-filled with any of the compositions (e.g., gels, e.g., hydrogels or organogels) provided herein). In some examples, the kit further includes a sealed vial (e.g., a sealed vial that is sterilely filled with any of the compositions (e.g., gels, e.g., hydrogels or organogels) provided herein). In some examples, the kit can further include instructions for preforming any of the methods of treating an inflammatory and/or pain condition in a subject or any of the methods of reducing inflammation, pain, or fever in a subject described herein.

Methods of Treating an Inflammatory and/or Pain Condition

Provided herein are methods of treating an inflammatory and/or pain condition in a subject that include administering a therapeutically effective amount of a composition (e.g., a gel, e.g., a hydrogel or organogel) including a benzenesulfonamide-containing NSAID (e.g., any of the benzenesulfonamide-containing NSAIDs described herein or known in the art), silk fibroin, and a gelling agent (e.g., any of the gelling agents described herein or known in the art). In some embodiments, the composition (e.g., gel, e.g., hydrogel or organogel) releases a therapeutically effective amount of the benzenesulfonamide-containing NSAID over a period of about 10 days to about 200 days (e.g., any of the subranges described herein) when incubated in phosphate buffered saline (PBS), 2% Tween-80 in PBS, or 2% Tween-80 and 0.05% sodium azide in PBS at 37° C.

The inflammatory and/or pain condition can be, e.g., selected from the group of: a joint pain and/or inflammatory diseases, musculoskeletal pain, back pain, neuropathic pain, nocioceptive pain, acute pain, chronic pain, inflammatory pain, operative pain, post-operative pain, visceral pain, incident pain, breakthrough pain, psychogenic pain, psoriasis, Crohn's disease, irritable bowel syndrome, Sjogren's disease, tissue graft rejection, asthma, systemic lupus erythematosus, glomerulonephritis, dermatomyositis, multiple sclerosis, scleroderma, vasculitis, Goodpasture's syndrome, atherosclerosis, chronic idiopathic thrombocytopenic purpura, Addison's disease, Parkinson's disease, Alzheimer's disease, diabetes, septic shock, myasthenia gravis, inflammatory pelvic disease, inflammatory bowel disease, urethritis, uveitis, sinusitis, pneumonitis, encephalitis, meningitis, myocarditis, nephritis, osteomyelitis, myositis, hepatitis, gastritis, enteritis, dermatitis, appendicitis, pancreatitis, cholocystitis, polycystic kidney disease, and a cancer. In some examples, the inflammatory condition is a joint disease. Non-limiting examples of a joint disease include osteoarthritis, rheumatoid arthritis, spondyloarthritis, systemic juvenile idiopathic arthritis, psoriatic arthritis, gout, ankylosing spondylitis, and juvenile rheumatoid arthritis.

Successful treatment of an inflammatory and/or pain condition can be determined (e.g., determined by a medical professional) by detecting one or more of: a reduction in the number of symptoms of the inflammatory and/or pain condition in the subject, a reduction of the severity of one or more symptoms of the inflammatory and/or pain condition in the subject, a reduction in the duration of one or more symptoms of the inflammatory and/or pain condition in the subject, and a reduction in the frequency of one or more symptoms of the inflammatory and/or pain condition in the subject. Symptoms of inflammatory and/or pain conditions are known in the art.

For example, successful treatment of an inflammatory condition can be determined by detecting a detectable reduction in the level/severity one or more (e.g., two or more) markers/symptoms of inflammation in a subject following administration of a therapeutic agent (e.g., any of the compositions (e.g., gels, e.g., hydrogels organogels) provided herein) as compared to the level/severity of the one or more (e.g., two or more) markers/symptoms of inflammation in the subject prior to administration of the therapeutic agent. Non-limiting examples of symptoms of inflammation include: pain, heat, redness, swelling, and loss of function. Non-limiting examples of markers of inflammation include prostaglandins (e.g., PGE2), erythrocyte sedimentation rate (ESR), white cell count, interleukin (IL)-1, IL-2, IL-3, IL-4, IL-5, IL-6, IL-7, IL-8, IL-9, IL-10, IL-11, IL-12, IL-13, tumor necrosis factor (TNF)-α, TNF-β, tumor growth factor-β, interferon-γ, GCSF, GM-CSF, C-reactive protein, serum amyloid P component, serum amyloid A, complement protein C3, complement protein C5a, factor XII, membrane attack complex (complement system), plasmin, thrombin, mannan-binding lectin, fibrinogen, prothrombin, factor VIII, von Willebrand factor, plasminogen activator inhibitor PAI-1, alpha 2-macroglobulin, ferritin, hecidin, ceruloplasmin, haptoglobin, orosomucoid, α-1-antitrpysin, α-1-antichymotrypsin, histamine, and leukotriene B4. For example, successful treatment of an inflammatory condition can result in a decrease in pain (e.g., inflammatory pain) in the subject. For example, treatment of an inflammatory condition can result in a decrease in the pain associated with the inflammatory condition.

For example, successful treatment of a pain condition can be determined by detecting a detectable decrease in a subject's pain intensity following administration of a therapeutic agent (e.g., any of the compositions (e.g., gels, e.g., hydrogels or organogels) provided herein) as compared to the intensity of the subject's pain prior to administration of the therapeutic agent. In some embodiments, the pain condition is one or more of nociceptive pain, neuropathic pain, psychogenic pain, breakthrough pain, incident pain, back pain, musculoskeletal pain, post-operative pain, operative pain, visceral pain, joint pain, acute pain, inflammatory pain, knee pain, dental pain, and chronic pain. The intensity of pain in a subject can be determined, e.g., using a pain scale. Non-limiting examples of pain scales that can be used to determine the intensity of pain in a subject include: Alder Hey Triage Pain Score, Behavioral Pain Scale (BPS), Brief Pain Inventory (BPI), Checklist of Nonverbal Pain Indicators (CNPI), Clinical Global Impression (CGI), Critical-Care Point Observation Tool (CPOT), COMFORT scale, Dallas Pain Questionnaire, Descriptor Differential Scale (DDS), Dolorimeter Pain Index (DPI), Edmonton Symptom Assessment System, Faces Pain Scale-Revised (FPS-R), Face Legs Activity Cry Consolability Scale, Lequesne Algofunctional Index, McGill Pain Questionnare (MPQ), Neck Pain and Disability Scale (NPAD), Numerical 11 Point Box (BS-11), Numeric Rating Scale (NRS-11), OSWESTRY Index, Palliative Care Outcome Scale (PCOS), Roland-Morris Back Pain Questionnare, Support Team Assessment Schedule (STAS), Wong-Baker FACES Pain Rating Scale, Visual Analog Scale (VAS), Australian/Canadian Osteoarthritis Hand Index (AUSCAN), Western Ontario and McMaster Universities Hand Index (WOMAC), and Osteoarthritis Research Society International-Outcome Measures in Rheumatoid Arthritis Clinical Trials (OARSI-OMERACT).

In some examples of these methods, the subject has previously been diagnosed as having an inflammatory and/or pain condition, or has been selected for administration of the composition (e.g., gel, e.g., hydrogel or organogel). In some examples, the subject is a juvenile. In other examples, the subject is an adult. In other examples, the subject is a human. In other examples, the subject is a non-human mammal (e.g., a domesticated animal).

The therapeutically effective amount of any of the compositions (e.g., gels, e.g., hydrogels or organogels) provided herein can be administered by auricular administration, intraarticular administration, intramuscular administration, intrathecal administration, extracorporeal administration, buccal administration, intrabronchial administration, conjunctival administration, cutaneous administration, dental administration, endocervical administration, endosinusial administration, endotracheal administration, enteral administration, epidural administration, intra-abdominal administration, intrabiliary administration, intrabursal administration, oropharyngeal administration, interstitial administration, intracardiac administration, intracartilaginous administration, intracaudal administration, intracavernous administration, intracerebral administration, intracorporous cavernosum, intracavitary administration, intracorneal administration, intracisternal administration, intracranial administration, intradermal administration, intralesional administration, intraileal administration, intratympanic administration, intragingival administration, intraovarian administration, intraocular administration, intradiscal administration, intraductal administration, intraduodenal administration, ophthalmic administration, intradural administration, intraepidermal administration, intraesophageal administration, nasogastric administration, nasal administration, laryngeal administration, intraventricular administration, intragastric administration, intrahepatic administration, intaluminal administration, intravitreal administration, intravesicular administration, intralymphatic administration, intamammary administration, intramedullary administration, intrasinal administration, intrameningeal administration, intranodal administration, intaovarian administration, intrapulmonary administration, intapericardial administration, intraperitoneal administration, intrapleural administration, intrapericardial administration, intraprostatic administration, intapulmonary administration, intraluminal administration, intraspinal administration, intrasynovial administration, intratendinous administration, intratesticular administration, subconjunctival administration, retrobulbar administration, periarticular administration, intrathoracic administration, subarachnoid administration, intratubular administration, periodontal administration, transtympanic administration, transtracheal administration, intratumor administration, vaginal administration, urethral administration, intrauterine administration, oral administration, parenteral administration, sublingual administration, ureteral administration, percutaneous administration, peridural administration, transmucosal administration, perineural administration, rectal administration, soft tissue administration, or subcutaneous administration. In some examples, the therapeutically effective amount of any of the compositions (e.g., gels, e.g., hydrogels or organogels) described herein can be administered by local administration (e.g., local to a site of inflammation in the subject). For example, a composition (e.g., gel, e.g., hydrogel or organogel) can be administered by intraarticular administration to a subject having a joint disease. For example, a composition (e.g., gel, e.g., hydrogel or organogel) can be administered by intra-disc or peri-disc administration to a subject having back pain. For example, a composition (e.g., gel, e.g., hydrogel or organogel) can be administered by inter-knee or peri-knee injection for a subject having knee pain.

For example, a composition (e.g., gel, e.g., hydrogel or organogel) can be administered to the ear, neck, muscle (e.g., skeletal muscle), joint (e.g., a knee, a hip, an elbow, ankle, a wrist, or a finger joint), breast, back, colon, intestine, sinus, eye, brain, spine, abdomen, pancreas, liver, dental pulp, lymph node, rectum, ovary, prostate gland, skin, lungs, subcutaneous tissue, skin, or heart.

In some examples, the subject is administered a therapeutically effective amount of any of the compositions (e.g., gels, e.g., hydrogels or organogels) described herein, once a day, once every two days, once every three days, once every four days, once every five days, once every six days, once a week, once every 8 days, once every 9 days, once every 10 days, once every 11 days, once every 12 days, once every 13 days, once every two weeks, once every three weeks, once a month, once every six weeks, once every two months, once every 10 weeks, once every three months, once every 14 weeks, once every 4 months, once every 18 weeks, once every 5 months, once every 22 weeks, once every 6 months, once every 7 months, once every 8 months, once every 9 months, once every 10 months, once every 11 months, or once a year.

A subject having an inflammatory and/or pain condition can be treated using the methods described herein (e.g., multiple administrations of a therapeutically effective amount of any of the compositions (e.g., gels, e.g., hydrogels or organogels) described herein or any of the compositions described herein) for a total period of time of about 1 month to about 80 years, about 75 years, about 70 years, about 65 years, about 60 years, about 55 years, about 50 years, about 45 years, about 40 years, about 35 years, about 30 years, about 25 years, about 20 years, about 15 years, about 10 years, about 8 years, about 6 years, about 4 years, about 2 years, about 1 year, or about 6 months; about 6 months to about 80 years, about 75 years, about 70 years, about 65 years, about 60 years, about 55 years, about 50 years, about 45 years, about 40 years, about 35 years, about 30 years, about 25 years, about 20 years, about 15 years, about 10 years, about 8 years, about 6 years, about 4 years, about 2 years, or about 1 year; about 1 year to about 80 years, about 75 years, about 70 years, about 65 years, about 60 years, about 55 years, about 50 years, about 45 years, about 40 years, about 35 years, about 30 years, about 25 years, about 20 years, about 15 years, about 10 years, about 8 years, about 6 years, about 4 years, or about 2 years; about 2 years to about 80 years, about 75 years, about 70 years, about 65 years, about 60 years, about 55 years, about 50 years, about 45 years, about 40 years, about 35 years, about 30 years, about 25 years, about 20 years, about 15 years, about 10 years, about 8 years, about 6 years, or about 4 years; about 4 years to about 80 years, about 75 years, about 70 years, about 65 years, about 60 years, about 55 years, about 50 years, about 45 years, about 40 years, about 35 years, about 30 years, about 25 years, about 20 years, about 15 years, about 10 years, about 8 years, or about 6 years; about 6 years to about 80 years, about 75 years, about 70 years, about 65 years, about 60 years, about 55 years, about 50 years, about 45 years, about 40 years, about 35 years, about 30 years, about 25 years, about 20 years, about 15 years, about 10 years, or about 8 years; about 8 years to about 80 years, about 75 years, about 70 years, about 65 years, about 60 years, about 55 years, about 50 years, about 45 years, about 40 years, about 35 years, about 30 years, about 25 years, about 20 years, about 15 years, or about 10 years; about 10 years to about 80 years, about 75 years, about 70 years, about 65 years, about 60 years, about 55 years, about 50 years, about 45 years, about 40 years, about 35 years, about 30 years, about 25 years, about 20 years, or about 15 years; about 15 years to about 80 years, about 75 years, about 70 years, about 65 years, about 60 years, about 55 years, about 50 years, about 45 years, about 40 years, about 35 years, about 30 years, about 25 years, or about 20 years; about 20 years to about 80 years, about 75 years, about 70 years, about 65 years, about 60 years, about 55 years, about 50 years, about 45 years, about 40 years, about 35 years, about 30 years, or about 25 years; about 25 years to about 80 years, about 75 years, about 70 years, about 65 years, about 60 years, about 55 years, about 50 years, about 45 years, about 40 years, about 35 years, or about 30 years; about 30 years to about 80 years, about 75 years, about 70 years, about 65 years, about 60 years, about 55 years, about 50 years, about 45 years, about 40 years, or about 35 years; about 35 years to about 80 years, about 75 years, about 70 years, about 65 years, about 60 years, about 55 years, about 50 years, about 45 years, or about 40 years; about 40 years to about 80 years, about 75 years, about 70 years, about 65 years, about 60 years, about 55 years, about 50 years, or about 45 years; about 45 years to about 80 years, about 75 years, about 70 years, about 65 years, about 60 years, about 55 years, or about 50 years; about 50 years to about 80 years, about 75 years, about 70 years, about 65 years, about 60 years, or about 55 years; about 55 years to about 80 years, about 75 years, about 70 years, about 65 years, or about 60 years; about 60 years to about 80 years, about 75 years, about 70 years, or about 65 years; about 65 years to about 80 years, about 75 years, or about 70 years; about 70 years to about 80 years or about 75 years; or about 75 years to about 80 years.

Any of the compositions (e.g., gels, e.g., hydrogels or organogels) described herein can be administered to the subject. Any of the compositions (e.g., gels, e.g., hydrogels or organogels) described herein can be administered to the subject (e.g., a subject having any of the inflammatory and/or pain conditions described herein) in any of the single dose volumes described herein. Any of the total single dose amounts of the benzenesulfonamide-containing NSAID (or any of the single dose amounts of each of the benzenesulfonamide-containing NSAIDs for compositions (e.g., gels, e.g., hydrogels or organogels) including two or more benzenesulfonamide-containing NSAIDs) described herein can be administered to the subject (e.g., a subject having any of the inflammatory and/or pain conditions described herein) in a single dose.

### Methods of Reducing Inflammation, Pain, or Fever

Also, provided herein are methods of reducing inflammation, pain, or fever in a subject in need thereof that include administering a therapeutically effective amount of a composition (e.g., gel, e.g., a hydrogel or organogel) including a benzenesulfonamide-containing NSAID (e.g., any of the benzenesulfonamide-containing NSAIDs described herein or known in the art), silk fibroin, and a gelling agent (e.g., any of the gelling agents described herein or known in the art). In some examples of any of these methods, the composition (e.g., gel, e.g., hydrogel or organogel) can release a therapeutically effective amount of the benzenesulfonamide-containing NSAID over a period of about 10 days to about 200 days when incubated in phosphate buffered saline (PBS), 2% Tween-80 in PBS, or 2% Tween-80 and 0.05% sodium azide in PBS at 37° C.

A reduction in inflammation in a subject can be detected by observing a reduction in the level/severity of one or more (e.g., two or more, e.g., three, four, or five) markers/symptoms of inflammation in a subject following administration of any of the compositions (e.g., gels, e.g., hydrogels or organogels) provided herein (as compared to the level/severity of the two or more markers/symptoms of inflammation in the subject prior to administration of the composition). Non-limiting examples of symptoms of inflammation include: pain, heat, redness, swelling, and loss of function. Non-limiting examples of markers of inflammation include prostaglandins (e.g., PGE2), erythrocyte sedimentation rate (ESR), white cell count, interleukin (IL)-1, IL-2, IL-3, IL-4, IL-5, IL-6, IL-7, IL-8, IL-9, IL-10, IL-11, IL-12, IL-13, tumor necrosis factor (TNF)-α, TNF-β, tumor growth factor-β, interferon-γ, GCSF, GM-CSF, C-reactive protein, serum amyloid P component, serum amyloid A, complement protein C3, complement protein C5a, factor XII, membrane attack complex (complement system), plasmin, thrombin, mannan-binding lectin, fibrinogen, prothrombin, factor VIII, von Willebrand factor, plasminogen activator inhibitor PAI-1, alpha 2-macroglobulin, ferritin, hecidin, ceruloplasmin, haptoglobin, orosomucoid, α-1-antitrpysin, α-1-antichymotrypsin, histamine, and leukotriene B4.

A reduction in pain can be detectable decrease in a subject's pain intensity following administration of any of the compositions (e.g., gels, e.g., hydrogels or organogels) provided herein (as compared to the intensity of the subject's pain prior to administration of the gel or the composition). In some embodiments, the pain is one or more of nociceptive pain, neuropathic pain, psychogenic pain, breakthrough pain, incident pain, back pain, musculoskeletal pain, post-operative pain, operative pain, visceral pain, joint pain, acute pain, inflammatory pain, knee pain, dental pain, and chronic pain. The intensity of pain in a subject can be determined, e.g., using a pain scale. Non-limiting examples of pain scales that can be used to determine the intensity of pain in a subject include: Alder Hey Triage Pain Score, Behavioral Pain Scale (BPS), Brief Pain Inventory (BPI), Checklist of Nonverbal Pain Indicators (CNPI), Clinical Global Impression (CGI), Critical-Care Point Observation Tool (CPOT), COMFORT scale, Dallas Pain Questionnaire, Descriptor Differential Scale (DDS), Dolorimeter Pain Index (DPI), Edmonton Symptom Assessment System, Faces Pain Scale-Revised (FPS-R), Face Legs Activity Cry Consolability Scale, Lequesne Algofunctional Index, McGill Pain Questionnare (MPQ), Neck Pain and Disability Scale (NPAD), Numerical 11 Point Box (BS-11), Numeric Rating Scale (NRS-11), OSWESTRY Index, Palliative Care Outcome Scale (PCOS), Roland-Morris Back Pain Questionnare, Support Team Assessment Schedule (STAS), Wong-Baker FACES Pain Rating Scale, Visual Analog Scale (VAS), Australian/Canadian Osteoarthritis Hand Index (AUSCAN), Western Ontario and McMaster Universities Hand Index (WOMAC), and Osteoarthritis Research Society International-Outcome Measures in Rhematoid Arthritis Clinical Trials (OARSI-OMERACT). In some examples, the pain is associated with any of the inflammatory conditions described herein. In some embodiments, the pain is associated with macular edema.

A reduction in fever in a subject (as compared to a normal body temperature range for a subject (e.g., a human)) can be detected using a thermometer (e.g., an oral thermometer).

In some examples of these methods, the subject has previously been diagnosed or identified as having inflammation, pain, or fever (e.g., inflammation, pain, or fever in a joint), or has been selected for administration of the composition (e.g., gel, e.g., hydrogel or organogel). In some examples, the subject is a juvenile. In other examples, the subject is an adult. In other examples, the subject is a human. In other examples, the subject is a non-human mammal (e.g., a domesticated animal).

The therapeutically effective amount of any of the compositions (e.g., gels, e.g., hydrogels or organogels) described herein can be administered by auricular administration, intraarticular administration, intramuscular administration, intrathecal administration, extracorporeal administration, buccal administration, intrabronchial administration, conjunctival administration, cutaneous administration, dental administration, endocervical administration, endosinusial administration, endotracheal administration, enteral administration, epidural administration, intra-abdominal administration, intrabiliary administration, intrabursal administration, oropharyngeal administration, interstitial administration, intracardiac administration, intracartilaginous administration, intracaudal administration, intracavernous administration, intracerebral administration, intracorporous cavernosum, intracavitary administration, intracorneal administration, intracisternal administration, intracranial administration, intradermal administration, intralesional administration, intraileal administration, intratympanic administration, intragingival administration, intraovarian administration, intraocular administration, intradiscal administration, intraductal administration, intraduodenal administration, ophthalmic administration, intradural administration, intraepidermal administration, intraesophageal administration, nasogastric administration, nasal administration, laryngeal administration, intraventricular administration, intragastric administration, intrahepatic administration, intaluminal administration, intravitreal administration, intravesicular administration, intralymphatic administration, intamammary administration, intramedullary administration, intrasinal administration, intrameningeal administration, intranodal administration, intaovarian administration, intrapulmonary administration, intapericardial administration, intraperitoneal administration, intrapleural administration, intrapericardial administration, intraprostatic administration, intapulmonary administration, intraluminal administration, intraspinal administration, intrasynovial administration, intratendinous administration, intratesticular administration, subconjunctival administration, retrobulbar administration, periarticular administration, intrathoracic administration, subarachnoid administration, intratubular administration, periodontal administration, transtympanic administration, transtracheal administration, intratumor administration, vaginal administration, urethral administration, intrauterine administration, oral administration, parenteral administration, sublingual administration, ureteral administration, percutaneous administration, peridural administration, transmucosal administration, perineural administration, rectal administration, soft tissue administration, or subcutaneous administration. In some examples, the therapeutically effective amount of any of the compositions (e.g., gels, e.g., hydrogels or organogels) described herein can be administered by local administration (e.g., local to a site of inflammation in the subject). For example, a composition (e.g., a gel, e.g., a hydrogel or organogel) described herein can be administered by intraarticular administration to a subject having inflammation or pain in a joint. For example, a composition (e.g., a gel, e.g., hydrogel or organogel) described herein can be administered by intra-disc or peri-disc administration to a subject having back pain. For example, a composition (e.g., a gel, e.g., a hydrogel or organogel) described herein can be administered by inter-knee or peri-knee injection for a subject having knee pain.

For example, a composition (e.g., a gel, e.g., a hydrogel or organogel) described herein can be administered to the ear, neck, muscle (e.g., skeletal muscle), joint (e.g., a knee, a hip, an elbow, ankle, a wrist, or a finger joint), breast, back, colon, intestine, sinus, eye, brain, spine, abdomen, pancreas, liver, dental pulp, lymph node, rectum, ovary, prostate gland, skin, lungs, subcutaneous tissue, skin, or heart.

In some examples, the subject is administered a therapeutically effective amount of any of the compositions (e.g., gels, e.g., hydrogels or organogels) described herein, once a day, once every two days, once every three days, once every four days, once every five days, once every six days, once a week, once every 8 days, once every 9 days, once every 10 days, once every 11 days, once every 12 days, once every 13 days, once every two weeks, once every three weeks, once a month, once every six weeks, once every two months, once every 10 weeks, once every three months, once every 14 weeks, once every 4 months, once every 18 weeks, once every 5 months, once every 22 weeks, once every 6 months, once every 7 months, once every 8 months, once every 9 months, once every 10 months, once every 11 months, or once a year.

A subject having inflammation, pain, or fever can be treated using the methods described herein (e.g., multiple administrations of a therapeutically effective amount of any of the compositions (e.g., gels, e.g., hydrogels or organogels) described herein) for a total period of time of about 1 month to about 80 years, about 75 years, about 70 years, about 65 years, about 60 years, about 55 years, about 50 years, about 45 years, about 40 years, about 35 years, about 30 years, about 25 years, about 20 years, about 15 years, about 10 years, about 8 years, about 6 years, about 4 years, about 2 years, about 1 year, or about 6 months; about 6 months to about 80 years, about 75 years, about 70 years, about 65 years, about 60 years, about 55 years, about 50 years, about 45 years, about 40 years, about 35 years, about 30 years, about 25 years, about 20 years, about 15 years, about 10 years, about 8 years, about 6 years, about 4 years, about 2 years, or about 1 year; about 1 year to about 80 years, about 75 years, about 70 years, about 65 years, about 60 years, about 55 years, about 50 years, about 45 years, about 40 years, about 35 years, about 30 years, about 25 years, about 20 years, about 15 years, about 10 years, about 8 years, about 6 years, about 4 years, or about 2 years; about 2 years to about 80 years, about 75 years, about 70 years, about 65 years, about 60 years, about 55 years, about 50 years, about 45 years, about 40 years, about 35 years, about 30 years, about 25 years, about 20 years, about 15 years, about 10 years, about 8 years, about 6 years, or about 4 years; about 4 years to about 80 years, about 75 years, about 70 years, about 65 years, about 60 years, about 55 years, about 50 years, about 45 years, about 40 years, about 35 years, about 30 years, about 25 years, about 20 years, about 15 years, about 10 years, about 8 years, or about 6 years; about 6 years to about 80 years, about 75 years, about 70 years, about 65 years, about 60 years, about 55 years, about 50 years, about 45 years, about 40 years, about 35 years, about 30 years, about 25 years, about 20 years, about 15 years, about 10 years, or about 8 years; about 8 years to about 80 years, about 75 years, about 70 years, about 65 years, about 60 years, about 55 years, about 50 years, about 45 years, about 40 years, about 35 years, about 30 years, about 25 years, about 20 years, about 15 years, or about 10 years; about 10 years to about 80 years, about 75 years, about 70 years, about 65 years, about 60 years, about 55 years, about 50 years, about 45 years, about 40 years, about 35 years, about 30 years, about 25 years, about 20 years, or about 15 years; about 15 years to about 80 years, about 75 years, about 70 years, about 65 years, about 60 years, about 55 years, about 50 years, about 45 years, about 40 years, about 35 years, about 30 years, about 25 years, or about 20 years; about 20 years to about 80 years, about 75 years, about 70 years, about 65 years, about 60 years, about 55 years, about 50 years, about 45 years, about 40 years, about 35 years, about 30 years, or about 25 years; about 25 years to about 80 years, about 75 years, about 70 years, about 65 years, about 60 years, about 55 years, about 50 years, about 45 years, about 40 years, about 35 years, or about 30 years; about 30 years to about 80 years, about 75 years, about 70 years, about 65 years, about 60 years, about 55 years, about 50 years, about 45 years, about 40 years, or about 35 years; about 35 years to about 80 years, about 75 years, about 70 years, about 65 years, about 60 years, about 55 years, about 50 years, about 45 years, or about 40 years; about 40 years to about 80 years, about 75 years, about 70 years, about 65 years, about 60 years, about 55 years, about 50 years, or about 45 years; about 45 years to about 80 years, about 75 years, about 70 years, about 65 years, about 60 years, about 55 years, or about 50 years; about 50 years to about 80 years, about 75 years, about 70 years, about 65 years, about 60 years, or about 55 years; about 55 years to about 80 years, about 75 years, about 70 years, about 65 years, or about 60 years; about 60 years to about 80 years, about 75 years, about 70 years, or about 65 years; about 65 years to about 80 years, about 75 years, or about 70 years; about 70 years to about 80 years or about 75 years; or about 75 years to about 80 years.

Any of the compositions (e.g., gels, e.g., hydrogels or organogels) described herein can be administered to the subject. Any of the compositions (e.g., gels, e.g., hydrogels or organogels) described herein can be administered to the subject (e.g., a subject having inflammation, pain, or fever) in any of the single dose volumes described herein. Any of the total single dose amounts of the benzenesulfonamide-containing NSAID (or any of the single dose amounts of each of the benzenesulfonamide-containing NSAIDs for compositions (e.g., gels, e.g., hydrogels or organogels) including two or more benzenesulfonamide-containing NSAIDs) described herein can be administered to the subject (e.g., a subject having inflammation, pain, or fever) in a single dose.

The invention is further described in the following examples, which do not limit the scope of the invention described in the claims.

### EXAMPLES

### Example 1. Sustained Release of a Benzenesulfonamide-Containing NSAID from a Gel Including Silk Fibroin and a PEG

A set of experiments were performed to evaluate the drug release profile of different gels including silk fibroin, a PEG, and one of several different NSAIDs. The materials and methods used to perform these studies are described below.

### Materials

Lyophilized silk fibroin (0.3 g per vial) was purchased from Simatech Inc. (Suzhou, China). Celecoxib was purchased from Matrix Scientific (Elgin, S.C.). The other NSAIDs, pharmaceutical grade (Kollisolv®) PEGs, and all other chemicals were purchased from Sigma-Aldrich (St. Louis, Mo.).

### Methods

### Drug Loading in Silk-PEG Hydrogel

Lyophilized silk was dissolved with ultrapure water to obtain concentrations of 1.3, 3.6, 7.0, 13.0, and 23.0% (w/v). Polyethylene glycol (PEG) with molecular weights of 300 and 400 Da (PEG300 and PEG400) was diluted with ultrapure water when necessary. Celecoxib (a benzenesulfonamide-containing NSAID) and other NSAIDs which are not benzenesulfonamide-containing NSAIDs (tenoxicam, meloxicam, and lomoxicam) were dissolved with 80% (v/v) PEG400 solution to obtain low concentration solutions (<40 mg/mL) or with 100% (v/v) PEG300 to obtain high concentration solutions (>100 mg/mL). Diclofenac sodium salt (another NSAID that is not a benzenesulfonamide-containing NSAID) was dissolved in the aqueous silk solution.

The PEG or PEG/drug solution was mixed with silk solution by pipetting up and down gently and aliquoted into a 2-mL Eppendorf tube. The detailed components of each tube is shown in Tables 1 to 7 below. Each tube was left at 37° C. to gel overnight (Wang et al., Acta Biomater. 12:51-61, 2015).

### In Vitro Release Study

To the 2-mL Eppendorf tube containing fibroin-drug hydrogel, 1.95 mL of release medium was added. The release medium contained 1× phosphate buffer, pH 7.4, 0.1% (v/v) Tween-80, and 0.02% (w/v) sodium azide. The samples were incubated at 37° C. with gentle shaking. The release medium was changed after 24 hours and then approximately once every 3 days. The release medium was subjected to UV detection using a UV transparent bottom plate (Corning) at the respective wavelengths listed in Table 8. Control release medium was measured under the same condition for background subtraction. A calibration curve was generated for each drug by dissolving a known amount of each drug in the release medium, and making dilutions with the release medium.

TABLE 8
UV Detection Wavelength (nm) Used to Detect NSAIDs

| Drug | Wavelength (nm) |
|---|---|
| Celecoxib | 260 |
| Diclofenac Sodium Salt | 280 |
| Lomoxicam | 380 |
| Meloxicam | 365 |
| Tenoxicam | 370 |

### Results

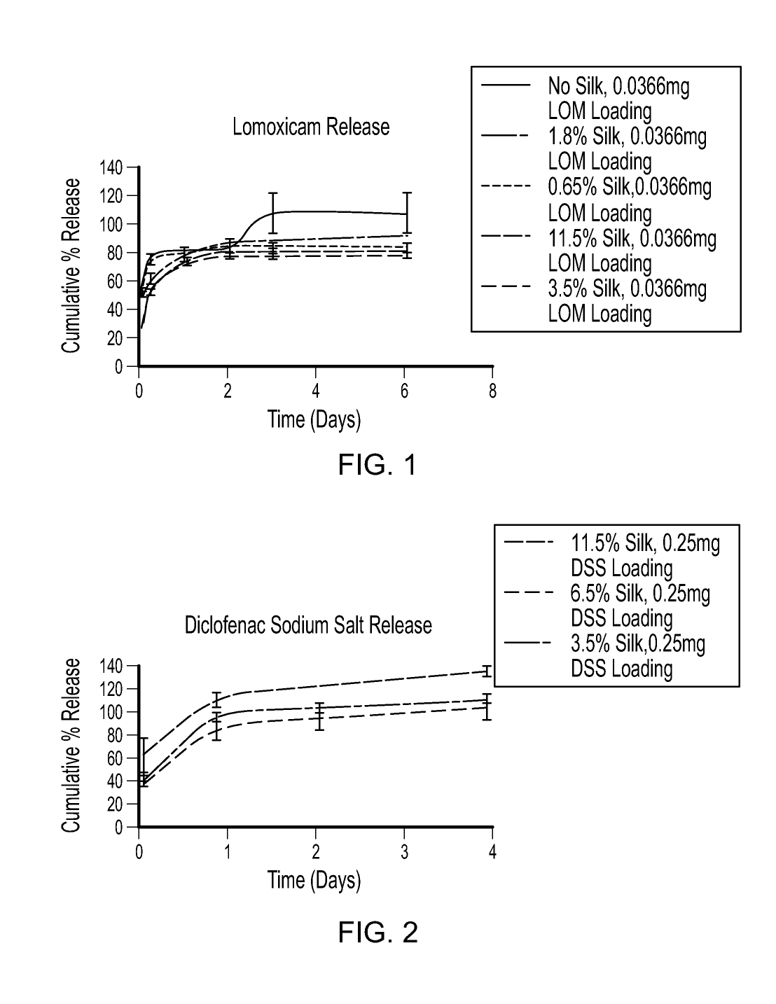

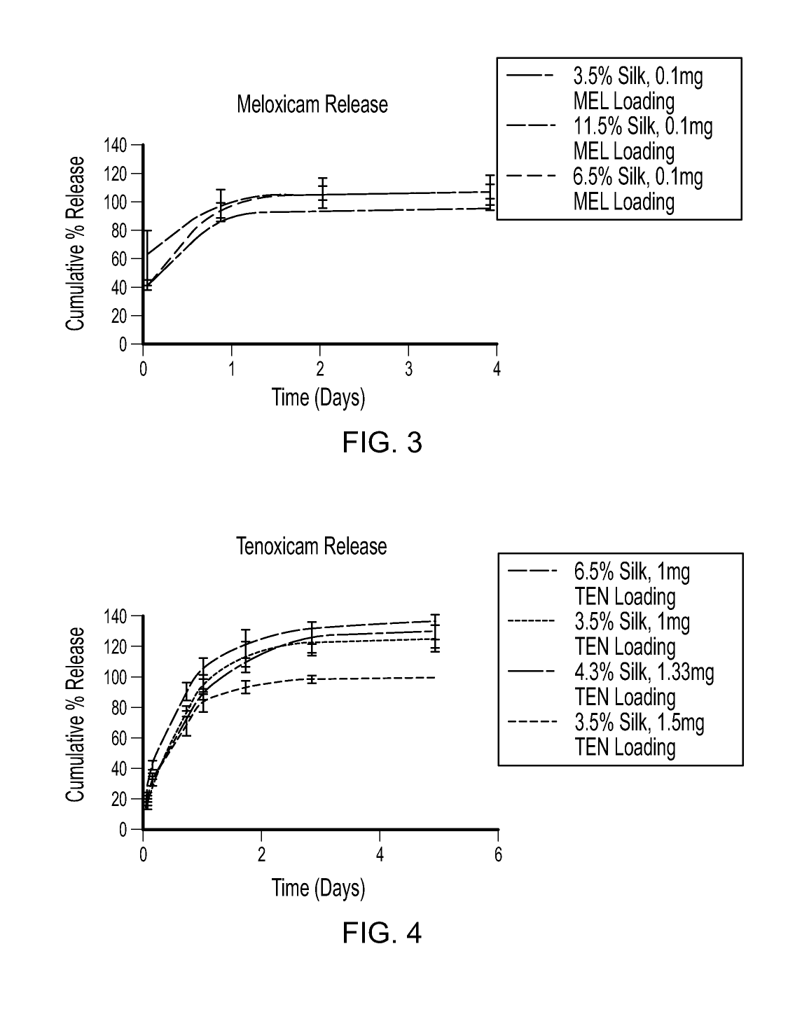

The cumulative release of lomoxican from silk-fibroin hydrogels plateaued at day 6 for all the tested silk-fibroin hydrogel formulations, while the release of the drug with no fibroin plateaued at day 2 (FIG. 1). The diclofenac sodium salt was completely released from all the tested silk-fibroin hydrogels within 4 days (FIG. 2). Meloxicam was completely released from all the tested silk-fibroin hydrogels within 2 days (FIG. 3). Tenoxicam was completely released from all the tested silk-fibroin hydrogels within 5 days (FIG. 4).

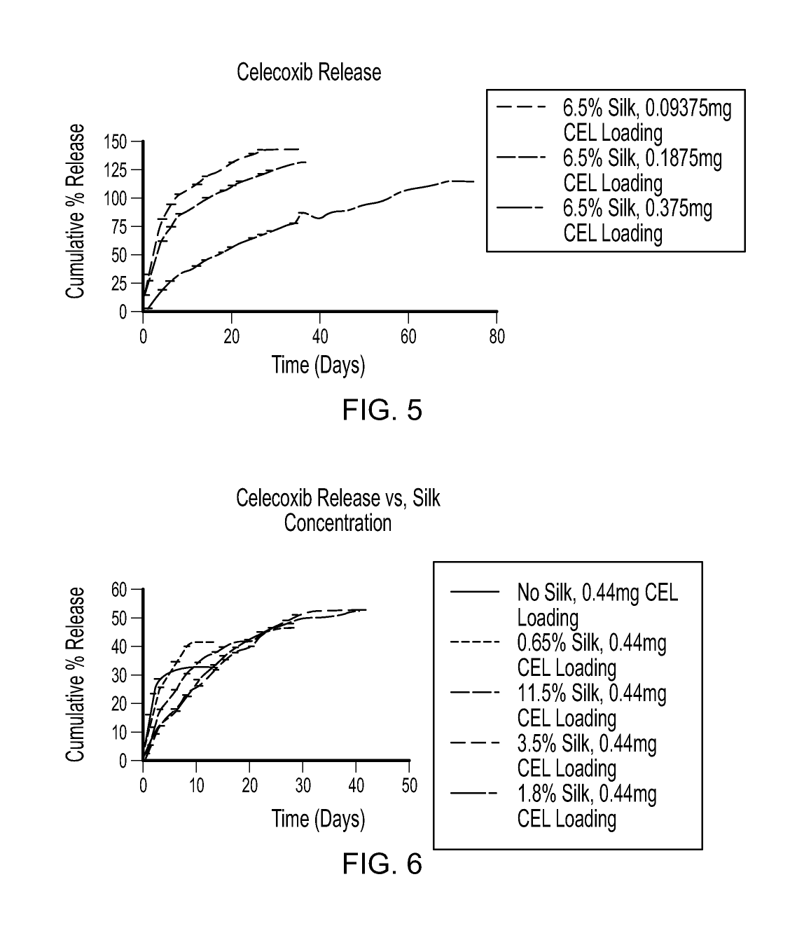

Celecoxib was released within 30 days in silk-fibroin hydrogels containing celecoxib concentrations of 0.09375% (w/v) and 0.1875% (w/v), while celecoxib was released within 75 days in silk-fibroin hydrogels having a celecoxib concentration of 0.375% (w/v) (FIG. 5). All silk-fibroin hydrogels were prepared at 6.5% (w/v) fibroin. The data show that higher drug loading resulted in a slower and more linear release of celecoxib from the silk-fibroin hydrogel, as compared to lower celecoxib drug loadings (FIG. 5). The greater than 100% release observed for some samples is likely due to mass balance issues due to background subtraction from controls (e.g., silk alone) during UV absorbance determinations.

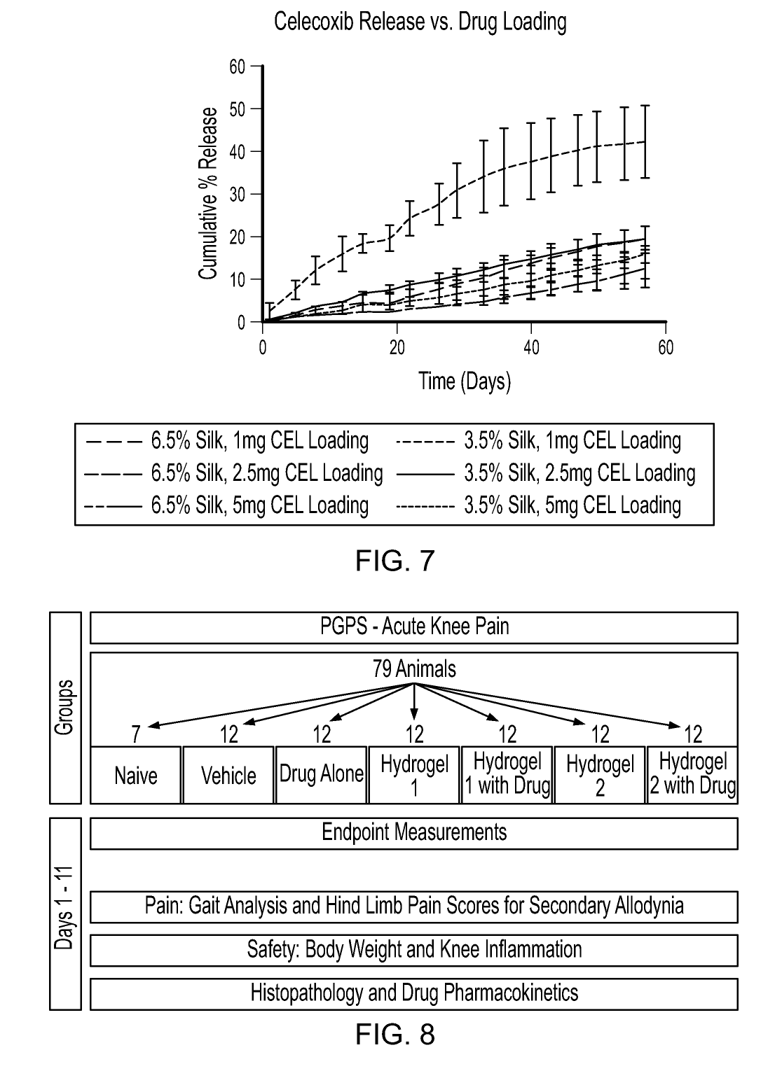

The data in FIG. 6 show that celecoxib release occurs over 41 days for hydrogels having fibroin concentrations of 12.5% (w/v) and 3.5% (w/v), while celecoxib release occurs over 30 days for hydrogels having a fibroin concentration of 1.3% (w/v), over 15 days for hydrogels having a fibroin concentration of 0.65% (w/v), and over 6 days for a no fibroin/“free” celecoxib sample (0% silk-fibroin hydrogels). The data in FIG. 7 show that all hydrogels released celecoxib for extended time period within 56 days, while hydrogels including low celecoxib concentration (1 mg) and low fiborin concentration (3.5% (w/v)) released celecoxib faster than the other samples.

In sum, these data show that the sustained release (e.g., greater than one week) of lomoxicam, meloxicam, tenoxicam, and diclofenac sodium salt could not be achieved using a silk-fibroin hydrogel delivery platform, i.e., these drugs were released within a one-week time period. Free lomoxican, meloxicam, tenoxicam, and diclofenac without encapsulation in silk-fibroin hydrogels were released within one day. In other words, the silk-fibroin hydrogel did slow the release of lomoxicam, meloxicam, tenoxicam, and diclofenac sodium salt to a limited extent.

Surprisingly, celecoxib interacted with the silk-fibroin in the gel in such a way that its release was sustained over two months when fibroin concentrations in the hydrogel were 3.5% (w/v) to 6.5% (w/v). The data show that the silk-fibroin hydrogels provide for the slow release of celecoxib at a therapeutically effective amount of ˜40 μg a day for more than 75 days.

### Example 2. Treatment of Acute Knee Inflammation and Pain in a Rat Model Using a Celecoxib Silk-Fibroin Hydrogel Composition

A set of experiments was performed to determine if intraarticular injection of a celecoxib silk-fibroin hydrogel composition would successfully treat acute knee inflammation and pain in a rat model.

### Materials and Methods

### PGPS Induction

A PGPS (peptidoglycan-polysaccharide) rat model of acute knee inflammation and pain as described in Esser et al., Arth. Rheum. 28(12): 1402-1411, 1985 was used in these experiments. Rats were either left untreated or received a single intraarticular injection of 50 μL at day 0 of vehicle (“vehicle”), celecoxib alone (“Drug alone”), 6.5% (w/v) silk-fibroin hydrogel not including celecoxib (“Hydrogel 1”), 6.5% (w/v) silk-fibroin hydrogel including 100 mg/mL celecoxib (“Hydrogel 1 with drug), 3.5% (w/v) silk-fibroin hydrogel not including celecoxib (“Hydrogel 2”), or 3.5% (w/v) silk-fibroin hydrogel including 100 mg/mL celecoxib (“Hydrogel 2 with drug”) (FIG. 8) (Table 9). The specific hydrogels administered in these experiments are shown in Table 10. The control and silk-fibroin hydrogels were intraarticularly administered in the rat model.

Formulations were prepared by dissolving lyophilized silk fibroin with ultrapure water to obtain concentrations of either 7.0 or 13.0% (w/v). Celecoxib was dissolved in PEG300 to obtain 200 mg/mL concentration. Silk fibroin solution and celecoxib solution were drawn into separate 1 cc. syringes, connected via a luer connector and mixed back and forth 25 times or until homogeneous. Hydrogels were left to form at room temperature for at least 16 hours.

For priming, animals were anesthetized with isoflurance and given an intraarticular injection of peptidoglycan polysaccharride (PGPS) (50 μL of 0.125 mg/mL) into the right knee. The knee arthritis was reactivated with an intravenous (IV) injection of PGPS (0.5 mL of 0.4 mg/mL).

### Gait Analysis

Gait analysis and hind limb pain (Von Frey) score for secondary allodynia, as well as knee inflammation (knee diameter), were measured over days 1-11 of the study. Gait analysis was performed by applying ink to the ventral surface of the foot and documenting weight bearing during movement (footprints) across paper. The rear feet of rats were placed in ink and then rats were placed on paper and allowed to walk the full length. This process was repeated as necessary to generate 4 clear, evenly inked footprint pairs representing the overall pattern of gait. Gait were scored visually as follows (descriptions refer to diseased leg):
0=Normal, approximately equal ink staining to normal paw
1=Slight limp/pain=reduced inking area relative to the normal paw, but no full regions or structures are missing
2=Mild limp/pain=Print extends to the end or near to the end of the “curlicue” structure. If normal paw has very little heel staining (rat walks mainly on toes/ball of foot), then slightly less staining
3=Moderate limp/pain=toes and full ball of foot, extending to the top of the “curlicue” foot. If normal paw has very little heel staining (rat walks mainly on toes/ball of foot), then toes with small portion of ball of foot.
4=Marked limp/pain=toes and partial ball of foot, no heel or posterior foot. If normal paw has very little heel staining (rat walks mainly on toes/ball of foot), then toes only.
5=Severe limp/pain=toes only, no ball of foot, no heel. If normal paw has very little heel staining (rat walks mainly on toes/ball of foot), then partial toes or non-specific marks
6=Hopping or Carrying leg, no footprint is evident.

### Knee Caliper Measurements

Caliper measurements of right and left knee diameters were done on assigned days using a Digitrix II micrometer caliper (Fowler & NSK). Baseline knee caliper measurements were taken using one knee with values rounded to one-thousandth of an inch. The measurements were confirmed as clinically normal by comparison with historical values for rats based on a range of body weights. Baseline measurements were then applied to both knees, and these values remained with the animal so long as the knee was clinically normal. Caliper measurements were done 48 and 96 hours post each reactivation.

TABLE 9
Group Designations from Rat Efficacy Study in PGPS Study

| | | | | | | Conc Fibroin |
|---|---|---|---|---|---|---|
| | | | | | | (% w/v): |
| | | | | Dosing | Dose | Celecoxib |
| Group | Treatment | Route | Regimen | Days | Vol | (mg/mL) |
| 1 | Naive | N/A | N/A | N/A | N/A | N/A |
| 2 | Vehicle | IA | Day 0 | Single Day | 50 μL | N/A |
| | | | | (day 0) | | |
| 3 | NSAID | IA | Day 0 | Single Day | 50 μL | N/A: 100 |
| | | | | (Day 0) | | |
| 4 | Gel 1 | IA | Day 0 | Single Day | 50 μL | 6.5%: N/A |
| | | | | (Day 0) | | |
| 5 | Gel 1 + NSAID | IA | Day 0 | Single Day | 50 μL | 6.5%: 100 |
| | | | | (Day 0) | | |
| 6 | Gel 2 | IA | Day 0 | Single Day | 50 μL | 3.5%: N/A |
| | | | | (Day 0) | | |
| 7 | Gel 2 + NSAID | IA | Day 0 | Single Day | 50 μL | 3.5%: 100 |
| | | | | (Day 0) | | |

TABLE 10
Celecoxib Hydrogel Formulations-Rat Efficacy Study in PGPS Model

| | | | 25 μL of | | | | |
|---|---|---|---|---|---|---|---|
| | | 25 μL fibroin | drug-PEG | | | Final | |
| | | solution | solution | | Final drug | fibroin | Final |
| | | Silk % | Drug | PEG % | loading | (w/v) | PEG |
| Group | Treatment | (w/v) | (mg/mL) | (v/v) | (mg) | % | % (v/v) |
| 1 | Naive | N/A | N/A | N/A | N/A | N/A | N/A |
| 2 | Vehicle | N/A | N/A | 100% | 0 | 0 | 50% |
| | | | | PEG-300 | | | PEG-300 |
| 3 | NSAID | N/A | 200 | 100% | 100 | 0 | 50% |
| | | | | PEG-300 | | | PEG-300 |
| 4 | Gel 1 | 13% | N/A | 100% | 0 | 6.5% | 50% |
| | | | | PEG-300 | | | PEG-300 |
| 5 | Gel 1 + | 13% | 200 | 100% | 100 | 6.5% | 50% |
| | NSAID | | | PEG-300 | | | PEG-300 |
| 6 | Gel 2 | 7% | N/A | 100% | 0 | 3.5% | 50% |
| | | | | PEG-300 | | | PEG-300 |
| 7 | Gel 2 + | 7% | 200 | 100% | 100 | 3.5% | 50% |
| | NSAID | | | PEG-300 | | | PEG-300 |

### Results

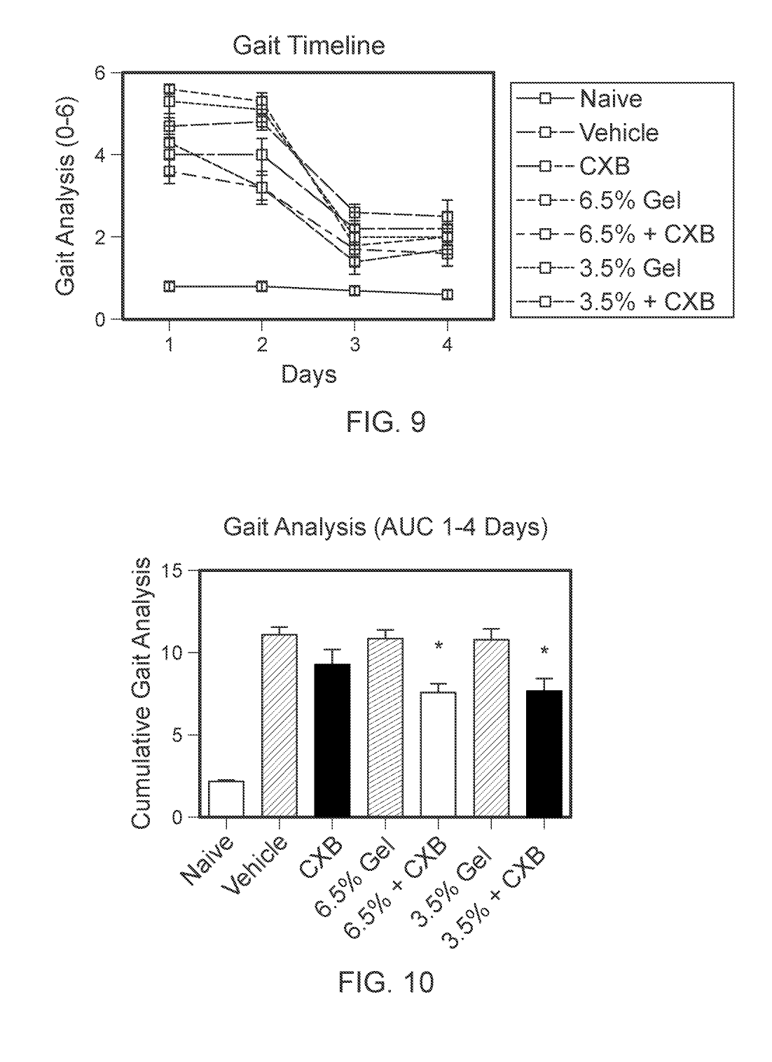

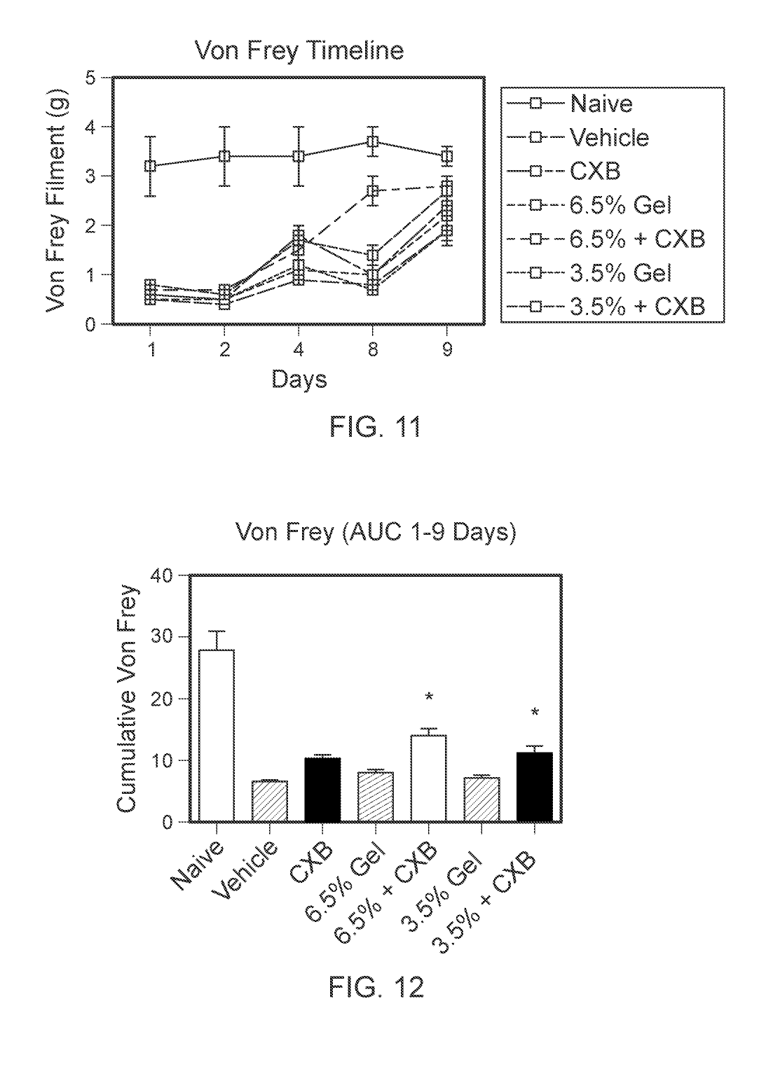

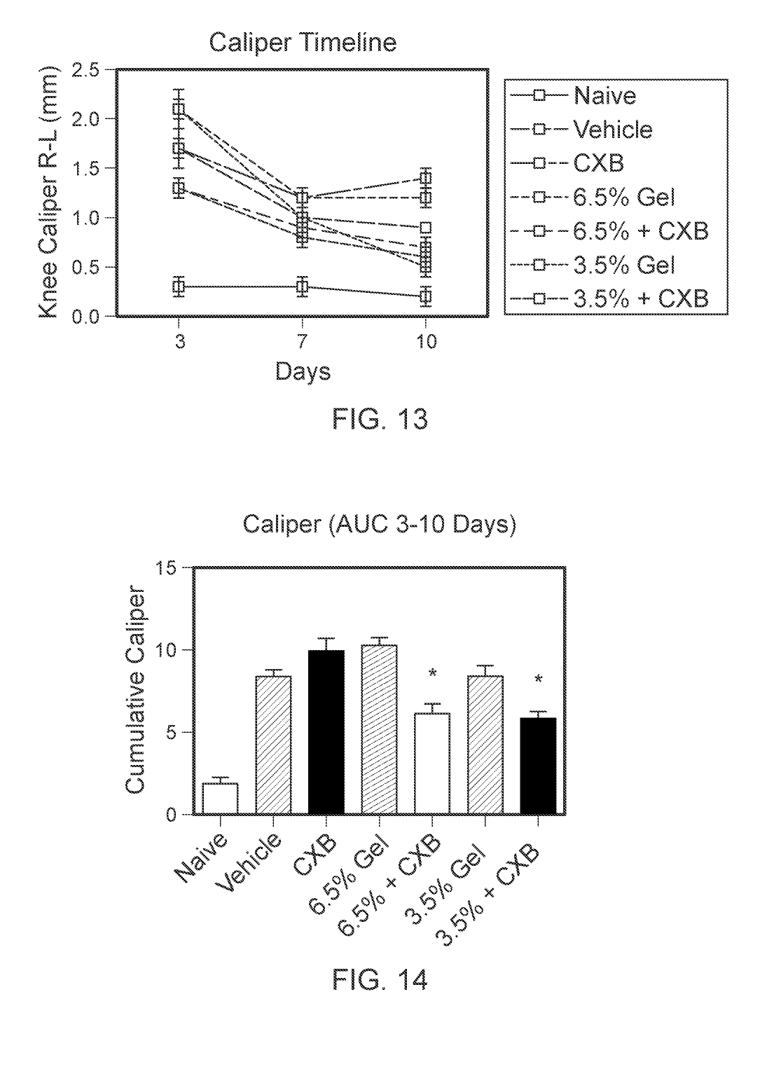

The data in FIGS. 9 and 10 show that intraarticular administration of the tested silk-fibroin celecoxib hydrogels to the knee of the rat model resulted in a decreased gait analysis score (which indicates a reduced amount of pain in the treated knee). The data in FIGS. 11 and 12 show that intraarticular administration of the tested silk-fibroin celecoxib gels to the knee of the rat model resulted in an increased Von Frey Filment (g) measurement, which indicates less pain in the knee. The data in FIGS. 13 and 14 show that intraarticular administration of the tested silk-fibroin celecoxib gels to the knee of the rat model results in a decrease in the diameter of the knee of the rat model, which indicates a decrease in the inflammation of the knee.

In sum, these data show that pain relief and a reduction in inflammation was achieved following intraarticular administration of a silk-fibroin celecoxib gel in rat model of acute knee inflammation and pain.

### Example 3. Determining Silk-Fibroin Molecular Weight Range for Gelation Summary

### Methods

Briefly, silk yarn from SOHO was degummed at 100° C. for either 15, 30, 45, 60, 90, 120, or 240 minutes in 0.02 M sodium carbonate solution to remove sericin and modify the molecular weight (MW) of the fibroin. The fibroin was then dried overnight, weighed, and dissolved at 20% (w/v) in a 9.3 M aqueous solution of lithium bromide for five hours at 60° C. The resulting fibroin solution was dialyzed against water in 3.5 kDa regenerated cellulose dialysis tubing for 48 hours with six water changes to remove the excess salt. The final solution was centrifuged three times for 20 minutes each at 9,000 RPM and 4° C. to remove insoluble particles. Solutions were diluted to a final concentration of 3% (w/v) in 10 mM phosphate buffer, pH 7.4. Half of these solutions were then autoclaved for 30 minutes. The solution that was degummed for 240 minutes and then autoclaved was then split and half was autoclaved for a second time. 10 mL of each solution was aliquoted into 50 mL conical tubes, frozen, and lyophilized for 72 hours.

To form the hydrogels, lyophilized silk-fibroin was reconstituted with 2 mL of deionized (DI) water. One half mL of this solution was then drawn into a 1 cc. syringe. One half mL of 100% PEG300 was drawn into a separate 1 cc. syringe. These syringes were then connected with a luer-lok connector and mixed back and forth roughly 50× or until homogeneous. The mixture was pushed into one syringe, capped with a 27 gauge needle, and left to cure at room temperature overnight.

The reconstitution efficiency of each of the degumming methods was then determined using the following assay. A sample of the reconstituted fibroin solution used to prepare the hydrogel was diluted 20× and 40× in 10 mM phosphate buffer, pH 7.4. Two hundred μL of this solution was read on a UV plate reader at 280 nm and compared to a standard curve of known silk concentrations. The reconstitution efficiency was calculated as the actual fibroin concentration read on the plate reader divided by the theoretical fibroin concentration multiplied by 100%. For example, if all of the fibroin dissolved with the addition of 2.0 mL DI water, the concentration measured via absorbance at 280 nm should read 13% (w/v) fibroin. This would be equivalent to a reconstitution efficiency of 100%. Complete and efficient fibroin reconstitution is required to reproducibly prepare fibroin-based hydrogels. The molecular weight of each solution was then determined using size exclusion chromatography (SEC)-UV. The silk-fibroin solutions were diluted to 3 mg/mL in 100 mM phosphate buffer at a pH of 6.8 to be analyzed on a SEC-HPLC-UV system. A Waters SEC 450 protein standard was used to generate a standard curve (based on protein MW and retention time) to determine silk-fibroin MW. The samples and standards (5 μL) were injected onto a WATERS XBridge Protein BEH SEC, 450 Å column. The mobile phase (100 mM phosphate buffer at a pH of 6.8) was pumped at 0.86 mL/min. The absorbance was recorded at 280 nm.

### Results

Table 11 shows that silk that was degummed for at least 90 minutes or had been autoclaved at least once achieved reconstitution efficiencies of 85% or greater.

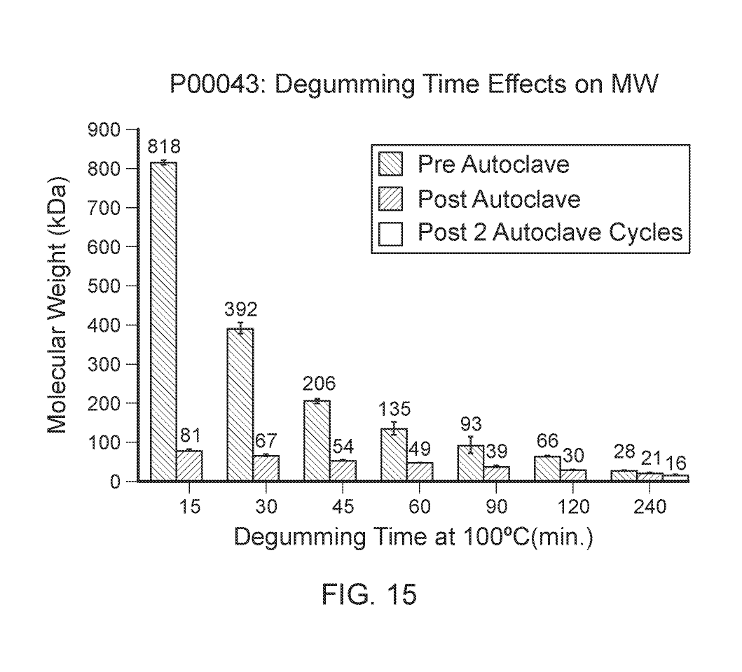

The size exclusion chromatography (SEC) results in FIG. 15 show that a wide range of molecular weights that can be achieved simply by degumming the silk at 100° C. in 0.02 M sodium carbonate solution for differing amounts of time (28-818 kDa). Also, the data show that the autoclave cycle leads to significant fibroin hydrolysis, drastically lowering the molecular weight. This data in combination with the reconstitution efficiency data suggests that silk-fibroin with a molecular weight on ≤˜100 kDa will have a reconstitution efficiency of ≥85% (FIG. 15).

TABLE 11
Reconstitution Efficiencies of the Silk-Fibroin Post-Lyophilization

| Sample | Degumming | | Reconstitution |
|---|---|---|---|
| Number | Time at 100° C. | Autoclave | efficiency (%) |
| 01 | 15 | No | 14.1 |
| 02 | 30 | No | 33.5 |
| 03 | 45 | No | 54.6 |
| 04 | 60 | No | 83.1 |
| 05 | 90 | No | 85.9 |
| 06 | 120 | No | 90.6 |
| 07 | 240 | No | 92.1 |
| 08 | 15 | Yes | 89.9 |
| 09 | 30 | Yes | 85.0 |
| 10 | 45 | Yes | 94.7 |
| 11 | 60 | Yes | 95.8 |
| 12 | 90 | Yes | 93.0 |
| 13 | 120 | Yes | 98.1 |
| 14 | 240 | Yes | 98.5 |
| 15 | 240 | Twice | 101.0 |

These data show that the longer silk-fibroin is heated at 100° C., the lower the molecular weight of the degummed silk-fibroin. Autoclaving the fibroin solution after degumming also drastically lowered the molecular weight. Additionally, this study did not find any lower limit of silk-fibroin molecular weight that would not allow for hydrogel formation (final PEG300 concentration of 50% (v/v)). The data show that silk fibroin with a molecular weight as low as 16 kDa was able to form a hydrogel. The data show that the reconstitution efficiency of lyophilized silk-fibroin increases as the molecular weight of silk-fibroin decreases.

### Example 4. Effect of Celecoxib Loading on Release Kinetics from Silk-Fibroin Hydrogels

### Methods

Silk cocoons from Treenway were degummed at 100° C. for 60 minutes in 0.02 M sodium carbonate solution to remove sericin. The fibroin was then dried overnight, weighed, and dissolved at 20% (w/v) in a 9.3 M aqueous lithium bromide solution for five hours at 60° C. The resulting solution was dialyzed against water with six water changes in 3.5 kDa regenerated cellulose dialysis membrane for 48 hours. The dialyzed solution was centrifuged three times at 9,000 RPM for 20 minutes and 4° C. to remove insoluble particles. The solutions were diluted to a final concentration of 3% in 10 mM phosphate buffer, pH 7.4, and autoclaved at 121° C. for 30 minutes. The solution was then filtered through a 0.22-μm filter aliquoted into 50-mL conical tubes, frozen, and lyophilized for 72 hours.

Purified fibroin was reconstituted with 2 mL of DI water. 0.5 mL of this solution was then drawn into a 1 cc. syringe. Separately, celecoxib was dissolved in PEG300 at either 200 or 60 mg/mL, for the 100 mg/mL and 30 mg/mL gels, respectively. One-half mL of the celecoxib/PEG300 solution was drawn into a separate 1 cc. syringe. These syringes were connected with a luer-lok connector and mixed back and forth approximately 50× or until homogeneous. The resulting mixture was then drawn into one syringe, capped with a 27 gauge needle, and left to gel at room temperature overnight.

In vitro release was carried out by weighing 50 mL-conical tubes and measuring approximately 50-100 mg of gel into the tubes and recording the actual weight for measurement. A suspension control was generated by mixing 25 μL of 200 mg/mL celecoxib in PEG-300 with 25 μL of DI water. Forty-five mL of release medium (1× phosphate buffered saline, 2% (v/v) Tween-80, and 0.05% sodium azide) was added to each tube. The samples were incubated at 37° C. under shaking. One mL of release media was collected at days 1, 3, 7, 10, 14, 21, 28, 36, and 42 and replaced with fresh media. The release media was analyzed by high performance liquid chromatography (HPLC) at 260 nm. The solubility of celecoxib in this release media is 850 μg/mL (data not shown). Forty-five mL of this release media allows 38 mg celecoxib to be dissolved. In this study, where samples contain either 5 mg or 1.5 mg celecoxib, this media will ensure sink conditions (≥5× celecoxib solubility) are maintained throughout the study. During the study, the tubes were placed on their sides and shook at 55 RPM and 37° C.

### Results

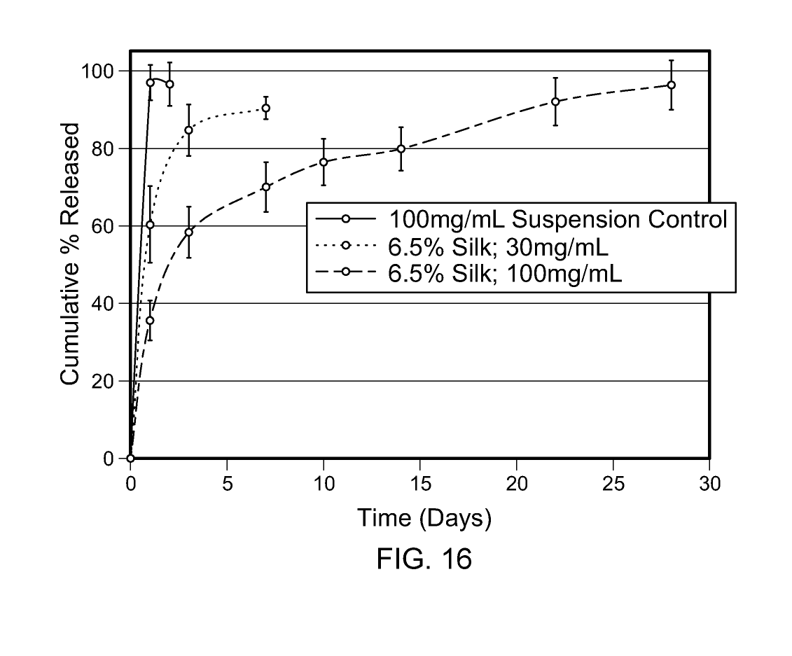

FIG. 16 shows that celecoxib release kinetics from silk fibroin hydrogels are related to celecoxib loading. The 30 mg/mL celecoxib hydrogels released celecoxib over 7 days, while the 100 mg/mL celecoxib hydrogels released celecoxib over 28 days. These data also shows the effect of the silk hydrogel on release kinetics. The suspension control and 100 mg/mL celecoxib-loaded fibroin hydrogel contained the same concentration of celecoxib. The suspension control displayed complete release in 24 hrs as compared to 28 days sustained release exhibited by the 6.5% (w/v) fibroin hydrogel.

Fibroin hydrogel formulations release celecoxib slower than drug suspensions alone, and the same volume of higher celecoxib-loaded hydrogel will release for a longer period of time. These data show clear differences between release rates of celecoxib from hydrogels which is related to celecoxib loading. A pure suspension of celecoxib will completely dissolve in the release medium in one day, as expected when under sink conditions. A silk fibroin hydrogel with 30 mg/mL celecoxib will release completely by day 7, and a silk fibroin hydrogel with 100 mg/mL celecoxib will release in approximately one month (under these test conditions).

These release curves are expected not to match in vivo release in the joint space as the solubility of celecoxib is much lower in synovial fluid than in 1×PBS and 2% Tween-80. As the solubility of celecoxib is much lower in synovial fluid (as compared to the buffer used in these studies), the same hydrogels are thought to have slower (more-extended) release profile of celecoxib as compared to the release profile in these in vitro studies.

### Example 5. Effect of Silk Fibroin Source on Release Kinetics of Celecoxib from Silk-Fibroin Hydrogels

### Methods

Silk was purchased from three sources: Treenway Cocoon, Treenway facial grade Cocoons, and SOHO silk yarn. The facial grade cocoons are similar to the standard cocoon except the worm has already been removed and the inside has been cleared of the majority of debris and particulates generally seen as a result of the worm's presence.

Silk yarn or cocoons were degummed at 100° C. in 0.02 M sodium carbonate for 60 minutes followed by three room temperature water washes. The fibroin was dried, dissolved at a final concentration of 20% (w/v) with 9.3 M lithium bromide, and dialyzed in 3.5 kDa regenerated cellulose dialysis membrane against deionized (DI) water for two days. The resulting silk fibroin solution was then diluted to a final concentration of 3% (w/v) and 10 mM phosphate buffer, pH 7.4, and then autoclaved at 121° C. for 30 minutes. The resulting fibroin was then filtered through a 0.22-μm filter unit and aseptically frozen on dry ice, followed by lyophilization for 72 hours. The molecular weight of each silk-fibroin sample before and after autoclaving was determined by size exclusion chromatography (SEC). The silk-fibroin solutions were diluted to 3 mg/mL in 100 mM phosphate buffer at a pH of 6.8 to be analyzed on SEC-HPLC-UV system. A Waters SEC 450 protein standard was used to generate a standard curve (based on protein MW and retention time) to determine silk fibroin molecular weight. The samples and standards (5 μL) were injected onto a WATERS XBridge Protein BEH SEC, 450 Å column. The mobile phase (100 mM phosphate buffer at a pH of 6.8) was pumped at 0.86 mL/min. The absorbance was recorded at 280 nm.

Silk-fibroin hydrogels were prepared by reconstituting the lyophilized fibroin from each source (Treenway cocoon, Treenway facial grade cocoons, and SOHO yarn) to 13% (w/v) or 7% (w/v) final fibroin concentration. The celecoxib/PEG solution was prepared by dissolving 200 mg/mL celecoxib in PEG300. Five hundred μL of fibroin solution was drawn up in a 1 cc. syringe and 500 μL of celecoxib/PEG solution was drawn up in a second 1 cc. syringe. The syringes were connected with a luer-lock connector and the contents were mixed back and forth 25× or until the hydrogel was homogenous. The hydrogels were left to cure at room temperature overnight. The composition of the hydrogels in this study are described in Table 12.

TABLE 12
The formulation conditions of the 12 hydrogels

| | Silk-fibroin | PEG300 | Celecoxib |
|---|---|---|---|
| | Conc. | Conc. | Conc. |
| Fibroin Source | % (w/v) | % (v/v) | (mg/mL) |
| Treenway Regular | 3.5 | 50 | 100 |
| Treenway Regular | 6.5 | 50 | 100 |
| Treenway Facial | 3.5 | 50 | 100 |
| Treenway Facial | 6.5 | 50 | 100 |
| SOHO Yarn | 3.5 | 50 | 100 |
| SOHO Yarn | 6.5 | 50 | 100 |

The in vitro release was carried out by weighing 50 mg of formulation into separate tubes containing 45 mL of release medium (1× phosphate buffered saline, 2% (v/v) Tween-80, and 0.05% (w/v) sodium azide). The solubility of celecoxib in this release media is 850 μg/mL. Forty-five mL of this release media allows for 38 mg celecoxib solubility. In this study, where samples contain 5 mg celecoxib, this media will ensure sink conditions (≥5× celecoxib solubility) are maintained throughout the study. The tubes were incubated at 37° C. while shaking. One mL of the release media was collected from each sample at each time point after standing the tubes up and allowing for settling of any large particulates for 15 minutes. One mL of fresh release medium was then added back to each tube. The release media was analyzed by HPLC at 260 nm.

### Results

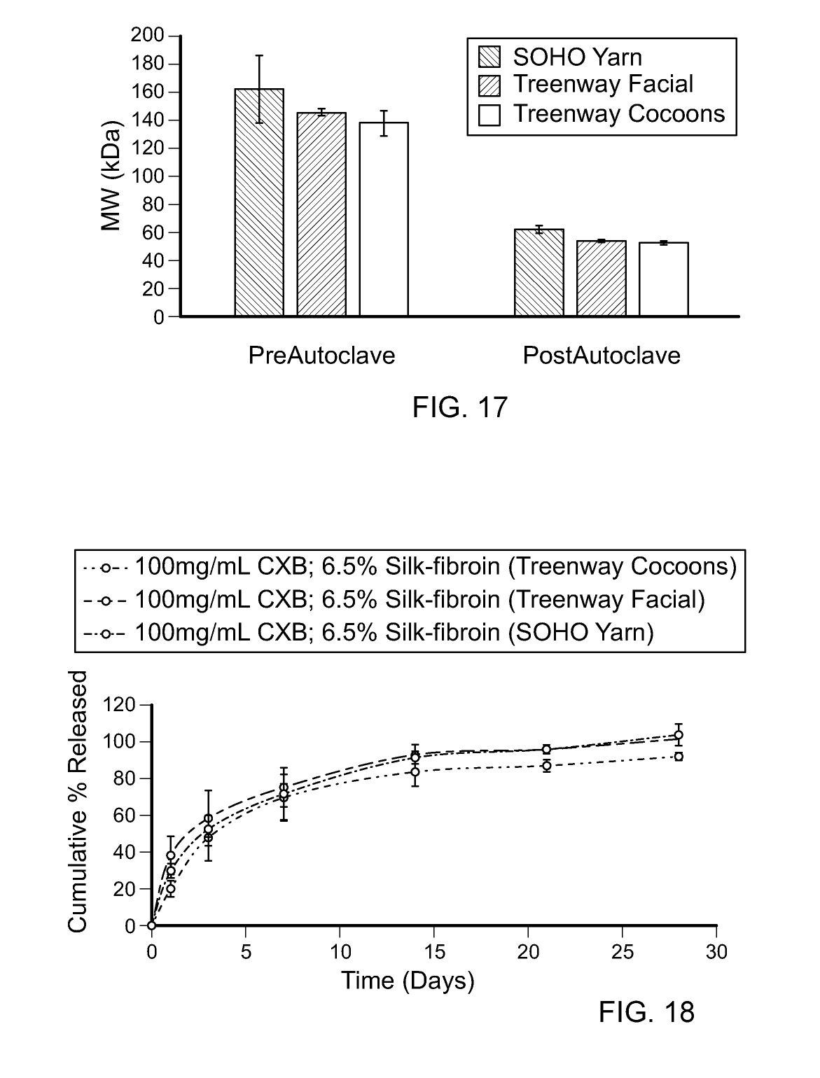

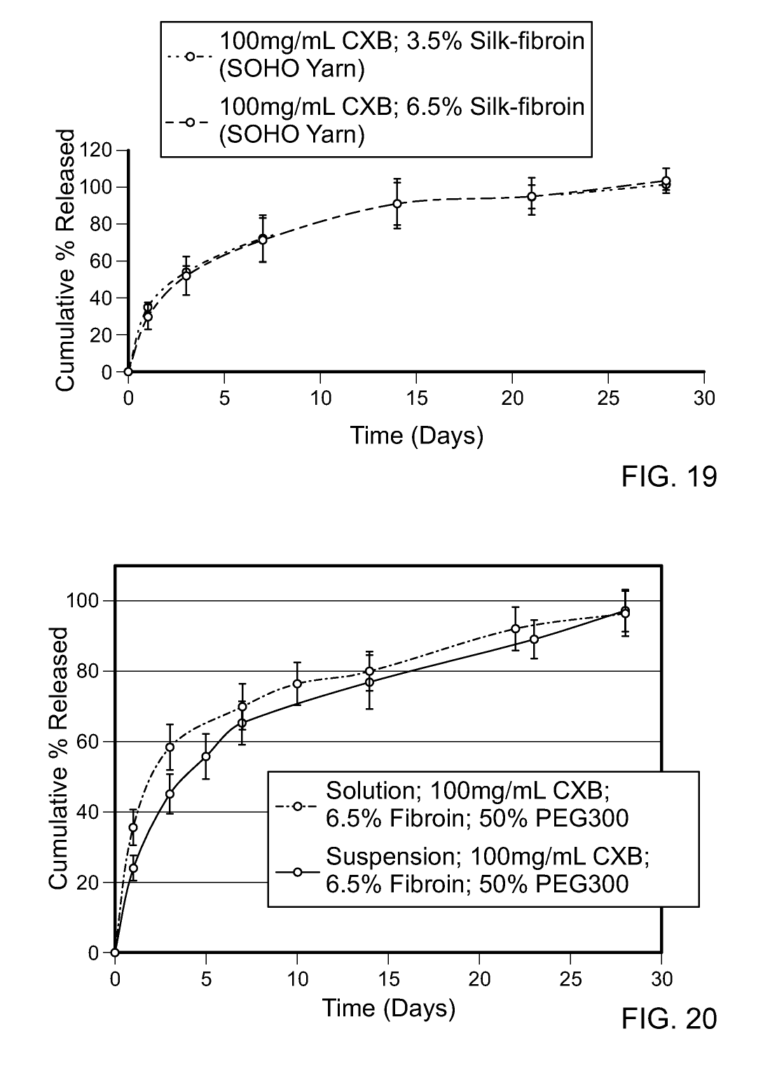

The size exclusion chromatography data for the three silk fibroin sources is shown below in FIG. 17. The silk fibroin source had no effect on the molecular weight of silk-fibroin after either initial degumming or autoclave. Autoclaving the silk caused a decrease in molecular weight of silk-fibroin to approximately one third of the original molecular weight of the degummed solution (from ˜150 kDa to ˜55 kDa). FIG. 18 shows that the source of the silk did not affect the release of celecoxib at 100 mg/mL from 6.5% (w/v) silk-fibroin hydrogels. The trend was the same in the 3.5% (w/v) silk-fibroin hydrogels. FIG. 19 shows that when the concentration of silk fibroin in the hydrogel is between 3.5% (w/v) and 6.5% (w/v), it did not affect the release of celecoxib at 100 mg/mL celecoxib. The trend was the same for the three different silk fibrion sources (Treenway cocoons, Treenway facial cocoons, and SOHO silk yarn).

The source of the silk does not impact celecoxib release kinetics from silk-fibroin hydrogels. Varying the percentage of silk fibroin from 3.5% (w/v) to 6.5% (w/v) in silk hydrogels with 100 mg/mL celecoxib did not impact release kinetics.

### Example 6. Effect of Fibroin Hydrogel Preparation Method on Release Kinetics of Celecoxib

### Methods

### Celecoxib Solution Hydrogel

Treenway silk cocoons were degummed at 100° C. for 60 minutes in 0.02 M sodium carbonate solution. The fibroin was dried overnight, weighed, and dissolved at 20% (w/v) in 9.3 M aqueous lithium bromide solution for five hours at 60° C. This solution was dialyzed against water with six water changes in 3.5 kDa regenerated cellulose dialysis membrane for 48 hours. The dialyzed solution was centrifuged three times at 9,000 RPM for 20 minutes and 4° C. to remove insoluble particles, diluted to a final concentration of 3% (w/v) in 10 mM phosphate buffer, pH 7.4, and autoclaved for 30 minutes. The solution was then filtered through a 0.22-μm filter, frozen, and lyophilized for 72 hours.

Purified fibroin (300 mg) was reconstituted with 2 mL of DI water. 0.5 mL of this solution was then drawn into a 1 cc. syringe. Separately, celecoxib was dissolved in PEG300 at 200 mg/mL. One-half mL of the celecoxib/PEG300 solution was drawn into a separate 1 cc. syringe. These syringes were connected with a luer-lok connector and mixed back and forth approximately 50× or until homogeneous. The resulting mixture was then drawn into one syringe, capped with a 27 gauge needle, and left to gel at room temperature overnight.

### Celecoxib Suspension Hydrogel

SOHO silk yarn was degummed at 100° C. for 120 minutes in 0.02 M sodium carbonate solution. The fibroin was dried overnight, weighed, and dissolved at 10% (w/v) in 9.3 M aqueous lithium bromide solution for five hours at 60° C. This solution was dialyzed against water with six water changes in 50 kDa cellulose ester dialysis membrane for 48 hours. The dialyzed solution was centrifuged at 9,000 RPM for 20 minutes and 4° C. to remove insoluble particles, diluted to a final concentration of 3% (w/v) in 10 mM phosphate buffer, pH 7.4. The solution was then filtered through a 0.22-μm filter, frozen, and lyophilized for 72 hours.

Purified fibroin (300 mg) was reconstituted with 1.55 mL of 30% (w/v) suspension of celecoxib in 0.4% (v/v) Tween 80. One mL of this solution was then drawn into a 6 cc. syringe. One-fourth mL water was then added to the syringe to dilute the fibroin/celecoxib suspension. Separately, 1.25 mL of PEG300 was drawn into a separate 6 cc. syringe. These syringes were connected with a luer-lok connector and mixed back and forth approximately 50× or until homogeneous. The resulting mixture was then drawn into one syringe, capped with a 27 gauge needle, and left to gel at 37° C. overnight.

TABLE 13
Formulation Details

| Group | | [Fibroin] | [PEG300] |
|---|---|---|---|
| # | Description | % (w/v) | % (v/v) |
| 1 | Solution; 100 mg/mL 003 50% PEG300 | 6.5% | 50 |
| 2 | Suspension; 100 mg/mL OCB 50% PEG300 | 6.5% | 50 |

The in vitro release was carried out by weighing 50 mg of formulation into separate tubes containing 45 mL of release medium (1× phosphate buffered saline, 2% (v/v) Tween-80, and 0.05% (w/v) sodium azide). The solubility of celecoxib in this release media is 850 μg/mL. Forty-five mL of this release media can dissolved 38 mg of celecoxib. In this study, where samples contain 5 mg celecoxib, this media will ensure sink conditions (≥5× celecoxib solubility) are maintained throughout the study. The tubes were incubated at 37° C. while shaking. One mL of the release media was collected from each sample at each time point after standing the tubes up and allowing for settling of any large particulates for 15 minutes. One mL of fresh release medium was then added back to each tube. The release media was analyzed by HPLC at 260 nm.

The controls were prepared at Day 0 by placing 50 mg of each formulation in 100% methanol to extract celecoxib. The samples were left shaking at RT for at least 24 hrs. The supernatant was analyzed by HPLC to determine celecoxib loading (mg/g). The data in FIG. 20 show that the release kinetics from silk fibroin hydrogels prepared with a solution of celecoxib had a slightly higher burst compared to silk fibroin hydrogels prepared with a suspension of celecoxib (35% vs. 24% (w/v)). Also, the release kinetics of the hydrogel made using a celecoxib solution are faster over the first 7 days when compared to hydrogel made using a celecoxib suspension. Both formulations display a first-order release profile with complete release at 28 days (FIG. 20). A decrease in the burst of celecoxib can be beneficial in a mammal in order to reduce systemic exposure of the mammal to high concentrations of celecoxib.

### Example 7. Effect of Fibroin Concentration on Release of Celecoxib from Silk-Fibroin Hydrogels Summary

### Methods

Briefly, silk yarn from SOHO was degummed at 100° C. for 90 minutes in 0.02 M sodium carbonate solution to remove sericin. The resulting fibroin was then dried overnight, weighed, and dissolved at 10% (w/v) in 9.3 M lithium bromide solution for five hours at 60° C. The resulting solution was dialyzed against water in 50 kDa regenerated cellulose dialysis membrane for 48 hours at 4° C. with six water changes. The final solution was centrifuged for 20 minutes at 9,000 RPM and 4° C. to remove insoluble particles. The solutions were then diluted to a final concentration of 3% (w/v) in 10 mM phosphate buffer, pH 7.4, and filtered through a 0.22-μm filter, frozen in a dry ice-isopropanol bath, and lyophilized for 72 hours.

TABLE 14
Group list with final hydrogel concentrations

| | | [Fibroin] | [PEG300] | |
|---|---|---|---|---|
| Group# | Description | % (w/v) | % (v/v) | pH |
| 1 | 50% PEG300 | 6% | 50 | Neutral (25 mM PB) |
| 2 | 50% PEG300 | 3% | 50 | Neutral (25 mM PB) |
| 3 | 50% PEG300 | 1.5% | 50 | Neutral (25 mM PB) |
| 4 | 50% PEG300 | 0.75% | 50 | Neutral (25 mM PB) |
| 5 | 50% PEG300 | 0.375% | 50 | Neutral (25 mM PB) |

To form the hydrogels, lyophilized fibroin was reconstituted with a 30% (w/v) celecoxib (CXB) suspension in 0.4% (v/v) polysorbate 80. Table 14 lists the groups of hydrogels formed for this in vitro study. For Group 1, enough 30% (w/v) celecoxib suspension was added to bring the fibroin to 15% (w/v). A portion of this solution was serially diluted with 30% (w/v) celecoxib suspension to prepare the fibroin solutions for Groups 2-5. For each group, the fibroin/celecoxib suspension was diluted 5:1 with water (i.e., 1 mL fibroin/drug solution with 0.2 mL water). The PEG solution for all groups was prepared by diluting PEG300 to 96.15% (v/v) with 0.95 M hydrochloric acid. Fibroin/celecoxib and PEG300/HCl solutions were then combined at a ratio of 1.2:1.3 and mixed until homogeneous. The formulations were incubated at 37° C. on an orbital mixer for 16 hours to induce gelation.

The in vitro release was carried out by weighing 50 mg of formulation into half of a #4 gelatin capsule. The capsules were added to separate tubes containing 45 mL of release medium (1× phosphate buffered saline, 2% (v/v) Tween-80, and 0.05% (w/v) sodium azide). The solubility of celecoxib in this release media is 850 μg/mL. Forty-five mL of this release media can dissolve 38 mg celecoxib. In this study, where the samples contain either 5 mg or 1.5 mg celecoxib, this media will ensure sink conditions (≥5× celecoxib solubility) are maintained throughout the study. The tubes were incubated at 37° C. while shaking. One mL of the release media was collected from each sample at days 1, 4, 7, 10, and 14 after standing the tubes up and allowing for settling of any large particulates for 15 minutes. One mL of fresh release medium was then added back to each tube. The release media was analyzed by HPLC at 260 nm.

The controls were prepared at Day 0 by placing 50 mg of each formulation in 100% methanol to extract celecoxib. The samples were left shaking at RT for at least 24 hrs. The supernatant was analyzed by HPLC to determine celecoxib loading (mg/g).

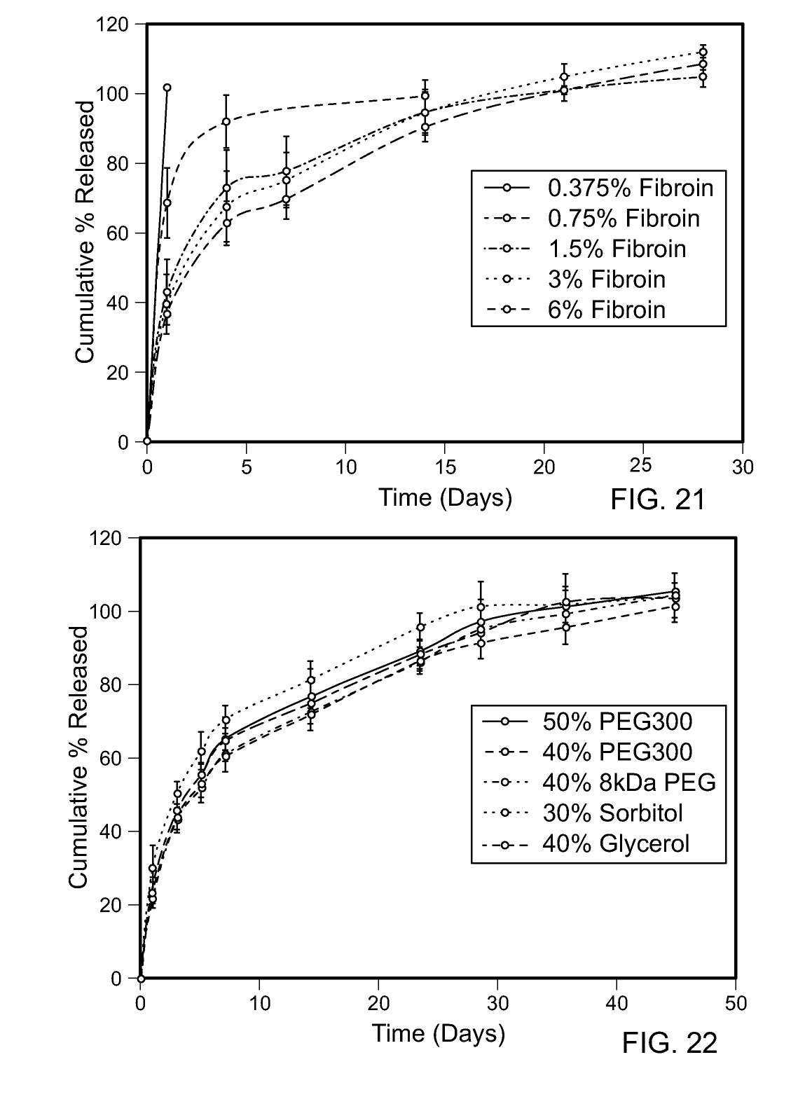

FIG. 21 shows the 0.375% (w/v) hydrogels released 100% of the loaded celecoxib in one day. The 0.75% (w/v) hydrogels had a burst release of 70% of the loaded celecoxib and complete release by day 14. The groups containing 1.5, 3, and 6% (w/v) fibroin had the lowest burst of ˜40% of the loaded celecoxib with first-order release kinetics that showed complete release by day 28.

These data show clear differences between release rates of hydrogels having different silk fibroin concentrations. The general trend is that hydrogels with lower amounts of silk fibroin have greater celecoxib burst and a shorter duration of release. As the fibroin concentration of the hydrogels was increased, the hydrogels became more viscous, the burst was more reduced, and the release of celecoxib more sustained. The celecoxib release rates for hydrogels including between 1.5% (w/v) and 6% (w/v) silk fibroin are similar.

### Example 8. Gelling Agent Screen for Silk-Fibroin Hydrogels

### Materials

Hydrochloric acid, sodium chloride, hydroxyethyl cellulose (HEC), glycerol formal, triethanolamine (TEA), Poloxamer-188, Poloxamer-407, Tween-80, PEG300, PEG400, PEG600, PEG 2 kDa, and PEG 10 kDa were purchased from Sigma Aldrich (St. Louis, Mo.). Glycerol, propylene glycol, and acetonitrile were purchased from Fisher Chemical (Waltham, Mass.). Celecoxib was purchased from Cipla (Mumbai, India). Sorbitol was purchased from Spectrum (New Brunswick, N.J.). PEG 4 kDa, PEG 6 kDa, and PEG 8 kDa were purchased from Clariant (Charlotte, N.C.). Polyvinylpyrrolidone-17 (PVP-17), polyvinylpyrrolidone-12 (PVP-12), carboxymethyl cellulose (CMC), and hydroxypropyl-β-cyclodextrin (HPBCD) were purchased from Ashland (Covington, Ky.). Super-refined PEG300 for P00066 was supplied by Croda (Snaith, UK).

Silk-fibroin was purified in two batches. For batch 004-02, briefly, silk cocoons from Treenway were cut into dime sized pieces and boiled for 60 minutes in 0.02 M sodium carbonate solution to remove sericin. The fibroin was then dried overnight, weighed, and dissolved at 20% (w/v) in 9.3 M lithium bromide solution for five hours at 60° C. The resulting solution was dialyzed against water in 3.5 kDa regenerated cellulose dialysis tubing for 48 hours to remove the excess salt with six water changes. The final solution was then centrifuged three times for 20 minutes each at 9,000 RPM and 4° C. to remove insoluble particles. The solutions were then diluted to a final concentration of 3% (w/v) and 10 mM phosphate, pH 7.4. These solutions were autoclaved for 30 minutes, aliquoted into 50-mL conical tubes, frozen in a dry ice-isopropanol bath, and lyophilized.

Batch 26-01 was prepared in a similar manner. Silk yarn from SOHO was cut and boiled for 120 minutes in 0.02 M sodium carbonate solution to remove sericin. The fibroin was then dried overnight, weighed, and dissolved at 10% (w/v) in 9.3 M lithium bromide solution for five hours at 60° C. The resulting solution was dialyzed against water in 50 kDa cellulose ester dialysis tubing with 6 water exchanges for 48 hours at 4° C. to remove the excess salt. The final solution was then centrifuged once for 20 minutes at 9,000 RPM and 4° C. to remove insoluble particles. The resulting fibroin solution was then diluted to a final concentration of 3% (w/v) and 10 mM phosphate, pH 7.4. This solution was filtered through a 0.22-μm filter unit, aliquoted into 50-mL conical tubes, frozen in a dry ice-isopropanol bath, and lyophilized.

### Methods

### Example 8.1.1 Hydrogels with Low (≤600 Da) Molecular Weight PEG

TABLE 15
Control PEG300 and Fibroin Hydrogels
PEG300 Control Fibroin Hydrogels

| | | | Gelling | Gelling | Fibroin |
|---|---|---|---|---|---|
| | | | Agent 1 | Agent 2 | Conc |
| Gelling | Gelling | Fibroin | Conc | Conc | % |
| Agent 1 | Agent 2 | Batch | % (v/v) | (%) | (w/v) |
| PEG300 | — | 004-02 | 50 | 0 | 6.5 |
| PEG300 | — | 004-02 | 40 | 0 | 6.5 |
| PEG300 | — | 004-02 | 30 | 0 | 6.5 |
| PEG300 | — | 004-02 | 20 | 0 | 6.5 |
| PEG300 | — | 004-02 | 10 | 0 | 6.5 |

TABLE 16
PEG300 and Fibroin Hydrogels with 87 (or 100) mg/mL CXB
PEG300 and Fibroin Hydrogels with 87 mg/mL CXB
(except 50%, which had 100 mg/mL CXB)

| | | | Gelling | Gelling | Fibroin |
|---|---|---|---|---|---|
| | | | Agent 1 | Agent 2 | Conc |
| Gelling | Gelling | Fibroin | Conc | Conc | % |
| Agent 1 | Agent 2 | Batch | % (v/v) | (%) (v/v) | (w/v) |
| PEG300 | N/A | 004-02 | 50 | N/A | 6.5 |
| PEG300 | Polysorbate-80 | 004-02 | 30 | 0.1 | 6.5 |
| PEG300 | Polysorbate-80 | 004-02 | 20 | 0.1 | 6.5 |
| PEG300 | Polysorbate-80 | 004-02 | 10 | 0.1 | 6.5 |

TABLE 17
Control PEG400 and Fibroin Hydrogels

| | | Gelling | Fibroin |
|---|---|---|---|
| Gelling | | Agent 1 | Conc |
| Agent | Fibroin | Conc | % |
| 1 | Batch | % (v/v) | (w/v) |
| PEG400 | 004-02 | 40 | 6.5 |
| PEG400 | 004-02 | 30 | 6.5 |
| PEG400 | 004-02 | 20 | 6.5 |
| PEG400 | 004-02 | 10 | 6.5 |

TABLE 18
PEG400 and Fibroin Hydrogels with 87 mg/mL CXB

| | | | Gelling | Gelling | Fibroin |
|---|---|---|---|---|---|
| | | | Agent 1 | Agent 2 | Conc |
| Gelling | Gelling | Fibroin | Conc | Conc | % |
| Agent 1 | Agent 2 | Batch | % (v/v) | (%) (v/v) | (w/v) |
| PEG400 | Polysorbate-80 | 004-02 | 30 | 0.1 | 6.5 |
| PEG400 | Polysorbate-80 | 004-02 | 20 | 0.1 | 6.5 |
| PEG400 | Polysorbate-80 | 004-02 | 10 | 0.1 | 6.5 |

TABLE 19
Control PEG600 and Fibroin Hydrogels

| | | | Gelling | Gelling | Fibroin |
|---|---|---|---|---|---|
| | | | Agent 1 | Agent 2 | Conc |
| Gelling | Gelling | Fibroin | Conc | Conc | % |
| Agent 1 | Agent 2 | Batch | % (v/v) | (%) (v/v) | (w/v) |
| PEG600 | N/A | 004-02 | 50 | N/A | 6.5 |
| PEG600 | N/A | 004-02 | 40 | N/A | 6.5 |
| PEG600 | N/A | 004-02 | 30 | N/A | 6.5 |
| PEG600 | N/A | 004-02 | 20 | N/A | 6.5 |
| PEG600 | N/A | 004-02 | 10 | N/A | 6.5 |
| PEG600 | Polysorbate-80 | 004-02 | 30 | 0.1 | 6.5 |
| PEG600 | Polysorbate-80 | 004-02 | 20 | 0.1 | 6.5 |
| PEG600 | Polysorbate-80 | 004-02 | 10 | 0.1 | 6.5 |

TABLE 20
Control PEG600 and Fibroin Hydrogels with 87 mg/mL CXB
PEG600 and Fibroin Hydrogels with 87 mg/mL CXB

| | | | Gelling | Gelling | Fibroin |
|---|---|---|---|---|---|
| | | | Agent 1 | Agent 2 | Conc |
| Gelling | Gelling | Fibroin | Conc | Conc | % |
| Agent 1 | Agent 2 | Batch | % (v/v) | (%) (v/v) | (w/v) |
| PEG600 | Polysorbate-80 | 004-02 | 30 | 0.1 | 6.5 |
| PEG600 | Polysorbate-80 | 004-02 | 20 | 0.1 | 6.5 |
| PEG600 | Polysorbate-80 | 004-02 | 10 | 0.1 | 6.5 |

For the hydrogels listed in Tables 15, 17, and 19, PEG300, 400, and 600 solutions were prepared by diluting the gelling agent, if necessary, to twice the final formulation concentration listed with water. The silk-fibroin solution was formed by adding DI water to the lyophilized cake to bring it to a final concentration of 13% (w/v) (2.0 mL for 300 mg fibroin). Equal volumes of both the fibroin and corresponding gelling agent solution were drawn into two syringes, which were then connected by a luer-lok syringe connector and mixed back and forth approximately 25 times, or until homogeneous. The resulting mixture was then drawn into one syringe, capped with a 27 gauge needle, and then either incubated at room temperature or 37° C. overnight. The formulations that did not result in a hydrogel were then extruded into a 2 mL glass HPLC vial and autoclaved at 121° C. for 30 minutes.

The hydrogels containing celecoxib listed in Tables 16, 18, and 20 were prepared by reconstituting purified silk-fibroin (300 mg) with 2.0 mL of 20% (w/v) suspension of celecoxib in 0.2% (v/v) Tween-80. The final celecoxib concentration in this reconstituted silk suspension was 174 mg/mL and the final silk concentration was 13% (w/v). The gelling agent solutions were prepared by diluting the gelling agent with water, if necessary, until it was twice the final formulation concentration listed. Equal volumes of both the fibroin and corresponding gelling agent solution were drawn into two syringes, which were then connected by a luer-lok syringe connector and mixed back approximately 25 times, or until homogeneous. For the first entry in Table 16 (50% (v/v) PEG300 with celecoxib), the PEG300 solution contained the dissolved drug at 200 mg/mL, which was then mixed with pure fibroin solution at 13% (w/v), resulting in final formulation concentrations of 6.5% (w/v) fibroin, 50% (v/v) PEG300, and 100 mg/mL celecoxib. The resulting mixture was then drawn into one syringe, capped with a 27 gauge needle, and then either incubated at room temperature or 37° C. overnight. The formulations that did not result in a hydrogel were then extruded into a 2 mL glass HPLC vial and autoclaved at 121° C. for 30 minutes.

### Example 8.1.2 Hydrogels with High (≥2 kDa) Molecular Weight PEG

TABLE 21
Control PEG 2 kDa and Fibroin Hydrogels

| | | | Gelling | Gelling | Fibroin |
|---|---|---|---|---|---|
| | | | Agent 1 | Agent 2 | Conc |
| Gelling | Gelling | Fibroin | Conc | Conc | % |
| Agent 1 | Agent 2 | Batch | % (v/v) | (%) (v/v) | (w/v) |
| PEG 2 kDa | N/A | 004-02 | 50 | N/A | 6.5 |
| PEG 2 kDa | N/A | 004-02 | 40 | N/A | 6.5 |
| PEG 2 kDa | Polysorbate-80 | 004-02 | 40 | 0.1 | 6.5 |
| PEG 2 kDa | Polysorbate-80 | 004-02 | 30 | 0.1 | 6.5 |
| PEG 2 kDa | Polysorbate-80 | 004-02 | 20 | 0.1 | 6.5 |
| PEG 2 kDa | Polysorbate-80 | 004-02 | 10 | 0.1 | 6.5 |

TABLE 22
PEG 2 kDa and Fibroin Hydrogels with 87 mg/mL CXB

| | | | Gelling | Gelling | Fibroin |
|---|---|---|---|---|---|
| | | | Agent 1 | Agent 2 | Conc |
| Gelling | Gelling | Fibroin | Conc | Conc | % |
| Agent 1 | Agent 2 | Batch | % (v/v) | (%) (v/v) | (w/v) |
| PEG 2 kDa | Polysorbate-80 | 004-02 | 30 | 0.1 | 6.5 |
| PEG 2 kDa | Polysorbate-80 | 004-02 | 20 | 0.1 | 6.5 |
| PEG 2 kDa | Polysorbate-80 | 004-02 | 10 | 0.1 | 6.5 |

TABLE 23
Control PEG 4 kDa and Fibroin Hydrogels

| | | Gelling | Fibroin |
|---|---|---|---|
| | | Agent 1 | Conc |
| Gelling | Fibroin | Conc | % |
| Agent 1 | Batch | % (w/v) | (w/v) |
| PEG 4 kDa | 004-02 | 40 | 6.5 |
| PEG 4 kDa | 004-02 | 30 | 6.5 |
| PEG 4 kDa | 004-02 | 20 | 6.5 |

TABLE 24
Control PEG 6 kDa and Fibroin Hydrogels
Control PEG 6 kDa and Fibroin Hydrogels

| | | Gelling | |
|---|---|---|---|
| | | Agent 1 | Fibroin |
| Gelling | Fibroin | Conc % | Conc % |
| Agent 1 | Batch | (w/v) | (w/v) |
| PEG 6 kDa | 004-02 | 40 | 6.5 |
| PEG 6 kDa | 004-02 | 30 | 6.5 |
| PEG 6 kDa | 004-02 | 20 | 6.5 |

TABLE 25
Control PEG 8 kDa and Fibroin Hydrogels
Control PEG 8 kDa and Fibroin Hydrogels

| | | Gelling | |
|---|---|---|---|
| | | Agent 1 | Fibroin |
| Gelling | Fibroin | Conc % | Conc % |
| Agent 1 | Batch | (w/v) | (w/v) |
| PEG 8 kDa | 004-02 | 40 | 6.5 |
| PEG 8 kDa | 004-02 | 30 | 6.5 |
| PEG 8 kDa | 004-02 | 20 | 6.5 |

TABLE 26
Control PEG 10 kDa and Fibroin Hydrogels
Control PEG 10 kDa and Fibroin Hydrogels

| | | Gelling | |
|---|---|---|---|
| | | Agent 1 | Fibroin |
| Gelling | Fibroin | Conc % | Conc % |
| Agent 1 | Batch | (w/v) | (w/v) |
| PEG 10 kDa | 004-02 | 40 | 6.5 |

TABLE 27
PEG 10 kDa and Fibroin Hydrogels with 87 mg/mL CXB
PEG 10 kDa and Fibroin Hydrogels with 87 mg/mL CXB

| | | | Gelling | Gelling | |
|---|---|---|---|---|---|
| | | | Agent 1 | Agent 2 | Fibroin |
| Gelling | Gelling | Fibroin | Conc % | Conc % | Conc % |
| Agent 1 | Agent 2 | Batch | (w/v) | (v/v) | (w/v) |
| PEG 10 kDa | Polysorbate-80 | 004-02 | 30 | 0.1 | 6.5 |
| PEG 10 kDa | Polysorbate-80 | 004-02 | 20 | 0.1 | 6.5 |
| PEG 10 kDa | Polysorbate-80 | 004-02 | 10 | 0.1 | 6.5 |

The hydrogels listed in Tables 21, 23, 24, and 25, the PEG 2 kDa, 4 kDa, 6 kDa, and 8 kDa solutions were prepared by dissolving the dry PEG in water to twice the final formulation concentration listed. If necessary, the solutions were briefly heated at 60° C. to facilitate dissolution. The lyophilized silk fibroin was reconstituted with DI water to a final concentration of 13% (w/v) (2.0 mL for 300 mg silk fibroin). Equal volumes of both the 13% (w/v) silk fibroin solution and corresponding gelling agent solution were mixed. For Table 26, PEG 10 kDa would not dissolve at any concentration higher than 60% (w/v), so the protocol was slightly adjusted. Dry 10 kDa PEG was dissolved to a final concentration of 60% (w/v) with heating to 60° C. The lyophilized fibroin was reconstituted with DI water to a final concentration of 19.5% (w/v) (1.24 mL for 300 mg fibroin). These solutions were mixed at a ratio of 2:1 60% (w/v) PEG 10 kDa:19.5% (w/v) fibroin solution. The solutions were drawn into two syringes, connected by a luer-lok syringe connector and mixed back and forth approximately 25 times, or until homogeneous. The resulting mixture was then drawn into one syringe, capped with a 27 gauge needle, and then either incubated at room temperature or 37° C. overnight. The formulations that did not result in a hydrogel were then extruded into a 2-mL glass HPLC vial and autoclaved at 121° C. for 30 minutes.

For the hydrogels containing celecoxib listed in Tables 22 and 27, purified fibroin (300 mg) was reconstituted with 2.0 mL of a 20% (w/v) suspension of celecoxib in 0.2% Tween 80, to bring the final celecoxib concentration to 174 mg/mL and the final silk concentration to 13% (w/v). The gelling agent solutions were prepared by dissolving the dry PEG in water until it was twice the final formulation concentration listed. Equal volumes of both the fibroin and corresponding gelling agent solution were drawn into two syringes, which were then connected by a luer-lok syringe connector and mixed back and forth approximately 25 times, or until homogeneous. The resulting mixture was then drawn into one syringe, capped with a 27 gauge needle, and then incubated at 37° C.

### Example 8.1.3 Hydrogels with Glycerol, Glycerol Formal, Triethanolamine, Propylene Glycol, and Sorbitol

TABLE 28
Control Glycerol and Fibroin Hydrogels
Control Glycerol and Fibroin Hydrogels

| | | | Gelling | Gelling | |
|---|---|---|---|---|---|
| | | | Agent 1 | Agent 2 | Fibroin |
| Gelling | Gelling | Fibroin | Conc % | Conc % | Conc % |
| Agent 1 | Agent 2 | Batch | (v/v) | (v/v) | (w/v) |
| Glycerol | N/A | 004-02 | 50 | N/A | 6.5 |
| Glycerol | N/A | 004-02 | 40 | N/A | 6.5 |
| Glycerol | Polysorbate-80 | 004-02 | 40 | 0.1 | 6.5 |
| Glycerol | Polysorbate-80 | 004-02 | 30 | 0.1 | 6.5 |
| Glycerol | Polysorbate-80 | 004-02 | 20 | 0.1 | 6.5 |
| Glycerol | Polysorbate-80 | 004-02 | 10 | 0.1 | 6.5 |

TABLE 29
Glycerol and Fibroin Hydrogels with 87 mg/mL CXB
Glycerol and Fibroin Hydrogels with 87 mg/mL CXB

| | | | Gelling | Gelling | |
|---|---|---|---|---|---|
| | | | Agent 1 | Agent 2 | Fibroin |
| Gelling | Gelling | Fibroin | Conc % | Conc % | Conc % |
| Agent 1 | Agent 2 | Batch | (v/v) | (v/v) | (w/v) |
| Glycerol | Polysorbate-80 | 004-02 | 40 | 0.1 | 6.5 |
| Glycerol | Polysorbate-80 | 004-02 | 30 | 0.1 | 6.5 |
| Glycerol | Polysorbate-80 | 004-02 | 20 | 0.1 | 6.5 |

TABLE 30
Control Glycerol Formal and Fibroin Hydrogels
Control Glycerol Formal and Fibroin Hydrogels

| | | Gelling | |
|---|---|---|---|
| | | Agent 1 | Fibroin |
| Gelling | Fibroin | Conc % | Conc % |
| Agent 1 | Batch | (v/v) | (w/v) |
| Glycerol Formal | 004-02 | 50 | 6.5 |
| Glycerol Formal | 004-02 | 40 | 6.5 |
| Glycerol Formal | 004-02 | 30 | 6.5 |
| Glycerol Formal | 004-02 | 20 | 6.5 |

TABLE 31
Control Triethanolamine and Fibroin Hydrogels
Control Triethanolamine and Fibroin Hydrogels

| | | Gelling | |
|---|---|---|---|
| | | Agent 1 | Fibroin |
| Gelling | Fibroin | Conc % | Conc % |
| Agent 1 | Batch | (v/v) | (w/v) |
| Triethanolamine | 004-02 | 50 | 6.5 |
| Triethanolamine | 004-02 | 40 | 6.5 |
| Triethanolamine | 004-02 | 30 | 6.5 |
| Triethanolamine | 004-02 | 20 | 6.5 |

TABLE 32
Control Propylene Glycol and Fibroin Hydrogels
Control Propylene Glycol and Fibroin Hydrogels

| | | Gelling | |
|---|---|---|---|
| | | Agent 1 | Fibroin |
| Gelling | Fibroin | Conc % | Conc % |
| Agent 1 | Batch | (v/v) | (w/v) |
| Propylene Glycol | 004-02 | 50 | 6.5 |
| Propylene Glycol | 004-02 | 40 | 6.5 |
| Propylene Glycol | 004-02 | 30 | 6.5 |
| Propylene Glycol | 004-02 | 20 | 6.5 |

TABLE 33
Propylene Glycol and Fibroin Hydrogels with 87 mg/mL CXB
Propylene Glycol and Fibroin Hydrogels with 87 mg/mL CXB

| | | | Gelling | Gelling | |
|---|---|---|---|---|---|
| | | | Agent 1 | Agent 2 | Fibroin |
| Gelling | Gelling | Fibroin | Conc % | Conc % | Conc % |
| Agent 1 | Agent 2 | Batch | (v/v) | (v/v) | (w/v) |
| Propylene Glycol | Polysorbate-80 | 004-02 | 40 | 0.1 | 6.5 |
| Propylene Glycol | Polysorbate-80 | 004-02 | 30 | 0.1 | 6.5 |
| Propylene Glycol | Polysorbate-80 | 004-02 | 20 | 0.1 | 6.5 |

TABLE 34
Control Sorbitol and Fibroin Hydrogels
Control Sorbitol and Fibroin Hydrogels

| | | | Gelling | Gelling | |
|---|---|---|---|---|---|
| | | | Agent 1 | Agent 2 | Fibroin |
| Gelling | Gelling | Fibroin | Conc % | Conc % | Conc % |
| Agent 1 | Agent 2 | Batch | (w/v) | (v/v) | (w/v) |
| Sorbitol | Polysorbate-80 | 004-02 | 50 | 0.1 | 6.5 |
| Sorbitol | Polysorbate-80 | 004-02 | 40 | 0.1 | 6.5 |
| Sorbitol | Polysorbate-80 | 004-02 | 35 | 0.1 | 6.5 |
| Sorbitol | Polysorbate-80 | 004-02 | 30 | 0.1 | 6.5 |
| Sorbitol | Polysorbate-80 | 004-02 | 25 | 0.1 | 6.5 |
| Sorbitol | Polysorbate-80 | 004-02 | 20 | 0.1 | 6.5 |
| Sorbitol | Polysorbate-80 | 004-02 | 10 | 0.1 | 6.5 |

For the hydrogels listed in Tables 28, 30, 31, and 32, the gelling agent solutions were prepared by diluting glycerol, glycerol formal, triethanolamine, or propylene glycol with water, if necessary, until they were twice the formulation concentrations listed. The fibroin solutions were prepared reconstituting lyophilized fibroin with DI water to a final concentration of 13% (w/v) (2.0 mL for 300 mg fibroin). Equal volumes of both the 13% (w/v) fibroin solution and corresponding gelling agent solution were mixed. For Table 34, the stock sorbitol was at 70% (w/v), so the first two entries had to have adjusted fibroin concentrations to reach 50 and 40% (w/v) final sorbitol concentration. The fibroin solutions were prepared reconstituting lyophilized fibroin with DI water to a final concentrations of either 22.7% (w/v) (1.02 mL for 300 mg fibroin) or 15.2% (w/v) (1.67 mL to 300 mg). The 50% (w/v) final sorbitol hydrogel was mixed at a ratio of 2.5:1 70% (w/v) sorbitol: 22.7% (w/v) fibroin solution, and the 40% (w/v) final hydrogel was mixed at a ratio of 1.33:1 70% (w/v) sorbitol to 15.2% (w/v) fibroin solution. For all hydrogels, gelling agent and fibroin solutions were drawn into separate syringes, which were then connected by a luer-lok syringe connector and mixed back approximately 25 times, or until homogeneous. The resulting mixture was then drawn into one syringe, capped with a 27 gauge needle, and then either incubated at room temperature or 37° C. overnight. The formulations that did not result in a hydrogel were then extruded into a 2-mL glass HPLC vial and autoclaved at 121° C. for 30 minutes.

For the hydrogels containing celecoxib listed in Tables 29 and 33, purified fibroin (300 mg) was reconstituted with 2.0 mL of 20% (w/v) suspension of celecoxib in 0.2% (v/v) Tween 80, to bring the final celecoxib concentration to 174 mg/mL and the final silk concentration to 13% (w/v). Gelling agent solutions were prepared by diluting stock solution in water until twice the desired final concentration was reached. Equal volumes of both the fibroin and corresponding gelling agent solution were drawn into two syringes, which were then connected by a luer-lok syringe connector and mixed back approximately 25 times, or until homogeneous. The resulting mixture was then drawn into one syringe, capped with a 27 gauge needle, and then incubated at 37° C. Formulations that did not result in a hydrogel were then extruded into a 2-mL glass HPLC vial and autoclaved at 121° C. for 30 minutes.

### Example 8.1.4 Hydrogels with Polysorbate-80 and Benzyl Alcohol

TABLE 35
Control Polysorbate-80 and Fibroin Hydrogels
Control Polysorbate-80 and Fibroin Hydrogels

| | | Gelling | |
|---|---|---|---|
| | | Agent 1 | Fibroin |
| Gelling | Fibroin | Conc % | Conc % |
| Agent 1 | Batch | (v/v) | (w/v) |
| Polysorbate-80 | 004-02 | 0.4 | 6.5 |
| Polysorbate-80 | 004-02 | 0.2 | 6.5 |
| Polysorbate-80 | 004-02 | 0.1 | 6.5 |
| Polysorbate-80 | 004-02 | 0.05 | 6.5 |
| Polysorbate-80 | 004-02 | 0.01 | 6.5 |
| Polysorbate-80 | 004-02 | 0.2 | 3.5 |
| Polysorbate-80 | 004-02 | 0.1 | 3.5 |
| Polysorbate-80 | 004-02 | 0.05 | 3.5 |
| Polysorbate-80 | 004-02 | 0.01 | 3.5 |

TABLE 36
Polysorbate-80 and Fibroin Hydrogels with 87 mg/mL CXB
Polysorbate-80 and Fibroin Hydrogels with 87 mg/mL CXB

| | | Gelling | |
|---|---|---|---|
| | | Agent 1 | Fibroin |
| Gelling | Fibroin | Conc % | Conc % |
| Agent 1 | Batch | (v/v) | (w/v) |
| Polysorbate-80 | 004-02 | 0.2 | 6.5 |

TABLE 37
Control Benzyl Alcohol and Fibroin Hydrogels
Control Benzyl Alcohol and Fibroin Hydrogels

| | | | Gelling | Gelling | |
|---|---|---|---|---|---|
| | | | Agent 1 | Agent 2 | Fibroin |
| Gelling | Gelling | Fibroin | Conc % | Conc % | Conc % |
| Agent 1 | Agent 2 | Batch | (v/v) | (v/v) | (w/v) |
| Benzyl Alcohol | N/A | 004-02 | 1 | N/A | 6.5 |
| Benzyl Alcohol | Polysorbate-80 | 004-02 | 1 | 0.2 | 6.5 |
| Benzyl Alcohol | Polysorbate-80 | 004-02 | 0.9 | 0.4 | 6.5 |
| Benzyl Alcohol | Polysorbate-80 | 004-02 | 0.9 | 0.2 | 6.5 |

For the hydrogels listed in Table 35, the fibroin solution was prepared by dissolving 300 mg of lyophilized fibroin with polysorbate-80 solution in water to the final concentrations listed above. For example, 6.5% (w/v) fibroin and 0.2% (v/v) polysorbate-80 was prepared by adding 4.3 mL of 0.21% (v/v) polysorbate-80 in water to 300 mg of lyophilized fibroin. One mL of these solutions was aliquoted into separate 2-mL glass HPLC vials and autoclaved at 121° C. for 30 minutes.

For the hydrogel sample in Table 36, celecoxib was added to the polysorbate-80 solution used to reconstitute the silk so that the final concentration of the resulting solution was 87 mg/mL celecoxib. One mL of this solution was then left in a 2 mL-HPLC vial at 37° C. overnight.

Several different techniques were used to generate the hydrogels in Table 37. The first entry (1% (v/v) benzyl alcohol and no polysorbate-80), equal volumes of 13% (w/v) fibroin solution and 2% (v/v) benzyl alcohol solution were mixed via syringe connector until homogenous and left at room temperature overnight. The second entry (1% (v/v) benzyl alcohol and 0.2% (v/v) polysorbate-80) was formed by reconstituting the silk fibroin in polysorbate-80 to bring the final concentrations to 13% (w/v) fibroin and 0.4% (v/v) polysorbate-80, before equal volumes of this solution and 2% (v/v) benzyl alcohol were mixed via syringe connector until homogeneous and left at room temperature. The final two hydrogels in Table 23 were formed like this, with equal volumes of 13% (w/v) fibroin with either 0.8% or 0.4% (v/v) polysorbate-80 and 1.8% (v/v) solution of benzyl alcohol mixed via syringe connector until homogeneous. One mL of these solutions was then placed into a 2-mL glass HPLC vial and autoclaved at 121° C. for 30 minutes.

### Example 8.1.5 Hydrogels with Poloxamer-188 and Poloxamer-407

TABLE 38
Control Poloxamer-188 and Fibroin Hydrogels
Control Poloxamer-188 and Fibroin Hydrogels

| | | Exceipint 1 | Fibroin |
|---|---|---|---|
| Gelling | Fibroin | Conc % | Conc % |
| Agent 1 | Batch | (w/v) | (w/v) |
| Poloxamer-188 | 004-02 | 10 | 13 |
| Poloxamer-188 | 004-02 | 10 | 6.5 |
| Poloxamer-188 | 004-02 | 10 | 3.5 |
| Poloxamer-188 | 004-02 | 5 | 6.5 |
| Poloxamer-188 | 004-02 | 2.5 | 6.5 |
| Poloxamer-188 | 004-02 | 1 | 6.5 |
| Poloxamer-188 | 004-02 | 0.5 | 6.5 |
| Poloxamer-188 | 004-02 | 0.25 | 6.5 |

TABLE 39
Poloxamer-188 and Fibroin Hydrogels with 87 mg/mL CXB
Poloxamer-188 and Fibroin Hydrogels with 87 mg/mL CXB

| | | Gelling | |
|---|---|---|---|
| | | Agent 1 | Fibroin |
| | Fibroin | Conc | Conc |
| Gelling Agent 1 | Batch | % (w/v) | % (w/v) |
| Poloxamer-188 | 004-02 | 10 | 6.5 |
| Poloxamer-188 | 004-02 | 5 | 6.5 |

TABLE 40
Control Poloxamer-407 and Fibroin Hydrogels
Control Poloxamer-407 and Fibroin Hydrogels

| | | Gelling | |
|---|---|---|---|
| | | Agent 1 | Fibroin |
| | Fibroin | Conc | Conc |
| Gelling Agent 1 | Batch | % (w/v) | % (w/v) |
| Poloxamer-407 | 004-02 | 10 | 13 |
| Poloxamer-407 | 004-02 | 10 | 6.5 |
| Poloxamer-407 | 004-02 | 10 | 3.5 |
| Poloxamer-407 | 004-02 | 5 | 6.5 |
| Poloxamer-407 | 004-02 | 2.5 | 6.5 |
| Poloxamer-407 | 004-02 | 1 | 6.5 |
| Poloxamer-407 | 004-02 | 0.5 | 6.5 |
| Poloxamer-407 | 004-02 | 0.25 | 6.5 |

TABLE 41
Poloxamer-407 and Fibroin Hydrogels with 87 mg/mL CXB
Poloxamer-407 and Fibroin Hydrogels with 87 mg/mL CXB

| | | Gelling | |
|---|---|---|---|
| | | Agent 1 | Fibroin |
| | Fibroin | Conc | Conc |
| Gelling Agent 1 | Batch | % (w/v) | % (w/v) |
| Poloxamer-407 | 004-02 | 10 | 6.5 |
| Poloxamer-407 | 004-02 | 5 | 6.5 |

For the hydrogels listed in Tables 38 and 40, the two dry poloxamer powders were dissolved in water to twice the final formulation concentration listed. If necessary, the solutions were briefly cooled on ice and vortexed to facilitate dissolution. The fibroin solution was formed by adding DI water to the lyophilized cake to bring silk fibroin to a final concentration of 26%, 13%, or 7% (w/v) (1.0, 2.0, or 4.0 mL water, respectively). Equal volumes of both the silk fibroin solution and corresponding gelling agent solution were mixed. The solutions were drawn into two syringes, which were then connected by a luer-lok syringe connector and mixed back and forth approximately 25 times, or until homogeneous. The resulting mixture was then drawn into one syringe, capped with a 27 gauge needle, and then either incubated at room temperature or 37° C. overnight. The formulations that did not result in a hydrogel were then extruded into a 2-mL glass HPLC vial and autoclaved at 121° C. for 30 minutes.

For the hydrogels containing celecoxib listed in Tables 25 and 27, purified fibroin (300 mg) was reconstituted with 2.0 mL of a 20% (w/v) suspension of celecoxib in 0.2% (v/v) Tween 80, to bring the final celecoxib concentration to 174 mg/mL and the final silk concentration to 13% (w/v). The gelling agent solutions were prepared by dissolving the dry poloxamer in water to twice the final formulation concentration listed. Equal volumes of both the fibroin and corresponding gelling agent solution were drawn into two syringes, which were then connected by a luer-lok syringe connector and mixed back and forth approximately 25 times, or until homogeneous. The resulting mixture was then drawn into one syringe, capped with a 27 gauge needle, and then incubated at 37° C.

### Example 8.1.6 Hydrogels with Hydroxyethyl Cellulose (HEC), Carboxymethyl Cellulose (CMC), Hydroxypropyl-β-cyclodextrin (HPBCD), Polyvinylpyrrolidone-12 (PVP-12), and -17 (PVP-17)

TABLE 42
Control Hydroxyethyl Cellulose (HEC) and Fibroin Hydrogels
Control HEC and Fibroin Hydrogels

| | | Gelling | |
|---|---|---|---|
| | | Agent 1 | Fibroin |
| Gelling | Fibroin | Conc | Conc |
| Agent 1 | Batch | % (w/v) | % (w/v) |
| HEC | 004-02 | 5 | 6.5 |
| HEC | 004-02 | 2.5 | 6.5 |
| HEC | 004-02 | 1 | 6.5 |

TABLE 43
Control Carboxymethyl Cellulose (CMC) and Fibroin Hydrogels
Control CMC and Fibroin Hydrogels

| | | Gelling | |
|---|---|---|---|
| | | Agent 1 | Fibroin |
| Gelling | Fibroin | Conc | Conc |
| Agent 1 | Batch | % (w/v) | % (w/v) |
| CMC | 004-02 | 5 | 6.5 |
| CMC | 004-02 | 2.5 | 6.5 |
| CMC | 004-02 | 1 | 6.5 |

TABLE 44
Control Hydroxypropyl-β-cyclodextrin (HPBCD) and Fibroin Hydrogels
Control HPBCD and Fibroin Hydrogels

| | | Gelling | |
|---|---|---|---|
| | | Agent 1 | Fibroin |
| Gelling | Fibroin | Conc | Conc |
| Agent 1 | Batch | % (w/v) | % (w/v) |
| HPBCD | 004-02 | 30 | 6.5 |
| HPBCD | 004-02 | 20 | 6.5 |
| HPBCD | 004-02 | 10 | 6.5 |

TABLE 45
Control Polyvinylpyrrolidone-12 (PVP-12) and Fibroin Hydrogels
Control PVP-12 and Fibroin Hydrogels

| | | Gelling | |
|---|---|---|---|
| | | Agent 1 | Fibroin |
| Gelling | Fibroin | Conc | Conc |
| Agent 1 | Batch | % (w/v) | % (w/v) |
| PVP-12 | 004-02 | 25 | 6.5 |
| PVP-12 | 004-02 | 10 | 6.5 |
| PVP-12 | 004-02 | 5 | 6.5 |

TABLE 46
Control Polyvinylpyrrolidone-17 (PVP-17) and Fibroin Hydrogels
Control PVP-17 and Fibroin Hydrogels

| | | Gelling | |
|---|---|---|---|
| Gelling | Fibroin | Agent 1 | Fibroin |
| Agent 1 | Batch | Conc (%) | Conc (%) |
| PVP-17 | 004-02 | 25 | 6.5 |
| PVP-17 | 004-02 | 10 | 6.5 |
| PVP-17 | 004-02 | 5 | 6.5 |

For the hydrogels listed in Tables 42 through 46, dry gelling agents were solubilized to 2× the final desired formulation concentration in water. Fibroin was reconstituted to a final concentration of 13% (w/v) (2.0 mL water to 300 mg fibroin). Equal volumes of both the fibroin solution and corresponding gelling agent solution were mixed. The solutions they were drawn into two syringes, which were then connected by a luer-lok syringe connector and mixed back and forth approximately 25 times, or until homogeneous. The resulting mixture was then drawn into one syringe, capped with a 27 gauge needle, and then incubated at 37° C. overnight.

### Example 8.2 In Vitro Release

TABLE 47
In vitro Release Sample List

| | | [Gelling | |
|---|---|---|---|
| | Gelling Agent | Agent] | |
| Description | (Manufacturer) | (%) | pH |
| 50% PEG300 | PEG 300 (Croda) | 50% (v/v) | Neutral (25 mM PB) |
| 40% PEG300 | PEG 300 (Croda) | 40% (v/v) | Neutral (25 mM PB) |
| 40% PEG 8 kDa | PEG 8 kDa | 40% (w/v) | Neutral (25 mM PB) |
| | (Clariant) | | |
| 30% Sorbitol | Sorbitol (Spectrum) | 30% (w/v) | Neutral (25 mM PB) |
| 40% Glycerol | Glycerol (Fisher) | 40% (v/v) | Neutral (25 mM PB) |

To perform the in vitro release comparing hydrogels with different gelling agents, the samples were prepared as follows. A 30% (w/v) celecoxib suspension in 0.4% (v/v) polysorbate-80 was formed. This suspension was used to reconstitute fibroin to 16.2% and 251 mg/mL celecoxib. This fibroin/celecoxib solution was used to prepare all formulations below. The formulations were prepared as follows:
50% (v/v) PEG300: The gelling agent solution (96.2% (v/v) PEG300, 3.65% (v/v) hydrochloric acid, and 0.15% (v/v) water) was prepared. Water was mixed with the fibroin/celecoxib solution in a ratio of 1:5 (i.e., 0.2 mL water to 1 mL fibroin/celecoxib). These gelling agent and fibroin/celecoxib solutions were mixed via syringe connector in a ratio of 1.3:1.2 until homogeneous. Syringes were filled with the mixture, capped with a 27 gauge needle, and left at 37° C. overnight.
40% (v/v) PEG300: The gelling agent solution (66.6% (v/v) PEG300, 2.5% (v/v) hydrochloric acid, and 30.9% (v/v) water) was prepared. This solution and the fibroin/celecoxib solution were then mixed via syringe connector in a ratio of 1.5:1 until homogenous. Syringes were filled with the mixture, capped with a 27 gauge needle, and left at 37° C. overnight.
40% (w/v) PEG 8 kDa: An HCl/salt solution was made by dissolving sodium chloride at 27 mg/mL in 0.068 N hydrochloric acid. A 66.6% (w/v) PEG 8 kDa solution was formed by dissolving dry PEG powder with the HCl/salt solution. This solution and the fibroin/celecoxib solution were then mixed via syringe connector in a ratio of 1.5:1 until homogenous. Syringes were filled with the mixture, capped with a 27 gauge needle, and left at 37° C. overnight.
30% (w/v) Sorbitol: A solution of 50% (w/v) sorbitol was made by diluting 70% (w/w) sorbitol USP with DI water. This solution and the fibroin/celecoxib solution were then mixed via syringe connector in a ratio of 1.5:1 until homogenous. Syringes were filled with the mixture, capped with a 27 gauge needle, and left at 37° C. overnight.
40% (v/v) Glycerol: Gylcerol was diluted to 66.6% (v/v) glycerol with DI water. This solution and the fibroin/celecoxib solution were then mixed via syringe connector in a ratio of 1.5:1 until homogenous. Syringes were filled with the mixture, capped with a 27 gauge needle, and left at 37° C. overnight.

The in vitro release was carried out by weighing 50 mg of formulation into half of a #4 gelatin capsule. The capsules were added to separate tubes containing 45 mL of release medium (1× phosphate buffered saline, 2% (v/v) Tween-80, and 0.05% (w/v) sodium azide). The solubility of celecoxib in this release media is 850 μg/mL. Forty-five mL of this release media dissolves 38 mg celecoxib. In this study then, where samples contain 5 mg celecoxib, this media will ensure sink conditions (≥5× celecoxib solubility) are maintained throughout the study. The tubes were incubated at 37° C. while shaking. One mL of the release media was collected from each sample at days 1, 4, 7, 14, 21, 28, 35, and 42 after standing the tubes up and allowing for settling of any large particulates for 15 minutes. One mL of fresh release medium was then added back to each tube. The Release media was analyzed by HPLC at 260 nm.

Peaks on the HPLC UV-Vis were integrated against a standard curve of known celecoxib amounts. This was used to generate concentrations of celecoxib in the release medium and total percent celecoxib release was then able to be calculated. The samples were formulated in triplicate which allowed for statistical analysis including standard deviation of release profiles.

### Results

Hydrogel formation of a variety of formulations was studied at different curing conditions. Twenty-two different gelling agents were investigated. The final silk fibroin percentage of each formulation ranged from 3.5% (w/v) to 13% (w/v). The concentration of gelling agents ranged from 0.01 to 50%. The gelling agents studied were: PEG300, PEG400, PEG600, PEG 2 kDa, PEG 4 kDa, PEG 6 kDa, PEG 8 kDa, PEG 10 kDa, glycerol, glycerol formal, triethanolamine (TEA), propylene glycol, sorbitol, polysorbate-80 (Tween-80), benzyl alcohol (BA), poloxamer-188, poloxamer-407, hydroxyethyl cellulose (HEC), carboxymethyl cellulose (CMC), hydroxypropyl-β-cyclodextrin (HPBCD), polyvinylpyrrolidone-12 (PVP-12), and polyvinylpyrrolidone-17 (PVP-17).

Tables 48-80 detail the fibroin, gelling agent, and celecoxib concentrations in each hydrogel, as well as the curing temperatures. Twenty-five degree and 37° C. samples were removed after 24 hours and checked for hydrogel formation. Autoclaved samples (30 minute cycle at 121° C.) were processed in glass vials. The sample was then removed, and checked for hydrogel formation.

In general, higher concentrations of either silk fibroin or gelling agent and higher curing temperatures were more likely to lead to full gelation. It also appears that the addition of celecoxib increased the propensity of formulations to gel. This effect can be seen in Tables 15 and 16, where the 20% and 10% PEG300 gels with drug gelled after autoclaving, whereas they did not without drug.

Example 8.3 Hydrogels with Low (≤600 Da) Molecular Weight PEG Structure of polyethylene glycol (PEG) of any molecular weight:

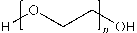

TABLE 48
Gelation of hydrogels with 6.5% (w/v) fibroin and varying
concentrations of PEG300. Gel formulation at 25 and 37° C.,
as well as after exposure to a 30 minute autoclave cycle (121° C.).
PEG300 6.5% (w/v) Fibroin

| Final % (v/v) Gelling | | | |
|---|---|---|---|
| Agent | 25° C. | 37° C. | Autoclave |
| 50 | Full | Full | N/A |
| 40 | Full | Full | N/A |
| 30 | None | None | Full |
| 20 | None | None | None |
| 10 | None | None | None |

“Full” = fully formed, injectable hydrogel;
“None” = no gelation, still liquid;
“N/A” = not tested

TABLE 49
Gelation of hydrogels with 6.5% (w/v) fibroin and varying
concentrations of PEG300, and 87 mg/mL CXB in 0.1% Tween-80.
Gel formulation at 25 and 37° C., as well as after exposure
to a 30 minute autoclave cycle (121° C.).
PEG300, 6.5% (w/v) Fibroin, 87 mg/mL CXB

| Final % (v/v) Gelling | | | |
|---|---|---|---|
| Agent | 25° C. | 37° C. | Autoclave |
| 50 | Full | N/A | N/A |
| 30 | N/A | Full | N/A |
| 20 | N/A | None | Full |
| 10 | N/A | None | Full |

“Full” = fully formed, injectable hydrogel;
“None” = no gelation, still liquid;
“N/A” = not tested

TABLE 50
Gelation of hydrogels with 6.5% fibroin and varying concentrations of
PEG400. Gel formulation at 25 and 37° C., as well as
after exposure to a 30 minute autoclave cycle (121° C.).
PEG400 6.5% (w/v) Fibroin

| Final % (v/v) Gelling | | | |
|---|---|---|---|
| Agent | 25° C. | 37° C. | Autoclave |
| 40 | Full | Full | N/A |
| 30 | None | None | Full |
| 20 | None | None | None |
| 10 | None | None | None |

“Full” = fully formed, injectable hydrogel;
“None” = no gelation, still liquid;
“N/A” = not tested

TABLE 51
Gelation of hydrogels with 6.5% (w/v) fibroin and varying
concentrations of PEG400, and 87 mg/mL CXB in 0.1% Tween-80.
Gel formulation at 25 and 37° C., as well as after exposure
to a 30 minute autoclave cycle (121° C.).
PEG400, 6.5% (w/v) Fibroin, 87 mg/mL

| Final % (v/v) Gelling | | |
|---|---|---|
| Agent | 37° C. | Autoclave |
| 40 | Full | N/A |
| 30 | Full | N/A |
| 20 | None | Full |
| 10 | None | Full |

“Full” = fully formed, injectable hydrogel;
“None” = no gelation, still liquid;
“N/A” = not tested

TABLE 52
Gelation of hydrogels with 6.5% fibroin and varying concentrations of
PEG600. Gel formulation at 25 and 37° C., as well as
after exposure to a 30 minute autoclave cycle (121° C.).
PEG600, 6.5% (w/v) Fibroin

| Final % (v/v) Gelling | | | |
|---|---|---|---|
| Agent | 25° C. | 37° C. | Autoclave |
| 50 | Full | Full | N/A |
| 40 | Full | Full | N/A |
| 30 | None | Full + T | N/A |
| 20 | None | None + T | Full + T |
| 10 | None | None + T | None + T |

“Full” = fully formed, injectable hydrogel;
“None” = no gelation, still liquid;
“N/A” = not tested

TABLE 53
Gelation of hydrogels with 6.5% (w/v) fibroin and varying
concentrations of PEG600, and 87 mg/mL CXB in 0.1% Tween-80.
Gel formulation at 25 and 37° C., as well as
after exposure to a 30 minute autoclave cycle (121° C.).
PEG600, 6.5% (w/v) Fibroin, 87 mg/mL

| Final % (v/v) Gelling | |
|---|---|
| Agent | 37° C. |
| 30 | Full |
| 20 | None |
| 10 | None |

“Full” = fully formed, injectable hydrogel;
“None” = no gelation, still liquid;
“N/A” = not tested

Tables 48-53 detail the gelation results using a lower molecular weight PEG as a gelling agent. These include PEG300, 400 and 600. All three of these PEGs resulted in hydrogels when formulated with PEG concentrations greater than or equal to 40% (v/v). The addition of celecoxib reduced the PEG concentration required to induce gelation to as low as 30% (v/v) at either 37° C. or RT. Autoclaving celecoxib loaded formulations reduced this even further. PEG300 and PEG400 hydrogels formed at both 20% (v/v) and 10% (v/v) PEG with celecoxib and autoclaving.

### Example 8.4 Hydrogels with High (≥2 kDa) Molecular Weight PEG

TABLE 54
Gelation of hydrogels with 6.5% (w/v) fibroin and varying
concentrations of PEG 2 kDa. Gel formulation at 25 and 37° C., as well as
after exposure to a 30 minute autoclave cycle (121° C.).
PEG 2 kDa, 6.5% (w/v) Fibroin

| Final % (w/v) Gelling | | | |
|---|---|---|---|
| Agent | 25° C. | 37° C. | Autoclave |
| 50 | Full | N/A | N/A |
| 40 | None | Full + T | N/A |
| 30 | N/A | Full + T | N/A |
| 20 | N/A | Partial + T | Full + T |
| 10 | N/A | None + T | Full + T |

“Full” = fully formed, injectable hydrogel;
“Partial” = gelation not uniform, still mostly liquid;
“None” = no gelation, still liquid;
“+T” = with the addition of 0.1% Tween-80;
“N/A” = not tested

TABLE 55
Gelation of hydrogels with 6.5% (w/v) fibroin and varying
concentrations of PEG 2 kDa, and 87 mg/mL CXB in 0.1% Tween-80.
Gel formulation at 25 and 37° C., as well as after exposure
to a 30 minute autoclave cycle (121° C.).
PEG 2 kDa, 6.5% (w/v) Fibroin, 87 mg/mL

| Final % (w/v) Gelling Agent | 37° C. |
|---|---|
| 30 | Full |
| 20 | None |
| 10 | None |

“Full” = fully formed, injectable hydrogel;
“None” = no gelation, still liquid;
“N/A” = not tested

TABLE 56
Gelation of hydrogels with 6.5% (w/v) fibroin and varying
concentrations of PEG 4 kDa. Gel formulation at 25 and 37° C.,
as well as after exposure to a 30 minute autoclave cycle (121° C.).
PEG 4 kDa, 6.5% (w/v) Fibroin

| Final % (w/v) Gelling | |
|---|---|
| Agent | 37° C. |
| 40 | Full |
| 30 | None |
| 20 | None |

“Full” = fully formed, injectable hydrogel;
“None” = no gelation, still liquid;
“N/A” = not tested

TABLE 57
Gelation of hydrogels with 6.5% (w/v) fibroin and varying
concentrations of PEG 6 kDa. Gel formulation at 25 and 37° C.,
as well as after exposure to a 30 minute autoclave cycle (121° C.).
PEG 6 kDa, 6.5% (w/v) Fibroin

| Final % (w/v) Gelling | |
|---|---|
| Agent | 37° C. |
| 40 | Full |
| 30 | None |
| 20 | None |

“Full” = fully formed, injectable hydrogel;
“None” = no gelation, still liquid;
“N/A” = not tested

TABLE 58
Gelation of hydrogels with 6.5% (w/v) fibroin and varying
concentrations of PEG 8 kDa. Gel formulation at 25 and 37° C.,
as well as after exposure to a 30 minute autoclave cycle (121° C.).
PEG 8 kDa, 6.5% (w/v) Fibroin

| Final % (w/v) Gelling | |
|---|---|
| Agent | 37° C. |
| 40 | Full |
| 30 | None |
| 20 | None |

“Full” = fully formed, injectable hydrogel;
“None” = no gelation, still liquid;
“N/A” = not tested

TABLE 59
Gelation of hydrogels with 6.5% (w/v) fibroin and varying
concentrations of PEG 10 kDa. Gel formulation at 25 and 37° C.,
as well as after exposure to a 30 minute autoclave cycle (121° C.).
PEG 10 kDa, 6.5% (w/v) Fibroin

| Final % (w/v) Gelling | | | |
|---|---|---|---|
| Agent | 25° C. | 37° C. | Autoclave |
| 50 | Full | | |
| 40 | None | N/A | N/A |
| 30 | N/A | Full + T | N/A |
| 20 | N/A | Partial + T | Full + T |
| 10 | N/A | None + T | Full + T |

“Full” = fully formed, injectable hydrogel;
“Partial” = gelation not uniform, still mostly liquid;
“None” = no gelation, still liquid;
“+T” = with the addition of 0.1% Tween-80;
“N/A” = not tested

TABLE 60
Gelation of hydrogels with 6.5% (w/v) fibroin and varying
concentrations of PEG 10 kDa, and 87 mg/mL CXB in 0.1%
Tween-80. Gel formulation at 25 and 37° C., as well as after
exposure to a 30 minute autoclave cycle (121° C.).
PEG 10 kDa, 6.5% (w/v) Fibroin, 87 mg/mL CXB

| Final % (w/v) Gelling | |
|---|---|
| Agent | 37° C. |
| 30 | Full |
| 20 | Partial |
| 10 | None |

“Full” = fully formed, injectable hydrogel;
“Partial” = gelation not uniform, still mostly liquid;
“None” = no gelation, still liquid;
“N/A” = not tested

Tables 54-60 detail the gelation results using a higher molecular weight PEG as a gelling agent. These include PEG 2 kDa, 4 kDa, 6 kDa, 8 kDa, and 10 kDa. In the absence of celecoxib, 2 kDa PEG formed a hydrogel at a concentration of 50% (w/v) at RT. The addition of 0.2% (v/v) Tween 80 as well as increasing the gelation temperature to 37° C., reduced the required PEG 2 kDa concentration to 30% (w/v). Autoclaving and 0.2% (v/v) Tween 80 allowed for hydrogel formation at 2 kDa PEG concentrations as low as 10% (w/v). Celecoxib (87 mg/mL) hydrogels were formed with 2 kDa PEG when the 2 kDa PEG concentration was 30% (w/v) and curing temperature was 37° C. Four kDa, 6 kDa, and 8 kDa PEG all performed similarly in that they formed hydrogels when used at a concentration of 40% (w/v) with a curing temperature of 37° C. In the absence of celecoxib, 10 kDa PEG formed a hydrogel when used at a concentration of 50% (w/v) at RT. The further addition of 0.2% Tween 80 increased the gelation temperature to 37° C., reduced the required 10 kDa PEG concentration to 30% (w/v). Autoclaving and the addition of 0.2% Tween 80 allowed for hydrogel formation using 10 kDa PEG concentrations as low as 10% (w/v). Celecoxib (87 mg/mL) hydrogels were formed with 10 kDa PEG when the 10 kDa PEG concentration was 30% (w/v) and the curing temperature was 37° C.

### Example 8.5 Hydrogels with Glycerol, Glycerol Formal, Triethanolamine, Propylene Glycol, and Sorbitol

TABLE 61
Gelation of hydrogels with 6.5% (w/v) fibroin and varying
concentrations of glycerol. Gel formulation at 25 and 37° C.,
as well as after exposure to a 30 minute autoclave cycle (121° C.).
Glycerol, 6.5% (w/v) Fibroin

| Final % (v/v) Gelling | | | |
|---|---|---|---|
| Agent | 25° C. | 37° C. | Autoclave |
| 50 | Full | N/A | N/A |
| 40 | None | Full + T | N/A |
| 30 | N/A | None + T | Full + T |
| 20 | N/A | None + T | Partial + T |
| 10 | N/A | None + T | None + T |

“Full” = fully formed, injectable hydrogel;
“Partial” = gelation not uniform, still mostly liquid;
“None” = no gelation, still liquid;
“N/A” = not tested

TABLE 62
Gelation of hydrogels with 6.5% (w/v) fibroin and varying
concentrations of glycerol, and 87 mg/mL CXB in 0.1%
Tween-80. Gel formulation at 25 and 37° C., as well as
after exposure to a 30 minute autoclave cycle (121° C.).
Glycerol, 6.5% (w/v) Fibroin, 87 mg/mL CXB

| Final % (v/v) Gelling Agent | 37° C. |
|---|---|
| 40 | Full |
| 30 | None |
| 20 | None |

“Full” = fully formed, injectable hydrogel;
“None” = no gelation, still liquid;
“N/A” = not tested

TABLE 63
Gelation of hydrogels with 6.5% (w/v) fibroin and varying
concentrations of glycerol formal. Gel formulation at 25 and 37° C.,
as well as after exposure to a 30 minute autoclave cycle (121° C.).
Glycerol Formal, 6.5% (w/v) Fibroin

| Final % (v/v) Gelling | | |
|---|---|---|
| Agent | 25° C. | Autoclave |
| 50 | Full | N/A |
| 40 | Full | N/A |
| 30 | None | Full |
| 20 | None | Full |

“Full” = fully formed, injectable hydrogel;
“None” = no gelation, still liquid;
“N/A” = not tested

TABLE 64
Gelation of hydrogels with 6.5% (w/v) fibroin and varying
concentrations of triethanolamine. Gel formulation at 25 and 37° C.,
as well as after exposure to a 30 minute autoclave cycle (121° C.).
Triethanolamine, 6.5% (w/v) Fibroin

| Final % (v/v) Gelling | | |
|---|---|---|
| Agent | 25° C. | Autoclave |
| 50 | Full | N/A |
| 40 | Full | N/A |
| 30 | None | Full |
| 20 | None | None |

“Full” = fully formed, injectable hydrogel;
“None” = no gelation, still liquid;
“N/A” = not tested

TABLE 65
Gelation of hydrogels with 6.5% (w/v) fibroin and varying
concentrations of propylene glycol. Gel formulation at 25 and 37° C., as
well as after exposure to a 30 minute autoclave cycle (121° C.).
Propylene Glycol, 6.5% (w/v) Fibroin

| Final % (v/v) Gelling | | | |
|---|---|---|---|
| Agent | 25° C. | 37° C. | Autoclave |
| 50 | Full | N/A | N/A |
| 40 | Full | N/A | N/A |
| 30 | None | N/A | Full |
| 20 | None | N/A | Full |
| 10 | N/A | None + T | Partial + T |

“Full” = fully formed, injectable hydrogel;
“None” = no gelation, still liquid;
“N/A” = not tested

TABLE 66
Gelation of hydrogels with 6.5% (w/v) fibroin and varying
concentrations of propylene glycol, and 87 mg/mL
CXB in 0.1% Tween-80. Gel formulation at 25 and 37° C.,
as well as after exposure to a 30 minute autoclave
cycle (121° C.).
Propylene Glycol, 6.5% (w/v) Fibroin, 87 mg/mL CXB

| Final % (v/v) Gelling | | |
|---|---|---|
| Agent | 37° C. | Autoclave |
| 30 | Full | N/A |
| 20 | None | Full |
| 10 | None | Full |

“Full” = fully formed, injectable hydrogel;
“None” = no gelation, still liquid;
“N/A” = not tested

TABLE 67
Gelation of hydrogels with 6.5% (w/v) fibroin and varying
concentrations of sorbitol. Gel formulation at 25 and 37° C.,
as well as after exposure to a 30 minute autoclave cycle (121° C.).
Sorbitol, 6.5% (w/v) Fibroin

| Final % (w/v) Gelling | | | |
|---|---|---|---|
| Agent | 25° C. | 37° C. | Autoclave |
| 50 | Partial + T | Partial + T | N/A |
| 40 | Partial + T | Partial + T | N/A |
| 35 | N/A | Partial + T | N/A |
| 30 | Full + T | Full + T | N/A |
| 25 | N/A | None + T | N/A |
| 20 | None + T | None + T | Partial + T |
| 10 | None + T | None + T | None + T |

“Full” = fully formed, injectable hydrogel;
“Partial” = gelation not uniform, still mostly liquid; 0.1%
“None” = no gelation, still liquid;
“+T” = with the addition of Tween-80;
“N/A” = not tested

Tables 61-67 detail the gelation results using a variety of alternative, non-PEG based gelling agents, including glycerol, glycerol formal, triethanolamine (TEA), propylene glycol, and sorbitol.

In the absence of celecoxib, glycerol induced fibroin gelation at decreasing concentrations as curing temperature was increased. Minimum concentrations of 50%, 40% and 30% (v/v) glycerol formed hydrogels when cured at RT, 37° C., and after autoclaving, respectively. Similar to the absence of celecoxib, formulations containing 87 mg/mL celecoxib and a curing temperature of 37° C. formed hydrogels with at a minimum glycerol concentration of 40% (v/v). Glycerol formal elicited gelation at a minimum concentration of 40% (v/v) in the absence of celecoxib and RT curing. Autoclaving reduced the required gelation glycerol formal concentration to 20% (v/v).

Triethanolamine elicited gelation at a minimum concentration of 40% (v/v) in the absence of celecoxib and RT curing. Autoclaving reduced the required gelation triethanolamine concentration to 30% (v/v). Propylene glycol, similar to glycerol formal, elicited gelation at a minimum concentration of 40% (v/v) in the absence of celecoxib and RT curing. Autoclaving reduced the required gelation propylene glycol concentration to 20% (v/v). The addition of 87 mg/mL celecoxib, reduced the RT minimum gelation propylene glycol concentration to 30% (v/v) and autoclave minimum gelation propylene glycol concentration to 10% (v/v). Sorbitol, in the absence of celecoxib, only formed hydrogels at a concentration of 30% (w/v) at both RT and 37° C.

### Example 8.6 Hydrogels with Polysorbate-80 and Benzyl Alcohol

TABLE 68
Gelation of hydrogels with 6.5% (w/v) fibroin and varying
concentrations of polysorbate-80. Gel formulation at 25 and 37° C.,
as well as after exposure to a 30 minute autoclave cycle (121° C.).
Polysorbate-80, 6.5% (w/v) Fibroin

| Final % (v/v) | |
|---|---|
| Gelling Agent | Autoclave |
| 0.4 | Full |
| 0.2 | Full |
| 0.1 | Partial |
| 0.05 | Partial |
| 0.01 | Full |

“Full” = fully formed, injectable hydrogel;
“None” = no gelation, still liquid;
“N/A” = not tested

TABLE 69
Gelation of hydrogels with 6.5% (w/v) fibroin and varying
concentrations of polysorbate-80, and 87 mg/mL CXB. Gel formulation at 25
and 37° C., as well as after exposure to a 30 minute autoclave cycle (121° C.).
Polysorbate-80, 6.5% (w/v) Fibroin, 87 mg/mL

| Final % (v/v) Gelling | |
|---|---|
| Agent | 37° C. |
| 0.2 | Partial |

“Partial” = gelation not uniform, still mostly liquid

TABLE 70
Gelation of hydrogels with 3.5% (w/v) fibroin and varying
concentrations of polysorbate-80. Gel formulation after
exposure to a 30 minute autoclave cycle (121° C.).
Polysorbate-80, 3.5% (w/v)
Fibroin

| Final % (v/v) | |
|---|---|
| Gelling Agent | Autoclave |
| 0.2 | None |
| 0.1 | None |
| 0.05 | None |
| 0.01 | None |

“None” = no gelation, still liquid

TABLE 71
Gelation of hydrogels with 6.5% (w/v) fibroin and varying
concentrations of benzyl alcohol (BA) and polysorbate-80 (T-80). Gel
formulation at 25 as well as after exposure to a 30 minute autoclave cycle
(121° C.).
Benzyl Alcohol and Polysorbate-80, 6.5% (w/v)
Fibroin

| Final % (v/v) | Final % (v/v) | | |
|---|---|---|---|
| BA | T-80 | 25° C. | Autoclave |
| 1 | 0.2 | None | N/A |
| 1 | 0 | None | N/A |
| 0.9 | 0.4 | N/A | Partial |
| 0.9 | 0.2 | N/A | Partial |

“Partial” = gelation not uniform, still mostly liquid;
“None” = no gelation, still liquid;
“N/A” = not tested.

Tables 68-71 look at a variety of different polysorbate-80 (Tween-80) and benzyl alcohol combinations. In general, with autoclaving, Tween-80 concentrations ranging from 0.01%-0.4% (v/v) were deemed effective at inducing gelation.

### Example 8.7 Hydrogels with Poloxamer-188 and Poloxamer-407

TABLE 72
Gelation of hydrogels with varying concentrations of poloxamer-188.
Gel formulation at 25 and 37° C., as well as after exposure to a 30 minute
autoclave cycle (121° C.).
Poloxamer-188

| Final % (w/v) | Final % (w/v) | | | |
|---|---|---|---|---|
| Gelling Agent | Fibroin | 25° C. | 37° C. | Autoclave |
| 10 | 13 | None | Full | N/A |
| 10 | 6.5 | None | Full | N/A |
| 10 | 3.5 | None | Full | N/A |
| 5 | 6.5 | N/A | None | Full |
| 2.5 | 6.5 | N/A | None | Full |
| 1 | 6.5 | N/A | None | Full |
| 0.5 | 6.5 | N/A | N/A | Full |
| 0.25 | 6.5 | N/A | N/A | Full |

“Full” = fully formed, injectable hydrogel,
“None” = no gelation, still liquid;
“N/A” = not tested

TABLE 73
Gelation of hydrogels with 6.5% (w/v) fibroin, varying
concentrations of poloxamer-188 and 87 mg/mL
CXB in 0.1% Tween-80. Gel formulation at 37° C.
Poloxamer-188, 6.5% (w/v) Fibroin, 87 mg/mL
CXB

| Final % (w/v) Gelling | 37° C. |
|---|---|
| 10 | Full |
| 5 | None |

“Full” = fully formed, injectable hydrogel;
“None” = no gelation, still liquid

TABLE 74
Gelation of hydrogels with varying concentrations of poloxamer-188.
Gel formulation at 25 and 37° C., as well as after exposure to a 30 minute
autoclave cycle (121° C.).
Poloxamer-407

| Final % (w/v) | Final % (w/v) | | | |
|---|---|---|---|---|
| Gelling Agent | Fibroin | 25° C. | 37° C. | Autoclave |
| 10 | 13 | None | None | N/A |
| 10 | 6.5 | None | Full | N/A |
| 10 | 3.5 | None | Full | N/A |
| 5 | 6.5 | N/A | None | Full |
| 2.5 | 6.5 | N/A | None | Full |
| 1 | 6.5 | N/A | None | Full |
| 0.5 | 6.5 | N/A | N/A | None |
| 0.25 | 6.5 | N/A | N/A | None |

“Full” = fully formed, injectable hydrogel;
“None” = no gelation, still liquid;
“N/A” = not tested

TABLE 75
Gelation of hydrogels with 6.5% (w/v) fibroin, varying concentrations
of poloxamer-407, and 87 mg/mL CXB in 0.1% Tween-80.
Gel formulation at 37° C.
Poloxamer-407, 6.5% (w/v) Fibroin 87 mg/mL
CXB

| Final % (w/v) Gelling | 37° C. |
|---|---|
| 10 | None |
| 5 | None |

“None” = no gelation still liquid

Tables 72-75 detail the gelation results using Poloxamer 188 and Poloxamer 407. These triblock polymers differ in properties based on their molecular weight and proportion of hydrophobic polypropylene oxide (PPO) and hydrophilic polyethylene oxide (PEO) blocks. In the absence of celecoxib and at a 6.5% (w/v) fibroin concentration, hydrogels could not be formed with either Poloxamer 188 or 407 at RT using Poloxamer 188 or 407 concentrations as high as 10% (w/v). Increasing the curing temperature to 37° C. allowed formulations with fibroin concentrations ranging from 13%-3.5% (w/v) to form hydrogels with Poloxamer 188 concentrations of 10% (w/v). Poloxamer 407 induced gelation at a concentration of 10% (w/v) when the formulations contained fibroin concentrations of 6.5% and 3.5% (w/v). Autoclaving reduced the required gelation Poloxamer 188 or Poloxamer 407 concentration to 0.25% (w/v) and 1% (w/v), respectively. Poloxamer 188 at a concentration of 10% (w/v) and a curing temperature of 37° C. allowed for the formation of a hydrogel with a final celecoxib concentration of 87 mg/mL celecoxib.

### Example 8.8 Hydrogels with Hydroxyethul Cellulose (HEC), Carboxymethyl Cellulose (CMC), Hydroxypropyl-β-cyclodextrin (HPBCD), Polyvinylpyrrolidone-12 (PVP-12), and -17 (PVP-17)

TABLE 76
Gelation of hydrogels with 6.5% (w/v) fibroin and varying
concentrations of hydroxyethyl cellulose. Gel formation at 37° C.
Hydroxyethyl cellulose, 6.5% (w/v)
Fibroin

| Final % (w/v) Gelling | |
|---|---|
| Agent | 37° C. |
| 5 | None |
| 2.5 | None |
| 1 | None |

“None” = no gelation, still liquid

TABLE 77
Gelation of hydrogels with 6.5% (w/v) fibroin and varying
concentrations of carboxymethyl cellulose. Gel formation at 37° C.
Carboxymethyl cellulose, 6.5% (w/v)
Fibroin

| Final % (w/v) Gelling | |
|---|---|
| Agent | 37° C. |
| 5 | None |
| 2.5 | None |
| 1 | None |

“None” = no gelation, still liquid

TABLE 78
Gelation of hydrogels with 6.5% (w/v) fibroin and varying
concentrations of hydroxypropyl-β-cyclodextrin. Gel formation
at 37° C.
Hydroxypropyl-β-cyclodextrin, 6.5% (w/v) Fibroin

| Final % (w/v) Gelling | |
|---|---|
| Agent | 37° C. |
| 30 | None |
| 20 | None |
| 10 | None |

“None” = no gelation, still liquid

TABLE 79
Gelation of hydrogels with 6.5% (w/v) fibroin and varying
concentrations of polyvinylpyrrolidone-12.
Gel formation at 37° C.
Polyvinylpyrrolidone-12, 6.5% (w/v) Fibroin

| Final % (w/v) Gelling | |
|---|---|
| Agent | 37° C. |
| 25 | None |
| 10 | None |
| 5 | None |

“None” = no gelation, still liquid

TABLE 80
Gelation of hydrogels with 6.5% (w/v) fibroin and varying
concentrations of polyvinylpyrrolidone-17.
Gel formation at 37° C.
Polyvinylpyrrolidone-17, 6.5% (w/v) Fibroin

| Final % (w/v) Gelling | |
|---|---|
| Agent | 37° C. |
| 25 | None |
| 10 | None |
| 5 | None |

“None” = no gelation, still liquid

Tables 76-80 detail the gelation results using HEC, CMC, HPBCD, PVP-12, and PVP-17 as gelling agents.

The results are shown in FIG. 22. All formulations contained a final 6.5% (w/v) fibroin and 100 mg/mL celecoxib. Under these accelerated, sink conditions of in vitro release, all formulations displayed similar release kinetics with the exception of sorbitol which trended toward a slightly higher initial burst (30% vs. 25%) and shorter duration (28 days vs. 42 days).

### Example 9. Degumming Method Effects on Human Whole Blood Activation Studies

Acute inflammatory responses are initiated via activation of a mammal's innate immune system. Human whole blood contains the principle repertoire of immune cells responsible for initiating an innate driven inflammatory response. Activation of inflammation in human whole blood is a useful in vitro assay for assessing the inflammatory potential of silk-fibroin hydrogel constituents. For this reason, whole human blood was exposed to silk fibroin solutions, three dimensional silk fibroin hydrogels, and a variety of different gelling agents for 24 hours at 37° C. and assessed for activation of inflammation.

Lipopolysaccharide (LPS) was used as a positive stimulator of the inflammatory marker TNF-α, in whole blood. Experiments to assess the inflammatory potential of formulation constituents were conducted both in the absence and the presence of lipopolysaccharide (LPS). The later condition to assess whether any formulation constituent had the activity of potentiating a known inflammatory signal. In each experiment, plasma was collected at the end of the incubation period and analyzed for TNF-α via enzyme-linked immunosorbent assay (ELISA).

In the absence of co-added LPS, soluble silk fibroin samples isolated using several different degumming techniques did not exhibit inflammatory signals beyond low baseline responses. In the presence of LPS, the TNF-α responses to silk fibroin prepared via variety of degumming methods were generally reduced compared to the response to LPS alone. Gelling agents PEG300, PEG400, 8 kDa PEG, glycerol, and sorbitol at relevant concentrations were similarly non-inflammatory +/−LPS, with some specific cases in which the gelling agent reduced the levels of LPS-induced TNF-α.

Finally, experiments were conducted to assess the affect of the three dimensional hydrogel structure on activation of the innate immune system. These experiments demonstrated that the three dimensional hydrogel structure had no effect on the LPS-induced TNF-α response irrespective of the original degumming method used to prepare the silk-fibroin hydrogels.

Additional experiments to assess cell viability in the whole blood experiments demonstrated that the conclusions based on TNF-α response would be unlikely to have been influenced by differential effects on cell viability. In sum, these data support the hypothesis that silk fibroin hydrogels and their constituents will have a low potential for inducing an acute inflammatory response in humans beyond an expected transient foreign body reaction.

### Example 10. Pharmacokinetics of Celecoxib Loaded Silk-Fibroin Hydrogels after Intraarticular Injection

### Methods

Celecoxib loaded silk-fibroin hydrogel formulations were prepared as previously described. Before dosing, animals were randomized into treatment groups. Rats will be dosed intra-articularly (I.A.), under isoflurane anesthesia, into the right knee joint on Day 1. At each timepoint (3, 7, 14, 28, 56, and 84 days) blood was collected from 4 animals per group and processed to serum using standard procedure. Serum was analyzed via LC/MS/MS to determine celecoxib concentrations.

TABLE 81
Group Designations from RAT PK Study

| | | | | Conc Silk (% w/v): |
|---|---|---|---|---|
| | | | Dose | Celecoxib |
| Group | Treatment | Route | Vol | (mg/mL) |
| 1 | 100 mg/mL celecoxib in | IA | 50 μL | 6.5%: 100 |
| | 6.5% (w/v) silk fibroin | | | |
| | hydrogel | | | |
| 2 | 100 mg/mL celecoxib in | IA | 50 μL | 3.5%: 100 |
| | 3.5% (w/v) silk fibroin | | | |
| | hydrogel | | | |

### Results

TABLE 82
Extrapolated AUC data of no observed adverse effect level
(NOAEL) oral dose from oral Celecoxib NDA package

| Dose | AUC at Day 1 | Extrapolated AUC for |
|---|---|---|
| (mg/kg) | (μg hr/mL) | 25 days (μg hr/mL) |
| 20-Male (M) | 18.7 | 467.5 |
| 20-Female (F) | 39.1 | 977.5 |

TABLE 83
AUC of IA silk-fibroin formulations and comparison to oral dosing

| Hydrogel | AUC for days 3-28 (25 days) | Fold less |
|---|---|---|
| Formulationc | (μg hr/mL) | compared to oral |
| 7.5% (w/v) silk | 0.22 | M: 2100/ F: 4400 |
| fiboin- | | |
| 100 mg/mL CXB | | |
| 3.75% silk fibroin | 0.35 | M: 1300 IF: 2800 |
| 100 mg/mL CXB | | |

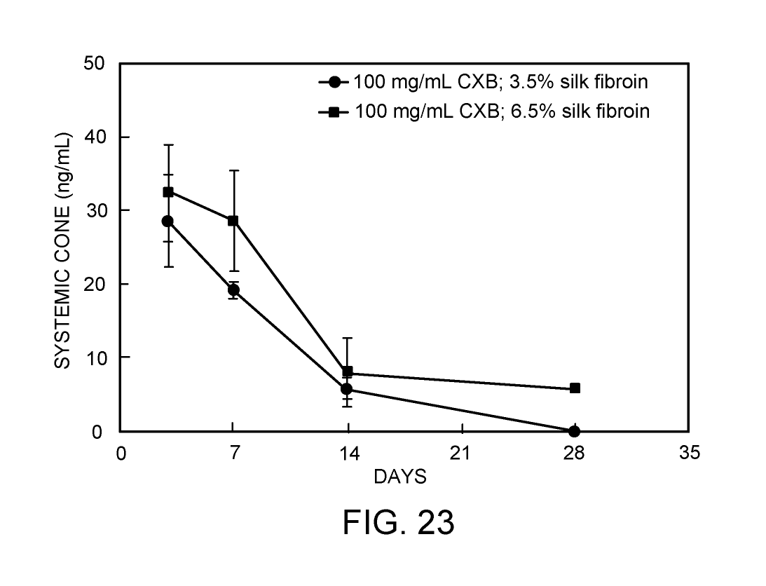

Both silk fibroin/celecoxib formulations exhibited systemic drug concentrations of approximately 30 ng/mL on day 3 with a rapid decrease to <10 ng/ml at day 14, and to levels near or below the LLOQ by week 4 and beyond (FIG. 23). Silk fibroin hydrogels, administered via IA injection, exhibited >1000 fold lower systemic exposure compared to NOAEL oral dosing (Tables 82-83).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-163. (canceled)

164. A composition comprising a benzenesulfonamide-containing non-steroidal anti-inflammatory drug (NSAID), silk fibroin, and a gelling agent.

165. The composition of claim 164, wherein the gelling agent is selected from the group consisting of glycerol (glycerin), polyethylene glycol (PEG), sorbitol, triethylamine, 2-pyrrolidone, alpha-cyclodextrin, benzyl alcohol, beta-cyclodextrin, dimethyl sulfoxide, dimethylacetamide (DMA), dimethylformamide, ethanol, gamma-cyclodextrin, glycerol formal, hydroxypropyl beta-cyclodextrin, kolliphor 124, kolliphor 181, kolliphor 188, kolliphor 407, kolliphor EL (cremaphor EL), cremaphor RH 40, cremophor RH 60, d-alpha-tocopherol, PEG 1000 succinate, polysorbate 20, polysorbate 80, solutol HS 15, sorbitan monooleate, poloxamer-407, poloxamer-188, Labrafil M-1944CS, Lrabfil M-2125CS, Labrasol, Gellucire 44/14, Softigen 767, mono- and di-fatty acid esters of PEG 300, PEG 400, or PEG 1750, kolliphor RH60, N-methyl-2-pyrrolidone, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil, beeswax, d-alpha-tocopherol, oleic acid, medium-chain mono- and di-glycerides, alpha-cyclodextrin, beta-cyclodextrin, hydroxypropyl-beta-cyclodextrin, sulfo-butylether-beta-cyclodextrin, hydrogenated soy phosphatidylcholine, distearoylphosphatidylglycerol, L-alpha-dimyristoylphosphatidylcholine, L-alpha-dimyristoylphosphatidylglycerol, PEG 300, PEG 300 caprylic/capric glycerides (Softigen 767), PEG 300 linoleic glycerides (Labrafil M-2125CS), PEG 300 oleic glycerides (Labrafil M-1944CS), PEG 400, PEG 400 caprylic/capric glycerides (Labrasol), polyoxyl 40 stearate (PEG 1750 monosterate), polyoxyl 8 stearate (PEG 400 monosterate), polysorbate 20, polysorbate 80, polyvinyl pyrrolidone, propylene carbonate, propylene glycol, solutol HS 15, sorbitan monooleate (Span 20), sulfobutylether-beta-cyclodextrin, transcutol, triacetin, 1-dodecylazacyclo-heptan-2-one, caprolactam, castor oil, cottonseed oil, ethyl acetate, medium chain triglycerides, methyl acetate, oleic acid, safflower oil, sesame oil, soybean oil, and tetrahydrofuran.

166. The composition of claim 165, wherein the gelling agent is PEG, and PEG is selected from the group consisting of PEG300, PEG400, or PEG600, or a PEG having a molecular weight of about 2 kDa, about 4 kDa, about 6 kDa, about 8 kDa, about 10 kDa, about 12 kDa, about 14 kDa, about 16 kDa, about 18 kDa, or about 20 kDa.

167. The composition of claim 166, wherein the PEG has a molecular weight of 4 kDa.

168. The composition of claim 164, wherein the composition comprises about 0.01% (w/v) to about 50% (w/v) of the gelling agent, or about 0.01% (v/v) to about 50% (v/v) of the gelling agent.

169. The composition of claim 167, wherein the composition comprises about 6% (w/v) to about 20% (w/v), about 20% (w/v) to about 50% (w/v) of the gelling agent, about 30% (w/v) to about 50% (w/v), or about 35% (w/v) to about 50% (w/v) of the gelling agent, or comprises about 6% (v/v) to about 20% (v/v), about 20% (v/v) to about 50% (v/v), about 30% (v/v) to about 50% (v/v), about 35% (v/v) to about 50% (v/v) of the gelling agent.

170. The composition of claim 164, wherein the composition comprises about 0.5% (w/v) to about 30% (w/v) silk fibroin.

171. The composition of claim 169, wherein the composition comprises about 2% (w/v) to about 30% (w/v) silk fibroin, about 5% (w/v) to about 30% (w/v) silk fibroin, or about 10% (w/v) to about 30% (w/v) silk fibroin.

172. The composition of claim 164, wherein the silk fibroin has a molecular weight range of about 10 kDa to about 500 kDa.

173. The composition of claim 164, wherein the composition comprises a plurality of particles, and wherein the plurality of particles comprise the benzenesulfonamide-containing NSAID.

174. The composition of claim 173, wherein the plurality of particles have a particle size selected from the group consisting of d90 of less than 200 μm, a d50 of less than 25 μm, and a d10 of less than 10 μm.

175. The composition of claim 164, wherein the concentration of benzenesulfonamide-containing NSAID in the composition is selected from the group consisting of about 0.5 mg/mL to about 200 mg/mL, about 1 mg/mL to about 100 mg/mL, about 1 mg/mL to about 50 mg/mL, about 1 mg/mL to about 20 mg/mL, and about 1 mg/mL to about 10 mg/mL.

176. The composition of claim 164, wherein the composition is a gel.

177. The composition of claim 176, wherein the gel is a hydrogel.

178. The composition of claim 164, wherein the composition, when incubated under simulated physiological conditions, has a release selected from the group consisting of (a) at least 15% of the total amount of the benzenesulfonamide-containing NSAID in the composition over a period of about 10 days to about 200 days, (b) at least 15% of the total amount of the benzenesulfonamide-containing NSAID in the composition over a period of about 20 days to about 200 days, (c) at least 15% of the total amount of the benzenesulfonamide-containing NSAID in the composition over a period of about 30 days to about 200 days, (d) at least 15% of the total amount of the benzenesulfonamide-containing NSAID in the composition over a period of about 50 days to about 300 days, and (e) at least 15% of the total amount of the benzenesulfonamide-containing NSAID in the composition over a period of about 100 days to about 300 days.

179. The composition of claim 164, wherein the benzenesulfonamide-containing NSAID comprises a pyrazole moiety.

180. The composition of claim 179, wherein the pyrazole moiety is a haloalkyl-substituted pyrazole moiety.

181. The composition of claim 180, wherein the haloalkyl-substituted pyrazole moiety is a trifluoromethyl-substituted pyrazole moiety.

182. The composition claim 164, wherein the benzenesulfonamide-containing NSAID is celecoxib.

183. A method of treating an inflammatory and/or pain condition in a subject or reducing inflammation, pain and/or fever in a subject, the method comprising administering a therapeutically effective amount of the composition of claim 164 to the subject.

184. The method of claim 183, wherein the inflammatory condition is treated, and the inflammatory condition is selected from the group consisting of: a joint disease, an ophthalmic disease, retinal disease, psoriasis, Crohn's disease, irritable bowel syndrome, Sjogren's disease, tissue graft rejection, asthma, systemic lupus erythematosus, glomerulonephritis, dermatomyositis, multiple sclerosis, scleroderma, vasculitis, Goodpasture's syndrome, atherosclerosis, chronic idiopathic thrombocytopenic purpura, Addison's disease, Parkinson's disease, Alzheimer's disease, diabetes, septic shock, myasthenia gravis, inflammatory pelvic disease, inflammatory bowel disease, urethritis, uveitis, sinusitis, pneumonitis, encephalitis, meningitis, myocarditis, nephritis, osteomyelitis, myositis, hepatitis, gastritis, enteritis, dermatitis, appendicitis, pancreatitis, cholocystitis, polycystic kidney disease, and cancer.

185. The method of claim 184, wherein the inflammatory condition is joint disease, and the joint disease is selected from the group consisting of: osteoarthritis, rheumatoid arthritis, spondyloarthritis, systemic juvenile idiopathic arthritis, psoriatic arthritis, gout, ankylosing spondylitis, and juvenile rheumatoid arthritis.

186. The method of claim 185, wherein the method comprises intraarticularly administering the gel to the joint in the subject.

187. A method of making a silk fibroin composition comprising:
(a) providing a first composition comprising silk fibroin;
(b) providing a second composition comprising about 0.02% (w/v) to about 100% (w/v), or about 0.02% (v/v) to about 100% (v/v), of a gelling agent; and
(c) mixing a volume of the second composition with an amount of the first composition, wherein at least one of the first composition or second composition comprises about 1 mg/mL to about 400 mg/mL of a benzenesulfonamide-containing NSAID; and
(d) incubating the mixture of a time sufficient to form the silk fibroin composition.

188. The method of claim 187, wherein the first composition comprises about 1% (w/v) to about 60% (w/v) silk fibroin.

189. The method of claim 187, wherein each of the first composition and second composition are independently selected from the group consisting of a solid, an aqueous solution and a suspension.

190. The method of claim 189, wherein the ratio of the volume of the first composition to the volume of the second composition is about 5:1 to about 1:5.

191. The method of claim 187, wherein the silk fibroin is generated by incubating a silk raw material in a solution comprising sodium carbonate (Na₂CO₃) for 5 minutes to 300 minutes at about 70° C. to about 100° C.

192. A method of making a composition comprising:
(a) providing a suspension comprising about 1 mg/mL to about 400 mg/mL of a benzenesulfonamide-containing NSAID;
(b) a solution comprising about 0.02% (w/v) to about 100% (w/v), or about 0.02% (v/v) to about 100% (v/v), of a gelling agent, and about 1% (w/v) to about 60% (w/v) silk fibroin;
(c) mixing a volume of the solution with a volume of the suspension, where the ratio of the volume of the solution to the volume of the suspension is about 5:1 to about 1:5 to provide a mixture; and
(d) incubating the mixture of a time sufficient to form the composition.

193. A method of making a composition comprising:
(a) providing a suspension comprising about 1 mg/mL to about 400 mg/mL of a benzenesulfonamide-containing NSAID;
(b) a first solution comprising about 0.02% (w/v) to about 100% (w/v), or about 0.02% (v/v) to about 100% (v/v), of a gelling agent;
(c) a second solution comprising about 1% (w/v) to about 60% (w/v) silk fibroin;
(d) mixing a volume of the first solution, a volume of the second solution, and a volume of the suspension to provide a mixture; and
(e) incubating the mixture of a time sufficient to form the composition.
